SEQUENCE LISTING (116 sequences)

SEQ ID NO: 1 - protein, 372 aa, Homo sapiens
KIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 2 - protein, 377 aa, Homo sapiens
IDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 3 - protein, 332 aa, Homo sapiens
IDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTF

SEQ ID NO: 4 - protein, 356 aa, Homo sapiens
WGAALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPD

SEQ ID NO: 5 - protein, 346 aa, Homo sapiens
IDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNT

SEQ ID NO: 6 - protein, 376 aa, Homo sapiens
ALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHS

SEQ ID NO: 7 - protein, 342 aa, Homo sapiens
KLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVI

SEQ ID NO: 8 - protein, 352 aa, Homo sapiens
HRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIY

SEQ ID NO: 9 - protein, 346 aa, Talpa occidentalis
KIADSSEGGVYLGFYGEQEVAVKRFYEGSTLGQKEVSCLQSCREKSNLVTFYESERQSDCLYVCLTLCEQTLEEHFNARKRADVGNKEDKFARKVLSSIFKAVKGLHQCGYAHQDLQPRNILIDSNDAICLADFDKSIKMTRKQEIKTDLKDLGLLVIYVVKKGDIPFERLKTKSNEDVVRLSPDVETENLIHHLIFPKEEFTDLSGLLGHPFFWSWESRYRALSEVGNENDIKIRKPNSKIFQLLEPKSSEHSKSFNRWKAKIDEYFMKQMDQFYKKDGRYQNTVGDLLRFIRNTRQHLHEKKNEKVKLRIGEPSQYFQEKFPDFFLYVYERLHSTEYGKHFVQD

SEQ ID NO: 10 - protein, 376 aa, Ochotona princeps
KIADTQGGGVYLGLLEDQEVAVKVFVQGSECAGRELSALKSGQDSSRLVTLLACESHEANLYVCMTLCEWTLEEFLEHRPQPAQGDEDKWARQVLHSVFEAVRELHLQRAHAHQDLQPQNILIDAAHGIRLADFDQSVQLAEPTKVSQAVRTDLQALGRLVVYVLLKGDVPFEQLSAMHHRKVWRLCLERETRDLTRCLLTPQQAQEDPLGSLLGHPFFWEWQSRYRTLRNVGNESDIKRCNLRGAIVQLLSLKPSEHSLSFAQWTSKVDSLVLGQMNDFYKNKPHLCYTDSVQDLLRFIRNIGEHIDEDCNREMKARLGDPAIYFLRTFPDLLMHVYKQLQDSEFRRHFPQLPLPAGPGVLETEVPGGQALGADP

SEQ ID NO: 11 - protein, 342 aa, Dromiciops gliroides
KVAETSEGGVYLGFYDGKEAAVKRFREDSKRAQQEMDCILHCQKNSNLVMFYGRESKDNCLYLCFSLCERTLEEHVTTRALENEDFQNIFESLFKAVQELHRHDYLHQDLHPGNILIDSEGKVHLADFDKSEKLNENRHKIKDDLETLGRLVLYVVQLGKDPFFKIQGQSIREVIDACINEEIKDLVQNLLMPGTQYQNLLSKMLSHPFFWNCNIRYRFLRDVGNESDIKRRNYESEILKALNCTDVFAEWTKKIDEHIMKKMNSFYKKKPMYCYKNEVGDLLKFIRNIGEHINEEQNKKMKLKIGEPAQYFQKMFPDLVIYVYKKLRNTVYEKHFPSVNST

SEQ ID NO: 12 - protein, 352 aa, Tachyglossus aculeatus
KIADTSEGGVYLGFFDGLETAVKIFRDDSERAEKEIDCILECRENRHLVKLYGTEINNNCRYVCLSLCERNLEEHLKNHRDADVESTKGILKSLFKAVHELHQFGFVHQDLHPKNILIDSKDEVCLADFDKSCRFANDQTLVHGDLRALGRLVQYVVTKGEDPFVEKGAAREPQELAGLAEIPDHMETLDLIQTLLSPGVTSQTQLSDILYHPFFWPCNMRYRFLRDVGNESDIKTRNTENKTLEKLNRNPSKSYSKWTSKIDKYVMTEMNHFYKKKKNPYQDTVTDLLKFIRNIGEHIDEKVNAQMKETIGDPAQYFQKTFPELVMHVYKNIQNTEFMKHIPNAQNSLLDN

SEQ ID NO: 13 - protein, 332 aa, Spheniscus demersus
RIQNTSQGGIYLGLHGGTEVAVRISRSTEGDKEKRFFEQCGNCEHLLKLFQFEKARGYMYLCFPLWEKNLEEHLQEPEDQMDYKDALRMIFQAVRELHSLGFAHQDLHPTNFFIDLGGKIYLADFDNKRKLIEGKKELVNSDLEALSRLVLYVLTGGRKPLQQVSTKDLTADSPDYEEALDLVTSLVSHDERGLEGLSKHPYFWSKQTRFKFLKGIWNKIKDLHNRKAVFKAANATERFPYPQWTEKIDKYVLKIMENPRKTKPLKYCNDVTDLLRLIRNLDEHPDTRISNRIGDHAEYFLKRFPALTIYVYNSLRQNPKYSHFADIQDPSL

SEQ ID NO: 14 - protein, 357 aa, Python bivittatus
WIQRTTQGGVYLGFYAGEEVAVKIFWKGAENAKREKVCLEKCRTSNHLVKFCGWEERKTCLYLCLSLCERNLQEYFKEEKKKTMESEEILRTVFQAVKELHTFGFGHQELHPSNILIDATGRIFLADFDKSREITDDIKYSIISEDLQGLQKLILYVVMRGQLDFEDLPTECPENVKNHKEIEDLRGRLGLSDQSTPADDLLEKLINHPYFWSKEQKYGVLKKVGNEIRAAKCCNKTSETNILEALKPMDNSFRDWKKKIDKTVLDSMFFLSTEKGQKGNQKGNKRSYGPCITELLKLIRNMGEHYNEKDDKVKDIIRDPADYFLDRFPDLTIYIYQRLCNTEYSIHFPNAKNPSHS

SEQ ID NO: 15 - protein, 360 aa, Microcaecilia unicolor
KIQETSHGGIYLGFYKGKEVAVKRVPKGNNKELVCLEQLRKNNHLVRYCGYEEDSSCSYLCLFLYERNLEEHISNIKDVTEIQDMLQSIFLGLHELHEIGFCHRDLHPSNILIDHEGKAYLADFSGSLKLEDSEDKEGLIKSDMKALGNLVLYVVTRGTACFQPDRERDVTEACPDSVEDFIEARDLVNKLMSPDTEYSSVIDFLNHPFFWSHTSRYKFLQDVGNENDVKMRTEDSALFRTLNDSHVTMTKTFHPWGCKVDQEVLNDMNRFHSKKKIYYQDTVTDLLKFIRNLGAHFDEKKTEIKEIIGDPCQYFLKKFPDLTVYVYETLRHLGKGYIKHFPNPVVLFYNCMQLNNSGMT

SEQ ID NO: 16 - protein, 356 aa, Latimeria chalumnae
KIAESSQGGVYLGFYGDGVEVAVKRMRKNSNEVKMEKLCLEHPKIMGNSCFVKLYGSETDATCHYLCLTLCECTLETYIQEEEGNLKTKVPHIFKQLIGAVQILHEAGFAHTDLHPSNILFNVEGKLQLADFDKCLRFEDCPHKGQSERSDKTEEELKNEDIQAIGNLLCYVTTEGKYVYKTEEEKLGDCYSAEVRDLIQKLSAPQGQPPPLKEALQHPFFWSQESKYRFLQDIGNENEVKVRNDKHEIVKSLNEKAQDPSSSFREWTKKVNDIVFKNMDSRNRKPELKYRNDTASLLKFIRNMGAHFEEKPLEIKNIVKNPCNYFLDLFPDLVISVYNVVKSTQWEEHFPKAETS

SEQ ID NO: 17 - protein, 375 aa, Carcharodon carcharias
KITKDNITEVYLGFYNNVIEVAVKCHRNGSEEAKNEMDCLRDPTINGSSLFVNLLHWESDEFCDYLCLNLYEYNLEECVTMWPKEIREKAPKIMKQLVDAVQVLHGAGFAHRDLHPTNVLRDVKENFRLADFDRSVNCKHPAVTISGKATWEASEILQKLELWPTATFEADELFKADLQALGRLLHYVMTGGKDPCSSENDLCENNLSLNEDLCEPKNAEARDFIERLLAPAEKRATLSEIRQHPLLWKDLGKSQFVQGLANENGVAKRDKSSPIVLILNEVGNNQERSFHKWTEKVHLKILNNMNKRQRTKYEDNTSDLLKFIRNLTQHFDEKEELHSLIKSPESYFCELFPDLTIATYNAVKDTEWVKHFPKE

SEQ ID NO: 18 - protein, 158 aa, Mesocricetus auratus
MASDTPGFYMDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKL

SEQ ID NO: 19 - protein, 174 aa, Mesocricetus auratus
MDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKLPEKSSMRVCRASSGLSKSSSSGHFN

SEQ ID NO: 20 - protein, 197 aa, Mesocricetus auratus
MDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKLPEKSSMRVCRASSGLSKSSSSGHFNSWSTANNFTSLSAPGSDFSETAS

SEQ ID NO: 21 - protein, 234 aa, Mesocricetus auratus
  VARIANT 189: note = X can be any amino acid
MDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKLPEKSSMRVCRASSGLSKSSSSGHFNSWSTANNFTSLSAPXSDFSETASNNHCVFFKSPLMNGLRENKRKPRVKLSSNDVQRNKYT

SEQ ID NO: 22 - protein, 518 aa, Mesocricetus auratus
MDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKLPEKSSMRVCRASSGLSKSSSSGHFNSWSTANNFTSLSAPGSDFSETASNNHCVFFKSPLMNGLRENKRKPRVKLSSNDVQRNKYTTDSRFNEDFEDIEEIGSGGFGQVFKAKHRIDGKTYAIKRVKYNSEKEVREVKALAALSHVNIVQYHFCWLGQDWEYDTEQSMNSATPRSKTECLFIQMEFCDKGTLEQWMTSVKQRKVDNDLFLELAEQITTGVDYIHSKGLIHRDLKPSNIFLVDEKHIKIGDFGLVTALENDGNRTKNTGSLLYMSPEQLSLQEYGKEVDIFALGLILAELLHICITIIEKSKFFTDLRDGIFHDDIFGSKEKRLLTKLLSKKPTDRPSASEILMTLAEWKNISEVQKRHTH

SEQ ID NO: 23 - protein, 517 aa, Mesocricetus auratus
MDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKLPEKSSMRVCRASSGLSKSSSSGHFNSWSTANNFTSLSAPGSDFSETASNNHCVFFKSPLMNGLRENKRKPRVKLSSNDVQRNKYTTDSRFNEDFEDIEEIGSGGFGQVFKAKHRIDGKTYAIRVKYNSEKEVREVKALAALSHVNIVQYHFCWLGQDWEYDTEQSMNSATPRSKTECLFIQMEFCDKGTLEQWMTSVKQRKVDNDLFLELAEQITTGVDYIHSKGLIHRDLKPSNIFLVDEKHIKIGDFGLVTALENDGNRTKNTGSLLYMSPEQLSLQEYGKEVDIFALGLILAELLHICITIIEKSKFFTDLRDGIFHDDIFGSKEKRLLTKLLSKKPTDRPSASEILMTLAEWKNISEVQKRHTH

SEQ ID NO: 24 - protein, 208 aa, Mus musculus
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFT

SEQ ID NO: 25 - protein, 233 aa, Mus musculus
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTNGLGENKRKSGVKVSPDDVQRNKYT

SEQ ID NO: 26 - protein, 515 aa, Mus musculus
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTNGLGENKRKSGVKVSPDDVQRNKYTLDARFNSDFEDIEEIGLGGFGQVFKAKHRIDGKRYAIKRVKYNTEKAEHEVQALAELNHVNIVQYHSCWEGVDYDPEHSMSDTSRYKTRCLFIQMEFCDKGTLEQWMRNRNQSKVDKALILDLYEQIVTGVEYIHSKGLIHRDLKPGNIFLVDERHIKIGDFGLATALENDGKSRTRRTGTLQYMSPEQLFLKHYGKEVDIFALGLILAELLHTCFTESEKIKFFESLRKGDFSNDIFDNKEKSLLKKLLSEKPKDRPETSEILKTLAEWRNISEKKKRNTC

SEQ ID NO: 27 - protein, 325 aa, Danio rerio
MESLSGNYTSLLNEYQQKTQCTVEFEEGPTDGPSHNKRFTMRAIVNGQKFPDGTGKTKKEAKQNAAKNALEGLKSTHSDEPTPSPVENISVSKIASHPNYTCWLNEHSQKSRLMFKACESTKMDPGNLTRLCTYVCKYVCDDKEFPEGYGKNKKEAKEAAALRVYEELNKTPNTEVLDENSNRAQRSETTSYSLDRRSSVADDSRSSTPDNNYIAYLNNYCQKKKRVYDFKLVDRIGPPHNPIFVYKVVMDGKEYPEAQGRNAKEAKQNAAQHAWSEIRDQSGWTTPSSEDGAFSQSQDTCKSEDVQRSANASSTSNFISFRDSA

SEQ ID NO: 28 - protein, 156 aa, Danio rerio
MAKMSAENEIEMRICDYLRRHGRSTVQDIFKELKLEKSTVNRHLYSLQASKQVFKTVEDNKRPVWDLVESMNDEQKAKPEQIKPEMTRDAIEEKHVKDLLKSGGLKASHIASKLGQQTQSTNKLLYGLMEKGEVRKCSKSHMWTLKNDQERNGSGS

SEQ ID NO: 29 - protein, 178 aa, Homo sapiens
MAQAPADPGREGHLEQRILQVLTEAGSPVKLAQLVKECQAPKRELNQVLYRMKKELKVSLTSPATWCLGGTDPEGEGPAELALSSPAERPQQHAATIPETPGPQFSQQREEDIYRFLKDNGPQRALVIAQALGMRTAKDVNRDLYRMKSRHLLDMDEQSKAWTIYRPEDSGRRAKSAS

SEQ ID NO: 30 - protein, 203 aa, Homo sapiens
MAQAPADPGREGHLEQRILQVLTEAGSPVKLAQLVKECQAPKRELNQVLYRMKKELKVSLTSPATWCLGGTDPEGEGPAELALSSPAERPQQHAATIPETPGPQFSQQREEDIYRFLKDNGPQRALVIAQALGMRTAKDVNRDLYRMKSRHLLDMDEQSKAWTIYRPEDSGRRAKSASIIYQHNPINMICQNGPNSWISIANS

SEQ ID NO: 31 - protein, 164 aa, Mus musculus
MAEAPVDLSTGDNLEQKILQVLSDDGGPVKIGQLVKKCQVPKKTLNQVLYRLKKEDRVSSPEPATWSIGGAASGDGAPAIPENSSAQPSLDERILRFLEANGPHRALHIAKALGMTTAKEVNPLLYSMRNKHLLSYDGQTWKIYHSRQEGQDIAHSGVTQESPA

SEQ ID NO: 32 - protein, 189 aa, Mus musculus
MAEAPVDLSTGDNLEQKILQVLSDDGGPVKIGQLVKKCQVPKKTLNQVLYRLKKEDRVSSPEPATWSIGGAASGDGAPAIPENSSAQPSLDERILRFLEANGPHRALHIAKALGMTTAKEVNPLLYSMRNKHLLSYDGQTWKIYHSRQEGQDIAHSGVTQESPAIICQHNPVNMICQQGANSHISIANS

SEQ ID NO: 33 - skipped

SEQ ID NO: 34 - protein, 28 aa, synthetic construct
PEKSSMRVCRASSGLSKSSSSGHFNSGS

SEQ ID NO: 35 - protein, 50 aa, synthetic construct
PEKSSMRVCRASSGLSKSSSSGHFNSWSTANNFTSLSAPGSDFSETASGS

SEQ ID NO: 36 - protein, 87 aa, synthetic construct
PEKSSMRVCRASSGLSKSSSSGHFNSWSTANNFTSLSAPGSDFSETASNNHCVFFKSPLMNGLRENKRKPRVKLSSNDVQRNKYTGS

SEQ ID NO: 37 - protein, 48 aa, synthetic construct
LKSPPKTAGTSSSVVTSTFSGFSSSSSMTSNGVSQSAPGSFSSENVFT

SEQ ID NO: 38 - protein, 73 aa, synthetic construct
LKSPPKTAGTSSSVVTSTFSGFSSSSSMTSNGVSQSAPGSFSSENVFTNGLGENKRKSGVKVSPDDVQRNKYT

SEQ ID NO: 39 - protein, 47 aa, synthetic construct
RDQSGWTTPSSEDGAFSQSQDTCKSEDVQRSANASSTSNFISFRDSA

SEQ ID NO: 40 - protein, 9 aa, synthetic construct
DQERNGSGS

SEQ ID NO: 41 - protein, 12 aa, synthetic construct
PEDSGRRAKSAS

SEQ ID NO: 42 - protein, 37 aa, synthetic construct
PEDSGRRAKSASIIYQHNPINMICQNGPNSWISIANS

SEQ ID NO: 43 - protein, 19 aa, synthetic construct
SRQEGQDIAHSGVTQESPA

SEQ ID NO: 44 - protein, 44 aa, synthetic construct
SRQEGQDIAHSGVTQESPAIICQHNPVNMICQQGANSHISIANS

SEQ ID NO: 45 - protein, 527 aa, synthetic construct
MASDTPGFYMDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKLGSSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 46 - protein, 544 aa, synthetic construct
MDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKLPEKSSMRVCRASSGLSKSSSSGHFNSGSSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 47 - protein, 566 aa, synthetic construct
MDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKLPEKSSMRVCRASSGLSKSSSSGHFNSWSTANNFTSLSAPGSDFSETASGSSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 48 - protein, 603 aa, synthetic construct
  VARIANT 189: note = X can be any amino acid
MDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKLPEKSSMRVCRASSGLSKSSSSGHFNSWSTANNFTSLSAPXSDFSETASNNHCVFFKSPLMNGLRENKRKPRVKLSSNDVQRNKYTGSSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 49 - protein, 887 aa, synthetic construct
MDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKLPEKSSMRVCRASSGLSKSSSSGHFNSWSTANNFTSLSAPGSDFSETASNNHCVFFKSPLMNGLRENKRKPRVKLSSNDVQRNKYTTDSRFNEDFEDIEEIGSGGFGQVFKAKHRIDGKTYAIKRVKYNSEKEVREVKALAALSHVNIVQYHFCWLGQDWEYDTEQSMNSATPRSKTECLFIQMEFCDKGTLEQWMTSVKQRKVDNDLFLELAEQITTGVDYIHSKGLIHRDLKPSNIFLVDEKHIKIGDFGLVTALENDGNRTKNTGSLLYMSPEQLSLQEYGKEVDIFALGLILAELLHICITIIEKSKFFTDLRDGIFHDDIFGSKEKRLLTKLLSKKPTDRPSASEILMTLAEWKNISEVQKRHTHGSSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 50 - protein, 887 aa, synthetic construct
MDKLNKYHQKHRVTITYKHLFTTGPPHDRRFTFQVIIDGEEFPEAEGKTKQEAKNAAAKLAVDTLNANKANSHTDALEGSFTGNYIGLVNSYAQKEKLSVNYQQCAFNTQSPQRFCYKCIIGLKTYGIGSGATKQEAKQLAAKEAYQKLPEKSSMRVCRASSGLSKSSSSGHFNSWSTANNFTSLSAPGSDFSETASNNHCVFFKSPLMNGLRENKRKPRVKLSSNDVQRNKYTTDSRFNEDFEDIEEIGSGGFGQVFKAKHRIDGKTYAIRRVKYNSEKEVREVKALAALSHVNIVQYHFCWLGQDWEYDTEQSMNSATPRSKTECLFIQMEFCDKGTLEQWMTSVKQRKVDNDLFLELAEQITTGVDYIHSKGLIHRDLKPSNIFLVDEKHIKIGDFGLVTALENDGNRTKNTGSLLYMSPEQLSLQEYGKEVDIFALGLILAELLHICITIIEKSKFFTDLRDGIFHDDIFGSKEKRLLTKLLSKKPTDRPSASEILMTLAEWKNISEVQKRHTHGSSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 51 - protein, 580 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 52 - protein, 605 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTNGLGENKRKSGVKVSPDDVQRNKYTKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 53 - protein, 887 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTNGLGENKRKSGVKVSPDDVQRNKYTLDARFNSDFEDIEEIGLGGFGQVFKAKHRIDGKRYAIKRVKYNTEKAEHEVQALAELNHVNIVQYHSCWEGVDYDPEHSMSDTSRYKTRCLFIQMEFCDKGTLEQWMRNRNQSKVDKALILDLYEQIVTGVEYIHSKGLIHRDLKPGNIFLVDERHIKIGDFGLATALENDGKSRTRRTGTLQYMSPEQLFLKHYGKEVDIFALGLILAELLHTCFTESEKIKFFESLRKGDFSNDIFDNKEKSLLKKLLSEKPKDRPETSEILKTLAEWRNISEKKKRNTCKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 54 - protein, 568 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTKIQETSHGGIYLGFYKGKEVAVKRVPKGNNKELVCLEQLRKNNHLVRYCGYEEDSSCSYLCLFLYERNLEEHISNIKDVTEIQDMLQSIFLGLHELHEIGFCHRDLHPSNILIDHEGKAYLADFSGSLKLEDSEDKEGLIKSDMKALGNLVLYVVTRGTACFQPDRERDVTEACPDSVEDFIEARDLVNKLMSPDTEYSSVIDFLNHPFFWSHTSRYKFLQDVGNENDVKMRTEDSALFRTLNDSHVTMTKTFHPWGCKVDQEVLNDMNRFHSKKKIYYQDTVTDLLKFIRNLGAHFDEKKTEIKEIIGDPCQYFLKKFPDLTVYVYETLRHLGKGYIKHFPNPVVLFYNCMQLNNSGMT

SEQ ID NO: 55 - protein, 540 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTRIQNTSQGGIYLGLHGGTEVAVRISRSTEGDKEKRFFEQCGNCEHLLKLFQFEKARGYMYLCFPLWEKNLEEHLQEPEDQMDYKDALRMIFQAVRELHSLGFAHQDLHPTNFFIDLGGKIYLADFDNKRKLIEGKKELVNSDLEALSRLVLYVLTGGRKPLQQVSTKDLTADSPDYEEALDLVTSLVSHDERGLEGLSKHPYFWSKQTRFKFLKGIWNKIKDLHNRKAVFKAANATERFPYPQWTEKIDKYVLKIMENPRKTKPLKYCNDVTDLLRLIRNLDEHPDTRISNRIGDHAEYFLKRFPALTIYVYNSLRQNPKYSHFADIQDPSL

SEQ ID NO: 56 - protein, 583 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTKITKDNITEVYLGFYNNVIEVAVKCHRNGSEEAKNEMDCLRDPTINGSSLFVNLLHWESDEFCDYLCLNLYEYNLEECVTMWPKEIREKAPKIMKQLVDAVQVLHGAGFAHRDLHPTNVLRDVKENFRLADFDRSVNCKHPAVTISGKATWEASEILQKLELWPTATFEADELFKADLQALGRLLHYVMTGGKDPCSSENDLCENNLSLNEDLCEPKNAEARDFIERLLAPAEKRATLSEIRQHPLLWKDLGKSQFVQGLANENGVAKRDKSSPIVLILNEVGNNQERSFHKWTEKVHLKILNNMNKRQRTKYEDNTSDLLKFIRNLTQHFDEKEELHSLIKSPESYFCELFPDLTIATYNAVKDTEWVKHFPKE

SEQ ID NO: 57 - protein, 564 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTWIQRTTQGGVYLGFYAGEEVAVKIFWKGAENAKREKVCLEKCRTSNHLVKFCGWEERKTCLYLCLSLCERNLQEYFKEEKKKTMESEEILRTVFQAVKELHTFGFGHQELHPSNILIDATGRIFLADFDKSREITDDIKYSIISEDLQGLQKLILYVVMRGQLDFEDLPTECPENVKNHKEIEDLRGRLGLSDQSTPADDLLEKLINHPYFWSKEQKYGVLKKVGNEIRAAKCCNKTSETNILEALKPMDNSFRDWKKKIDKTVLDSMFFLSTEKGQKGNQKGNKRSYGPCITELLKLIRNMGEHYNEKDDKVKDIIRDPADYFLDRFPDLTIYIYQRLCNTEYSIHFPNANPSHS

SEQ ID NO: 58 - protein, 564 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTKIAESSQGGVYLGFYGDGVEVAVKRMRKNSNEVKMEKLCLEHPKIMGNSCFVKLYGSETDATCHYLCLTLCECTLETYIQEEEGNLKTKVPHIFKQLIGAVQILHEAGFAHTDLHPSNILFNVEGKLQLADFDKCLRFEDCPHKGQSERSDKTEEELKNEDIQAIGNLLCYVTTEGKYVYKTEEEKLGDCYSAEVRDLIQKLSAPQGQPPPLKEALQHPFFWSQESKYRFLQDIGNENEVKVRNDKHEIVKSLNEKAQDPSSSFREWTKKVNDIVFKNMDSRNRKPELKYRNDTASLLKFIRNMGAHFEEKPLEIKNIVKNPCNYFLDLFPDLVISVYNVVKSTQWEEHFPKAETS

SEQ ID NO: 59 - protein, 554 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTKIADSSEGGVYLGFYGEQEVAVKRFYEGSTLGQKEVSCLQSCREKSNLVTFYESERQSDCLYVCLTLCEQTLEEHFNARKRADVGNKEDKFARKVLSSIFKAVKGLHQCGYAHQDLQPRNILIDSNDAICLADFDKSIKMTRKQEIKTDLKDLGLLVIYVVKKGDIPFERLKTKSNEDVVRLSPDVETENLIHHLIFPKEEFTDLSGLLGHPFFWSWESRYRALSEVGNENDIKIRKPNSKIFQLLEPKSSEHSKSFNRWKAKIDEYFMKQMDQFYKKDGRYQNTVGDLLRFIRNTRQHLHEKKNEKVKLRIGEPSQYFQEKFPDFFLYVYERLHSTEYGKHFVQD

SEQ ID NO: 60 - protein, 584 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTKIADTQGGGVYLGLLEDQEVAVKVFVQGSECAGRELSALKSGQDSSRLVTLLACESHEANLYVCMTLCEWTLEEFLEHRPQPAQGDEDKWARQVLHSVFEAVRELHLQRAHAHQDLQPQNILIDAAHGIRLADFDQSVQLAEPTKVSQAVRTDLQALGRLVVYVLLKGDVPFEQLSAMHHRKVWRLCLERETRDLTRCLLTPQQAQEDPLGSLLGHPFFWEWQSRYRTLRNVGNESDIKRCNLRGAIVQLLSLKPSEHSLSFAQWTSKVDSLVLGQMNDFYKNKPHLCYTDSVQDLLRFIRNIGEHIDEDCNREMKARLGDPAIYFLRTFPDLLMHVYKQLQDSEFRRHFPQLPLPAGPGVLETEVPGGQALGADP

SEQ ID NO: 61 - protein, 550 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTKVAETSEGGVYLGFYDGKEAAVKRFREDSKRAQQEMDCILHCQKNSNLVMFYGRESKDNCLYLCFSLCERTLEEHVTTRALENEDFQNIFESLFKAVQELHRHDYLHQDLHPGNILIDSEGKVHLADFDKSEKLNENRHKIKDDLETLGRLVLYVVQLGKDPFFKIQGQSIREVIDACINEEIKDLVQNLLMPGTQYQNLLSKMLSHPFFWNCNIRYRFLRDVGNESDIKRRNYESEILKALNCTDVFAEWTKKIDEHIMKKMNSFYKKKPMYCYKNEVGDLLKFIRNIGEHINEEQNKKMKLKIGEPAQYFQKMFPDLVIYVYKKLRNTVYEKHFPSVNST

SEQ ID NO: 62 - protein, 560 aa, synthetic construct
MASDTPGFYMDKLNKYRQMHGVAITYKELSTSGPPHDRRFTFQVLIDEKEFPEAKGKSKQEARNAAAKLAVDILDNENKVDCHTSASEQGLPYGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTKIADTSEGGVYLGFFDGLETAVKIFRDDSERAEKEIDCILECRENRHLVKLYGTEINNNCRYVCLSLCERNLEEHLKNHRDADVESTKGILKSLFKAVHELHQFGFVHQDLHPKNILIDSKDEVCLADFDKSCRFANDQTLVHGDLRALGRLVQYVVTKGEDPFVEKGAAREPQELAGLAEIPDHMETLDLIQTLLSPGVTSQTQLSDILYHPFFWPCNMRYRFLRDVGNESDIKTRNTENKTLEKLNRNPSKSYSKWTSKIDKYVMTEMNHFYKKKKNPYQDTVTDLLKFIRNIGEHIDEKVNAQMKETIGDPAQYFQKTFPELVMHVYKNIQNTEFMKHIPNAQNSLLDN

SEQ ID NO: 63 - protein, 697 aa, synthetic construct
MESLSGNYTSLLNEYQQKTQCTVEFEEGPTDGPSHNKRFTMRAIVNGQKFPDGTGKTKKEAKQNAAKNALEGLKSTHSDEPTPSPVENISVSKIASHPNYTCWLNEHSQKSRLMFKACESTKMDPGNLTRLCTYVCKYVCDDKEFPEGYGKNKKEAKEAAALRVYEELNKTPNTEVLDENSNRAQRSETTSYSLDRRSSVADDSRSSTPDNNYIAYLNNYCQKKKRVYDFKLVDRIGPPHNPIFVYKVVMDGKEYPEAQGRNAKEAKQNAAQHAWSEIRDQSGWTTPSSEDGAFSQSQDTCKSEDVQRSANASSTSNFISFRDSAKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 64 - protein, 528 aa, synthetic construct
MAKMSAENEIEMRICDYLRRHGRSTVQDIFKELKLEKSTVNRHLYSLQASKQVFKTVEDNKRPVWDLVESMNDEQKAKPEQIKPEMTRDAIEEKHVKDLLKSGGLKASHIASKLGQQTQSTNKLLYGLMEKGEVRKCSKSHMWTLKNDQERNGSGSKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 65 - protein, 550 aa, synthetic construct
MAQAPADPGREGHLEQRILQVLTEAGSPVKLAQLVKECQAPKRELNQVLYRMKKELKVSLTSPATWCLGGTDPEGEGPAELALSSPAERPQQHAATIPETPGPQFSQQREEDIYRFLKDNGPQRALVIAQALGMRTAKDVNRDLYRMKSRHLLDMDEQSKAWTIYRPEDSGRRAKSASKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 66 - protein, 575 aa, synthetic construct
MAQAPADPGREGHLEQRILQVLTEAGSPVKLAQLVKECQAPKRELNQVLYRMKKELKVSLTSPATWCLGGTDPEGEGPAELALSSPAERPQQHAATIPETPGPQFSQQREEDIYRFLKDNGPQRALVIAQALGMRTAKDVNRDLYRMKSRHLLDMDEQSKAWTIYRPEDSGRRAKSASIIYQHNPINMICQNGPNSWISIANSKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 67 - protein, 536 aa, synthetic construct
MAEAPVDLSTGDNLEQKILQVLSDDGGPVKIGQLVKKCQVPKKTLNQVLYRLKKEDRVSSPEPATWSIGGAASGDGAPAIPENSSAQPSLDERILRFLEANGPHRALHIAKALGMTTAKEVNPLLYSMRNKHLLSYDGQTWKIYHSRQEGQDIAHSGVTQESPAKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 68 - protein, 561 aa, synthetic construct
MAEAPVDLSTGDNLEQKILQVLSDDGGPVKIGQLVKKCQVPKKTLNQVLYRLKKEDRVSSPEPATWSIGGAASGDGAPAIPENSSAQPSLDERILRFLEANGPHRALHIAKALGMTTAKEVNPLLYSMRNKHLLSYDGQTWKIYHSRQEGQDIAHSGVTQESPAIICQHNPVNMICQQGANSHISIANSKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 69 - DNA, 1581 bp, synthetic construct
  source 1..1581: mol_type = other DNA
atggccagtgatactcccggtttctacatggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 70 - DNA, 1632 bp, synthetic construct
  source 1..1632: mol_type = other DNA
atggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaccagagaaaagctcaatgagagtttgcagagcatcctctggtcttagcaaatcttcatccagtggccacttcaacagcggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 71 - DNA, 1698 bp, synthetic construct
  source 1..1698: mol_type = other DNA
atggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaccagagaaaagctcaatgagagtttgcagagcatcctctggtcttagcaaatcttcatccagtggccacttcaacagctggtccacagcaaacaattttacttctctgtcagcgcctggaagtgatttctcagagactgcatcaggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 72 - DNA, 1809 bp, synthetic construct
  source 1..1809: mol_type = other DNA
atggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaccagagaaaagctcaatgagagtttgcagagcatcctctggtcttagcaaatcttcatccagtggccacttcaacagctggtccacagcaaacaattttacttctctgtcagcgcctgnaagtgatttctcagagactgcatcaaacaaccactgtgtttttttcaagtctcctctcatgaatgggctcagagaaaataaaaggaagccacgagtaaaactgtcatctaatgatgtgcaaagaaataaatataccggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 73 - DNA, 2661 bp, synthetic construct
  source 1..2661: mol_type = other DNA
atggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaccagagaaaagctcaatgagagtttgcagagcatcctctggtcttagcaaatcttcatccagtggccacttcaacagctggtccacagcaaacaattttacttctctgtcagcgcctggaagtgatttctcagagactgcatcaaacaaccactgtgtttttttcaagtctcctctcatgaatgggctcagagaaaataaaaggaagccacgagtaaaactgtcatctaatgatgtgcaaagaaataaatataccacggactccaggtttaacgaggattttgaagacatagaagaaattggctcaggtggatttggccaagttttcaaagcaaaacacagaatcgatgggaagacttacgctattaagcgagttaaatataactcagagaaggaagtgcgtgaagtaaaggcgctggcggcactcagccatgtcaatattgtccagtaccatttttgttggctgggacaggactgggagtacgatacagagcagagcatgaacagtgccaccccacgatcaaagacagagtgccttttcattcaaatggaattctgtgataaagggactctggagcaatggatgacaagtgtcaagcagaggaaagtagacaatgatttgtttttggaattagctgaacaaataacaacaggggtggattatatacactcaaaaggcttaattcacagagaccttaagccaagtaatatatttttagtggatgaaaaacacataaagattggagacttcggcctcgtaacagccctggaaaatgatggaaatcgaacaaagaatacgggatctcttctatacatgagcccagaacagttatctttacaggaatatggaaaagaagtggacatctttgctctaggacttattctagctgaactccttcacatatgcatcactattatagaaaagtcaaagtttttcacggacctaagagatggcatcttccatgacgacatatttggcagcaaggaaaaaaggcttctaacgaaattactctcgaagaaacccacggatcgacctagtgcttctgaaattctgatgaccttggctgaatggaagaatatctcagaagtacagaaacgacacacacatggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 74 - DNA, 2661 bp, synthetic construct
  source 1..2661: mol_type = other DNA
atggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaccagagaaaagctcaatgagagtttgcagagcatcctctggtcttagcaaatcttcatccagtggccacttcaacagctggtccacagcaaacaattttacttctctgtcagcgcctggaagtgatttctcagagactgcatcaaacaaccactgtgtttttttcaagtctcctctcatgaatgggctcagagaaaataaaaggaagccacgagtaaaactgtcatctaatgatgtgcaaagaaataaatataccacggactccaggtttaacgaggattttgaagacatagaagaaattggctcaggtggatttggccaagttttcaaagcaaaacacagaatcgatgggaagacttacgctattaggcgagttaaatataactcagagaaggaagtgcgtgaagtaaaggcgctggcggcactcagccatgtcaatattgtccagtaccatttttgttggctgggacaggactgggagtacgatacagagcagagcatgaacagtgccaccccacgatcaaagacagagtgccttttcattcaaatggaattctgtgataaagggactctggagcaatggatgacaagtgtcaagcagaggaaagtagacaatgatttgtttttggaattagctgaacaaataacaacaggggtggattatatacactcaaaaggcttaattcacagagaccttaagccaagtaatatatttttagtggatgaaaaacacataaagattggagacttcggcctcgtaacagccctggaaaatgatggaaatcgaacaaagaatacgggatctcttctatacatgagcccagaacagttatctttacaggaatatggaaaagaagtggacatctttgctctaggacttattctagctgaactccttcacatatgcatcactattatagaaaagtcaaagtttttcacggacctaagagatggcatcttccatgacgacatatttggcagcaaggaaaaaaggcttctaacgaaattactctcgaagaaacccacggatcgacctagtgcttctgaaattctgatgaccttggctgaatggaagaatatctcagaagtacagaaacgacacacacatggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 75 - DNA, 1740 bp, synthetic construct
  source 1..1740: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 76 - DNA, 1815 bp, synthetic construct
  source 1..1815: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaacggtctcggagaaaataaaaggaaatcaggagtaaaagtatcccctgatgatgtgcaaagaaataaatataccaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 77 - DNA, 2661 bp, synthetic construct
  source 1..2661: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaacggtctcggagaaaataaaaggaaatcaggagtaaaagtatcccctgatgatgtgcaaagaaataaatataccttggacgccaggtttaacagcgattttgaagacatagaagaaattggcttaggtggatttggtcaagttttcaaagcgaaacacagaattgatggaaagagatacgctattaagcgcgttaaatataacacggagaaggcggagcacgaagtacaagcgctggcagaactcaatcacgtcaacattgtccaataccatagttgttgggagggagttgactatgatcctgagcacagcatgagtgatacaagtcgatacaaaacccggtgcctctttattcaaatggaattctgtgataaaggaactttggagcaatggatgagaaacagaaatcagagtaaagtggacaaagctttgattttggacttatatgaacaaatcgtgaccggagtggagtatatacactcgaaagggttaattcacagagatcttaagccaggtaatatatttttagtagatgaaagacacattaagatcggagactttggccttgcaacagccctggaaaatgatggaaaatcccgaacaaggagaacaggaactcttcaatatatgagtccagaacagttatttttaaagcactatggaaaagaagtggacatctttgctttgggccttattctagctgaacttcttcacacgtgcttcacggagtcagagaaaataaagtttttcgaaagtctaagaaaaggcgacttctctaatgatatattcgacaacaaagaaaaaagccttctaaaaaaactactctcagagaaacccaaggaccgacctgagacatctgaaatcctgaagaccttggctgaatggaggaacatctcagagaaaaagaaaagaaacacatgtaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 78 - DNA, 1704 bp, synthetic construct
  source 1..1704: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaaaattcaagaaacctcccatggtggcatctacctaggtttctataaagggaaagaagtagctgtgaagagggttccaaaaggtaataataaagagctggtctgtctggagcaattacggaagaataatcatttagtacgttactgtggatatgaagaagatagctcatgttcatatctatgtctttttctgtatgagagaaaccttgaagaacacattagtaatattaaagatgtaactgagatccaagacatgcttcagtctatattcctgggcttgcatgagctacatgagataggattttgccatcgggaccttcatccatccaatattttaattgatcatgagggtaaagcttacctggcagatttcagcgggagtctgaagttggaagattctgaagacaaggaagggttgatcaagagtgacatgaaggcattgggaaaccttgtcttgtatgtagtaacaagaggaacggcctgcttccaaccagatagagaaagggatgtgacggaagcttgtccagattcagtggaagattttatagaagcaagggatctagtaaataaattaatgtctccagatacagagtattcatctgtgattgatttccttaaccatcccttcttctggagtcacacaagcaggtataagtttcttcaagatgttgggaatgagaatgatgtcaaaatgcgcacagaggacagtgcccttttcagaaccctgaatgattctcatgtgacaatgacaaaaaccttccacccctggggttgtaaggttgaccaggaggtcctgaatgacatgaacaggttccacagtaagaagaaaatctattatcaggacacagtcactgacctgttgaagttcattagaaacttgggtgctcacttcgatgagaaaaaaacagaaataaaggagatcattggagatccctgccagtattttctcaagaagtttccagatctcacggtttatgtctatgaaactctgcgacatttggggaagggatacattaagcattttccaaaccccgtagttctcttctataattgcatgcagctcaacaattcgggtatgact

SEQ ID NO: 79 - DNA, 1620 bp, synthetic construct
  source 1..1620: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgagaattcagaacacttcccagggtggcatctacttggggctccacggtgggacggaggtagcagtaagaataagccgcagtacagagggtgacaaagagaaaaggttcttcgaacaatgtggtaactgtgaacatctactgaagctcttccagtttgagaaggcaagaggctacatgtacttgtgcttccccctctgggagaaaaaccttgaagaacatctgcaggaaccagaagaccaaatggattacaaagatgctctgaggatgatcttccaggcagtgagagagctgcactcccttggatttgctcaccaggatctgcatcccaccaactttttcatagatttaggtggcaaaatttacttggcagactttgataataaaagaaagttgattgaaggcaaaaaagaactcgtaaactcagatttagaggccctcagcaggctcgtgctgtatgtcttaacagggggtaggaaaccccttcagcaagtcagtaccaaggatttgactgctgattccccagattatgaggaggctctggaccttgtaactagcctggtctctcatgatgaacgaggcttggaaggtttgagcaaacatccctatttctggagcaaacagaccaggttcaagttcctgaagggtatatggaataaaatcaaagatctccacaaccgaaaagctgtctttaaagctgctaatgctactgaacgttttccttatccacagtggaccgagaagattgacaaatatgttctgaaaatcatggaaaatcctaggaaaacaaaaccactcaaatattgcaatgatgtcaccgacctactgaggctcatcagaaacctggatgaacacccagataccaggatcagcaacagaataggagaccatgctgagtatttcttgaagcgctttccagcacttaccatttatgtctacaacagtttacgtcagaatcccaaatacagtcactttgcagacattcaggacccttctctg

SEQ ID NO: 80 - DNA, 1749 bp, synthetic construct
  source 1..1749: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaagattacgaaagacaacataacggaagtctaccttggtttctataacaatgtgattgaagtggcagtgaaatgtcacaggaacggcagtgaagaagccaaaaatgaaatggattgcctcagagatcctacgatcaatggtagcagcctgtttgttaacctcctgcattgggaaagtgatgagttttgcgattacttatgcctgaatctctatgagtataatctcgaggaatgtgtgaccatgtggcctaaggaaattcgagagaaggctcctaaaataatgaagcagttggttgacgctgttcaggttttgcatggagctggatttgctcaccgggatttacatccaacaaatgtcctcagagatgttaaagaaaattttcgcctggcagactttgataggagtgtgaattgtaagcatccagctgtgaccatatccggcaaagcgacctgggaggcttccgaaatccttcagaaactggaactctggccgacagcaaccttcgaagcggatgagcttttcaaagcggatttacaggctcttggacggcttttgcattatgttatgaccggagggaaggatccctgtagcagtgaaaatgatctgtgcgagaataatctttctctcaatgaggatctttgcgaacccaagaatgccgaagccagagatttcatcgagcgcttgttggcgccagcagagaaaagagctacattaagtgagatcagacaacatccgctgctgtggaaagatttgggaaagagccagtttgtccaaggccttgcaaatgaaaacggtgtggcaaaacgggataaatccagtccgatagtcctgattctgaatgaggttggcaacaaccaggaaagatcatttcacaagtggacagagaaggtccaccttaaaatcctaaataatatgaataagagacagcgcacaaaatatgaggacaacaccagtgacctcctgaaattcataagaaacctgacccagcattttgatgaaaaggaagaattacacagtttgattaaaagtcccgaatcttatttctgtgaattatttcctgatctcaccatcgctacctacaatgctgtgaaggacaccgagtgggtaaagcactttccaaaagaa

SEQ ID NO: 81 - DNA, 1695 bp, synthetic construct
  source 1..1695: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgtggatccagaggactacccaaggaggggtctacctaggattctatgctggagaagaggtggctgtgaagatattctggaaaggtgcagaaaatgccaagcgagagaaagtgtgccttgagaaatgtcgaaccagcaaccacctggtgaagttttgcggctgggaagagaggaagacctgtctctacctgtgcctctccttgtgtgagaggaatcttcaagagtacttcaaggaagaaaagaaaaaaactatggaaagcgaagagattcttcgaacagtatttcaggccgtaaaggaacttcacacatttggatttggccatcaggaactgcacccaagtaacattttgatagatgccactggcaggattttcctagccgactttgataagagcagagaaataactgatgatataaaatattcaataatctcagaagacctacagggcctccaaaaacttatcctgtatgttgtgatgagaggccagttggactttgaagatttgcctacagaatgcccagaaaatgtaaagaaccataaggaaattgaagacctccgaggaaggctgggactctctgaccaaagtaccccagccgatgacctactggaaaagctgatcaatcatccatatttttggtccaaggagcaaaagtacggagtgttaaaaaaggtcgggaatgagatacgtgccgctaagtgctgtaataaaaccagtgaaacaaatattctggaagctttgaaacctatggataattctttcagggactggaaaaaaaagattgataaaacagtactggactctatgttttttctgtccactgagaagggacagaaggggaatcagaagggcaacaaacgaagttatggcccctgcataacagagttactaaagttgattaggaatatgggtgagcactacaatgagaaggatgacaaggtgaaagacataattcgggacccggcagactattttctggatcgctttccagatttaaccatttatatctatcagcgtttgtgtaatacggaatacagtatacattttccaaatgctaagaatccttctcactca

SEQ ID NO: 82 - DNA, 1692 bp, synthetic construct
  source 1..1692: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaagattgctgagagctctcaagggggtgtctacctgggcttctatggggatggagttgaggttgcagtgaaacgcatgagaaagaacagtaatgaagtgaaaatggaaaagctgtgtcttgaacacccaaagattatgggcaacagctgttttgttaaactctatggcagtgaaacggacgctacatgtcattatttgtgcttgaccctctgtgaatgcactctagagacatacatacaagaggaagaaggaaatctaaagactaaagtgcctcacatatttaagcagctaatcggagcagtgcagatattacatgaagctggatttgcacacacagaccttcatccttcaaatatcctatttaatgtagaagggaagcttcaactggctgattttgataagtgtttaaggtttgaagactgtccacataaaggccaatcagaaagaagtgataaaactgaagaggagctgaaaaatgaggacattcaggcaattggaaatctgctttgttatgtcaccactgaaggaaaatatgtatacaagacagaagaggagaagcttggagactgctatagtgctgaggtgagggatcttatacagaaactttcggccccacagggacaaccacctccactaaaggaggccctgcaacatccattcttctggagtcaagaaagcaaatacagatttctacaagacattggtaatgagaatgaagtaaaagtcagaaacgacaaacatgaaatagtaaagagtcttaatgaaaaggcacaagacccttcttcatcattccgtgaatggaccaaaaaggtaaatgacattgtttttaaaaacatggatagtcgtaataggaaaccagaactcaagtataggaatgacactgcaagcctcttgaagtttatacgaaatatgggagcccattttgaggagaagccactagaaattaagaatattgtcaagaatccgtgcaactacttccttgacttgtttccagatcttgttatttctgtctacaatgttgtgaaatcaactcaatgggaagaacattttccaaaagctgaaacttct

SEQ ID NO: 83 - DNA, 1662 bp, synthetic construct
  source 1..1662: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaaaattgcagacagttctgaagggggtgtgtacttggggttctatggagaacaggaggtagctgtgaagcgattctatgaaggcagtaccctggggcaaaaggaagtctcctgtttgcagagttgccgggagaagagtaacttggtgacattctatgagagtgagaggcaaagtgactgcctgtatgtgtgcctcaccctctgcgagcagacactggaggagcacttcaatgcccgcaaaagggcagacgtgggaaacaaggaagataagtttgcccggaaagtcctctcttctatatttaaagcagtcaaagggctgcaccagtgtggatatgctcaccaagacctgcagccacgaaacatcttaatagattccaatgatgctatttgcctggcagattttgataaaagcatcaagatgactagaaaacaggaaatcaaaacagatctaaaggacctcggactactggtcatatatgttgtaaaaaagggagacatcccttttgaaagactgaagactaaaagtaatgaagacgtggttcgactttctccagatgtggaaactgaaaacctcattcaccatctgatctttcctaaggaagaatttacagacctaagtggcctgctggggcatcccttcttttggagttgggagagtcgctatagggccctgagtgaagtgggaaatgaaaatgacatcaaaattcgaaagcctaatagcaagatctttcaactgctggagcctaaatcttctgaacattccaaaagttttaaccgctggaaggctaagattgatgagtattttatgaaacaaatggatcagttttacaaaaaagacggtaggtatcagaacactgtgggtgatctgctaagattcatccggaatacaagacaacatcttcatgaaaaaaagaatgaaaaggtgaagttgagaattggagaaccttcccaatattttcaggaaaaatttccggatttctttttatatgtctatgaaagactacacagcactgaatatggaaagcattttgtccaagac

SEQ ID NO: 84 - DNA, 1752 bp, synthetic construct
  source 1..1752: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaaaatcgccgacacgcaggggggcggcgtctacctggggctcctcgaggaccaggaggtggccgtgaaggtcttcgttcagggcagtgaatgtgccggcagggagctgtccgccctcaagagtggccaggacagcagccgcctggtgaccctgcttgcctgcgagagccacgaagccaacctgtatgtgtgtatgaccctgtgtgagtggacgctggaggagttcctggagcaccgacctcagcctgcccaaggcgacgaggacaagtgggcccgccaggtcttgcactccgtgtttgaggcagttcgagagctgcacctacagcgtgctcatgcccaccaggacctgcagccacagaacattctcatagacgctgcgcacggcatccgcctggctgactttgatcagagcgtccagctggctgaacccaccaaagtctcacaggccgtcaggacagacctgcaggccctgggccggctggtcgtgtacgtgctgttgaagggggatgttccgtttgagcagctgagtgccatgcatcatcgaaaggtctggcgtctctgtcttgaacgggagaccagggacctcacgcgctgcctgctgaccccccagcaggctcaggaggaccctctgggcagcctgttgggccaccccttcttctgggagtggcagagccgctacaggacgctccggaacgtggggaacgaatctgacatcaaaaggtgcaatctcaggggcgccatcgtgcagctgctgagtctgaagccctctgagcacagcctcagcttcgcccagtggacgagcaaggtggacagcctcgtgctgggacaaatgaacgacttctacaagaacaaacctcacctgtgctacacggacagcgtgcaggacctgctgaggttcatccgcaacattggggagcacattgacgaggactgcaaccgagagatgaaggcccggctcggggaccccgctatctacttcctgcgcacatttccagacctgctgatgcacgtgtacaagcagctgcaggattcggaattccggaggcacttcccacagctccctctgcctgcgggccccggcgtgttggagacggaggtgccagggggccaggcgctgggggctgatccc

SEQ ID NO: 85 - DNA, 1650 bp, synthetic construct
  source 1..1650: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaaggttgcagaaacttctgaaggtggtgtgtacctggggttctatgatgggaaagaggcagccgtgaagcgattccgagaagacagtaaacgggctcagcaagagatggactgcatcctccattgccaaaagaatagcaacttggtgatgttttatgggagagaaagcaaagacaactgtttgtatttgtgcttttctctttgtgagaggactctggaggaacatgtgacaacaagagccttggagaatgaggacttccaaaacatttttgagtccttatttaaggcagtgcaagaactacacagacacgactatctccaccaggacctgcacccagggaatatcttgatagattctgaaggtaaagttcacttggcagattttgataagagtgaaaaattgaatgaaaatcggcataaaattaaggatgatctagagactcttggaaggctggttctatatgtggtacagttgggaaaagacccctttttcaaaattcaaggccaaagtatccgtgaggtcatagatgcttgtataaatgaggaaattaaagatcttgtgcaaaacctgcttatgcctggaacacaataccaaaacctgctaagtaagatgctctctcatcctttcttctggaattgtaatattcgttacagattccttcgagatgtggggaatgaatctgacatcaaaagacgaaattatgagagtgaaatcctcaaggcactaaactgtactgatgtttttgcagagtggacaaaaaagattgatgaacatattatgaaaaaaatgaattcattttataaaaagaagcctatgtactgctacaagaatgaagttggagacttgctaaaattcatccgtaatataggtgaacacattaatgaagaacaaaataaaaaaatgaagttaaaaattggggaacctgctcagtattttcagaagatgtttcctgatctggtgatttatgtttacaagaaactgcggaatacggtctacgagaagcattttcccagtgttaacagtact

SEQ ID NO: 86 - DNA, 1680 bp, synthetic construct
  source 1..1680: mol_type = other DNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaagattgcagacacctctgaaggtggtgtctacttggggttctttgacggactagagacggctgtgaagatattccgtgatgacagtgaaagagccgagaaggaaatcgactgtatcctagagtgccgagagaaccgccatcttgtaaagctctatggaaccgagataaataacaactgtaggtacgtgtgcctctccctctgtgagaggaacttggaggagcacctgaaaaatcacagagatgcagatgtcgaatccaccaaaggcattctcaagtccctgtttaaggcagtgcacgaattacatcagtttggattcgtccatcaggacctgcatccaaagaacatcttgatagattctaaagatgaagtgtgcctggcagattttgataaaagctgcaggtttgctaatgatcagacattagtccatggagacctccgggcacttggacgactggtccagtatgttgtaaccaagggagaggacccctttgtggagaaaggggctgcaagagagcctcaggagctagcgggtctagctgaaatcccggatcacatggagacgctggacctcatccagacactgctctcccctggggtcaccagccagactcagctgagcgacattctctaccatcccttcttctggccctgtaacatgcgctacaggttcctaagggatgttgggaacgaatcagacatcaaaactcggaatactgaaaacaagaccctggaaaaattgaatcgtaatccttctaagagttactcaaaatggacttcaaagattgataaatatgttatgactgagatgaatcatttctacaaaaagaaaaaaaacccctatcaagacacagtgactgacctgctgaaattcatccgcaacataggcgagcacattgatgagaaagtcaatgcacaaatgaaggaaactatcggggatcctgctcagtattttcagaagacctttcctgagctggtgatgcacgtctacaagaacatacagaacacagaattcatgaaacatattccgaatgcccagaattctctcctggataac

SEQ ID NO: 87 - DNA, 2091 bp, synthetic construct
  source 1..2091: mol_type = other DNA
atggagtctctgtcagggaattatacttctctgctgaatgagtatcagcagaaaacccaatgcactgttgagtttgaggagggacccactgatggaccaagccacaataaaaggtttactatgagagctattgtgaatggacagaagtttcctgatggtactggtaaaaccaagaaggaggcaaagcaaaatgctgctaaaaacgcactggaaggcttgaagagcactcacagtgatgaaccgacaccttcacccgttgaaaacatttcagttagtaaaatagcatctcatccgaattacacctgctggctcaatgagcattcgcagaagagcaggttaatgttcaaggcctgcgagtcaactaagatggatccaggaaacctcactcggttatgcacgtatgtgtgtaagtatgtttgcgatgataaagagtttcctgaaggttatgggaaaaataagaaggaagccaaagaggcagcggctcttcgagtttatgaagagctcaacaaaacaccaaatacagaggtcttggatgagaacagcaatagagctcagaggtcagaaactacatcttatagcctggaccgccgttcgagtgttgcagatgattctcgaagcagcacgcctgacaacaactacatcgcatatctcaataactactgccagaagaaaaaacgtgtatatgacttcaaattagtggacagaataggtcctcctcacaacccaatatttgtttataaagttgtgatggatgggaaggaataccctgaagcacagggaagaaacgctaaagaagcgaaacaaaatgcggctcaacacgcttggagtgaaattcgggatcagtctggctggaccacaccgagttctgaagatggcgcattctcacaatcccaagatacatgtaaatctgaagacgtccagcgatcagcgaacgcctccagcacaagtaatttcatctccttcagagactctgctaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 88 - DNA, 1584 bp, synthetic construct
  source 1..1584: mol_type = other DNA
atggcaaaaatgtccgcagaaaatgaaatagagatgagaatctgtgattacctgaggagacacggcaggagcaccgtacaggacattttcaaggaattaaaattagagaaatccaccgtgaacaggcatctatacagcctacaggcgtcaaagcaggtttttaaaactgtggaagataataagcggccggtctgggatctggtggagagcatgaatgacgaacaaaaagccaaaccagagcaaataaagcccgagatgacaagagacgcaattgaagaaaaacatgtgaaggatctgctgaaatcaggaggtttaaaagcctctcatatcgccagcaaactgggacaacaaacacaatcaactaacaaactactgtacggtttgatggagaaaggtgaagtgcgcaaatgcagtaaaagccacatgtggactctgaaaaatgaccaggagaggaatggaagtggcagtaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 89 - DNA, 1650 bp, synthetic construct
  source 1..1650: mol_type = other DNA
atggcccaggctcctgctgacccgggcagagaaggccaccttgaacaaagaatcctgcaggtgctgacagaggctggctccccggtgaaacttgcccagctggtgaaggaatgccaagcacccaagagggagctcaaccaagtcctctaccgaatgaaaaaggagttgaaagtctccctcacatcccctgccacctggtgcttgggcgggactgatcctgaaggcgagggtcctgcagagctggccttgtccagccctgccgagaggccccagcaacatgcagctacaattccagagacccctggccctcagttcagccaacaacgggaggaagacatctacaggtttctcaaagacaatggtccccagagggccctggtcatcgcccaagcactgggaatgaggacagcaaaagatgtgaaccgagacttgtacaggatgaagagcaggcaccttctggacatggatgagcagtccaaagcatggacgatttaccgcccagaagattctggaagaagagcaaagtcagcctcaaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 90 - DNA, 1725 bp, synthetic construct
  source 1..1725: mol_type = other DNA
atggcccaggctcctgctgacccgggcagagaaggccaccttgaacaaagaatcctgcaggtgctgacagaggctggctccccggtgaaacttgcccagctggtgaaggaatgccaagcacccaagagggagctcaaccaagtcctctaccgaatgaaaaaggagttgaaagtctccctcacatcccctgccacctggtgcttgggcgggactgatcctgaaggcgagggtcctgcagagctggccttgtccagccctgccgagaggccccagcaacatgcagctacaattccagagacccctggccctcagttcagccaacaacgggaggaagacatctacaggtttctcaaagacaatggtccccagagggccctggtcatcgcccaagcactgggaatgaggacagcaaaagatgtgaaccgagacttgtacaggatgaagagcaggcaccttctggacatggatgagcagtccaaagcatggacgatttaccgcccagaagattctggaagaagagcaaagtcagcctcaattatttaccagcacaatccaatcaacatgatctgccagaatggacccaacagctggatttccattgcaaactccaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 91 - DNA, 1608 bp, synthetic construct
  source 1..1608: mol_type = other DNA
atggcagaagctcctgttgacttgagcacaggagacaatctggagcaaaagatcctgcaggtgttgagcgatgacggcggccctgtgaagattggccagctggtgaagaaatgccaagtgcccaagaaaaccctcaatcaagtcctttaccgcctgaagaaggaggacagagtgtcctccccagagcctgcaacatggagcataggcggggctgcttctggagatggggctcctgcaatccctgagaactccagtgcccagcctagccttgatgaaagaatattaagattcctggaagccaatgggcctcacagggccctgcacatcgccaaggctctgggaatgacgacagccaaagaagtgaacccactcctgtattccatgagaaataagcaccttctgagctatgacggacagacgtggaagatctaccactcacgtcaggaaggccaagacatagctcattctggagtcacacaagagtcccctgcgaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 92 - DNA, 1683 bp, synthetic construct
  source 1..1683: mol_type = other DNA
atggcagaagctcctgttgacttgagcacaggagacaatctggagcaaaagatcctgcaggtgttgagcgatgacggcggccctgtgaagattggccagctggtgaagaaatgccaagtgcccaagaaaaccctcaatcaagtcctttaccgcctgaagaaggaggacagagtgtcctccccagagcctgcaacatggagcataggcggggctgcttctggagatggggctcctgcaatccctgagaactccagtgcccagcctagccttgatgaaagaatattaagattcctggaagccaatgggcctcacagggccctgcacatcgccaaggctctgggaatgacgacagccaaagaagtgaacccactcctgtattccatgagaaataagcaccttctgagctatgacggacagacgtggaagatctaccactcacgtcaggaaggccaagacatagctcattctggagtcacacaagagtcccctgcgattatttgtcagcacaatccggtcaacatgatctgccaacaaggagccaacagccacatctccattgccaattcaaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 93 - RNA, 1581 nt, synthetic construct
  source 1..1581: mol_type = other RNA
atggccagtgatactcccggtttctacatggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 94 - RNA, 1632 nt, synthetic construct
  source 1..1632: mol_type = other RNA
atggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaccagagaaaagctcaatgagagtttgcagagcatcctctggtcttagcaaatcttcatccagtggccacttcaacagcggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 95 - RNA, 1698 nt, synthetic construct
  source 1..1698: mol_type = other RNA
atggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaccagagaaaagctcaatgagagtttgcagagcatcctctggtcttagcaaatcttcatccagtggccacttcaacagctggtccacagcaaacaattttacttctctgtcagcgcctggaagtgatttctcagagactgcatcaggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 96 - RNA, 1809 nt, synthetic construct
  source 1..1809: mol_type = other RNA
atggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaccagagaaaagctcaatgagagtttgcagagcatcctctggtcttagcaaatcttcatccagtggccacttcaacagctggtccacagcaaacaattttacttctctgtcagcgcctgnaagtgatttctcagagactgcatcaaacaaccactgtgtttttttcaagtctcctctcatgaatgggctcagagaaaataaaaggaagccacgagtaaaactgtcatctaatgatgtgcaaagaaataaatataccggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 97 - RNA, 2661 nt, synthetic construct
  source 1..2661: mol_type = other RNA
atggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaccagagaaaagctcaatgagagtttgcagagcatcctctggtcttagcaaatcttcatccagtggccacttcaacagctggtccacagcaaacaattttacttctctgtcagcgcctggaagtgatttctcagagactgcatcaaacaaccactgtgtttttttcaagtctcctctcatgaatgggctcagagaaaataaaaggaagccacgagtaaaactgtcatctaatgatgtgcaaagaaataaatataccacggactccaggtttaacgaggattttgaagacatagaagaaattggctcaggtggatttggccaagttttcaaagcaaaacacagaatcgatgggaagacttacgctattaagcgagttaaatataactcagagaaggaagtgcgtgaagtaaaggcgctggcggcactcagccatgtcaatattgtccagtaccatttttgttggctgggacaggactgggagtacgatacagagcagagcatgaacagtgccaccccacgatcaaagacagagtgccttttcattcaaatggaattctgtgataaagggactctggagcaatggatgacaagtgtcaagcagaggaaagtagacaatgatttgtttttggaattagctgaacaaataacaacaggggtggattatatacactcaaaaggcttaattcacagagaccttaagccaagtaatatatttttagtggatgaaaaacacataaagattggagacttcggcctcgtaacagccctggaaaatgatggaaatcgaacaaagaatacgggatctcttctatacatgagcccagaacagttatctttacaggaatatggaaaagaagtggacatctttgctctaggacttattctagctgaactccttcacatatgcatcactattatagaaaagtcaaagtttttcacggacctaagagatggcatcttccatgacgacatatttggcagcaaggaaaaaaggcttctaacgaaattactctcgaagaaacccacggatcgacctagtgcttctgaaattctgatgaccttggctgaatggaagaatatctcagaagtacagaaacgacacacacatggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 98 - RNA, 2661 nt, synthetic construct
  source 1..2661: mol_type = other RNA
atggacaaacttaataaataccaccagaagcatagagtaacgattacgtataaacacctttttactacaggacctccacatgacagaaggtttacatttcaagttataatagacggagaagaatttccagaagccgaaggcaaaacaaagcaggaagcaaaaaacgctgctgccaaattggctgttgatacacttaacgcaaataaggcaaatagtcatacagacgctttggaaggttcattcactgggaattacataggccttgtcaacagttatgctcagaaggagaagttgtctgtaaattaccaacagtgtgccttcaatacccagtcaccccaaagattttgttataaatgcataattgggctgaaaacctacggtattggttcaggcgctaccaagcaggaggcaaagcagctggctgctaaagaggcgtatcagaagctaccagagaaaagctcaatgagagtttgcagagcatcctctggtcttagcaaatcttcatccagtggccacttcaacagctggtccacagcaaacaattttacttctctgtcagcgcctggaagtgatttctcagagactgcatcaaacaaccactgtgtttttttcaagtctcctctcatgaatgggctcagagaaaataaaaggaagccacgagtaaaactgtcatctaatgatgtgcaaagaaataaatataccacggactccaggtttaacgaggattttgaagacatagaagaaattggctcaggtggatttggccaagttttcaaagcaaaacacagaatcgatgggaagacttacgctattaggcgagttaaatataactcagagaaggaagtgcgtgaagtaaaggcgctggcggcactcagccatgtcaatattgtccagtaccatttttgttggctgggacaggactgggagtacgatacagagcagagcatgaacagtgccaccccacgatcaaagacagagtgccttttcattcaaatggaattctgtgataaagggactctggagcaatggatgacaagtgtcaagcagaggaaagtagacaatgatttgtttttggaattagctgaacaaataacaacaggggtggattatatacactcaaaaggcttaattcacagagaccttaagccaagtaatatatttttagtggatgaaaaacacataaagattggagacttcggcctcgtaacagccctggaaaatgatggaaatcgaacaaagaatacgggatctcttctatacatgagcccagaacagttatctttacaggaatatggaaaagaagtggacatctttgctctaggacttattctagctgaactccttcacatatgcatcactattatagaaaagtcaaagtttttcacggacctaagagatggcatcttccatgacgacatatttggcagcaaggaaaaaaggcttctaacgaaattactctcgaagaaacccacggatcgacctagtgcttctgaaattctgatgaccttggctgaatggaagaatatctcagaagtacagaaacgacacacacatggatcctcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 99 - RNA, 1740 nt, synthetic construct
  source 1..1740: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 100 - RNA, 1815 nt, synthetic construct
  source 1..1815: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaacggtctcggagaaaataaaaggaaatcaggagtaaaagtatcccctgatgatgtgcaaagaaataaatataccaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 101 - RNA, 2661 nt, synthetic construct
  source 1..2661: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaacggtctcggagaaaataaaaggaaatcaggagtaaaagtatcccctgatgatgtgcaaagaaataaatataccttggacgccaggtttaacagcgattttgaagacatagaagaaattggcttaggtggatttggtcaagttttcaaagcgaaacacagaattgatggaaagagatacgctattaagcgcgttaaatataacacggagaaggcggagcacgaagtacaagcgctggcagaactcaatcacgtcaacattgtccaataccatagttgttgggagggagttgactatgatcctgagcacagcatgagtgatacaagtcgatacaaaacccggtgcctctttattcaaatggaattctgtgataaaggaactttggagcaatggatgagaaacagaaatcagagtaaagtggacaaagctttgattttggacttatatgaacaaatcgtgaccggagtggagtatatacactcgaaagggttaattcacagagatcttaagccaggtaatatatttttagtagatgaaagacacattaagatcggagactttggccttgcaacagccctggaaaatgatggaaaatcccgaacaaggagaacaggaactcttcaatatatgagtccagaacagttatttttaaagcactatggaaaagaagtggacatctttgctttgggccttattctagctgaacttcttcacacgtgcttcacggagtcagagaaaataaagtttttcgaaagtctaagaaaaggcgacttctctaatgatatattcgacaacaaagaaaaaagccttctaaaaaaactactctcagagaaacccaaggaccgacctgagacatctgaaatcctgaagaccttggctgaatggaggaacatctcagagaaaaagaaaagaaacacatgtaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 102 - RNA, 1704 nt, synthetic construct
  source 1..1704: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaaaattcaagaaacctcccatggtggcatctacctaggtttctataaagggaaagaagtagctgtgaagagggttccaaaaggtaataataaagagctggtctgtctggagcaattacggaagaataatcatttagtacgttactgtggatatgaagaagatagctcatgttcatatctatgtctttttctgtatgagagaaaccttgaagaacacattagtaatattaaagatgtaactgagatccaagacatgcttcagtctatattcctgggcttgcatgagctacatgagataggattttgccatcgggaccttcatccatccaatattttaattgatcatgagggtaaagcttacctggcagatttcagcgggagtctgaagttggaagattctgaagacaaggaagggttgatcaagagtgacatgaaggcattgggaaaccttgtcttgtatgtagtaacaagaggaacggcctgcttccaaccagatagagaaagggatgtgacggaagcttgtccagattcagtggaagattttatagaagcaagggatctagtaaataaattaatgtctccagatacagagtattcatctgtgattgatttccttaaccatcccttcttctggagtcacacaagcaggtataagtttcttcaagatgttgggaatgagaatgatgtcaaaatgcgcacagaggacagtgcccttttcagaaccctgaatgattctcatgtgacaatgacaaaaaccttccacccctggggttgtaaggttgaccaggaggtcctgaatgacatgaacaggttccacagtaagaagaaaatctattatcaggacacagtcactgacctgttgaagttcattagaaacttgggtgctcacttcgatgagaaaaaaacagaaataaaggagatcattggagatccctgccagtattttctcaagaagtttccagatctcacggtttatgtctatgaaactctgcgacatttggggaagggatacattaagcattttccaaaccccgtagttctcttctataattgcatgcagctcaacaattcgggtatgact

SEQ ID NO: 103 - RNA, 1620 nt, synthetic construct
  source 1..1620: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgagaattcagaacacttcccagggtggcatctacttggggctccacggtgggacggaggtagcagtaagaataagccgcagtacagagggtgacaaagagaaaaggttcttcgaacaatgtggtaactgtgaacatctactgaagctcttccagtttgagaaggcaagaggctacatgtacttgtgcttccccctctgggagaaaaaccttgaagaacatctgcaggaaccagaagaccaaatggattacaaagatgctctgaggatgatcttccaggcagtgagagagctgcactcccttggatttgctcaccaggatctgcatcccaccaactttttcatagatttaggtggcaaaatttacttggcagactttgataataaaagaaagttgattgaaggcaaaaaagaactcgtaaactcagatttagaggccctcagcaggctcgtgctgtatgtcttaacagggggtaggaaaccccttcagcaagtcagtaccaaggatttgactgctgattccccagattatgaggaggctctggaccttgtaactagcctggtctctcatgatgaacgaggcttggaaggtttgagcaaacatccctatttctggagcaaacagaccaggttcaagttcctgaagggtatatggaataaaatcaaagatctccacaaccgaaaagctgtctttaaagctgctaatgctactgaacgttttccttatccacagtggaccgagaagattgacaaatatgttctgaaaatcatggaaaatcctaggaaaacaaaaccactcaaatattgcaatgatgtcaccgacctactgaggctcatcagaaacctggatgaacacccagataccaggatcagcaacagaataggagaccatgctgagtatttcttgaagcgctttccagcacttaccatttatgtctacaacagtttacgtcagaatcccaaatacagtcactttgcagacattcaggacccttctctg

SEQ ID NO: 104 - RNA, 1749 nt, synthetic construct
  source 1..1749: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaagattacgaaagacaacataacggaagtctaccttggtttctataacaatgtgattgaagtggcagtgaaatgtcacaggaacggcagtgaagaagccaaaaatgaaatggattgcctcagagatcctacgatcaatggtagcagcctgtttgttaacctcctgcattgggaaagtgatgagttttgcgattacttatgcctgaatctctatgagtataatctcgaggaatgtgtgaccatgtggcctaaggaaattcgagagaaggctcctaaaataatgaagcagttggttgacgctgttcaggttttgcatggagctggatttgctcaccgggatttacatccaacaaatgtcctcagagatgttaaagaaaattttcgcctggcagactttgataggagtgtgaattgtaagcatccagctgtgaccatatccggcaaagcgacctgggaggcttccgaaatccttcagaaactggaactctggccgacagcaaccttcgaagcggatgagcttttcaaagcggatttacaggctcttggacggcttttgcattatgttatgaccggagggaaggatccctgtagcagtgaaaatgatctgtgcgagaataatctttctctcaatgaggatctttgcgaacccaagaatgccgaagccagagatttcatcgagcgcttgttggcgccagcagagaaaagagctacattaagtgagatcagacaacatccgctgctgtggaaagatttgggaaagagccagtttgtccaaggccttgcaaatgaaaacggtgtggcaaaacgggataaatccagtccgatagtcctgattctgaatgaggttggcaacaaccaggaaagatcatttcacaagtggacagagaaggtccaccttaaaatcctaaataatatgaataagagacagcgcacaaaatatgaggacaacaccagtgacctcctgaaattcataagaaacctgacccagcattttgatgaaaaggaagaattacacagtttgattaaaagtcccgaatcttatttctgtgaattatttcctgatctcaccatcgctacctacaatgctgtgaaggacaccgagtgggtaaagcactttccaaaagaa

SEQ ID NO: 105 - RNA, 1695 nt, synthetic construct
  source 1..1695: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgtggatccagaggactacccaaggaggggtctacctaggattctatgctggagaagaggtggctgtgaagatattctggaaaggtgcagaaaatgccaagcgagagaaagtgtgccttgagaaatgtcgaaccagcaaccacctggtgaagttttgcggctgggaagagaggaagacctgtctctacctgtgcctctccttgtgtgagaggaatcttcaagagtacttcaaggaagaaaagaaaaaaactatggaaagcgaagagattcttcgaacagtatttcaggccgtaaaggaacttcacacatttggatttggccatcaggaactgcacccaagtaacattttgatagatgccactggcaggattttcctagccgactttgataagagcagagaaataactgatgatataaaatattcaataatctcagaagacctacagggcctccaaaaacttatcctgtatgttgtgatgagaggccagttggactttgaagatttgcctacagaatgcccagaaaatgtaaagaaccataaggaaattgaagacctccgaggaaggctgggactctctgaccaaagtaccccagccgatgacctactggaaaagctgatcaatcatccatatttttggtccaaggagcaaaagtacggagtgttaaaaaaggtcgggaatgagatacgtgccgctaagtgctgtaataaaaccagtgaaacaaatattctggaagctttgaaacctatggataattctttcagggactggaaaaaaaagattgataaaacagtactggactctatgttttttctgtccactgagaagggacagaaggggaatcagaagggcaacaaacgaagttatggcccctgcataacagagttactaaagttgattaggaatatgggtgagcactacaatgagaaggatgacaaggtgaaagacataattcgggacccggcagactattttctggatcgctttccagatttaaccatttatatctatcagcgtttgtgtaatacggaatacagtatacattttccaaatgctaagaatccttctcactca

SEQ ID NO: 106 - RNA, 1692 nt, synthetic construct
  source 1..1692: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaagattgctgagagctctcaagggggtgtctacctgggcttctatggggatggagttgaggttgcagtgaaacgcatgagaaagaacagtaatgaagtgaaaatggaaaagctgtgtcttgaacacccaaagattatgggcaacagctgttttgttaaactctatggcagtgaaacggacgctacatgtcattatttgtgcttgaccctctgtgaatgcactctagagacatacatacaagaggaagaaggaaatctaaagactaaagtgcctcacatatttaagcagctaatcggagcagtgcagatattacatgaagctggatttgcacacacagaccttcatccttcaaatatcctatttaatgtagaagggaagcttcaactggctgattttgataagtgtttaaggtttgaagactgtccacataaaggccaatcagaaagaagtgataaaactgaagaggagctgaaaaatgaggacattcaggcaattggaaatctgctttgttatgtcaccactgaaggaaaatatgtatacaagacagaagaggagaagcttggagactgctatagtgctgaggtgagggatcttatacagaaactttcggccccacagggacaaccacctccactaaaggaggccctgcaacatccattcttctggagtcaagaaagcaaatacagatttctacaagacattggtaatgagaatgaagtaaaagtcagaaacgacaaacatgaaatagtaaagagtcttaatgaaaaggcacaagacccttcttcatcattccgtgaatggaccaaaaaggtaaatgacattgtttttaaaaacatggatagtcgtaataggaaaccagaactcaagtataggaatgacactgcaagcctcttgaagtttatacgaaatatgggagcccattttgaggagaagccactagaaattaagaatattgtcaagaatccgtgcaactacttccttgacttgtttccagatcttgttatttctgtctacaatgttgtgaaatcaactcaatgggaagaacattttccaaaagctgaaacttct

SEQ ID NO: 107 - RNA, 1662 nt, synthetic construct
  source 1..1662: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaaaattgcagacagttctgaagggggtgtgtacttggggttctatggagaacaggaggtagctgtgaagcgattctatgaaggcagtaccctggggcaaaaggaagtctcctgtttgcagagttgccgggagaagagtaacttggtgacattctatgagagtgagaggcaaagtgactgcctgtatgtgtgcctcaccctctgcgagcagacactggaggagcacttcaatgcccgcaaaagggcagacgtgggaaacaaggaagataagtttgcccggaaagtcctctcttctatatttaaagcagtcaaagggctgcaccagtgtggatatgctcaccaagacctgcagccacgaaacatcttaatagattccaatgatgctatttgcctggcagattttgataaaagcatcaagatgactagaaaacaggaaatcaaaacagatctaaaggacctcggactactggtcatatatgttgtaaaaaagggagacatcccttttgaaagactgaagactaaaagtaatgaagacgtggttcgactttctccagatgtggaaactgaaaacctcattcaccatctgatctttcctaaggaagaatttacagacctaagtggcctgctggggcatcccttcttttggagttgggagagtcgctatagggccctgagtgaagtgggaaatgaaaatgacatcaaaattcgaaagcctaatagcaagatctttcaactgctggagcctaaatcttctgaacattccaaaagttttaaccgctggaaggctaagattgatgagtattttatgaaacaaatggatcagttttacaaaaaagacggtaggtatcagaacactgtgggtgatctgctaagattcatccggaatacaagacaacatcttcatgaaaaaaagaatgaaaaggtgaagttgagaattggagaaccttcccaatattttcaggaaaaatttccggatttctttttatatgtctatgaaagactacacagcactgaatatggaaagcattttgtccaagac

SEQ ID NO: 108 - RNA, 1752 nt, synthetic construct
  source 1..1752: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaaaatcgccgacacgcaggggggcggcgtctacctggggctcctcgaggaccaggaggtggccgtgaaggtcttcgttcagggcagtgaatgtgccggcagggagctgtccgccctcaagagtggccaggacagcagccgcctggtgaccctgcttgcctgcgagagccacgaagccaacctgtatgtgtgtatgaccctgtgtgagtggacgctggaggagttcctggagcaccgacctcagcctgcccaaggcgacgaggacaagtgggcccgccaggtcttgcactccgtgtttgaggcagttcgagagctgcacctacagcgtgctcatgcccaccaggacctgcagccacagaacattctcatagacgctgcgcacggcatccgcctggctgactttgatcagagcgtccagctggctgaacccaccaaagtctcacaggccgtcaggacagacctgcaggccctgggccggctggtcgtgtacgtgctgttgaagggggatgttccgtttgagcagctgagtgccatgcatcatcgaaaggtctggcgtctctgtcttgaacgggagaccagggacctcacgcgctgcctgctgaccccccagcaggctcaggaggaccctctgggcagcctgttgggccaccccttcttctgggagtggcagagccgctacaggacgctccggaacgtggggaacgaatctgacatcaaaaggtgcaatctcaggggcgccatcgtgcagctgctgagtctgaagccctctgagcacagcctcagcttcgcccagtggacgagcaaggtggacagcctcgtgctgggacaaatgaacgacttctacaagaacaaacctcacctgtgctacacggacagcgtgcaggacctgctgaggttcatccgcaacattggggagcacattgacgaggactgcaaccgagagatgaaggcccggctcggggaccccgctatctacttcctgcgcacatttccagacctgctgatgcacgtgtacaagcagctgcaggattcggaattccggaggcacttcccacagctccctctgcctgcgggccccggcgtgttggagacggaggtgccagggggccaggcgctgggggctgatccc

SEQ ID NO: 109 - RNA, 1650 nt, synthetic construct
  source 1..1650: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaaggttgcagaaacttctgaaggtggtgtgtacctggggttctatgatgggaaagaggcagccgtgaagcgattccgagaagacagtaaacgggctcagcaagagatggactgcatcctccattgccaaaagaatagcaacttggtgatgttttatgggagagaaagcaaagacaactgtttgtatttgtgcttttctctttgtgagaggactctggaggaacatgtgacaacaagagccttggagaatgaggacttccaaaacatttttgagtccttatttaaggcagtgcaagaactacacagacacgactatctccaccaggacctgcacccagggaatatcttgatagattctgaaggtaaagttcacttggcagattttgataagagtgaaaaattgaatgaaaatcggcataaaattaaggatgatctagagactcttggaaggctggttctatatgtggtacagttgggaaaagacccctttttcaaaattcaaggccaaagtatccgtgaggtcatagatgcttgtataaatgaggaaattaaagatcttgtgcaaaacctgcttatgcctggaacacaataccaaaacctgctaagtaagatgctctctcatcctttcttctggaattgtaatattcgttacagattccttcgagatgtggggaatgaatctgacatcaaaagacgaaattatgagagtgaaatcctcaaggcactaaactgtactgatgtttttgcagagtggacaaaaaagattgatgaacatattatgaaaaaaatgaattcattttataaaaagaagcctatgtactgctacaagaatgaagttggagacttgctaaaattcatccgtaatataggtgaacacattaatgaagaacaaaataaaaaaatgaagttaaaaattggggaacctgctcagtattttcagaagatgtttcctgatctggtgatttatgtttacaagaaactgcggaatacggtctacgagaagcattttcccagtgttaacagtact

SEQ ID NO: 110 - RNA, 1680 nt, synthetic construct
  source 1..1680: mol_type = other RNA
atggccagtgataccccaggtttctacatggacaaacttaataaataccgccagatgcacggagtagccattacgtataaagaacttagtacttcgggacctccacatgacagaaggtttacatttcaagttttaatagatgagaaggaatttccagaagccaaaggtaaatcaaagcaggaggcaagaaacgctgcagccaaattagctgttgatatacttgataacgaaaacaaggtggattgtcacacgagtgcatctgagcaaggcttgccctatggtaactacataggccttgtcaatagctttgcccagaagaaaaagctgtctgtaaattatgaacagtgtgagcccaactctgagttgcctcaaagatttatttgtaaatgcaaaattgggcagacgatgtatggtactggttcaggtgtcaccaaacaggaggcaaagcagttggctgcgaaagaagcctatcagaagctgttaaagagcccgccgaaaactgccggaacatcctctagcgttgtcacatctacattcagtggcttttccagcagctcgtctatgacaagtaatggtgtttcccagtcagcacctggaagtttttcctcagagaacgtgtttacgaagattgcagacacctctgaaggtggtgtctacttggggttctttgacggactagagacggctgtgaagatattccgtgatgacagtgaaagagccgagaaggaaatcgactgtatcctagagtgccgagagaaccgccatcttgtaaagctctatggaaccgagataaataacaactgtaggtacgtgtgcctctccctctgtgagaggaacttggaggagcacctgaaaaatcacagagatgcagatgtcgaatccaccaaaggcattctcaagtccctgtttaaggcagtgcacgaattacatcagtttggattcgtccatcaggacctgcatccaaagaacatcttgatagattctaaagatgaagtgtgcctggcagattttgataaaagctgcaggtttgctaatgatcagacattagtccatggagacctccgggcacttggacgactggtccagtatgttgtaaccaagggagaggacccctttgtggagaaaggggctgcaagagagcctcaggagctagcgggtctagctgaaatcccggatcacatggagacgctggacctcatccagacactgctctcccctggggtcaccagccagactcagctgagcgacattctctaccatcccttcttctggccctgtaacatgcgctacaggttcctaagggatgttgggaacgaatcagacatcaaaactcggaatactgaaaacaagaccctggaaaaattgaatcgtaatccttctaagagttactcaaaatggacttcaaagattgataaatatgttatgactgagatgaatcatttctacaaaaagaaaaaaaacccctatcaagacacagtgactgacctgctgaaattcatccgcaacataggcgagcacattgatgagaaagtcaatgcacaaatgaaggaaactatcggggatcctgctcagtattttcagaagacctttcctgagctggtgatgcacgtctacaagaacatacagaacacagaattcatgaaacatattccgaatgcccagaattctctcctggataac

SEQ ID NO: 111 - RNA, 2091 nt, synthetic construct
  source 1..2091: mol_type = other RNA
atggagtctctgtcagggaattatacttctctgctgaatgagtatcagcagaaaacccaatgcactgttgagtttgaggagggacccactgatggaccaagccacaataaaaggtttactatgagagctattgtgaatggacagaagtttcctgatggtactggtaaaaccaagaaggaggcaaagcaaaatgctgctaaaaacgcactggaaggcttgaagagcactcacagtgatgaaccgacaccttcacccgttgaaaacatttcagttagtaaaatagcatctcatccgaattacacctgctggctcaatgagcattcgcagaagagcaggttaatgttcaaggcctgcgagtcaactaagatggatccaggaaacctcactcggttatgcacgtatgtgtgtaagtatgtttgcgatgataaagagtttcctgaaggttatgggaaaaataagaaggaagccaaagaggcagcggctcttcgagtttatgaagagctcaacaaaacaccaaatacagaggtcttggatgagaacagcaatagagctcagaggtcagaaactacatcttatagcctggaccgccgttcgagtgttgcagatgattctcgaagcagcacgcctgacaacaactacatcgcatatctcaataactactgccagaagaaaaaacgtgtatatgacttcaaattagtggacagaataggtcctcctcacaacccaatatttgtttataaagttgtgatggatgggaaggaataccctgaagcacagggaagaaacgctaaagaagcgaaacaaaatgcggctcaacacgcttggagtgaaattcgggatcagtctggctggaccacaccgagttctgaagatggcgcattctcacaatcccaagatacatgtaaatctgaagacgtccagcgatcagcgaacgcctccagcacaagtaatttcatctccttcagagactctgctaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 112 - RNA, 1584 nt, synthetic construct
  source 1..1584: mol_type = other RNA
atggcaaaaatgtccgcagaaaatgaaatagagatgagaatctgtgattacctgaggagacacggcaggagcaccgtacaggacattttcaaggaattaaaattagagaaatccaccgtgaacaggcatctatacagcctacaggcgtcaaagcaggtttttaaaactgtggaagataataagcggccggtctgggatctggtggagagcatgaatgacgaacaaaaagccaaaccagagcaaataaagcccgagatgacaagagacgcaattgaagaaaaacatgtgaaggatctgctgaaatcaggaggtttaaaagcctctcatatcgccagcaaactgggacaacaaacacaatcaactaacaaactactgtacggtttgatggagaaaggtgaagtgcgcaaatgcagtaaaagccacatgtggactctgaaaaatgaccaggagaggaatggaagtggcagtaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 113 - RNA, 1650 nt, synthetic construct
  source 1..1650: mol_type = other RNA
atggcccaggctcctgctgacccgggcagagaaggccaccttgaacaaagaatcctgcaggtgctgacagaggctggctccccggtgaaacttgcccagctggtgaaggaatgccaagcacccaagagggagctcaaccaagtcctctaccgaatgaaaaaggagttgaaagtctccctcacatcccctgccacctggtgcttgggcgggactgatcctgaaggcgagggtcctgcagagctggccttgtccagccctgccgagaggccccagcaacatgcagctacaattccagagacccctggccctcagttcagccaacaacgggaggaagacatctacaggtttctcaaagacaatggtccccagagggccctggtcatcgcccaagcactgggaatgaggacagcaaaagatgtgaaccgagacttgtacaggatgaagagcaggcaccttctggacatggatgagcagtccaaagcatggacgatttaccgcccagaagattctggaagaagagcaaagtcagcctcaaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 114 - RNA, 1725 nt, synthetic construct
  source 1..1725: mol_type = other RNA
atggcccaggctcctgctgacccgggcagagaaggccaccttgaacaaagaatcctgcaggtgctgacagaggctggctccccggtgaaacttgcccagctggtgaaggaatgccaagcacccaagagggagctcaaccaagtcctctaccgaatgaaaaaggagttgaaagtctccctcacatcccctgccacctggtgcttgggcgggactgatcctgaaggcgagggtcctgcagagctggccttgtccagccctgccgagaggccccagcaacatgcagctacaattccagagacccctggccctcagttcagccaacaacgggaggaagacatctacaggtttctcaaagacaatggtccccagagggccctggtcatcgcccaagcactgggaatgaggacagcaaaagatgtgaaccgagacttgtacaggatgaagagcaggcaccttctggacatggatgagcagtccaaagcatggacgatttaccgcccagaagattctggaagaagagcaaagtcagcctcaattatttaccagcacaatccaatcaacatgatctgccagaatggacccaacagctggatttccattgcaaactccaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 115 - RNA, 1608 nt, synthetic construct
  source 1..1608: mol_type = other RNA
atggcagaagctcctgttgacttgagcacaggagacaatctggagcaaaagatcctgcaggtgttgagcgatgacggcggccctgtgaagattggccagctggtgaagaaatgccaagtgcccaagaaaaccctcaatcaagtcctttaccgcctgaagaaggaggacagagtgtcctccccagagcctgcaacatggagcataggcggggctgcttctggagatggggctcctgcaatccctgagaactccagtgcccagcctagccttgatgaaagaatattaagattcctggaagccaatgggcctcacagggccctgcacatcgccaaggctctgggaatgacgacagccaaagaagtgaacccactcctgtattccatgagaaataagcaccttctgagctatgacggacagacgtggaagatctaccactcacgtcaggaaggccaagacatagctcattctggagtcacacaagagtcccctgcgaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

SEQ ID NO: 116 - RNA, 1683 nt, synthetic construct
  source 1..1683: mol_type = other RNA
atggcagaagctcctgttgacttgagcacaggagacaatctggagcaaaagatcctgcaggtgttgagcgatgacggcggccctgtgaagattggccagctggtgaagaaatgccaagtgcccaagaaaaccctcaatcaagtcctttaccgcctgaagaaggaggacagagtgtcctccccagagcctgcaacatggagcataggcggggctgcttctggagatggggctcctgcaatccctgagaactccagtgcccagcctagccttgatgaaagaatattaagattcctggaagccaatgggcctcacagggccctgcacatcgccaaggctctgggaatgacgacagccaaagaagtgaacccactcctgtattccatgagaaataagcaccttctgagctatgacggacagacgtggaagatctaccactcacgtcaggaaggccaagacatagctcattctggagtcacacaagagtcccctgcgattatttgtcagcacaatccggtcaacatgatctgccaacaaggagccaacagccacatctccattgccaattcaaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgc

(19) United States
(12) Patent Application Publication
(10) Pub. No.: US 2025/0382596 A1
(43) Pub. Date: Dec. 18, 2025

(54) RECOMBINANT ENHANCED ANTIVIRAL RESTRICTORS

(71) Applicant: The Regents of the University of California, Oakland, CA (US)

(72) Inventors: Stefan ROTHENBURG, Davis, CA (US); Anak Agung Dewi MEGAWATI, Davis, CA (US)

(21) Appl. No.: 19/124,809

(22) PCT Filed: Oct. 27, 2023
(86) PCT No.: PCT/US2023/078082

§ 371 Date: Apr. 25, 2025

Related U.S. Application Data
(60) Provisional application No. 63/420,502, filed on Oct. 28, 2022.

(51) Int. Cl. C12N 9/22; A61K 38/00; A61P 31/14; C12N 9/10

(52) U.S. Cl. CPC C12N 9/22; A61P 31/14; C12N 9/10; A61K 38/00; C07K 2319/80; C07K 2319/85; C12Y 207/11013

(57) ABSTRACT

Provided herein are fusion proteins having a sensor domain that binds double-stranded nucleic acids, and an effector domain that cleaves RNA. The fusion proteins are surprising effective in inhibiting the replication of a broad variety of viruses. The fusion proteins are designed to have effector domains of particular sequence lengths, thereby improving the antiviral activity of the fusion protein. Also provided are nucleic acids encoding the fusion proteins; vectors, cells, multicellular organisms including the fusion proteins; and treatment methods using the fusion proteins.

### CROSS-REFERENCES TO RELATED APPLICATIONS

The present application is a national stage entry under 35 U.S.C. § 371 of International Patent Application No. PCT/US2023/078082, filed Oct. 27, 2023, which claims priority to U.S. Provisional Application No. 63/420,502, filed Oct. 28, 2022, the full disclosures of which are incorporated by reference in their entireties for all purposes.

### SEQUENCE LISTING

This application contains a Sequence Listing which has been submitted electronically in XML format and is hereby incorporated by reference in its entirety. Said XML copy, created on Jan. 29, 2024, is named 070772-232410PC-1411582.xml, and is 213,674 bytes in size.

### BACKGROUND

The world has been through major pandemics and epidemics caused by virus infections such as smallpox, 1918 influenza, SARS-COV, MERS-COV and currently the ongoing SARS-CoV-2 pandemic (D. M. Morens, P. Daszak, H. Markel & J. K. Taubenberger, mBio 11, (2020)). Infectious diseases continue to present significant challenges because viruses acquire the ability to overcome host immune responses and to spread efficiently. Further, as SARS-COV-2 spreads globally, tropical and subtropical countries are also battling the additional challenge of vector-borne diseases that have been endemic for decades (C. Rückert et al., Nat. Commun. 8, (2017): 15412).

As an example, viruses transmitted by vectors such as mosquitoes to humans or to other animals constitute major public health threats. The geographically most wide-spread and medically most important mosquito-borne viruses are flaviviruses such as the four serotypes of dengue virus (DENV1-4), yellow fever virus (YFV), Zika virus (ZIKV), Japanese encephalitis virus (JEV), and West Nile virus (WNV) (E. Gould, J. Pettersson, S. Higgs, R. Charrel & X. de Lamballerie, One Health 4, (2017): 1; S. Bhatt et al., Nature 496, (2013): 504; B. J. Blitvich, Anim. Health Res. Rev. 9, (2008): 71; C. Chang, K. Ortiz, A. Ansari & M. E. Gershwin, J. Autoimmun 68, (2016): 1; and M. G. Guzman et al., Nat. Rev. Microbiol. 8, (2010): S7). These viruses collectively infect hundreds of millions of people annually, are expanding their geographic ranges, and have vectors that are hard to control using conventional methods (N. M. Bowman et al., PloS One 13, (2018): e0196410; L. C. Harrington et al., Am. J. Trop. Med. Hyg. 72, (2005): 209; and J. Herrera-Bojorquez et al., J. Med. Entomol. J. Med. Entomol. 57, (2020): 503). Current state-of-the-art strategies to prevent arbovirus transmission are either resource intensive, e.g., genetically engineering mosquitoes to manipulate fertility/fecundity, sex ratio, or vector competence for arboviruses (A. E. Williams, A. W. E. Franz, W. R. Reid & K. E. Olson, Insects 11, (2020); and W. R. Reid, K. E. Olson & A. W. E. Franz, J. Med. Entomol. 58, (2021): 1987), or virus specific, e. g. RNAi-, ribozyme-, or single-chain variable fragment-based strategies (N. Jasinskiene et al., Proc. Natl Acad. Sci. USA. 95, (1998): 3743; G. Mathur et al., Insect Mol. Biol. 19, (2010): 753; A. W. Franz et al., Proc. Natl Acad. Sci. USA 103, (2006): 4198; A. W. Franz et al., PLOS Negl. Trop. Dis. 8, (2014): e2833; A. E. Williams et al., Viruses 12, (2020); P. S. Yen, A. James, J. C. Li, C. H. Chen & A. B. Failloux, Commun. Biol. 1, (2018): 11; A. Buchman et al., Proc. Natl Acad. Sci. USA 116, (2019): 3656; A. Buchman, et al., PloS Pathog. 16, (2020): e1008103; and P. Mishra, C. Furey, V. Balaraman & M. J. Fraser, Viruses 8, (2016)). The majority of these synthetic antiviral effector genes that have been designed so far target individual arboviruses, such as ZIKV, DENV, or CHIKV, which are transmitted by Aedes aegypti mosquitoes. However, exceptions are antiviral effectors targeting all four DENV serotypes simultaneously in the mosquito (A. Buchman et al., PloS Pathog. 16, (2020): e1008103; and W. L. Liu et al., Sci. Rep. 11, (2021): 23865). Nevertheless, these approaches often do not target viruses from different families and cannot prevent transmission of unidentified viruses.

The innate immune system acts as one of the first lines of defense against viral infections in both invertebrates and vertebrates by providing both sensors for virus infections and effectors against viruses. Some gene families of the innate immune system are widespread in many animal phyla (F. L. Rock, G. Hardiman, J. C. Timans, R. A. Kastelein & J. F. Bazan, Proc. Natl Acad. Sci. USA 95, (1998): 588; and J. L. Imler & L. Zheng, J. Leukoc. Biol. 75, (2004): 18), whereas others are only present in distinct lineages. For example, protein kinase R (PKR) evolved in an early vertebrate ancestor but is absent from invertebrates (S. Rothenburg, N. Deigendesch, M. Dey, T. E. Dever & L. Tazi, BMC Biol 6, (2008): 12). Mammalian PKRs contain two N-terminal dsRNA binding domains (RBD) and a C-terminal kinase domain, both of which are involved in PKR dimerization during activation. PKR is activated by sensing and binding double-stranded (ds) RNA, which is produced by most virus families at some stage during their replication cycle. Once activated, PKR phosphorylates the alpha subunit of the eukaryotic translation initiation factor 2 (eIF2α), which is highly conserved in eukaryotes. The phosphorylation of eIF2α prevents recycling of eIF2 after translation initiation, thus inhibiting cap-dependent translation (F. Zhang, et al., J. Biol. Chem. 276, (2001): 24946; and R. C. Wek, H. Y. Jiang & T. G. Anthony, Biochem. Soc. Trans. 34, (2006): 7). Consequently, PKR cannot restrict viruses that initiate translation independent of eIF2a, e.g., viruses that contain internal ribosome entry sites or other RNA structures that directly recruit ribosomes. Therefore, many arboviruses such as DENV1-4 and Sindbis virus (Togaviridae) strongly induce PKR activation as a result of dsRNA production, yet they can still replicate in PKR-competent cells due to internal ribosomal entry site-like elements (I. Ventoso, et al., Genes Dev. 20, (2006): 87; E. Domingo-Gil, R. Toribio, J. L. Najera, M. Esteban & I. Ventoso, PLOS One 6, (2011): e16711; and J. Pena & E. Harris, J. Biol. Chem. 286, (2011): 14226).

Another important antiviral mediator in mammals is the interferon-inducible 2′-5′-oligoadenylate synthetase (OAS)/RNase L pathway. RNase L genes are only found in vertebrates, specifically in mammals, birds, reptiles, and some fish and amphibian lineages, but are missing in ray-finned fishes, frogs, and salamanders (A., Chakrabarti, B. K. Jha & R. H. Silverman, J. Interferon Cytokine Res. 31, (2011): 49; and J. Hu et al., BMC Evol. Biol. 18, (2018): 201). OAS proteins are activated by dsRNA and produce 2′-5′-oligoadenylate (2-5A), a second messenger which binds to the ankyrin (ANK) repeats of inactive, monomeric RNase L to promote its dimerization and activation (B. Dong & R. H. Silverman, J. Biol. Chem. 272, (1997): 22236). Activated RNase L cleaves single-stranded viral and cellular RNA, with a preference for UNN sequence motifs. In addition to the N-terminal ANK repeats, RNase L contains a pseudokinase domain, which is also involved in its dimerization, and a C-terminal RNase domain. Crucially, many viruses antagonize this pathway by targeting either OAS or 2-5A, rather than through direct interactions with RNase L (R. Zhang et al., Proc. Natl Acad. Sci. USA 110, (2013): 13114; M. Drappier & T. Michiels, Curr. Opin. Virol. 15, (2015): 19; and M. Drappier et al. PLOS Pathog 14, (2018): e1006989). In vitro experiments utilizing heterologous N-terminal dimerization domains have demonstrated that RNase L can be activated by dimerization alone, and these chimeric RNase L constructs are independent of both OAS and 2-5A (C. W. Garvie, K. Vasanthavada & Q. Xiang, Biochim. Biophys. Acta 1834, (2013): 1562).

Because of the importance of the antiviral effects of PKR and OAS/RNase L to the innate immune system, many viruses have evolved strategies to evade PKR and OAS/RNase L activation by encoding viral antagonists (J. O. Langland, J. M. Cameron, M. C. Heck, J. K. Jancovich & B. L. Jacobs, Virus Res. 119, (2006): 100; M. Drappier & T. Michiels, Curr. Opin. Virol. 15, (2015): 19). Accordingly, there is a need for improved techniques for addressing the significant health challenges associated with viral infections. In particular, broadly active, potent strategies that do not require foreknowledge of potential pathogens are urgently needed. The present disclosure addresses this need and provides associated and other advantages.

### BRIEF SUMMARY

In general, provided herein are materials and methods related to fusion proteins effective in inhibiting the replication of diverse groups of viruses. The fusion proteins, also termed Recombinant Enhanced Anti Viral Restrictors (REAVRs), can therefore be used to treat or prevent transmission of multiple different viruses to, for example, humans. The virus sensor and effector domains of different antiviral proteins are combined to generate fusion proteins that detect nucleic acids, e.g., RNA types, which are formed by most virus families. The generated fusion proteins further inhibit a broader range of viruses compared to existing control strategies. Thus, the provided materials and methods can be used to detect and exert broad antiviral effects on various types of viruses.

In one aspect, the disclosure is to a non-naturally occurring fusion protein including a sensor domain and an effector domain. The sensor domain binds double-stranded nucleic acids. The effector domain cleaves RNA. In some embodiments, the effector domain has a length of less than 385 amino acids.

In another aspect, the disclosure is to a nucleic acid. The nucleic acid encodes a fusion protein as disclosed herein.

In another aspect, the disclosure is to a vector. The vector includes a nucleic acid as disclosed herein.

In another aspect, the disclosure is to a cell. The cell includes a fusion protein, nucleic acid, or vector as disclosed herein.

In another aspect, the disclosure is to a method of treating a subject infected with a virus. The method includes administering to the subject a therapeutically effective amount of a fusion protein, nucleic acid, vector, or cell as disclosed herein.

In another aspect, the disclosure is to a non-human multicellular organism. The multicellular organism includes a fusion protein, nucleic acid, vector, or cell as disclosed herein.

### BRIEF DESCRIPTION OF THE DRAWINGS

FIG. 1 provides schematic illustrations of antiviral proteins, including fusion proteins in accordance with provided embodiments. REAVR 1-6 are composed of different parts of Syrian hamster PKR and the human RNase L effector domain. REAVR 7-9 are composed of parts of mouse PKR and the human RNase L effector domain. REAVRs 10-18 contain parts of mouse PKR fused and the RNase L domain from a diverse set of vertebrate species as indicated above the domain structure. REAVR 21 contains parts of zebrafish PKR fused and the human RNase L effector domain. REAVR 22 contains the parts of zebrafish protein kinase Z (PKZ) fused and the human RNase L effector domain. REAVR 23-26 contains parts of human or mouse Z-DNA binding protein 1 (ZBP1) fused and the human RNase L effector domain. dsRB=double-stranded RNA binding domain; PKR KD=PKR kinase domain; ANK=ankyrin repeat domain; Ψ kinase=pseudokinase domain. Zα=Z-NA binding domain.

FIG. 2 provides schematic illustrations of PKR, RNase L, and GyrB-RNase L hybrid protein constructs. To determine which domain of RNase L is critical, the coumermycin-inducible dimerization domain of E. coli GyrB was combined with pseudokinase and RNase domain of human RNase L. As a control, GyrB-human PKR proteins (wild type PKR and kinase-inactivated PKR mutant (K296H)) were used. Three hybrid constructs were generated: GyrB-RNase L short (starting from RNase domain), GyrB-RNase L medium (starting from β1 region of pseudokinase), and GyrB-RNase L long (starting from the αA region of pseudokinase domain).

FIG. 3 is a graph showing inhibition of luciferase activity by fusion constructs. The effects of the constructs of FIG. 2 on a reporter assay in HeLa PKR knock-down cells were analyzed by treating the cells with 10 ng/ml or 50 ng/ml coumermycin.

FIG. 4 is a graph showing inhibition of luciferase expression by REAVRs 1-9. Human HeLa-PKR knock-down cells were co-transfected with firefly luciferase pGL3 and either pSG5 empty vector, Syrian hamster PKR, mouse PKR or the indicated REAVRs. Luciferase activities were measured 48 hours after transfection.

FIG. 5 is an image of a gel showing RNA cleavage activity of REAVRs in A549^{RNase L-/-/PKR-/-} cells. The cells were transiently transfected with either pSG5 empty vector, expression vector for RNase L, or the indicated REAVRs. The next day, the cells were infected with vaccinia virus ΔK3LΔE3L at an MOI of 5. RNA integrity was assessed at 6 hours or 24 hours after infection using a 2100 Bioanalyzer.

FIG. 6 is an image of a blot showing REAVR expression in A549^{RNase L-/-/PKR-/-} cells. The same amount of the indicated plasmids was transfected into the A549^{RNase L-/-/PKR-/-} cells. Protein lysates were collected at 48 hours after transfection and subjected to Western blot analysis to detect RNase L and REAVRs expressions using RNase L-specific antibodies.

FIG. 7 is a schematic illustration of REAVR expression under control of a doxycycline-inducible promoter.

FIG. 8 is an image of a blot showing expression of REAVRs after doxycycline induction. The parental T-REX WT and T-REX WT stably expressing REAVRs were treated with 0.5 μg/ml doxycycline or left untreated. At 24 hours post-treatment, cell lysates were harvested, and proteins were subjected to a Western blot analysis. REAVRs expression was detected using anti-human RNase L. Anti β-actin was used as a control (β-actin 48 kDa, REA VR3 64.88 kDa, REAVR4 71.52 kDa, REAVR5 98.56 kDa, REAVR7 65 kDa, REAVR8 68.04 kDa and REAVR9 100.9 kDa).

FIG. 9 is a graph showing the effect of REAVRs expression on the cell viability. The indicated cell lines were treated with 0.5 μg/ml doxycycline for 48 hours. T-REX PKR^{ko} cells were infected with VC2 (MOI of 0.1) as a positive control. Cell viability was assessed with ToxGlo Assay (Promega TM393). Luminescent signals are proportional to viable cell numbers. Standard deviations were determined from four independent replicates. RLU values were normalized to media only. Data were analyzed by two-way ANOVA followed by Sidak's multiple comparisons test. Significant value showed as asterisk (NS=p>0.05, *p<0.05, **p<0.01 ***p<0.001, ****P<0.0001).

FIG. 10 is a graph showing differential sensitivity of REAVRs to viral antagonists. Human HeLa-PKR-knock-out cells were transfected with expression vectors for vaccinia virus viral antagonists E3L or K3L, and pGL3 firefly luciferase, and the indicated REAVRs. As baseline, HeLa-PKR-knock-out were transfected with the indicated REAVRs, pSG5, and pGL3 firefly luciferase without viral antagonists. Syrian hamster PKR and mouse PKR served as positive controls. Luciferase activity was measured 48 hours after transfection. Luciferase light units were normalized to PKR or REAVR-only transfected cells to obtain relative luciferase activity. Standard deviations of three independent transfections are shown. Data are representative of two biological replicates.

FIG. 11 is a graph showing virus titers for T-REX WT and T-REX WT cells expressing REAVRs and infected with vaccinia virus vP872 at an MOI of 0.1. Cell lysates were collected at 48 hours post infection and virus titers were determined by plaque assay on RK13+K3L+E3L cells. The virus titer is presented in log 10 pfu/mL. Fold-change of the virus titer relative to untreated doxycycline are indicated. Data presented as mean and standard deviations were determined from three independent infections.

FIG. 12 is a graph showing virus titers for T-REX WT and T-REX WT cells expressing REAVRs and infected with vaccinia virus VC-2 strain Copenhagen at an MOI of 0.1. Cell lysates were collected at 48 hours post infection and virus titers were determined by plaque assay on RK13+K3L+E3L cells. The virus titer is presented in log 10 pfu/mL. Fold-change of the virus titer relative to untreated doxycycline are indicated. Data presented as mean and standard deviations were determined from three independent infections.

FIG. 13 presents blot images showing time dependent REAVRs expression. T-REX PKR^{ko} and T-REX PKR^{ko} stably transfected with REAVRs were grown to a confluence of 80-90% and treated with 0.5 μg/ml doxycycline. At 8-, 16-, and 24-hours post-treatment, cell lysates were harvested and 10 μg of protein was subjected to a Western blot analysis. Membranes were probed with primary antibodies against RNase L and beta-actin.

FIG. 14 is a graph showing virus titers for T-REX PKR^{ko} and T-REX PKR^{ko} cells expressing REA VRs and infected with vP872 at an MOI of 0.1. Cell lysates were collected at 48 hours post infection and virus titers were determined by plaque assay on RK13+K3L+E3L cells. The virus titer is presented in log 10 pfu/ml. Fold-change of the virus titer relative to untreated doxycycline is indicated. Data presented as mean and standard deviations were determined from three independent infections.

FIG. 15 is a graph showing virus titers for T-REX PKR^{ko} and T-REX PKR^{ko} cells expressing REAVRs and infected with VC-2 strain Copenhagen at an MOI of 0.1. Cell lysates were collected at 48 hours post infection and virus titers were determined by plaque assay on RK13+K3L+E3L cells. The virus titer is presented in log 10 pfu/ml. Fold-change of the virus titer relative to untreated doxycycline is indicated. Data presented as mean and standard deviations were determined from three independent infections.

FIG. 16 is a series of pictures showing replication of vaccinia virus VC-R4 in REAVR 5 and 9-expressing T-REX PKR^{ko} cells as indicated by expression of virus-encoded EGFP. T-REX PKR^{ko} served as negative control, T-REX PKR^{ko}-human PKR served as positive control. All cells were treated with 0.5 μg of doxycycline prior to VC-R4 infection at an MOI of 0.1. Pictures were taken at 24 hours post infection.

FIG. 17 presents blot images showing phosphorylation of eIF2α by REAVR 5 and 9. T-REX PKR^{ko} served as negative control, T-REX WT and T-REX PKR^{ko}-human PKR served as positive control. All cells were treated with 0.5 μg of doxycycline prior to VC-R4 or vP872 virus infection at an MOI of 3. Cell lysates were harvested, and the proteins were subjected to a Western blot analysis. The membranes were probed with primary antibodies against phospho-eIF2α, total eIF2α, PKR and RNase L.

FIG. 18 is a graph showing antiviral effects of REAVRs against dengue virus. Parental T-REX WT and T-REX WT cells expressing REAVRs were infected with dengue serotype 2 strain 16681 at an MOI of 0.1. Supernatant was collected at 48 hours post infection and the virus titers were determined by plaque assay on Vero CCL-81 cells. The virus titer is presented in log 10 pfu/mL. Data presented as mean and standard deviations were determined from three independent infections. Fold-change of the virus titer relative to doxycycline untreated is indicated.

FIG. 19 is a graph showing a time course of dengue virus replication in REAVR 7 expressing cells. Parental T-REX WT and T-REx WT expressing REAVR 7 as well as T-REX PKR^{ko} and T-REx PKR^{ko} expressing REAVR7 were infected with dengue serotype 2 at an MOI of 0.1. The supernatant was collected at indicated time points. The virus titer from four replicate experiments are presented with error bars indicating standard deviations.

FIG. 20 is an image of a gel showing REAVR-induced RNA cleavage after dengue virus infection. A549^{RNase L-/-/PKR-/-} cells were transiently transfected with either pSG5 empty vector, expression vector for RNase L, or the indicated REAVRs. The next day, the cells were infected with dengue serotype 2 strain 16681 at an MOI of 3. RNA integrity was assessed at 24 hours after infection using a 2100 Bioanalyzer.

FIG. 21 is a graph showing antiviral effects of REAVRs against Zika virus. Parental T-REX WT and T-REX WT cells expressing REAVRs were infected with Zika virus strain PRVABC59 at an MOI of 1. Supernatants were collected at 72 hours after infection and virus titers were performed similarly to dengue plaque assay.

FIG. 22 presents a series of flow cytometry plots showing expression of human ACE2 in T-REX WT and REAVR 7 cells. Monoclonal cells T-REX WT C9 and T-REX REAVR7 C1 stably expressing ACE2 were generated. ACE2 expression was confirmed by flow cytometry.

FIG. 23 is an image of a blot showing expression of human ACE2 in T-REX WT C9 and REAVR 7 C1 cells. ACE2 expression was confirmed by Western blot analysis.

FIG. 24 presents a series of pictures showing antiviral effects of REAVR 7 against SARS-COV-2. Parental T-REX WT and T-REX WT cells expressing REAVR 7 were infected with SARS-COV-2 mNG at an MOI of 1. Pictures were taken at 48 hours post infection.

FIG. 25 is a graph showing antiviral effects of REAVR 7 against SARS-COV-2. Parental T-REX WT and T-REX WT cells expressing REAVRs were infected with SARS-COV-2 mNG at an MOI of 1. Supernatants were collected, and the virus titers were determined by plaque assay on Vero E6 cells. The virus titer is presented in log 10 pfu/mL. Data presented as mean and standard deviations were determined from three independent infections. Fold-change of the virus titer relative to doxycycline untreated is indicated in the figure.

FIG. 26 presents a series of pictures showing antiviral effects of REAVRs against vesicular stomatitis virus strain VSV-12′GFP. T-REX PKR^{ko}, T-REX WT and stably T-REX WT cells expressing REAVRs were infected with VSV12′GFP at an MOI of 0.01. Pictures were taken at 24 hours post infection.

FIG. 27 is a graph showing antiviral effects of REAVRs against vesicular stomatitis virus strain VSV-12′GFP. T-REX PKR^{ko}, T-REX WT and T-REX WT cells stably expressing REAVRs were infected with VSV12′GFP at an MOI of 0.01. Supernatants were collected and virus titers were determined by plaque assay on Vero E6 cells. The virus titer is presented as log 10 pfu/mL. Standard deviations were determined from three independent infections. Fold-change of the virus titer relative to doxycycline untreated cells is indicated.

FIG. 28 presents a series of pictures showing the antiviral effects of REAVRs against vesicular stomatitis virus strain VSV-12′GFP. Indicated cells were grown to a confluence of 80% and were treated with 0.5 μg/ml doxycycline for about 6 hours. The cells were then infected with VSV12′GFP at an MOI of 0.1. GFP expressions were monitored at 14 hours after infection.

FIG. 29 is an image of blots showing the eIF2α phosphorylation status after VSV-12′GFP infection. Cell lysates were harvested at 14 hours after infection and the proteins were subjected to a Western blot analysis. The membrane was probed with primary antibodies against phospho-eIF2α, total eIF2α, PKR and RNase L.

FIG. 30 is an image of a gel showing rRNA degradation status after VSV-12′GFP infection. A549^{RNase L-/-/PKR-/-} cells were transiently transfected with either pSG5 empty vector, expression vector for RNase L, or the indicated REAVRs. The next day, the cells were infected with VSV-12′GFP at an MOI of 1. RNA integrity was assessed at 24 hours post infection using a 2100 Bioanalyzer.

FIG. 31 is a table showing fold reduction in virus titers after induction of REAVR expression. For all viruses, 48-hour timepoints after infection are shown, except VSV (24-hour) and CHIKV (72-hour).

FIG. 32 is a graph showing activities of selected REAVRs. HeLa^{PKRko} cells were co-transfected with a luciferase reporter and the indicated vectors for 48 hours. Luciferase values inversely correlate with REAVR activity.

FIG. 33 is a graph showing activities of selected REAVRs. HeLa^{PKRko} cells were co-transfected with a luciferase reporter and the indicated vectors for 48 hours. Luciferase values inversely correlate with REAVR activity.

FIG. 34 is a graph showing antiviral effects of REAVR expression on Dengue virus 2 replication. T-REX wild type (WT), REAVR expressing cells were infected with Dengue virus 2 at MOI=0.1, 6 hours after doxycycline (Dox, black bars) or mock (white bars) treatment. Supernatants were titered after 48 h on Vero cells. Numbers above bars represent fold differences between mock treatment and Dox treatment.

### DETAILED DESCRIPTION

### I. General

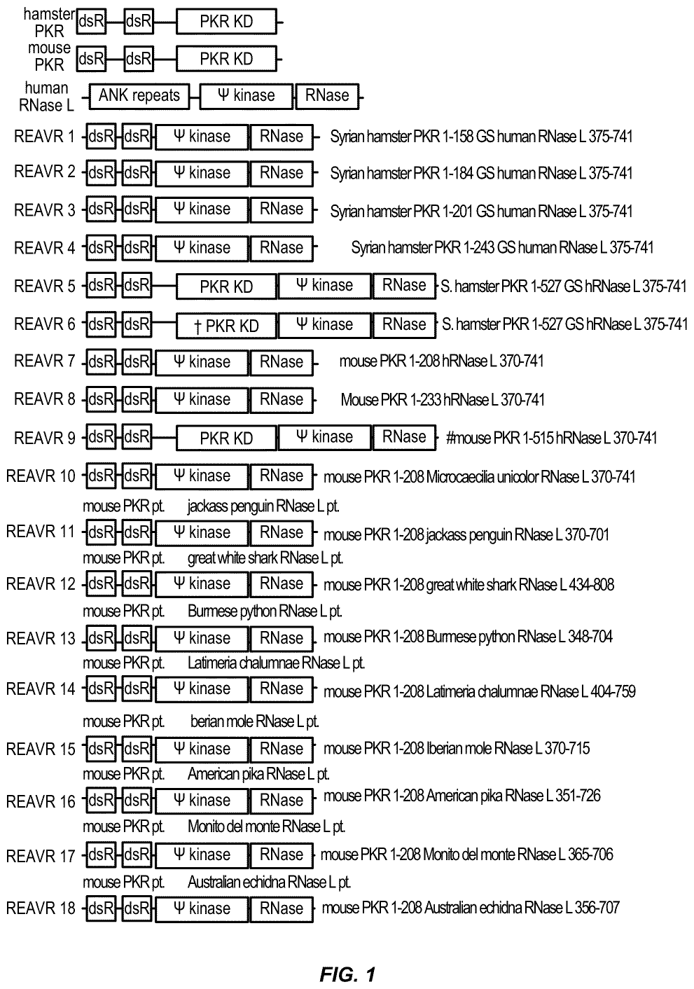

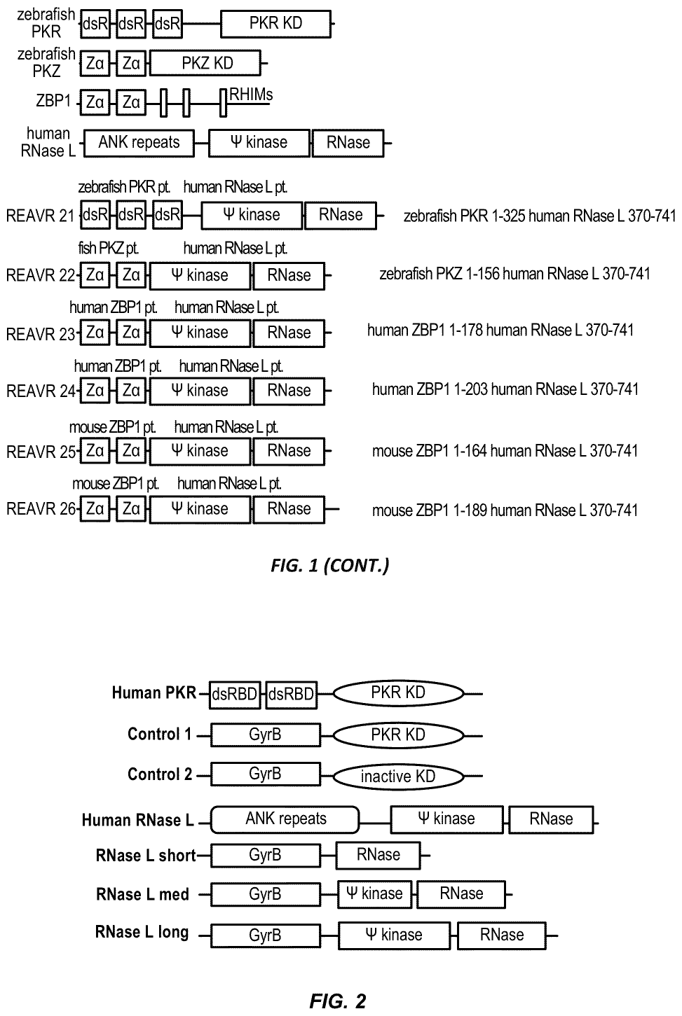

The present disclosure provides compositions and strategies for inhibiting replication of viruses. The provided materials and approaches can therefore be particularly useful in controlling vector-borne virus transmission as well as reducing vector populations. Aspects of the disclosure can also be applied to organisms to reduce their susceptibility to virus infections, or to treat infections after their occurrence. This is accomplished by creating and using surprisingly effective chimeric fusion proteins (FIG. 1) that take advantage of specific interactions between the innate immune system and viruses to make cells more resistant to a broad range of viruses. These fusion proteins, or REAVRs, have unique and broadly acting antiviral activities and simultaneously act as virus sensors and antiviral effectors. Beneficially, REAVRs exert potent antiviral activities against multiple RNA and DNA viruses, including those not inhibited by naturally occurring proteins. The REAVRs can therefore be advantageously used to generate transgenic organisms with increased viral resistance, and can additionally be used as surprisingly effective therapeutics for viral infections.

### II. Definitions

As used herein, the term “fusion protein” refers to a polypeptide engineered to include two or more sequences of amino acids corresponding to amino acid sequences from distinct polypeptides. Accordingly, a fusion protein refers to a chimeric protein containing at least portions of two or more domains corresponding to domains from other proteins or peptides, wherein these domains are connected directly or indirectly via peptide bonds in the fusion protein. The two or more domains of the fusion protein can be adjacent in the construct or can be separated by a linker or spacer.

As used herein, the term “polypeptide” refers to a polymer comprised of covalently linked natural or chemically modified amino acid residues.

As used herein, the term “sensor domain” refers to a region of a polypeptide, e.g., a fusion protein, wherein at least a portion of the region forms a sensor, e.g., binding, site of the polypeptide.

As used herein, the term “effector domain” refers to a region of a polypeptide, e.g., a fusion protein, wherein at least a portion of the region forms an effector, e.g., catalytic, site of the polypeptide.

As used herein, the terms “nucleic acid” and “polynucleotide” refer to deoxyribonucleic acids (DNA) or ribonucleic acids (RNA) and polymers thereof in either single- or double-stranded form. Unless specifically limited, the term encompasses nucleic acids containing known nucleotide analogs or modified backbone residues or linkages, which are synthetic, naturally occurring, and non-naturally occurring, that have similar binding properties as the reference nucleic acid and are metabolized in a manner similar to reference nucleotides. Examples of such analogs include, without limitation, phosphorothioates, phosphoramidates, methyl phosphonates, chiral-methyl phosphonates, 2-O-methyl ribonucleotides, and peptide nucleic acids (PNAs). Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences, as well as the sequence explicitly indicated. Specifically, degenerate codon substitutions may be achieved by generating sequences in which the third position of one or more selected (or all) codons is substituted with mixed-base and/or deoxyinosine (Batzer et al., Nucleic Acid Res. 19, (1991): 5081; Ohtsuka et al., J. Biol. Chem. 260, (1985): 2605; and Rossolini et al., Mol. Cell. Probes 8, (1994): 91).

Non-limiting examples of polynucleotides or nucleic acids include DNA, RNA, coding or noncoding regions of a gene or gene fragment, intergenic DNA, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), small nucleolar RNA (snoRNA), ribozymes, deoxynucleotides (dNTPs), or dideoxynucleotides (ddNTPs). Polynucleotides can also include complementary DNA (cDNA), which is a DNA representation of mRNA, usually obtained by reverse transcription of messenger RNA (mRNA) or by amplification. Polynucleotides can also include DNA molecules produced synthetically or by amplification, genomic DNA (gDNA), recombinant polynucleotides, branched polynucleotides, plasmids, vectors, isolated DNA of any sequence, isolated RNA of any sequence, nucleic acid probes, or primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. Polynucleotide sequences, when provided, are listed in the 5′ to 3′ direction, unless stated otherwise.

Nucleic acids or polynucleotides can be double- or triple-stranded nucleic acids, as well as single-stranded molecules. In double- or triple-stranded nucleic acids, the nucleic acid strands need not be coextensive, for example, a double-stranded nucleic acid need not be double-stranded along the entire length of both strands.

Nucleic acid modifications can include addition of chemical groups that incorporate additional charge, polarizability, hydrogen bonding, electrostatic interaction, and functionality to the individual nucleic acid bases or to the nucleic acid as a whole. Such modifications include base modifications such as 2′-position sugar modifications, 5-position pyrimidine modifications, 8-position purine modifications, modifications at cytosine exocyclic amines, substitutions of S-bromo-uracil, backbone modifications, unusual base pairing combinations such as the isobases isocytidine and isoguanidine, and the like.

Nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “connected” when used in reference to polypeptide domains, modules, regions, and the like indicates that these features are elements of the same polypeptide. Recited domains, modules, or other polypeptide elements can be connected directly adjacent to one another, such that the N or C terminus of one recited domain, module, or other element can be fused, e.g., covalently joined, to the C or N terminus of another recited domain, module, or element. Recited domains, modules, or other polypeptide elements can be connected indirectly to one another, such that the recited domains, modules, or other polypeptide elements can be separated by intervening sequences of the polypeptide, including those of one or more additional domains, modules, or other polypeptide elements.

As used herein, the term “linker” refers to nucleotide sequences that are attached to another sequence of DNA or RNA. The linker can be single-stranded or double-stranded. The linker can comprise both single- and double-stranded regions. The linker can comprise RNA nucleotides. The linker or adapter can comprise DNA nucleotides. The linker can comprise both RNA and DNA nucleotides. The linker can comprise non-naturally occurring nucleotides.

As used herein, the term “vector” refers to a nucleic acid molecule capable of propagating another nucleic acid to which it is linked. The term includes the vector as a self-replicating nucleic acid structure as well as a vector incorporated into the genome of a host cell into which it has been introduced. A vector can therefore refer to a recombinant construct in which a nucleic acid sequence of interest is inserted into the vector. Certain vectors are capable of directing the expression of nucleic acids to which they are operatively linked.

As used herein, the term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens, and the like), seaweeds (e.g., kelp), a fungal cell (e.g., a yeast cell, a cell from a mushroom), a cell from an invertebrate animal (e.g., fruit fly, cnidarian, echinoderm, nematode, mollusk, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), etc. Sometimes a cell does not originate from a natural organism (e.g., a cell can be a synthetically made, sometimes termed an artificial cell).

As used herein, the term “treatment” refers to an approach for obtaining beneficial or desired results including but not limited to a therapeutic benefit and/or a prophylactic benefit. A therapeutic benefit imparts any relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested. A treatment can involve any of ameliorating one or more symptoms of disease, e.g., cancer, preventing the manifestation of such symptoms before they occur; slowing down or completely preventing the progression of the disease (as may be evident by longer periods between reoccurrence episodes, slowing down or prevention of the deterioration of symptoms, etc.), enhancing the onset of a remission period, slowing down the irreversible damage caused in the progressive-chronic stage of the disease (both in the primary and secondary stages), delaying the onset of said progressive stage, or any combination thereof.

As used herein, the term “therapeutically effective amount” refers to the quantity of a composition that is sufficient to result in a desired activity upon administration to a subject in need thereof. Within the context of the present disclosure, the term “therapeutically effective” refers to that quantity of a composition that is sufficient to delay the manifestation, arrest the progression, or relieve or alleviate at least one symptom of a disorder treated by the methods of the present disclosure.

As used herein, the terms “including,” “comprising,” “having,”, “containing,” and variations thereof, are inclusive and open-ended and do not exclude additional, unrecited elements or method steps beyond those explicitly recited. As used herein, the phrase “consisting of” is closed and excludes any element, step, or ingredient not explicitly specified. As used herein, the phrase “consisting essentially of” limits the scope of the described feature to the specified materials or steps and those that do not materially affect the basic and novel characteristics of the disclosed feature.

### III. Fusion Proteins

In one aspect, the present disclosure provides various fusion proteins that generally include a sensor domain and an effector domain, each originating from a different protein, e.g., a different antiviral protein. The particular selection, composition, and arrangement of the sensor domain and the effector domain in the fusion protein provides surprising improvements in the efficacy of the antiviral properties of the fusion protein. In particular, the fusion protein can be significantly more effective than the source proteins of the sensor domain and the effector domain in inhibiting the replication of a broader range of viruses.

The sensor domain of the provided fusion protein binds double-stranded nucleic acids. In other words, at least a portion of the fusion protein sensor domain forms a binding site of the fusion protein, where the binding site binds to double-stranded nucleic acids. In some embodiments, the sensor domain binds double-stranded RNA. In some embodiments, the sensor domain binds Z-DNA, Z-RNA (Z-DNA/Z-RNA), or Z-nucleic acid-like structures, such as single-stranded DNA or RNA mimicking Z-DNA/Z-RNA. In some embodiments, the sensor domain binds A-DNA. In some embodiments, the sensor domain binds B-DNA. The sensor domain of the fusion protein can include one double-stranded nucleic acid binding domain or region, or can include two or more double-stranded nucleic acid binding domains or regions. In some embodiments, the senor domain includes a combination of different nucleic-acid-sensing domains such as a combination of dsRNA and Z-DNA/Z-RNA-binding domains. In some embodiments, the sensor domain of the fusion protein includes one or more other domains in addition to one or more double-stranded nucleic acid binding domains. For example, the fusion protein sensor domain can include a kinase domain in addition to one or more double-stranded nucleic acid binding domains.

The sensor domain of the provided fusion protein can be an amino acid sequence that corresponds to at least a portion of a sequence of a first polypeptide, where the full sequence of the first polypeptide is different from the full sequence of the fusion protein. The sensor domain can, for example, be identical or similar to a portion of a sequence of a wild-type or naturally occurring polypeptide. The sensor domain of the fusion protein can, for example, have at least 80% amino acid sequence identity, e.g., at least 82% identity, at least 84% identity, at least 86% identity, at least 88% identity, at least 90% identity, at least 92% identity, at least 94% identity, at least 96% identity, at least 98% identity, or at least 99% identity, with the sensor domain of a naturally occurring polypeptide, e.g., with the sequence of any one of SEQ ID NOS: 18-32.

In some embodiments, the sensor domain of the fusion protein corresponds to a sensor domain from a protein naturally occurring in a human. In some embodiments, the fusion protein sensor domain corresponds to a sensor domain from a protein naturally occurring in a non-human animal. In some embodiments, the fusion protein sensor domain corresponds to a sensor domain from a protein naturally occurring in a vertebrate. In some embodiments, the fusion protein sensor domain corresponds to a sensor domain from an invertebrate. The fusion protein sensor domain can correspond to a sensor domain from, for example and without limitation, Syrian hamster, mouse, or zebrafish.

In some embodiments, the sensor domain of the provided fusion protein is a protein kinase R domain, i.e., the sensor domain is identical or similar to a portion of the amino acid sequence of a protein kinase R protein. In some embodiments, the sensor domain of the fusion protein includes an amino acid sequence corresponding to all or part of one or all of the double-stranded RNA binding domains of a protein kinase R protein. In some embodiments, the sensor domain of the fusion protein further includes an amino acid sequence corresponding to all or part of the kinase domain of a protein kinase R protein. In some embodiments, the sensor domain of the provided fusion protein is a protein kinase Z domain, i.e., the sensor domain is identical or similar to a portion of the amino acid sequence of a protein kinase Z protein. In some embodiments, the sensor domain of the fusion protein includes an amino acid sequence corresponding to all or part of one or all of the Z-DNA/Z-RNA binding domains of a protein kinase Z protein. In some embodiments, the sensor domain of the fusion protein further includes an amino acid sequence corresponding to all or part of the kinase domain of a protein kinase Z protein. In some embodiments, the sensor domain of the provided fusion protein is a Z-DNA binding protein domain, i.e., the sensor domain is identical or similar to a portion of the amino acid sequence of a Z-DNA binding protein. In some embodiments, the sensor domain of the fusion protein includes an amino acid sequence corresponding to all or part of one or all of the Z-DNA/Z-RNA binding domains of a Z-DNA binding protein. In some embodiments, the sensor domain of the fusion protein further includes an amino acid sequence corresponding to all or part of the kinase domain of a Z-DNA binding protein.

The effector domain of the provided fusion protein cleaves RNA. In other words, at least a portion of the fusion protein effector domain forms a catalytic site of the fusion protein, where the catalytic site catalyzes cleavage of RNA. In some embodiments, the effector domain cleaves single-stranded RNA. In some embodiments, the effector domain cleaves double-stranded RNA. The effector domain of the fusion protein can include one RNA cleaving domain or region, or can include two or more RNA cleaving domains or regions. In some embodiments, the effector domain of the fusion protein includes one or more other domains in addition to one or more RNA cleaving domains. For example, the fusion protein effector domain can include a pseudokinase domain in addition to one or more RNA cleaving domains.

The effector domain of the provided fusion protein can be an amino acid sequence that corresponds to at least a portion of a sequence of a second polypeptide, where the full sequence of the second polypeptide is different from the full sequences of the first polypeptide and the fusion protein. The effector domain can, for example, be identical or similar to a portion of a sequence of a wild-type or naturally occurring polypeptide. The effector domain of the fusion protein can, for example, have at least 80% amino acid sequence identity, e.g., at least 82% identity, at least 84% identity, at least 86% identity, at least 88% identity, at least 90% identity, at least 92% identity, at least 94% identity, at least 96% identity, at least 98% identity, or at least 99% identity, with the effector domain of a naturally occurring polypeptide, e.g., with the sequence of any one of SEQ ID NOS: 1-17.

In some embodiments, the effector domain of the fusion protein corresponds to an effector domain from a protein naturally occurring in a human. In some embodiments, the fusion protein effector domain corresponds to an effector domain from a protein naturally occurring in a non-human animal. In some embodiments, the fusion protein effector domain corresponds to an effector domain from a protein naturally occurring in a vertebrate. In some embodiments, the fusion protein effector domain corresponds to an effector domain from an invertebrate. The fusion protein effector domain can correspond to an effector domain from, for example and without limitation, Iberian mole, American pika, monito del monte, Australian echidna, African penguin Burmese python, great white shark, Latimeria chalumnae, or Microcaecilia unicolor.

In some embodiments, the effector domain of the provided fusion protein is an RNase L domain, i.e., the effector domain is identical or similar to a portion of the amino acid sequence of an RNase L protein. In some embodiments, the effector domain of the fusion protein includes an amino acid sequence corresponding to all or part of the RNase domain of an RNase L protein. In some embodiments, the effector domain of the fusion protein further includes an amino acid sequence corresponding to all or part of the pseudokinase domain of an RNase L protein.

As described in further detail in the Examples, the length of the effector domain of the provided fusion protein can be significant in determining the antiviral activity profile of the fusion protein. For example, a fusion protein having an effector domain that is smaller or larger than a particularly determined size can have reduced or no antiviral activity against one or more types of viruses. Accordingly, in some embodiments the provided fusion protein is designed or engineered to have a particular effector domain length. The length of the effector domain can be, for example, between 225 amino acids and 385 amino acids, e.g., between 225 amino acids and 321 amino acids, between 241 amino acids and 337 amino acids, between 257 amino acids and 353 amino acids, between 273 amino acids and 369 amino acids, or between 289 amino acids and 385 amino acids. In terms of upper limits, the effector domain length can be, for example, less than 385 amino acids, e.g., less than 369 amino acids, less than 353 amino acids, less than 337 amino acids, less than 321 amino acids, less than 305 amino acids, less than 289 amino acids, less than 273 amino acids, less than 257 amino acids, or less than 241 amino acids. In terms of lower limits, the length of the effector domain can be, for example, greater than 225 amino acids, e.g., greater than 241 amino acids, greater than 257 amino acids, greater than 273 amino acids, greater than 289 amino acids, greater than 305 amino acids, greater than 321 amino acids, greater than 337 amino acids, greater than 353 amino acids, or greater than 369 amino acids. Longer lengths, e.g., greater than 385 amino acids, and shorter lengths, e.g., less than 225 amino acids, are also contemplated.

In some embodiments, the fusion protein provided herein further includes a linker sequence between a nucleic acid binding domain of the sensor domain, and an RNA cleaving domain of the effector domain. As described in further detail in the Examples, the length of the linker of the provided fusion protein can be significant in determining the antiviral activity profile of the fusion protein. For example, a fusion protein having a linker that is smaller or larger than a particularly determined size can have reduced or no antiviral activity against one or more types of viruses. Accordingly, in some embodiments the provided fusion protein is designed or engineered to have a particular linker length. The length of the linker sequence can be, for example, from 2 amino acids to 112 amino acids, e.g., from 2 amino acids to 68 amino acids, from 13 amino acids to 79 amino acids, from 24 amino acids to 90 amino acids, from 35 amino acids to 101 amino acids, or from 46 amino acids to 112 amino acids. In terms of upper limits, the linker length can be, for example, at most 112 amino acids, e.g., at most 101 amino acids, at most 90 amino acids, at most 79 amino acids, at most 68 amino acids, at most 57 amino acids, at most 46 amino acids, at most 35 amino acids, at most 24 amino acids, or at most 13 amino acids. In terms of lower limits, the linker length can be, for example, at least 2 amino acids, at least 13 amino acids, at least 24 amino acids, at least 35 amino acids, at least 46 amino acids, at least 57 amino acids, at least 68 amino acids, at least 79 amino acids, at least 90 amino acids, or at least 101 amino acids. Longer lengths, e.g., greater than 112 amino acids, are also contemplated.

In some embodiments, the linker region of the provided fusion protein is identical or similar to a portion of a sequence of a wild-type or naturally occurring polypeptide. The linker region of the fusion protein can, for example, have at least 80% amino acid sequence identity, e.g., at least 82% identity, at least 84% identity, at least 86% identity, at least 88% identity, at least 90% identity, at least 92% identity, at least 94% identity, at least 96% identity, at least 98% identity, or at least 99% identity, with a linker region of a naturally occurring polypeptide, e.g., with the sequence of any one of SEQ ID NOS: 33-44. In some embodiments, the linker region of the fusion protein corresponds to a linker region from a protein naturally occurring in a human. In some embodiments, the fusion protein linker corresponds to a linker from a protein naturally occurring in a non-human animal. In some embodiments, the fusion protein linker corresponds to a linker from a protein naturally occurring in a vertebrate. In some embodiments, the fusion protein linker corresponds to a linker from an invertebrate.

In some embodiments, the provided fusion protein has at least 80% amino acid sequence identity, e.g., at least 82% identity, at least 84% identity, at least 86% identity, at least 88% identity, at least 90% identity, at least 92% identity, at least 94% identity, at least 96% identity, at least 98% identity, or at least 99% identity, with the sequence of any one of SEQ ID NOS: 45-68.

### IV. Nucleic Acids, Vectors, Cells, and Organisms

In further aspects, the present disclosure provides nucleic acids, e.g., isolated nucleic acids, encoding any of the fusion proteins as described herein; vectors including such nucleic acids, and host cells and multicellular organisms into which the nucleic acids or vectors are introduced.

In some embodiments, the provided nucleic acid includes DNA. The DNA can have a nucleotide sequence that has, for example, at least 80% sequence identity, e.g., at least 82% identity, at least 84% identity, at least 86% identity, at least 88% identity, at least 90% identity, at least 92% identity, at least 94% identity, at least 96% identity, at least 98% identity, or at least 99% identity, with the sequence of any one of SEQ ID NOS: 69-92. In some embodiments, the provided nucleic acid includes RNA, e.g., mRNA. The RNA can have a nucleotide sequence that has, for example, at least 80% sequence identity, e.g., at least 82% identity, at least 84% identity, at least 86% identity, at least 88% identity, at least 90% identity, at least 92% identity, at least 94% identity, at least 96% identity, at least 98% identity, or at least 99% identity, with the sequence of any one of SEQ ID NOS: 93-116.

In some embodiments, the provided vector includes, in addition to a nucleic acid encoding a disclosed fusion protein, a promoter regulating expression of this nucleic acid. In some embodiments, the promoter is a constitutive promoter. In some embodiments, the promoter is an inducible promoter. For certain applications, an inducible promoter can provide advantages relative to a constitutive promoter. For example, the constitutive expression of a provided fusion protein in a cell or a multicellular organism can in certain circumstances have negative effects on the development of the cell or multicellular organism. In such cases, the use of an inducible promoter, such as virus-inducible promoter, can be beneficial. Examples of virus-inducible promoters suitable for use with the provided vectors include, without limitation, promoters upregulated by double-stranded RNA, by double-stranded DNA, or by bloodmeal.

### V. Methods of Treatment

Another aspect of the present disclosure relates to methods for treating a subject infected with a virus. The methods generally include administering to the subject a therapeutically effective amount of a fusion protein, nucleic acid, vector, or cell as disclosed herein. In some embodiments, the subject is a vertebrate. In some embodiments, the subject is a mammal. Mammalian subjects for which the provided methods are suitable include, but are not limited to, mice, rats, simians, humans, farm animals, sport animals, and pets. In some embodiments, the subject is human. In some embodiments, the subject is male. In some embodiments, the subject is female. In some embodiments, the subject is an adult. In some embodiments, the subject is an adolescent. In some embodiments, the subject is a child. In some embodiments, the subject is above about 10 years of age, e.g., above about 20 years of age, above about 30 years of age, above about 40 years of age, above about 50 years of age, above about 60 years of age, above about 70 years of age, or above about 80 years of age. In some embodiments, the subject is less than about 80 years of age, e.g., less than about 70 years of age, less than about 60 years of age, less than about 50 years of age, less than about 40 years of age, less than about 30 years of age, less than about 20 years of age, or less than about 10 years of age. In some embodiments, the method further includes evaluating the subject to determine the nature of the viral infection. In some embodiments, the method further includes selecting a provided nucleic acid, vector, or cell to be administered to the subject in view of the determined viral infection.

### VI. Exemplary Embodiments

The following embodiments are contemplated. All combinations of features and embodiments are contemplated.

Embodiment 1: A fusion protein comprising: a sensor domain that binds double-stranded nucleic acids; and an effector domain that cleaves RNA, wherein the effector domain has a length of less than 385 amino acids, and wherein the fusion protein is not naturally occurring.

Embodiment 2: An embodiment of embodiment 1, wherein the effector domain has a length greater than 225 amino acids.

Embodiment 3: An embodiment of embodiment 1 or 2, wherein the effector domain cleaves single-stranded RNA.

Embodiment 4: An embodiment of embodiment 3, wherein the effector domain is an RNase L domain.

Embodiment 5: An embodiment of embodiment 4, wherein the effector domain is a vertebrate RNase L domain.

Embodiment 6: An embodiment of any embodiment of embodiment 1-5, wherein the effector domain is a non-human effector domain.

Embodiment 7: An embodiment of any embodiment of embodiment 1-6, wherein the effector domain has at least 80% sequence identity with an amino acid sequence selected from the group consisting of SEQ ID NOS: 1-17.

Embodiment 8: An embodiment of any embodiment of embodiment 1-7, wherein the sensor domain binds double-stranded RNA or Z-DNA.

Embodiment 9: An embodiment of embodiment 8, wherein the sensor domain is a protein kinase R domain, a Z-DNA binding protein domain, or a protein kinase Z domain.

Embodiment 10: An embodiment of embodiment 9, wherein the sensor domain is a protein kinase R domain.

Embodiment 11: An embodiment of any embodiment of embodiment 1-10, wherein the sensor domain is a non-human sensor domain.

Embodiment 12: An embodiment of any embodiment of embodiment 1-11, wherein the sensor domain has at least 80% sequence identity with an amino acid sequence selected from the group consisting of SEQ ID NOS: 18-32.

Embodiment 13: An embodiment of any embodiment of embodiment 1-12, wherein the fusion protein further comprises a linker region connecting the sensor domain and effector domain.

Embodiment 14: An embodiment of embodiment 13, wherein the linker region has a length between 2 amino acids and 112 amino acids.

Embodiment 15: An embodiment of embodiment 13 or 14, wherein the linker region has at least 80% sequence identity with an amino acid sequence selected from the group consisting of SEQ ID NOS: 33-44.

Embodiment 16: An embodiment of any embodiment of embodiment 1-15, wherein the fusion protein has at least 80% sequence identity with an amino acid sequence selected from the group consisting of SEQ ID NOS: 45-68.

Embodiment 17: A nucleic acid encoding the fusion protein of an embodiment of any embodiment of embodiment 1-16.

Embodiment 18: An embodiment of embodiment 17, wherein the nucleic acid comprises DNA.

Embodiment 19: An embodiment of embodiment 18, wherein the nucleic acid has at least 80% sequence identity with a nucleotide sequence selected from the group consisting of SEQ ID NOS: 69-92.

Embodiment 20: An embodiment of embodiment 17, wherein the nucleic acid comprises mRNA.

Embodiment 21: An embodiment of embodiment 20, wherein the nucleic acid has at least 80% sequence identity with a nucleotide sequence selected from the group consisting of SEQ ID NOS: 93-116.

Embodiment 22: A vector comprising the nucleic acid of an embodiment of any embodiment of embodiment 17-19.

Embodiment 23: An embodiment of embodiment 22, wherein the vector further comprises a promoter regulating expression of the nucleic acid.

Embodiment 24: An embodiment of embodiment 23, wherein the promoter is a constitutive promoter.

Embodiment 25: An embodiment of embodiment 23, wherein the promoter is an inducible promoter.

Embodiment 26: An embodiment of embodiment 25, wherein the inducible promoter is a virus-inducible promoter.

Embodiment 27: An embodiment of embodiment 25 or 26, wherein the inducible promoter is upregulated by double-stranded RNA, double-stranded DNA or by bloodmeal.

Embodiment 28: An embodiment of embodiment 22-27, wherein: the nucleic acid is a first nucleic acid, the sensor domain is a first sensor domain, and the effector domain is a first effector domain; the vector further comprises a second nucleic acid encoding a second fusion protein; the second fusion protein comprises a second sensor domain that binds double-stranded nucleic acids, and a second effector domain that cleaves RNA; the second fusion protein has a different amino acid sequence than the first fusion protein; and the second fusion protein is not naturally occurring.

Embodiment 29: A cell comprising the fusion protein of an embodiment of any embodiment of embodiment 1-16, the nucleic acid of an embodiment of any embodiment of embodiment 17-21, or the vector of an embodiment of any embodiment of embodiment 22-28.

Embodiment 30: A method of treating a subject infected with a virus, the method comprising administering to the subject a therapeutically effective amount of the fusion protein of an embodiment of any embodiment of embodiment 1-16, the nucleic acid of an embodiment of any embodiment of embodiment 17-21, the vector of an embodiment of any embodiment of embodiment 22-28, or the cell of embodiment 29

Embodiment 31: An embodiment of embodiment 30, wherein the virus is an arbovirus.

Embodiment 32: An embodiment of embodiment 30 or 31, wherein the virus is a member of the Coronaviridae family, the Rhabdoviridae family, the Poxviridae family, the Flaviviridae family, the Togaviridiae family, the Orthomyxoviridiae family, or the Peribunyaviridae family.

Embodiment 33: An embodiment of embodiment 32, wherein the virus is a SARS-COV-2 virus, a vesicular stomatitis virus, a vaccinia virus, a dengue virus, a Zika virus, a Chikungunya virus, an influenza A virus, or a La Crosse virus.

Embodiment 34: A multicellular organism comprising the fusion protein of an embodiment of any embodiment of embodiment 1-16, the nucleic acid of an embodiment of any embodiment of embodiment 17-21, the vector of an embodiment of any embodiment of embodiment 22-28, or the cell of embodiment 29, wherein the multicellular organism is non-human.

Embodiment 35: An embodiment of embodiment 34, wherein the multicellular organism is an arthropod.

Embodiment 36: An embodiment of embodiment 35, wherein the arthropod is a mosquito, fly or a tick.

Embodiment 37: An embodiment of embodiment 34, wherein the multicellular organism is a vertebrate.

Embodiment 38: An embodiment of embodiment 37, wherein the vertebrate is a livestock animal.

Embodiment 39: An embodiment of embodiment 38, wherein the livestock animal is porcine or avian.

Embodiment 40: An embodiment of embodiment 34, wherein the multicellular organism is a plant.

Embodiment 41: An embodiment of 40, wherein the plant is agricultural, aquacultural, or horticultural.

### EXAMPLES

The present disclosure will be better understood in view of the following non-limiting examples. The following examples are intended for illustrative purposes only and do not limit in any way the scope of the present invention

### Example 1. Generation and Functional Analysis of Recombinant Enhanced Antiviral Restrictors (REAVRs)

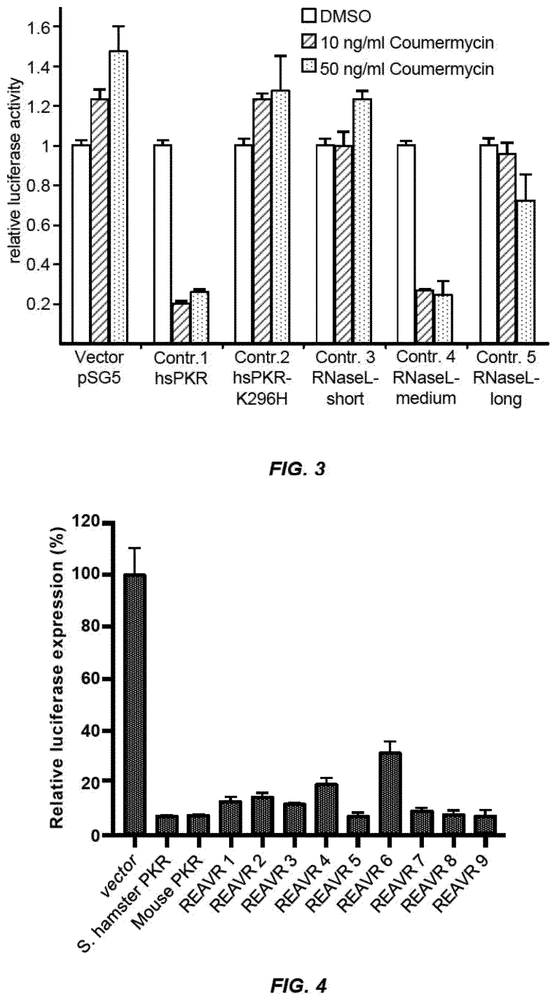

To determine which domains of RNase L are critical for the generation of active fusion proteins, three GyrB-RNase L hybrid constructs were constructed. These constructs included of the first 221 amino acids of Escherichia coli (E. coli) DNA gyrase subunit B (GyrB) fused to different lengths of the human RNase L effector domain: GyrB-RNase L short (containing residues 588-741), GyrB-RNase L medium (starting from the β1 region of pseudokinase residues 375-741), and GyrB-RNase L long (starting from the αA region of pseudokinase domain residues 333-741) (FIG. 2). Control constructs expressed fusion proteins including the GyrB dimerization domain fused to either the wild-type or inactivated (K296H) human PKR kinase domain (residues 258-551), which were previously shown to confer coumermycin dimerization-dependent eIF2α kinase activity in a mammalian cell line (T. L. Ung, C. Cao, J. Lu, K. Ozato & T. E. Dever, EMBO J. 20, (2001): 3728; I. Friedrich, M. Eizenbach, J. Sajman, H. Ben-Bassat & A. Levitzki A, Mol. Ther. 12, (2005): 969). Cells were transfected with indicated plasmids and two different coumermycin concentrations were added to promote GyrB dimerization. The amount of light produced by transfected cells upon the addition of luciferin was then measured. In this assay, luciferase activity is inversely correlated with PKR activity. Both 10 ng/ml and 50 ng/ml of coumermycin led to a reduction of luciferase activity for the GyrBPKR construct, whereas the addition of coumermycin had no effects on luciferase activity for the kinase-inactivated construct (FIG. 3). Among the three GyrB-RNase L hybrids, only GyrB-RNase L medium resulted in a marked reduction of luciferase activity comparable to that of human PKR, indicating that RNase L medium is the optimal length of RNase L effector domain for retaining RNase L activity.

Multiple REAVRs were then generated by combining the sensor domain of PKR with the medium-length effector domain of human RNase L. First, the dsRNA sensing domains of Syrian hamster PKR was fused with the effector domain of human RNase L. Syrian hamster PKR was selected as sensor domain source because Syrian hamster PKR is resistant to some viral antagonists that can inhibit PKR activation including vaccinia virus (VACV) E3L, herpes simplex virus 1 (HSV-1) US11, and mammalian reovirus #sigma3. As was demonstrated with GyrB constructs, the linker region connecting the PKR dsRBDs and the RNase L effector domain can influence the activity of fusion proteins. Therefore, six structural variants of REAVRs were generated. Specifically, these variants included both dsRBDs with no linker region (REAVR 1, residues 1-158), a portion of the linker region (REAVR 2 residues 1-183; REAVR 3 residues 1-206), the entire linker region (REAVR 4 residues 1-243), the entire Syrian hamster PKR sequence (REAVR 5 residues 1-527), and kinase-inactivated Syrian hamster PKR K272R (REAVR 6) (FIG. 1). In addition, REAVRs 7-9 were designed to contain parts of mouse PKR as sensors, with structures corresponding to REAVRs 3-5 (FIG. 1). Mouse PKR was selected as a sensor domain origin because mouse PKR is susceptible to E3L, US11, and #sigma3, whereas Syrian hamster PKR was resistant to these antagonists. The effects of REAVRs on protein expression in a luciferase-based reporter system were first compared. Plasmids encoding PKR and a luciferase reporter were co-transfected into PKR-deficient HeLa cells. In this assay, luciferase activity is inversely correlated with PKR activity. PKR and REAVRs are activated by dsRNA that is formed by overlapping transcripts generated from plasmids (See J. Nejepinska, R. Malik, S. Wagner & P. Svoboda, PLOS One 9, (2014): e87517). All REAVRs. as well as Syrian hamster and mouse PKR, were found to substantially reduced luciferase expression, indicating that they are functional (FIG. 4). REAVR 6, which contains a kinase-inactivating mutation in PKR, showed the lowest activity, but still reduced luciferase expression ˜3-fold.

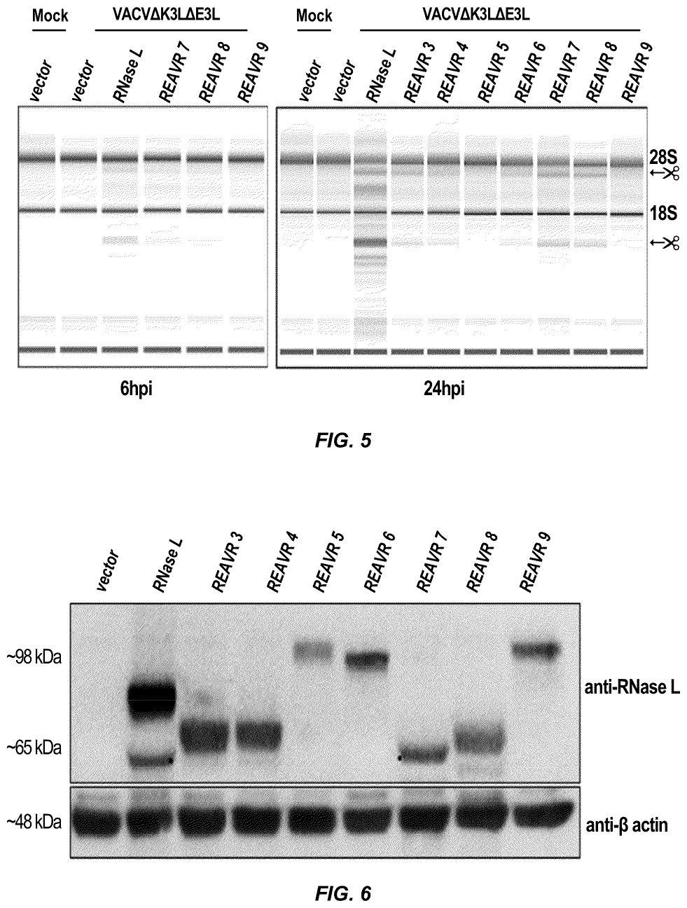

Ribosomal RNA (rRNA) degradation is a hallmark of RNase L endonuclease activity (R. H. Silverman, J. Virol. 81, (2007): 12720). Therefore, to determine whether REAVRs exhibit endonuclease activity, we transiently transfected A549^{RNase L-/-/PKR-/-} cells were transfected with either pSG5 (empty vector), or expression vectors encoding either RNase L or the indicated REAVRs. The rRNA degradation products were assessed at 6 and 24 hr after infection with vaccinia virus (VACV) VC-R4, which lacks PKR antagonists K3L and E3L. RNA cleavage products were observed in cells expressing RNase L and REAVRs as early as 6 hours post infection (FIG. 5). The RNA degradation was more pronounced at 24 hours post infection, while empty vector-transfected cells did not show rRNA degradation products. RNA cleavage products were detected in all cells expressing REAVRs, to a lesser extend in REAVR 5, which contains the entire sequence of Syrian hamster PKR. Interestingly, REAVR 6, including full-length inactivated Syrian hamster PKR and human RNase L, showed rRNA cleavage products, indicating that the RNase L effector domain in this construct is active. Altogether, this rRNA degradation assay indicates the RNase L effector domain of REAVRs is functionally active.

To further investigate whether the level of protein expression contributes to the degree of rRNA degradation, A549^{RNase L-/-/PKR-/-} cells were transfected with the same amount of plasmid, and Western blots analyses were performed. These blots did not detect endogenous RNase L expression in the pSG5 transfected cells (FIG. 6). RNase L was expressed at higher level than REAVRs, which might explain why the cells expressing RNase L had the highest RNA degradation activity. Interestingly, despite a lower level of expression in cells transfected with either REAVR 7 and 8, the RNA cleavage assay showed a slightly higher degree of RNA degradation than for REAVR 3 and 4. In addition, REAVR 6, which contains an inactive PKR, was observed to migrate faster on the gel than REAVR 5, which contains wild-type Syrian hamster PKR. The slower mobility of wild-type PKR compared to inactive PKR is most likely due to autophosphorylation of the wild-type protein (F. Zheng et al., J. Biol. Chem. 276, (2001): 24946). Overall, these transfection-based luciferase reporters and RNA-integrity assays show that these REAVRs decreased luciferase expression and led to RNA degradation, indicating that the REAVRs are functionally active.

### Example 2. Expression of a Single Copy of Each REAVR in T-REX™ Stable Cell Line Systems and its Effect on Cell Viability

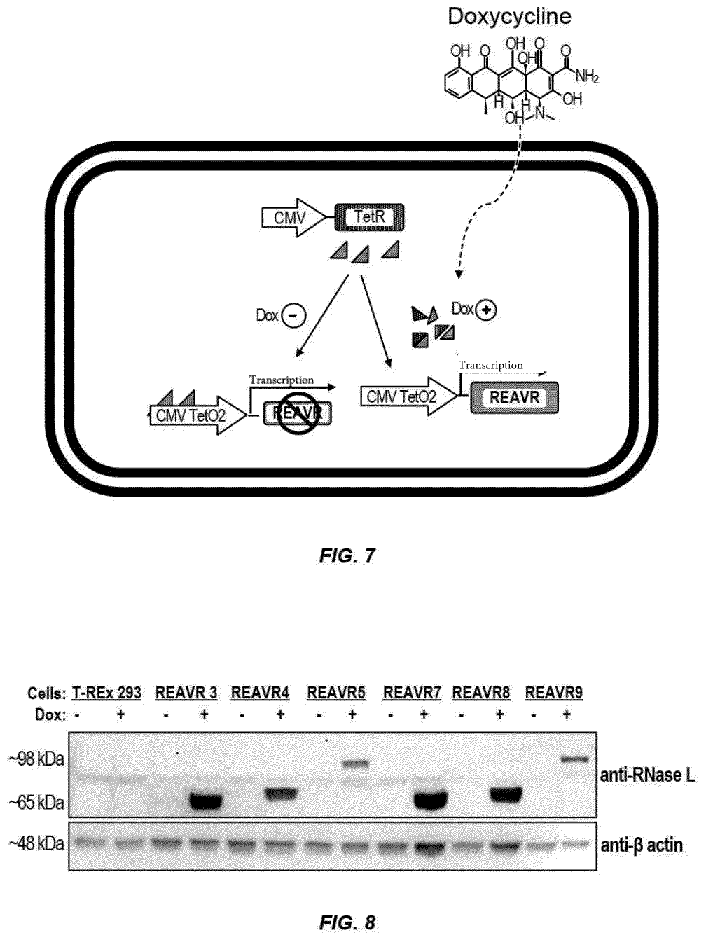

In order to determine whether REAVRs have antiviral effects in mammalian cells, Flp-In™ T-REX™ (T-REX) cells (Invitrogen) were generated, where the cells stably expressed a single copy of each REAVR under the control of a doxycycline-inducible promoter. The T-REX system includes a stably expressed tetracycline repressor (TetR) gene from E. coli, a promoter with two tetracycline repressor binding sites, and a single gene-of-interest Flp Recombination Target (FRT) integration site. Integration of the gene of interest into the FRT site is mediated by Flp recombinase from Saccharomyces cerevisiae (S. O'Gorman, D. T. Fox & G. M. Wahl, Science 251, (1991): 1351). In this Tet-On system, the TetR repressor constitutively inhibits expression of the gene of interest unless tetracycline or doxycycline is present to bind the TetR repressor (FIG. 7). Thus, REAVR expression in the T-REX cell system is controlled by the presence of tetracycline or doxycycline in the media. Western blot analyses showed that there is little if any leaky expression in the absence of doxycycline. All REAVRs were expressed at 24 hours post doxycycline induction with relatively weaker expression of REAVRs 5 and 9 (FIG. 8).

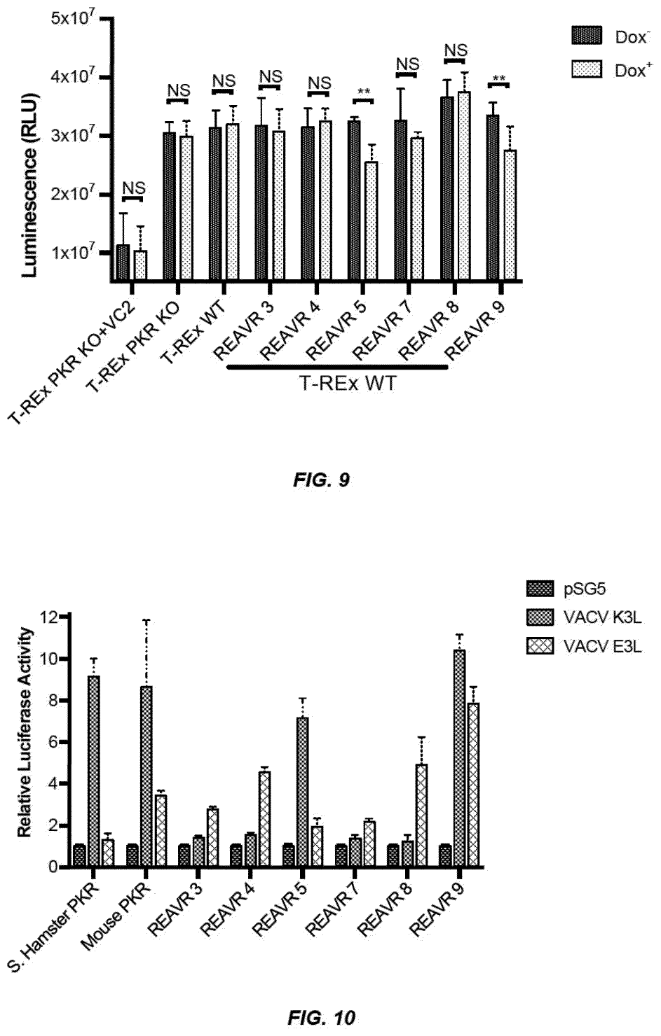

To determine the effects of REAVRs on cell viability, the Viral ToxGlo™ assay (Promega) was used to determine the number of viable cells in culture. The assay quantifies ATP as a proxy for the presence of metabolically active cells. The luminescence readout is proportional to the number of viable cells in culture. T-REX PKR^{ko} infected with vaccinia virus (VACV) Copenhagen strain (VC-2) served as positive control and T-REX WT cells with and without doxycycline treatment served as a negative control. As expected, relative light unit (RLU) doxycycline-treated and untreated parental T-REX WT cells was not significantly different, suggesting no significant effects of doxycycline treatment on cell viability. In contrast, low RLU was observed in T-REX PKR^{ko} cells infected with VACV at 48 hours post infection (FIG. 9). Similar to the parental T-REX WT, the RLU values between doxycycline and non-doxycycline treated cells were not significantly different in REAVR 3-, 4-, 7-, and 8-expressing cells. These data indicate that expression of REAVR 3, 4, 7 and 8 did not cause cytotoxicity at 0.5 μg/ml doxycycline induction for 48 hours. Notably, the RLU value of REAVR 7 expressing cells was slightly lower than in untreated cells, indicating REAVR 7 might slightly affect cell health. In contrast, the RLU values of doxycycline-treated T-REX WT REAVR 5 and 9 cells were significantly lower than the untreated cells, suggesting that expression of REAVR 5 and 9 might negatively affect cell viability. Overall, the cell viability test confirmed that the integrated REAVRs had no adverse effects on cells without doxycycline, while control cells infected with VACV had substantially reduced viability (FIG. 9). Furthermore, REAVRs did not significantly reduce cell viability even 48 hours after doxycycline induction, with exception of REAVR 5 and 9, which both contain full PKR protein.

### Example 3. Antiviral Effects of REAVRs on Vaccinia Virus

Luciferase-based assays were performed to test the sensitivity of REAVRs to the VACV innate immune antagonists E3L and K3L. Human HeLa PKR^{ko} cells were transfected with expression vectors for individual REAVRs, vaccinia virus viral antagonists E3L or K3L, and firefly luciferase. As a control, cells were also transfected without viral antagonists to determine fully active REAVR luciferase inhibition. Syrian hamster PKR and mouse PKR served as positive controls. Results at 48 hours after showed differential sensitivity of the REAVRs to these viral antagonists (FIG. 10). REAVRs 3 and 4, including Syrian hamster PKR sensor domain and human RNase L effector domain, were sensitive to E3L but resistant to K3L. REAVR 5, which includes full-length Syrian hamster PKR and human RNase L, was sensitive to K3L and largely resistant to E3L, resembling the Syrian hamster PKR sensitivity profile. Similarly, REAVR 9, which includes full-length mouse PKR and human RNase L effector domain, also resembled the sensitivity profile of mouse PKR. Interestingly, REAVR 7 was largely resistant to E3L while REAVR 8 was sensitive. Since K3L targets the kinase domain of PKR, the lack of inhibition of REAVRs devoid of the PKR kinase domain was predicted, whereas the relative resistance of REAVR 7 to E3L was surprising and indicates that the linker region of PKR is involved in the inhibition by E3L.

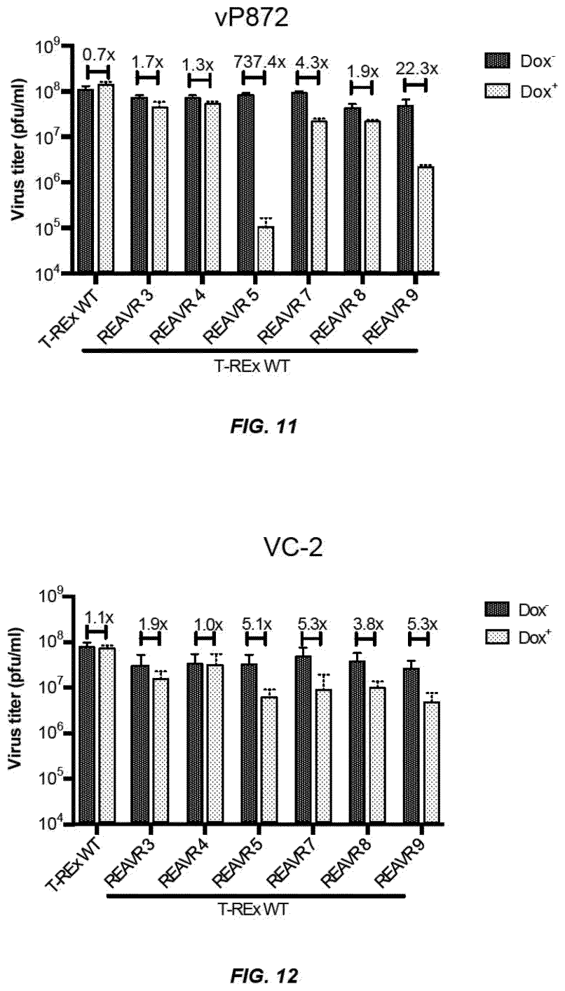

Additional assays tested the ability of REAVRs to restrict a variant vaccinia virus that lacks the K3L gene but expresses the E3L gene (vP872), and wild-type vaccinia virus that is expressing both K3L and E3L genes. First, parental T-REX WT and T-Rex WT cells stably expressing REAVRs were infected with vP872. No difference in the virus titer of doxycycline-treated and untreated parental T-REX WT was observed. REAVR 3 and 4 appear to have minimal or no effect on vP872 (FIG. 11). In contrast, REAVR 5 expression diminished virus titer by about 700-fold compared to REAVR 5 untreated cells. To a lesser extent, REAVR 9 diminished virus titer by about 20-fold compared to REAVR 9 untreated cells. REAVR 7 showed minimal effect on vP872 with 4.3-fold reduction of virus titer. This result indicated that the PKR kinase activity was more important in inhibiting vP872 than the RNase activity. This finding was not surprising for REAVR 5 that contains active Syrian hamster PKR kinase domain, as Syrian hamster PKR was shown to be resistant to VACV E3L. However, this is unique for REAVR 9 as it includes mouse PKR, which was known to be sensitive to VACV E3L (S. Rothenburg, E. J. Seo, J. S. Gibbs, T. E. Dever & K. Dittmar, Nat. Struct. Mol. Biol. 16, (2009): 63).

As a further test of the effect of vaccinia E3L and K3L on virus replication in REAVR expressing cells, these cells were infected with wild-type VACV strain Copenhagen (VC-2). No difference in the virus titer of doxycycline-treated and untreated parental T-REx WT and REAVR 4 expressing cell lines was observed (FIG. 12). REAVR 5 expression only slightly reduced the VC-2 virus titer by about 5-fold, suggesting that VACV K3L dramatically rescued virus replication. There was also about 5-fold reduction of VC-2 virus titer in doxycycline-treated REAVR 7, 8, and 9 stable cell lines compared to doxycycline untreated cells.

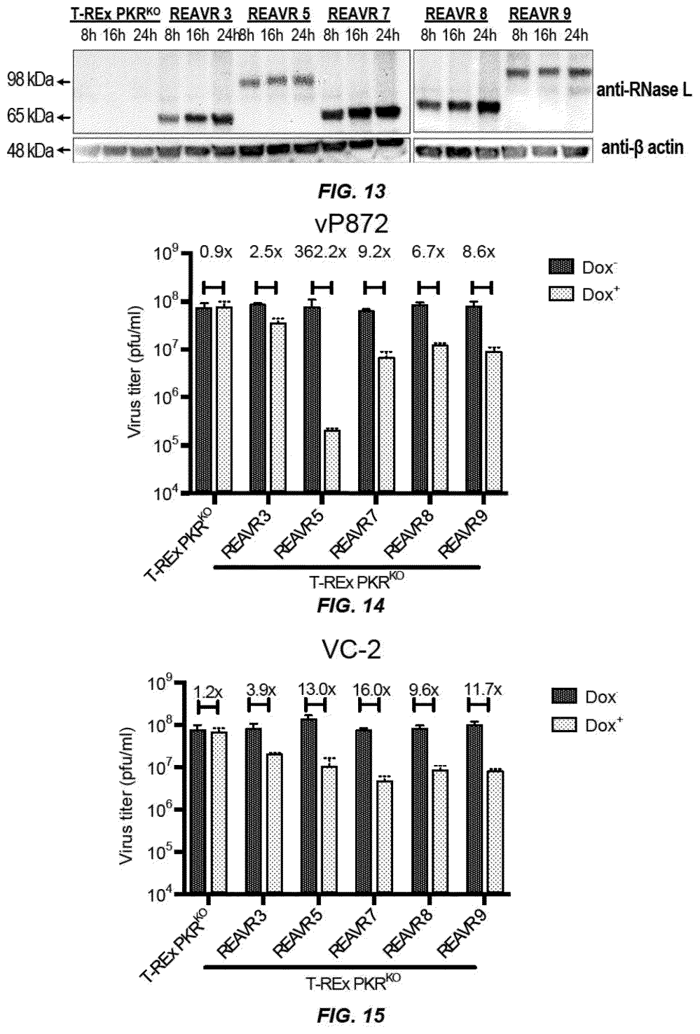

Human PKR has been shown to be sensitive to VACV E3L but resistant to VACV K3L (C. Park et al., PLOS Pathog. 17, (2021): e1009183; S. Rothenburg, E. J. Seo, J. S. Gibbs, T. E. Dever & K. Dittmar, Nat. Struct. Mol. Biol. 16, (2009): 63; and S. Rothenburg, V. G. Chinchar & T. E. Dever, BMC Microbiol. 11, (2011): 56). To avoid confounding effects with PKR, a PKR-deficient T-REX cell line was used in which the PKR was knocked out by CRISPR-Cas9, and then stably transfected cells were generated with REAVRs 3, 5, 7, 8 and 9. Time-dependent doxycycline induction was conducted to see REAVRs expression at 8, 16, and 24 hours. Immunoblot analysis showed that all REAVRs were expressed at 8 hours post-induction and the expression increased over time (FIG. 13). Both parental T-REX PKR^{ko} and T-REX PKR^{ko} cells stably expressing REAVRs were infected with either vP872 or VC-2 (FIGS. 14 and 15). Although the general trend of virus inhibition obtained from this infection assay was similar to that of T-REx WT cells, there was a notably stronger reduction of viral titer between doxycycline-treated and untreated cells for most REAVR-expressing cells, suggesting endogenous PKR might interfere with the antiviral activity of REAVRs.

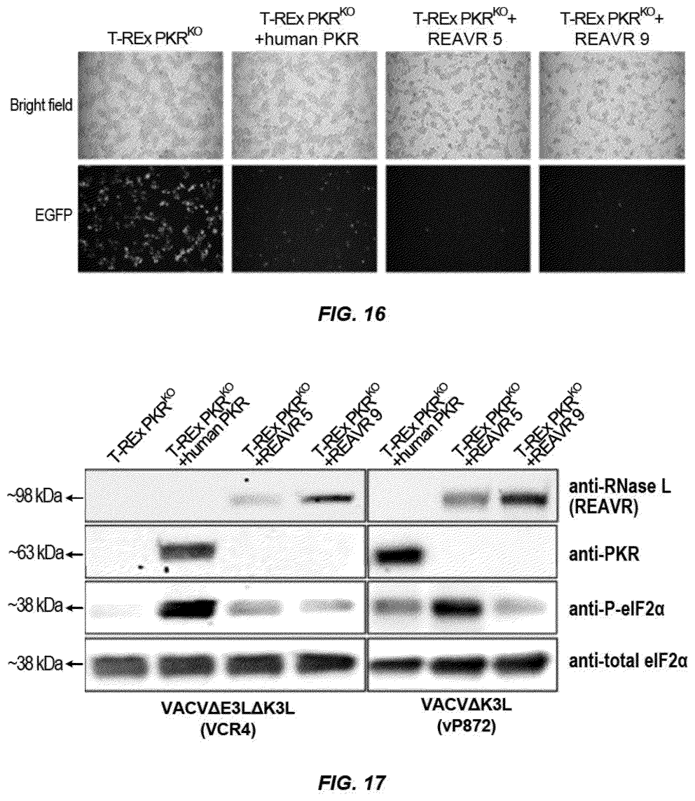

Because of the notable antiviral effects of REAVRs 5 and 9 on vaccinia virus, further tests were run to determine whether REAVR 5 and REAVR 9 phosphorylate eIF2α. T-Rex PKR^{ko} was used as a negative control, and T-REX PKR^{ko} stably expressing human PKR served as positive control. All cells were treated with 0.5 μg of doxycycline at 24 hours before being infected with the VACV E3L and K3L double-knockout strain VC-R4 (VC-R4) or vP872 at an MOI of 3. Use of VC-R4, which expresses EGFP under control of the natural E3L promoter, allowed the monitoring of virus replication using fluorescence microscopy. A robust EGFP signal was observed in T-REX PKR^{ko} cells, with a considerably lower EGFP signal observed in T-REX PKR^{ko} expressing human PKR, as expected. Remarkably less EGFP was observed in REAVR 5 and 9 expressing cells at 6 hours post infection relative to T-REX PKR^{ko} and T-REX PKR^{ko}-human PKR (FIG. 16). Cells were photographed, and then lysates were harvested for western blot analysis using an antibody specific for eIF2a phosphorylation at S51. Only little eIF2a phosphorylation was observed in the absence of PKR (FIG. 17). Human PKR induced a high level of eIF2α phosphorylation in VC-R4 infected cells. Although eIF2α phosphorylation levels in both REAVR 5 and 9 expressing cells infected with VC-R4 were lower than in human PKR-expressing cells, the levels of eIF2a phosphorylation in REAVR 5 and 9 cells were comparable. Upon infection with vP872, the eIF2α phosphorylation level was lower in human PKR-expressing cells infected with vP872 than VCR4 infected cells, which can be explained by the sensitivity of human PKR to E3L. In contrast, eIF2a phosphorylation levels where higher in REAVR 5-expressing cells, which correlates with the high inhibition of virus replication (FIG. 14) and can be explained by the resistance of Syrian hamster PKR to E3L. eIF2α phosphorylation levels in in REAVR 9-expressing cells, was more similar to that found in human PKR-expressing cells.

### Example 4. Antiviral Effects of REAVRs on Dengue Virus and Zika Virus

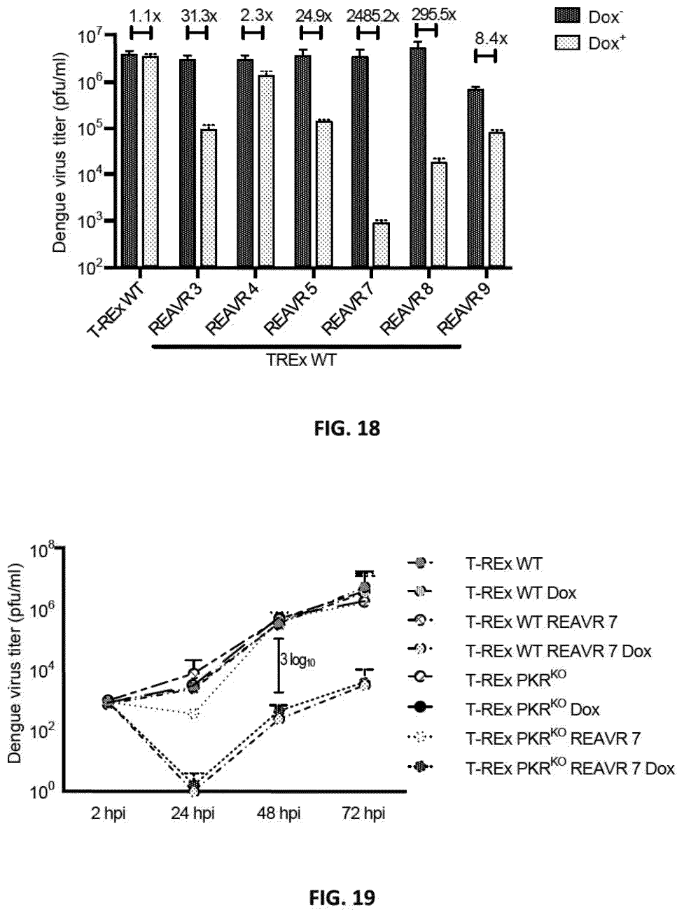

The antiviral effects of REAVRs against two members of flaviviruses, dengue virus serotype 2 strain 16681 (DENV2) and Zika virus (ZIKV) strain PRVABC59, which are both +ssRNA viruses, were also tested. Replication of DENV2 was assessed by infecting REAVR-expressing T-REX cells and determining the virus titers at 48 hours post infection. There was no substantial difference in the DENV2 titer of doxycycline-treated and untreated parental T-REX WT (FIG. 18). REAVR 4 and 9 showed a minimal to moderate antiviral effects on the DENV2 with a 2- to 8-fold reduction in viral titers, respectively. Induction of REAVRs 3 and 5 showed stronger effects on DENV2 with about 31- and 25-fold reduction in virus titer, respectively. REAVRs 7 and 8 diminished virus titer by 2,485 and 295-fold compared to doxycycline untreated cells, respectively. Distinct antiviral effects of REAVR 7 and 8 suggests the linker region between the PKR and RNase L domains influence the antiviral activity of REA VRs. To test if endogenous PKR influences the effect of REAVR 7 on the viral replication, T-REX WT, T-REX PKR^{ko}, T-REX WT-REAVR7 and TREX PKR^{ko}-REAVR7 were infected with DENV2 at MOI of 0.1 in the presence and absence of doxycycline. Infectious particle production was monitored over time by measuring viral titers from supernatants (FIG. 19). Viral titers were similar across cell lines at 2 hours post infection. Both T-REX PKR^{ko}-REAVR7 and T-REX WT-REAVR7 doxycycline treated-cells showed about 1,000 less virus titer compared to doxycycline untreated cells, at 24-, 48-, and 72-hours post infection. There was no substantial difference between virus titers in T-REX PKR^{ko} and T-REX WT cells. This finding supports the conclusion that DENV replication is independent of PKR (M. S. Diamond, & E. Harris, Virology 289, (2001): 297) and that REAVR 7 activity was not influenced by endogenous PKR.

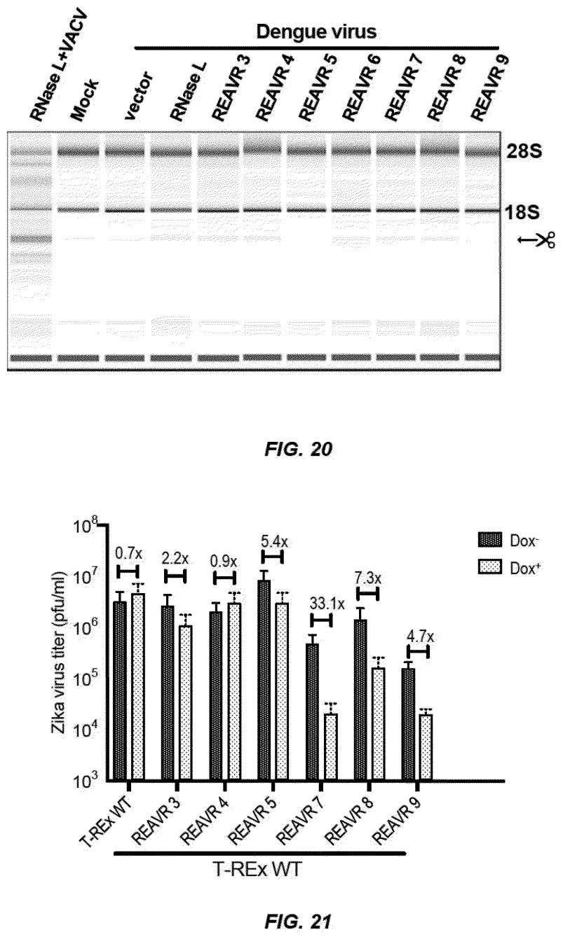

RNase L activity of REAVRs after dengue virus infection was next assessed by monitoring rRNA degradation products using a 2100 Bioanalyzer to test whether the reduction of virus titers correlated with rRNA degradation. Because T-REX cells have endogenous RNase L, A549^{RNase L-/-/PKR-/-} cells were used to test rRNA cleavage activities, as described above for VACV. A549^{RNase L-/-/PKR-/-} cells were transiently transfected with the indicated plasmids. About 24 hours after transfections, the cells were mock-infected or infected with dengue serotype 2 at an MOI of 3. RNA degradation was assessed at 24 hours post infection (FIG. 20). Surprisingly, the rRNA degradation products were not as prominent as when the cells were infected with VACV. However, there were noticeable rRNA cleavage products in all cells expressing REAVRs, except for REAVRs 5 and 9. Of note, cleavage products in REAVR 7 and 8 transfected cells were not more prominent than in REA VR 3 and 4 transfected cells, indicating a lack of correlation between rRNA cleavage and antiviral activities.

Because some REAVRs showed strong antiviral effects against DENV, another assessment was used to test whether REAVRs exhibit antiviral effects against ZIKV. There was minimal or no effect of REAVR 3 and 4 against ZIKV (FIG. 21). REAVR 5, 7, 8, and 9 showed a moderate antiviral effect on ZIKV replication. REAVR 7 mediated the strongest antiviral effect on ZIKV replication (33-fold reduction). None of the REAVRs had pro-viral effects on ZIKV and there was a similar trend of virus inhibition as for DENV2, although the antiviral effects were lower than observed for DENV2.

### Example 5. Inhibition of SARS-COV-2 Replication by Expression of REAVR 7

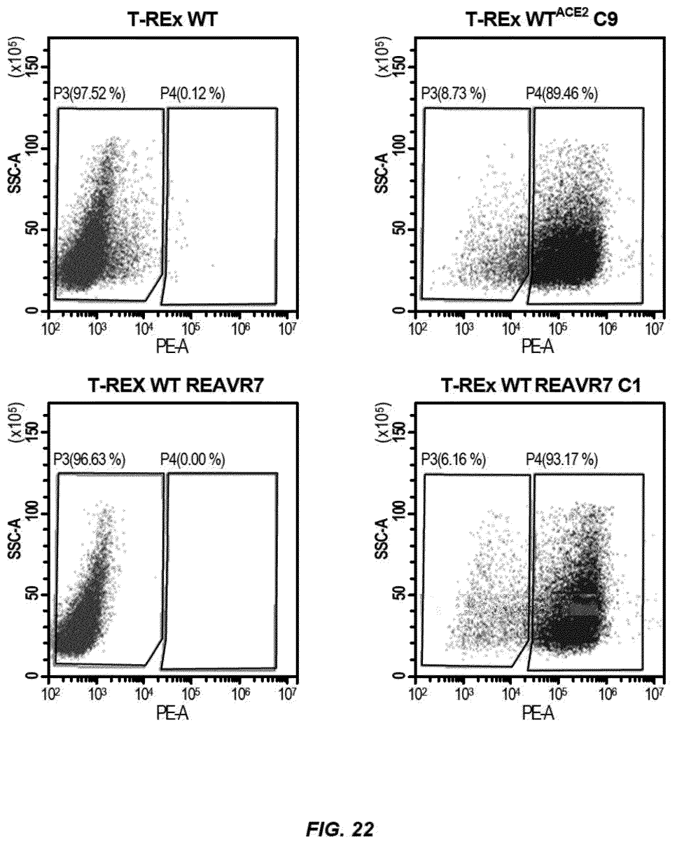

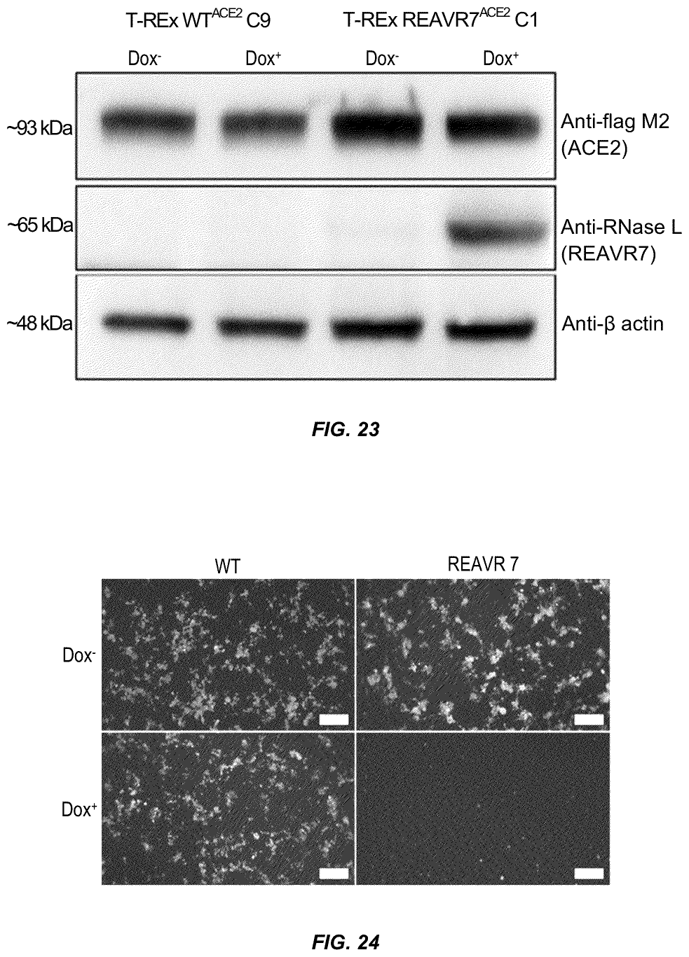

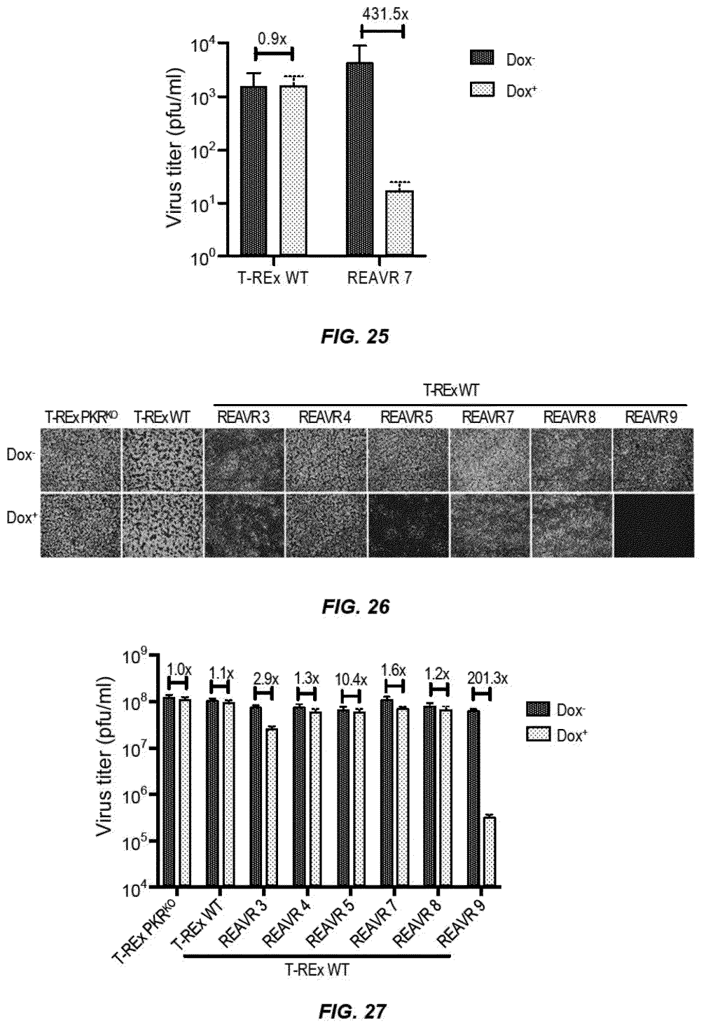

SARS-COV-2, arguably one of the most intensely studied viruses since 2020, has been shown to promote PKR and RNase L activation in a lung cancer cell lines A549 (Li et al., Proc. Natl Acad. Sci. USA 118, (2021)). Because SARS-COV-2 and DENV2 are both +ssRNA viruses, and due to the strong antiviral effect of REAVR 7 on DENV2 and ZIKV, the ability of REAVR 7 to inhibit SARS-COV-2 replication was also assessed. To facilitate SARS-COV-2 infection, T-REX WT and T-Rex REAVR 7 cells stably expressing the human ACE2 receptor were generated. Selected monoclonal cell lines T-REX WT^{ACE2} C9 and T-REX REAVR7ACE2 C1 were then used for experiments. ACE2 expression in both cell lines was robustly detected by flow cytometry using antibodies against human ACE2, which showed about 89.46% cells expressing ACE2 in T-REX WT^{ACE2} C9 and about 93.17% cells expressing ACE2 in T-REX REAVR7ACE2 C1 (FIG. 22). In contrast, there was no ACE2 expression in untransfected cells. The level of ACE2 expression was also validated using Western blot analysis (FIG. 23), which showed ACE2 expression in all cell lines. In addition, Western blot analysis confirmed REAVR7 expression when the T-REX REAVR7^{ACE2} C1 cells were treated with doxycycline. An infection assay with SARS-COV-2 mNG at an MOI of 1 showed SARSCoV2 mNG replicated robustly in both doxycycline-treated and control T-REX WT^{ACE2} C9 cells (FIG. 24). In contrast, SARS-COV-2 only replicated robustly in T-Rex REAVR7^{ACE2} C1 control cells but not in doxycycline-treated cells. Consistent with the green florescence signal, there was an about 431-fold (2.7-log) reduction in virus titer in TREx REAVR7^{ACE2} C1 doxycycline-treated cells compared to the control T-Rex REAVR7^{ACE2} C1 cells (FIG. 25). This finding demonstrate REAVR 7 is a potent antiviral protein against SARS-COV-2.

### Example 6. Antiviral Effects of REAVRs on Vesicular Stomatitis Virus

To test the antiviral effects of REAVRs on an -ssRNA virus, T-REX PKR^{ko}, the parental T-REX WT and T-REX WT expressing REAVRs were infected with VSV-12′GFP at an MOI of 0.01. VSV-12′GFP is an attenuated vesicular stomatitis virus derived from the Indiana serotype (A. N. van den Pol & J. N. Davis, J. Virol. 87, (2013): 1019). VSV-12′GFP replicated robustly in T-REX PKR^{ko} and T-REX WT cells with almost all cells showing a strong GFP signal 24 hours after infection (FIG. 26). Among the REAVRs, only REAVRs that contain full-length PKR restricted VSV-12′GFP replication. Interestingly, REAVR 9 that contains mouse PKR had a more substantial effect, resulting in a 201-fold reduction, while in REAVR 5 that contains Syrian hamster PKR caused a 10-fold reduction compared to doxycycline untreated cells (FIG. 27). Notably, REAVR 7, which had a strong effect on dengue virus, Zika virus, and SARSCoV2 mNG appeared to have minimal effect on VSV-12′GFP, indicating that VSV replication is independent of RNase L activation.

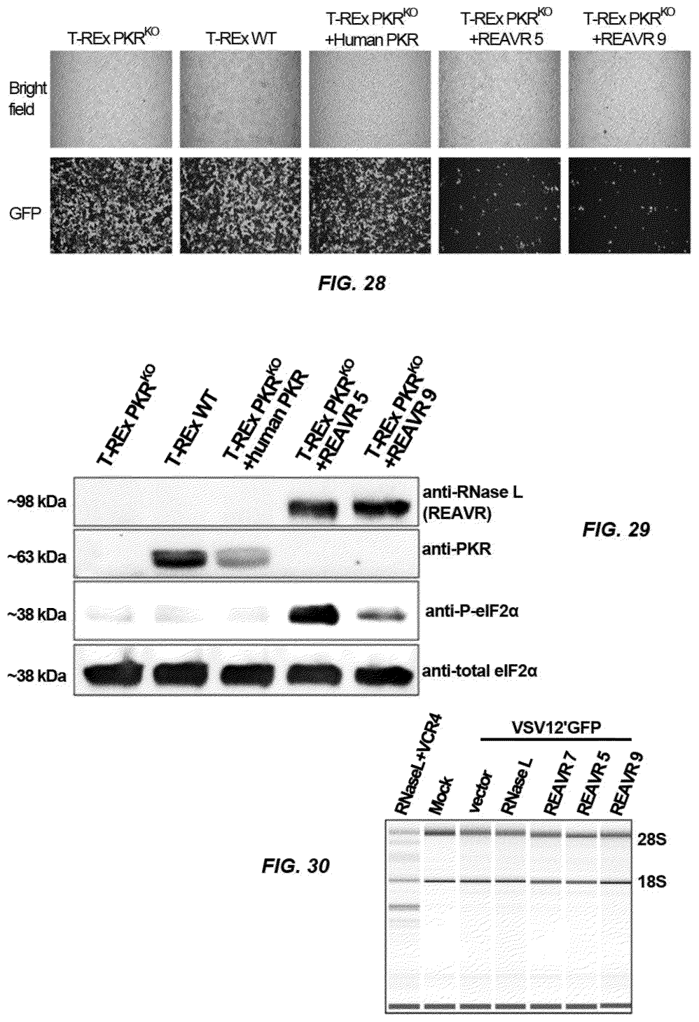

Additional tests determined whether the inhibition of VSV-12′GFP replication correlated with eIF2a phosphorylation. The indicated cells were infected with VSV-12′GFP MOI of 0.1 (FIG. 28). All cells were treated with 0.5 μg of doxycycline for 6 hours before VSV-12′GFP infections. Strong GFP signals were observed in T-REx PKR^{ko}, T-REX WT, and TREx PKR^{ko} expressing human PKR after infection with VSV-12′GFP, whereas considerably less GFP signal was observed in REAVR 5 and 9 expressing cells at 14 hours post infection. In addition, Western blotting results showed high levels of eIF2a phosphorylation only in REAVR 5 and 9 expressing cells, with a stronger signal in the former (FIG. 29).

Unlike the T-REX WT expressing REAVR 5 that showed a higher GFP signal than REAVR 9 expressing cells, there was a comparable GFP signal in T-REX PKR^{ko} expressing REAVR 5 and 9. RNase L activity of REAVRs was also assessed by monitoring rRNA degradation products using a 2100 Bioanalyzer. A549 RNase L and PKR double knockout cells were transiently transfected with either pSG5 empty vector, RNase L encoding plasmid, or the indicated REAVR encoding plasmids. About 24 hours after transfections, the cells were mock-infected or infected with VSV-12′GFP MOI of 1. RNA degradation was assessed at 24 hours post infection (FIG. 30). No rRNA cleavage products were observed in cells transfected with RNase L or REAVRs. This result is consistent with a previous finding that showed that VSV infection in A549 cells failed to generate detectable levels of RNase L-mediated rRNA cleavage, indicating minimal or no antiviral effect of RNase L (Li et al., Proc. Natl Acad. Sci. USA 118, (2021)).

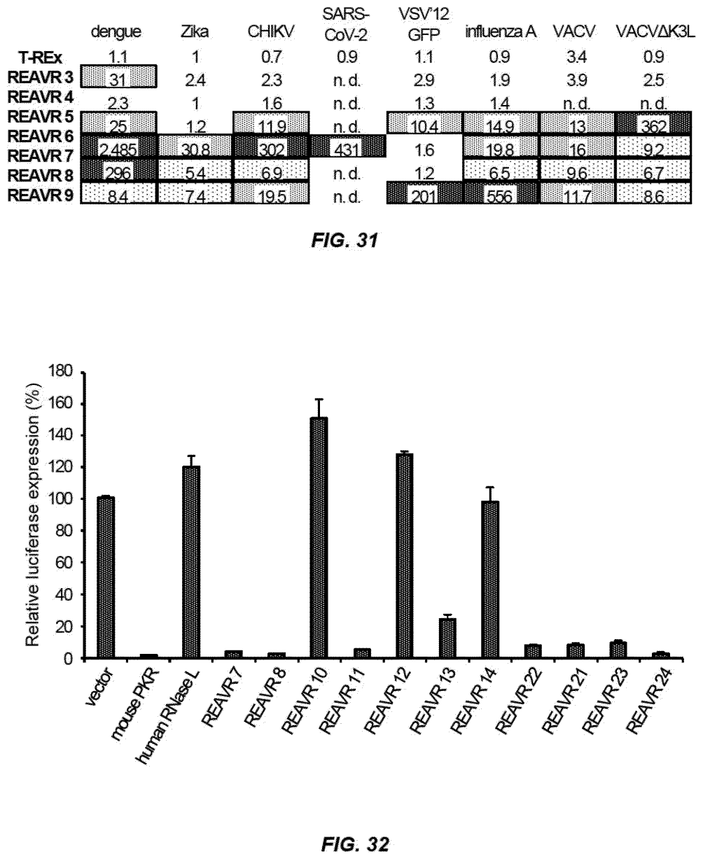

In summary, all tested viruses showed sensitivity to the REAVR fusion proteins provided by this disclosure (FIG. 31). DENV2 was the most sensitive to REAVR-mediated antiviral activity, with titers reduced by more than 100-(REAVR 8) or 1,000-fold (REAVR 7). REAVR 7 also mediated the strongest antiviral effect on ZIKV replication (30-fold reduction). In contrast, VSV replication was most strongly inhibited by REAVR 9. REAVR 7 in particular restricted 6 different viruses from 5 virus families. These restricted viruses include wild type vaccinia and influenza A viruses, which were tested because both encode well-defined inhibitors (VACV E3L and IAV NS1) of both the PKR and RNase L pathways. These data therefore demonstrate that the provided REAVR fusion proteins have broad activities and can evade multiple viral escape strategies.

### Example 7. Activities of REAVRs Containing Diverse Effector Domains

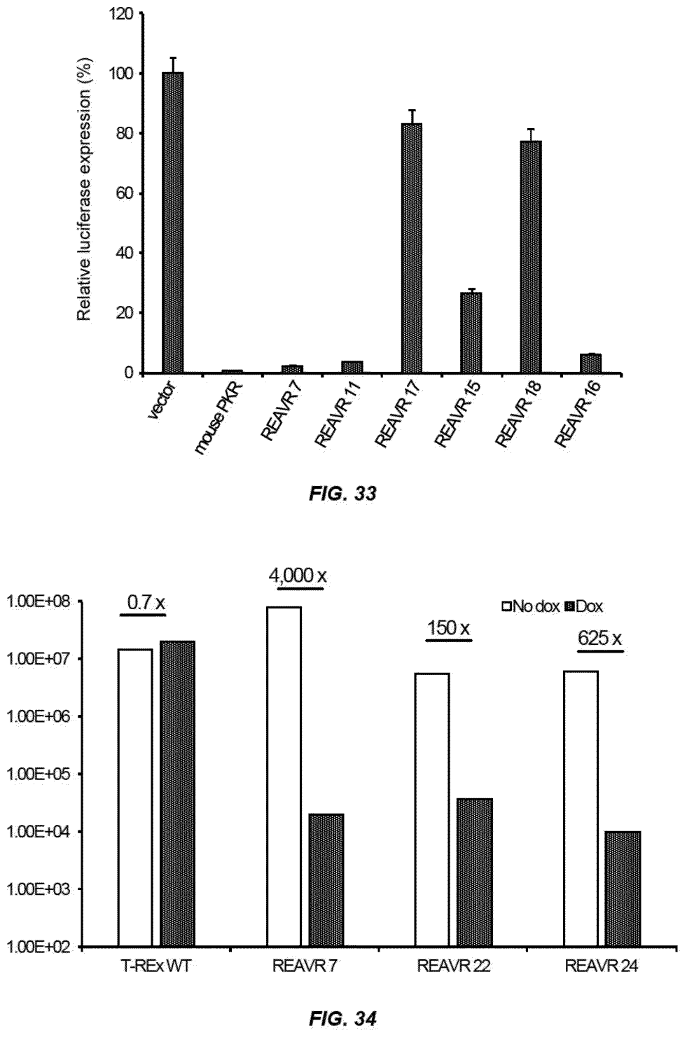

Further tests were conducted using additional REAVRs that contain RNase L domains from Microcaecilia unicolor (REAVR10), African penguin (REAVR11), great white shark (REAVR12), Burmese python (REAVR13), and Latimeria chalumnae (REAVR14), fused to the dsRNA-biding domains of mouse PKR. FIG. 32 presents a graph plotting results from testing the activities of these additional REAVRs in the luciferase-based assay. Of these constructs, REAVR11 and REAVR13 showed considerable activity. Other tests were conducted using REAVRs that contain RNase L domains from Iberian mole (REAVR15), American pika (REAVR16), monito del monte (REAVR17), and Australian echidna (REAVR18) (FIG. 33). Each of these REAVRs showed activity, with strongest signals detected for REAVR15 and REAVR16. Additional assays tested REAVRs including the fusion protein of the nucleic acid binding domains of zebrafish PKR (REAVR21), zebrafish PKZ (REAVR22), human ZBP1 F1 (REAVR23), and human ZBP1 F2 (REAVR24) (FIG. 32). Each of these additional REAVRs showed strong activity as evidenced by the reduction of luciferase expression.

T-REX cell lines expressing stably expressing REAVR 22 and REAVR 24 were constructed and used to infect cells in which REAVR expression was uninduced (no dox) or induced (+Dox) with Dengue virus 2 for 48 hours. Induction of REAVR expression led to a strong inhibition of Dengue virus replication in both REAVR 22 (150 fold) and REAVR 24 (624 fold) after doxycycline induction (FIG. 34).

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only and is not intended to limit the disclosure. In addition, each reference provided herein is incorporated by reference in its entirety for all purposes to the same extent as if each reference was individually incorporated by reference.

SEQUENCE LISTING

| SEQ | | |
|---|---|---|
| ID | | |
| NO | Sequence | Description |
| 1 | KIADTSEGGIYLGFYEKQEVAVKTFCEGSPRAQREVSCLQSSRENSHLVTFYGSESHRGHLFV | Human RNase L part |
| | CVTLCEQTLEACLDVHRGEDVENEEDEFARNVLSSIFKAVQELHLSCGYTHQDLQPQNILID | amino acids 375-741 |
| | SKKAAHLADFDKSIKWAGDPQEVKRDLEDLGRLVLYVVKKGSISFEDLKAQSNEEVVQLSPD | (starts with β-sheet 1) |
| | EETKDLIHRLFHPGEHVRDCLSDLLGHPFFWTWESRYRTLRNVGNESDIKTRKSESEILRLLQ | |
| | PGPSEHSKSFDKWTTKINECVMKKMNKFYEKRGNFYQNTVGDLLKFIRNLGEHIDEEKHKK | |
| | MKLKIGDPSLYFQKTFPDLVIYVYTKLQNTEYRKHFPQTHSPNKPQCDGAGGASGLASPGC | |
| 2 | IDEKYKIADTSEGGIYLGFYEKQEVAVKTFCEGSPRAQREVSCLQSSRENSHLVTFYGSESHR | Human RNase L part |
| | GHLFVCVTLCEQTLEACLDVHRGEDVENEEDEFARNVLSSIFKAVQELHLSCGYTHQDLQP | amino acids 370-741 |
| | QNILIDSKKAAHLADFDKSIKWAGDPQEVKRDLEDLGRLVLYVVKKGSISFEDLKAQSNEEV | |
| | VQLSPDEETKDLIHRLFHPGEHVRDCLSDLLGHPFFWTWESRYRTLRNVGNESDIKTRKSES | |
| | EILRLLQPGPSEHSKSFDKWTTKINECVMKKMNKFYEKRGNFYQNTVGDLLKFIRNLGEHID | |
| | EEKHKKMKLKIGDPSLYFQKTFPDLVIYVYTKLQNTEYRKHFPQTHSPNKPQCDGAGGASG | |
| | LASPGC | |
| 3 | IDEKYKIADTSEGGIYLGFYEKQEVAVKTFCEGSPRAQREVSCLQSSRENSHLVTFYGSESHR | Human RNase L part |
| | GHLFVCVTLCEQTLEACLDVHRGEDVENEEDEFARNVLSSIFKAVQELHLSCGYTHQDLQP | amino acids 370-701 |
| | QNILIDSKKAAHLADFDKSIKWAGDPQEVKRDLEDLGRLVLYVVKKGSISFEDLKAQSNEEV | |
| | VQLSPDEETKDLIHRLFHPGEHVRDCLSDLLGHPFFWTWESRYRTLRNVGNESDIKTRKSES | |
| | EILRLLQPGPSEHSKSFDKWTTKINECVMKKMNKFYEKRGNFYQNTVGDLLKFIRNLGEHID | |
| | EEKHKKMKLKIGDPSLYFQKTF | |
| 4 | WGAALKDLHRIYRPMIGKLKFFIDEKYKIADTSEGGIYLGFYEKQEVAVKTFCEGSPRAQREV | Human RNase L part |
| | SCLQSSRENSHLVTFYGSESHRGHLFVCVTLCEQTLEACLDVHRGEDVENEEDEFARNVLSSI | amino acids 348-703 |
| | FKAVQELHLSCGYTHQDLQPQNILIDSKKAAHLADFDKSIKWAGDPQEVKRDLEDLGRLVL | |
| | YVVKKGSISFEDLKAQSNEEVVQLSPDEETKDLIHRLFHPGEHVRDCLSDLLGHPFFWTWES | |
| | RYRTLRNVGNESDIKTRKSESEILRLLQPGPSEHSKSFDKWTTKINECVMKKMNKFYEKRGN | |
| | FYQNTVGDLLKFIRNLGEHIDEEKHKKMKLKIGDPSLYFQKTFPD | |
| 5 | IDEKYKIADTSEGGIYLGFYEKQEVAVKTFCEGSPRAQREVSCLQSSRENSHLVTFYGSESHR | Human RNase L part |
| | GHLFVCVTLCEQTLEACLDVHRGEDVENEEDEFARNVLSSIFKAVQELHLSCGYTHQDLQP | amino acids 370-715 |
| | QNILIDSKKAAHLADFDKSIKWAGDPQEVKRDLEDLGRLVLYVVKKGSISFEDLKAQSNEEV | |
| | VQLSPDEETKDLIHRLFHPGEHVRDCLSDLLGHPFFWTWESRYRTLRNVGNESDIKTRKSES | |
| | EILRLLQPGPSEHSKSFDKWTTKINECVMKKMNKFYEKRGNFYQNTVGDLLKFIRNLGEHID | |
| | EEKHKKMKLKIGDPSLYFQKTFPDLVIYVYTKLQNT | |
| 6 | ALKDLHRIYRPMIGKLKFFIDEKYKIADTSEGGIYLGFYEKQEVAVKTFCEGSPRAQREVSCLQ | Human RNase L part |
| | SSRENSHLVTFYGSESHRGHLFVCVTLCEQTLEACLDVHRGEDVENEEDEFARNVLSSIFKA | amino acids 351-726 |
| | VQELHLSCGYTHQDLQPQNILIDSKKAAHLADFDKSIKWAGDPQEVKRDLEDLGRLVLYVV | |
| | KKGSISFEDLKAQSNEEVVQLSPDEETKDLIHRLFHPGEHVRDCLSDLLGHPFFWTWESRYR | |
| | TLRNVGNESDIKTRKSESEILRLLQPGPSEHSKSFDKWTTKINECVMKKMNKFYEKRGNFYQ | |
| | NTVGDLLKFIRNLGEHIDEEKHKKMKLKIGDPSLYFQKTFPDLVIYVYTKLQNTEYRKHFPQT | |
| | HS | |
| 7 | KLKFFIDEKYKIADTSEGGIYLGFYEKQEVAVKTFCEGSPRAQREVSCLQSSRENSHLVTFYGS | Human RNase L part |
| | ESHRGHLFVCVTLCEQTLEACLDVHRGEDVENEEDEFARNVLSSIFKAVQELHLSCGYTHQD | amino acids 365-706 |
| | LQPQNILIDSKKAAHLADFDKSIKWAGDPQEVKRDLEDLGRLVLYVVKKGSISFEDLKAQSN | |
| | EEVVQLSPDEETKDLIHRLFHPGEHVRDCLSDLLGHPFFWTWESRYRTLRNVGNESDIKTRK | |
| | SESEILRLLQPGPSEHSKSFDKWTTKINECVMKKMNKFYEKRGNFYQNTVGDLLKFIRNLGE | |
| | HIDEEKHKKMKLKIGDPSLYFQKTFPDLVI | |
| 8 | HRIYRPMIGKLKFFIDEKYKIADTSEGGIYLGFYEKQEVAVKTFCEGSPRAQREVSCLQSSREN | Human RNase L part |
| | SHLVTFYGSESHRGHLFVCVTLCEQTLEACLDVHRGEDVENEEDEFARNVLSSIFKAVQELH | amino acids 356-707 |
| | LSCGYTHQDLQPQNILIDSKKAAHLADFDKSIKWAGDPQEVKRDLEDLGRLVLYVVKKGSIS | |
| | FEDLKAQSNEEVVQLSPDEETKDLIHRLFHPGEHVRDCLSDLLGHPFFWTWESRYRTLRNV | |
| | GNESDIKTRKSESEILRLLQPGPSEHSKSFDKWTTKINECVMKKMNKFYEKRGNFYQNTVG | |
| | DLLKFIRNLGEHIDEEKHKKMKLKIGDPSLYFQKTFPDLVIY | |
| 9 | KIADSSEGGVYLGFYGEQEVAVKRFYEGSTLGQKEVSCLQSCREKSNLVTFYESERQSDCLYV | Iberian mole RNase L |
| | CLTLCEQTLEEHFNARKRADVGNKEDKFARKVLSSIFKAVKGLHQCGYAHQDLQPRNILIDS | part amino acids 370- |
| | NDAICLADFDKSIKMTRKQEIKTDLKDLGLLVIYVVKKGDIPFERLKTKSNEDVVRLSPDVETE | 715 (starts with β- |
| | NLIHHLIFPKEEFTDLSGLLGHPFFWSWESRYRALSEVGNENDIKIRKPNSKIFQLLEPKSSEH | sheet 1) |
| | SKSFNRWKAKIDEYFMKQMDQFYKKDGRYQNTVGDLLRFIRNTRQHLHEKKNEKVKLRIG | |
| | EPSQYFQEKFPDFFLYVYERLHSTEYGKHFVQD | |
| 10 | KIADTQGGGVYLGLLEDQEVAVKVFVQGSECAGRELSALKSGQDSSRLVTLLACESHEANLY | American pika RNase L |
| | VCMTLCEWTLEEFLEHRPQPAQGDEDKWARQVLHSVFEAVRELHLQRAHAHQDLQPQN | part amino acids 351- |
| | ILIDAAHGIRLADFDQSVQLAEPTKVSQAVRTDLQALGRLVVYVLLKGDVPFEQLSAMHHR | 726 (starts with β- |
| | KVWRLCLERETRDLTRCLLTPQQAQEDPLGSLLGHPFFWEWQSRYRTLRNVGNESDIKRC | sheet 1) |
| | NLRGAIVQLLSLKPSEHSLSFAQWTSKVDSLVLGQMNDFYKNKPHLCYTDSVQDLLRFIRNI | |
| | GEHIDEDCNREMKARLGDPAIYFLRTFPDLLMHVYKQLQDSEFRRHFPQLPLPAGPGVLET | |
| | EVPGGQALGADP | |
| 11 | KVAETSEGGVYLGFYDGKEAAVKRFREDSKRAQQEMDCILHCQKNSNLVMFYGRESKDNC | Monito del monte |
| | LYLCFSLCERTLEEHVTTRALENEDFQNIFESLFKAVQELHRHDYLHQDLHPGNILIDSEGKV | RNase L part amino |
| | HLADFDKSEKLNENRHKIKDDLETLGRLVLYVVQLGKDPFFKIQGQSIREVIDACINEEIKDLV | acids 365-706 (starts |
| | QNLLMPGTQYQNLLSKMLSHPFFWNCNIRYRFLRDVGNESDIKRRNYESEILKALNCTDVF | with β-sheet 1) |
| | AEWTKKIDEHIMKKMNSFYKKKPMYCYKNEVGDLLKFIRNIGEHINEEQNKKMKLKIGEPA | |
| | QYFQKMFPDLVIYVYKKLRNTVYEKHFPSVNST | |
| 12 | KIADTSEGGVYLGFFDGLETAVKIFRDDSERAEKEIDCILECRENRHLVKLYGTEINNNCRYVC | Australian echidna |
| | LSLCERNLEEHLKNHRDADVESTKGILKSLFKAVHELHQFGFVHQDLHPKNILIDSKDEVCLA | RNase L part amino |
| | DFDKSCRFANDQTLVHGDLRALGRLVQYVVTKGEDPFVEKGAAREPQELAGLAEIPDHME | acids 356-707 (starts |
| | TLDLIQTLLSPGVTSQTQLSDILYHPFFWPCNMRYRFLRDVGNESDIKTRNTENKTLEKLNR | with β-sheet 1) |
| | NPSKSYSKWTSKIDKYVMTEMNHFYKKKKNPYQDTVTDLLKFIRNIGEHIDEKVNAQMKET | |
| | IGDPAQYFQKTFPELVMHVYKNIQNTEFMKHIPNAQNSLLDN | |
| 13 | RIQNTSQGGIYLGLHGGTEVAVRISRSTEGDKEKRFFEQCGNCEHLLKLFQFEKARGYMYLC | African penguin RNase |
| | FPLWEKNLEEHLQEPEDQMDYKDALRMIFQAVRELHSLGFAHQDLHPTNFFIDLGGKIYLA | L part amino acids 370- |
| | DFDNKRKLIEGKKELVNSDLEALSRLVLYVLTGGRKPLQQVSTKDLTADSPDYEEALDLVTSL | 701 (starts with β- |
| | VSHDERGLEGLSKHPYFWSKQTRFKFLKGIWNKIKDLHNRKAVFKAANATERFPYPQWTE | sheet 1) |
| | KIDKYVLKIMENPRKTKPLKYCNDVTDLLRLIRNLDEHPDTRISNRIGDHAEYFLKRFPALTIYV | |
| | YNSLRQNPKYSHFADIQDPSL | |
| 14 | WIQRTTQGGVYLGFYAGEEVAVKIFWKGAENAKREKVCLEKCRTSNHLVKFCGWEERKTC | Burmese python RNase |
| | LYLCLSLCERNLQEYFKEEKKKTMESEEILRTVFQAVKELHTFGFGHQELHPSNILIDATGRIFL | L part amino acids 348- |
| | ADFDKSREITDDIKYSIISEDLQGLQKLILYVVMRGQLDFEDLPTECPENVKNHKEIEDLRGRL | 704 (starts with β- |
| | GLSDQSTPADDLLEKLINHPYFWSKEQKYGVLKKVGNEIRAAKCCNKTSETNILEALKPMDN | sheet 1) |
| | SFRDWKKKIDKTVLDSMFFLSTEKGQKGNQKGNKRSYGPCITELLKLIRNMGEHYNEKDDK | |
| | VKDIIRDPADYFLDRFPDLTIYIYQRLCNTEYSIHFPNAKNPSHS | |
| 15 | KIQETSHGGIYLGFYKGKEVAVKRVPKGNNKELVCLEQLRKNNHLVRYCGYEEDSSCSYLCLF | Microcaecilia unicolor |
| | LYERNLEEHISNIKDVTEIQDMLQSIFLGLHELHEIGFCHRDLHPSNILIDHEGKAYLADFSGSL | RNase L part amino |
| | KLEDSEDKEGLIKSDMKALGNLVLYVVTRGTACFQPDRERDVTEACPDSVEDFIEARDLVNK | acids 344-703 (starts |
| | LMSPDTEYSSVIDFLNHPFFWSHTSRYKFLQDVGNENDVKMRTEDSALFRTLNDSHVTMT | with β-sheet 1) |
| | KTFHPWGCKVDQEVLNDMNRFHSKKKIYYQDTVTDLLKFIRNLGAHFDEKKTEIKEIIGDPC | |
| | QYFLKKFPDLTVYVYETLRHLGKGYIKHFPNPVVLFYNCMQLNNSGMT | |
| 16 | KIAESSQGGVYLGFYGDGVEVAVKRMRKNSNEVKMEKLCLEHPKIMGNSCFVKLYGSETD | Latimeria chalumnae |
| | ATCHYLCLTLCECTLETYIQEEEGNLKTKVPHIFKQLIGAVQILHEAGFAHTDLHPSNILFNVE | RNase L part amino |
| | GKLQLADFDKCLRFEDCPHKGQSERSDKTEEELKNEDIQAIGNLLCYVTTEGKYVYKTEEEKL | acids 404-759 (starts |
| | GDCYSAEVRDLIQKLSAPQGQPPPLKEALQHPFFWSQESKYRFLQDIGNENEVKVRNDKHE | with β-sheet 1) |
| | IVKSLNEKAQDPSSSFREWTKKVNDIVFKNMDSRNRKPELKYRNDTASLLKFIRNMGAHFE | |
| | EKPLEIKNIVKNPCNYFLDLFPDLVISVYNVVKSTQWEEHFPKAETS | |
| 17 | KITKDNITEVYLGFYNNVIEVAVKCHRNGSEEAKNEMDCLRDPTINGSSLFVNLLHWESDEF | great white shark |
| | CDYLCLNLYEYNLEECVTMWPKEIREKAPKIMKQLVDAVQVLHGAGFAHRDLHPTNVLRD | RNase L part amino |
| | VKENFRLADFDRSVNCKHPAVTISGKATWEASEILQKLELWPTATFEADELFKADLQALGRL | acids 434-808 (starts |
| | LHYVMTGGKDPCSSENDLCENNLSLNEDLCEPKNAEARDFIERLLAPAEKRATLSEIRQHPLL | with β-sheet 1): |
| | WKDLGKSQFVQGLANENGVAKRDKSSPIVLILNEVGNNQERSFHKWTEKVHLKILNNMN | |
| | KRQRTKYEDNTSDLLKFIRNLTQHFDEKEELHSLIKSPESYFCELFPDLTIATYNAVKDTEWVK | |
| | HFPKE | |
| 18 | MASDTPGFYMDKLNKYHQKHRVTITYKHLFTTGPPHDRRFTFQVIIDGEEFPEAEGKTKQE | Syrian hamster PKR |
| | AKNAAAKLAVDTLNANKANSHTDALEGSFTGNYIGLVNSYAQKEKLSVNYQQCAFNTQSP | part amino acids 1-158 |
| | QRFCYKCIIGLKTYGIGSGATKQEAKQLAAKEAYQKL | |
| 19 | MDKLNKYHQKHRVTITYKHLFTTGPPHDRRFTFQVIIDGEEFPEAEGKTKQEAKNAAAKLA | Syrian hamster PKR |
| | VDTLNANKANSHTDALEGSFTGNYIGLVNSYAQKEKLSVNYQQCAFNTQSPQRFCYKCIIGL | part amino acids 1-174 |
| | KTYGIGSGATKQEAKQLAAKEAYQKLPEKSSMRVCRASSGLSKSSSSGHFN | |
| 20 | MDKLNKYHQKHRVTITYKHLFTTGPPHDRRFTFQVIIDGEEFPEAEGKTKQEAKNAAAKLA | Syrian hamster PKR |
| | VDTLNANKANSHTDALEGSFTGNYIGLVNSYAQKEKLSVNYQQCAFNTQSPQRFCYKCIIGL | part amino acids 1-197 |
| | KTYGIGSGATKQEAKQLAAKEAYQKLPEKSSMRVCRASSGLSKSSSSGHFNSWSTANNFTS | |
| | LSAPGSDFSETAS | |
| 21 | MDKLNKYHQKHRVTITYKHLFTTGPPHDRRFTFQVIIDGEEFPEAEGKTKQEAKNAAAKLA | Syrian hamster PKR |
| | VDTLNANKANSHTDALEGSFTGNYIGLVNSYAQKEKLSVNYQQCAFNTQSPQRFCYKCIIGL | part amino acids 1-234 |
| | KTYGIGSGATKQEAKQLAAKEAYQKLPEKSSMRVCRASSGLSKSSSSGHFNSWSTANNFTS | |
| | LSAPXSDFSETASNNHCVFFKSPLMNGLRENKRKPRVKLSSNDVQRNKYT | |
| 22 | MDKLNKYHQKHRVTITYKHLFTTGPPHDRRFTFQVIIDGEEFPEAEGKTKQEAKNAAAKLA | Syrian hamster PKR |
| | VDTLNANKANSHTDALEGSFTGNYIGLVNSYAQKEKLSVNYQQCAFNTQSPQRFCYKCIIGL | part amino acids 1-518 |
| | KTYGIGSGATKQEAKQLAAKEAYQKLPEKSSMRVCRASSGLSKSSSSGHFNSWSTANNFTS | (full length PKR) |
| | LSAPGSDFSETASNNHCVFFKSPLMNGLRENKRKPRVKLSSNDVQRNKYTTDSRFNEDFED | |
| | IEEIGSGGFGQVFKAKHRIDGKTYAIKRVKYNSEKEVREVKALAALSHVNIVQYHFCWLGQD | |
| | WEYDTEQSMNSATPRSKTECLFIQMEFCDKGTLEQWMTSVKQRKVDNDLFLELAEQITTG | |
| | VDYIHSKGLIHRDLKPSNIFLVDEKHIKIGDFGLVTALENDGNRTKNTGSLLYMSPEQLSLQE | |
| | YGKEVDIFALGLILAELLHICITIIEKSKFFTDLRDGIFHDDIFGSKEKRLLTKLLSKKPTDRP | |
| | SASEILMTLAEWKNISEVQKRHTH | |
| 23 | MDKLNKYHQKHRVTITYKHLFTTGPPHDRRFTFQVIIDGEEFPEAEGKTKQEAKNAAAKLA | Syrian hamster PKR |
| | VDTLNANKANSHTDALEGSFTGNYIGLVNSYAQKEKLSVNYQQCAFNTQSPQRFCYKCIIGL | part amino acids 1-518 |
| | KTYGIGSGATKQEAKQLAAKEAYQKLPEKSSMRVCRASSGLSKSSSSGHFNSWSTANNFTS | (full length PKR-kinase |
| | LSAPGSDFSETASNNHCVFFKSPLMNGLRENKRKPRVKLSSNDVQRNKYTTDSRFNEDFED | inactivated) |
| | IEEIGSGGFGQVFKAKHRIDGKTYAI.RVKYNSEKEVREVKALAALSHVNIVQYHFCWLGQD | |
| | WEYDTEQSMNSATPRSKTECLFIQMEFCDKGTLEQWMTSVKQRKVDNDLFLELAEQITTG | |
| | VDYIHSKGLIHRDLKPSNIFLVDEKHIKIGDFGLVTALENDGNRTKNTGSLLYMSPEQLSLQE | |
| | YGKEVDIFALGLILAELLHICITIIEKSKFFTDLRDGIFHDDIFGSKEKRLLTKLLSKKPTDRP | |
| | SASEILMTLAEWKNISEVQKRHTH | |
| 24 | MASDTPGFYMDKLNKYRQMHGVAITYKELSTSGPPHDRRFTFQVLIDEKEFPEAKGKSKQE | Mouse PKR part amino |
| | ARNAAAKLAVDILDNENKVDCHTSASEQGLPYGNYIGLVNSFAQKKKLSVNYEQCEPNSEL | acids 1-208 |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFT | |
| 25 | MASDTPGFYMDKLNKYRQMHGVAITYKELSTSGPPHDRRFTFQVLIDEKEFPEAKGKSKQE | Mouse PKR part amino |
| | ARNAAAKLAVDILDNENKVDCHTSASEQGLPYGNYIGLVNSFAQKKKLSVNYEQCEPNSEL | acids 1-233 |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTNGLGENKRKSGVKVSPDDVQRNKYT | |
| 26 | MASDTPGFYMDKLNKYRQMHGVAITYKELSTSGPPHDRRFTFQVLIDEKEFPEAKGKSKQE | Mouse PKR part amino |
| | ARNAAAKLAVDILDNENKVDCHTSASEQGLPYGNYIGLVNSFAQKKKLSVNYEQCEPNSEL | acids 1-515 (full |
| | PQRFICKCKIGQTMYGTGSGVTKQEAKQLAAKEAYQKLLKSPPKTAGTSSSVVTSTFSGFSS | length PKR) |
| | SSSMTSNGVSQSAPGSFSSENVFTNGLGENKRKSGVKVSPDDVQRNKYTLDARFNSDFEDI | |
| | EEIGLGGFGQVFKAKHRIDGKRYAIKRVKYNTEKAEHEVQALAELNHVNIVQYHSCWEGVD | |
| | YDPEHSMSDTSRYKTRCLFIQMEFCDKGTLEQWMRNRNQSKVDKALILDLYEQIVTGVEYI | |
| | HSKGLIHRDLKPGNIFLVDERHIKIGDFGLATALENDGKSRTRRTGTLQYMSPEQLFLKHYGK | |
| | EVDIFALGLILAELLHTCFTESEKIKFFESLRKGDFSNDIFDNKEKSLLKKLLSEKPKDRPETS | |
| | EILKTLAEWRNISEKKKRNTC | |
| 27 | MESLSGNYTSLLNEYQQKTQCTVEFEEGPTDGPSHNKRFTMRAIVNGQKFPDGTGKTKKE | Zebrafish PKR part |
| | AKQNAAKNALEGLKSTHSDEPTPSPVENISVSKIASHPNYTCWLNEHSQKSRLMFKACEST | amino acids 1-325 |
| | KMDPGNLTRLCTYVCKYVCDDKEFPEGYGKNKKEAKEAAALRVYEELNKTPNTEVLDENSN | |
| | RAQRSETTSYSLDRRSSVADDSRSSTPDNNYIAYLNNYCQKKKRVYDFKLVDRIGPPHNPIFV | |
| | YKVVMDGKEYPEAQGRNAKEAKQNAAQHAWSEIRDQSGWTTPSSEDGAFSQSQDTCKS | |
| | EDVQRSANASSTSNFISFRDSA | |
| 28 | MAKMSAENEIEMRICDYLRRHGRSTVQDIFKELKLEKSTVNRHLYSLQASKQVFKTVEDNK | Zebrafish PKZ part |
| | RPVWDLVESMNDEQKAKPEQIKPEMTRDAIEEKHVKDLLKSGGLKASHIASKLGQQTQST | amino acids 1-156 |
| | NKLLYGLMEKGEVRKCSKSHMWTLKNDQERNGSGS | |
| 29 | MAQAPADPGREGHLEQRILQVLTEAGSPVKLAQLVKECQAPKRELNQVLYRMKKELKVSL | Human ZBP1 part |
| | TSPATWCLGGTDPEGEGPAELALSSPAERPQQHAATIPETPGPQFSQQREEDIYRFLKDNG | amino acids 1-178 |
| | PQRALVIAQALGMRTAKDVNRDLYRMKSRHLLDMDEQSKAWTIYRPEDSGRRAKSAS | |
| 30 | MAQAPADPGREGHLEQRILQVLTEAGSPVKLAQLVKECQAPKRELNQVLYRMKKELKVSL | Human ZBP1 part |
| | TSPATWCLGGTDPEGEGPAELALSSPAERPQQHAATIPETPGPQFSQQREEDIYRFLKDNG | amino acids 1-203 |
| | PQRALVIAQALGMRTAKDVNRDLYRMKSRHLLDMDEQSKAWTIYRPEDSGRRAKSASIIY | |
| | QHNPINMICQNGPNSWISIANS | |
| 31 | MAEAPVDLSTGDNLEQKILQVLSDDGGPVKIGQLVKKCQVPKKTLNQVLYRLKKEDRVSSP | Mouse ZBP1 part |
| | EPATWSIGGAASGDGAPAIPENSSAQPSLDERILRFLEANGPHRALHIAKALGMTTAKEVN | amino acids 1-164 |
| | PLLYSMRNKHLLSYDGQTWKIYHSRQEGQDIAHSGVTQESPA | |
| 32 | MAEAPVDLSTGDNLEQKILQVLSDDGGPVKIGQLVKKCQVPKKTLNQVLYRLKKEDRVSSP | Mouse ZBP1 part |
| | EPATWSIGGAASGDGAPAIPENSSAQPSLDERILRFLEANGPHRALHIAKALGMTTAKEVN | amino acids 1-189 |
| | PLLYSMRNKHLLSYDGQTWKIYHSRQEGQDIAHSGVTQESPAIICQHNPVNMICQQGANS | |
| | HISIANS | |
| 33 | GS | Linker |
| 34 | PEKSSMRVCRASSGLSKSSSSGHFNSGS | Linker |
| 35 | PEKSSMRVCRASSGLSKSSSSGHFNSWSTANNFTSLSAPGSDFSETASGS | Linker |
| 36 | PEKSSMRVCRASSGLSKSSSSGHFNSWSTANNFTSLSAPGSDFSETASNNHCVFFKSPLMN | Linker |
| | GLRENKRKPRVKLSSNDVQRNKYTGS | |
| 37 | LKSPPKTAGTSSSVVTSTFSGFSSSSSMTSNGVSQSAPGSFSSENVFT | Linker |
| 38 | LKSPPKTAGTSSSVVTSTFSGFSSSSSMTSNGVSQSAPGSFSSENVFTNGLGENKRKSGVKV | Linker |
| | SPDDVQRNKYT | |
| 39 | RDQSGWTTPSSEDGAFSQSQDTCKSEDVQRSANASSTSNFISFRDSA | Linker |
| 40 | DQERNGSGS | Linker |
| 41 | PEDSGRRAKSAS | Linker |
| 42 | PEDSGRRAKSASIIYQHNPINMICQNGPNSWISIANS | Linker |
| 43 | SRQEGQDIAHSGVTQESPA | Linker |
| 44 | SRQEGQDIAHSGVTQESPAIICQHNPVNMICQQGANSHISIANS | Linker |
| 45 | MASDTPGFYMDKLNKYHQKHRVTITYKHLFTTGPPHDRRFTFQVIIDGEEFPEAEGKTKQE | REAVR1 amino acid |
| | AKNAAAKLAVDTLNANKANSHTDALEGSFTGNYIGLVNSYAQKEKLSVNYQQCAFNTQSP | sequence |
| | QRFCYKCIIGLKTYGIGSGATKQEAKQLAAKEAYQKLGSSEGGIYLGFYEKQEVAVKTFCEGS | |
| | PRAQREVSCLQSSRENSHLVTFYGSESHRGHLFVCVTLCEQTLEACLDVHRGEDVENEEDEF | |
| | ARNVLSSIFKAVQELHLSCGYTHQDLQPQNILIDSKKAAHLADFDKSIKWAGDPQEVKRDLE | |
| | DLGRLVLYVVKKGSISFEDLKAQSNEEVVQLSPDEETKDLIHRLFHPGEHVRDCLSDLLGHPF | |
| | FWTWESRYRTLRNVGNESDIKTRKSESEILRLLQPGPSEHSKSFDKWTTKINECVMKKMNK | |
| | FYEKRGNFYQNTVGDLLKFIRNLGEHIDEEKHKKMKLKIGDPSLYFQKTFPDLVIYVYTKLQN | |
| | TEYRKHFPQTHSPNKPQCDGAGGASGLASPGC | |
| 46 | MDKLNKYHQKHRVTITYKHLFTTGPPHDRRFTFQVIIDGEEFPEAEGKTKQEAKNAAAKLA | REAVR2 amino acid |
| | VDTLNANKANSHTDALEGSFTGNYIGLVNSYAQKEKLSVNYQQCAFNTQSPQRFCYKCIIGL | sequence |
| | KTYGIGSGATKQEAKQLAAKEAYQKLPEKSSMRVCRASSGLSKSSSSGHFNSGSSEGGIYLG | |
| | FYEKQEVAVKTFCEGSPRAQREVSCLQSSRENSHLVTFYGSESHRGHLFVCVTLCEQTLEAC | |
| | LDVHRGEDVENEEDEFARNVLSSIFKAVQELHLSCGYTHQDLQPQNILIDSKKAAHLADFDK | |
| | SIKWAGDPQEVKRDLEDLGRLVLYVVKKGSISFEDLKAQSNEEVVQLSPDEETKDLIHRLFHP | |
| | GEHVRDCLSDLLGHPFFWTWESRYRTLRNVGNESDIKTRKSESEILRLLQPGPSEHSKSFDK | |
| | WTTKINECVMKKMNKFYEKRGNFYQNTVGDLLKFIRNLGEHIDEEKHKKMKLKIGDPSLYF | |
| | QKTFPDLVIYVYTKLQNTEYRKHFPQTHSPNKPQCDGAGGASGLASPGC | |
| 47 | MDKLNKYHQKHRVTITYKHLFTTGPPHDRRFTFQVIIDGEEFPEAEGKTKQEAKNAAAKLA | REAVR3 amino acid |
| | VDTLNANKANSHTDALEGSFTGNYIGLVNSYAQKEKLSVNYQQCAFNTQSPQRFCYKCIIGL | sequence |
| | KTYGIGSGATKQEAKQLAAKEAYQKLPEKSSMRVCRASSGLSKSSSSGHFNSWSTANNFTS | |
| | LSAPGSDFSETASGSSEGGIYLGFYEKQEVAVKTFCEGSPRAQREVSCLQSSRENSHLVTFYG | |
| | SESHRGHLFVCVTLCEQTLEACLDVHRGEDVENEEDEFARNVLSSIFKAVQELHLSCGYTHQ | |
| | DLQPQNILIDSKKAAHLADFDKSIKWAGDPQEVKRDLEDLGRLVLYVVKKGSISFEDLKAQS | |
| | NEEVVQLSPDEETKDLIHRLFHPGEHVRDCLSDLLGHPFFWTWESRYRTLRNVGNESDIKT | |
| | RKSESEILRLLQPGPSEHSKSFDKWTTKINECVMKKMNKFYEKRGNFYQNTVGDLLKFIRNL | |
| | GEHIDEEKHKKMKLKIGDPSLYFQKTFPDLVIYVYTKLQNTEYRKHFPQTHSPNKPQCDGAG | |
| | GASGLASPGC | |
| 48 | MDKLNKYHQKHRVTITYKHLFTTGPPHDRRFTFQVIIDGEEFPEAEGKTKQEAKNAAAKLA | REAVR4 amino acid |
| | VDTLNANKANSHTDALEGSFTGNYIGLVNSYAQKEKLSVNYQQCAFNTQSPQRFCYKCIIGL | sequence |
| | KTYGIGSGATKQEAKQLAAKEAYQKLPEKSSMRVCRASSGLSKSSSSGHFNSWSTANNFTS | |
| | LSAPXSDFSETASNNHCVFFKSPLMNGLRENKRKPRVKLSSNDVQRNKYTGSSEGGIYLGFY | |
| | EKQEVAVKTFCEGSPRAQREVSCLQSSRENSHLVTFYGSESHRGHLFVCVTLCEQTLEACLD | |
| | VHRGEDVENEEDEFARNVLSSIFKAVQELHLSCGYTHQDLQPQNILIDSKKAAHLADFDKSI | |
| | KWAGDPQEVKRDLEDLGRLVLYVVKKGSISFEDLKAQSNEEVVQLSPDEETKDLIHRLFHPG | |
| | EHVRDCLSDLLGHPFFWTWESRYRTLRNVGNESDIKTRKSESEILRLLQPGPSEHSKSFDKW | |
| | TTKINECVMKKMNKFYEKRGNFYQNTVGDLLKFIRNLGEHIDEEKHKKMKLKIGDPSLYFQ | |
| | KTFPDLVIYVYTKLQNTEYRKHFPQTHSPNKPQCDGAGGASGLASPGC | |
| 49 | MDKLNKYHQKHRVTITYKHLFTTGPPHDRRFTFQVIIDGEEFPEAEGKTKQEAKNAAAKLA | REAVR5 amino acid |
| | VDTLNANKANSHTDALEGSFTGNYIGLVNSYAQKEKLSVNYQQCAFNTQSPQRFCYKCIIGL | sequence |
| | KTYGIGSGATKQEAKQLAAKEAYQKLPEKSSMRVCRASSGLSKSSSSGHFNSWSTANNFTS | |
| | LSAPGSDFSETASNNHCVFFKSPLMNGLRENKRKPRVKLSSNDVQRNKYTTDSRFNEDFED | |
| | IEEIGSGGFGQVFKAKHRIDGKTYAIKRVKYNSEKEVREVKALAALSHVNIVQYHFCWLGQD | |
| | WEYDTEQSMNSATPRSKTECLFIQMEFCDKGTLEQWMTSVKQRKVDNDLFLELAEQITTG | |
| | VDYIHSKGLIHRDLKPSNIFLVDEKHIKIGDFGLVTALENDGNRTKNTGSLLYMSPEQLSLQE | |
| | YGKEVDIFALGLILAELLHICITIIEKSKFFTDLRDGIFHDDIFGSKEKRLLTKLLSKKPTDRP | |
| | SASEILMTLAEWKNISEVQKRHTHGSSEGGIYLGFYEKQEVAVKTFCEGSPRAQREVSCLQSSRE | |
| | NSHLVTFYGSESHRGHLFVCVTLCEQTLEACLDVHRGEDVENEEDEFARNVLSSIFKAVQELH | |
| | LSCGYTHQDLQPQNILIDSKKAAHLADFDKSIKWAGDPQEVKRDLEDLGRLVLYVVKKGSIS | |
| | FEDLKAQSNEEVVQLSPDEETKDLIHRLFHPGEHVRDCLSDLLGHPFFWTWESRYRTLRNV | |
| | GNESDIKTRKSESEILRLLQPGPSEHSKSFDKWTTKINECVMKKMNKFYEKRGNFYQNTVG | |
| | DLLKFIRNLGEHIDEEKHKKMKLKIGDPSLYFQKTFPDLVIYVYTKLQNTEYRKHFPQTHSPN | |
| | KPQCDGAGGASGLASPGC | |
| 50 | MDKLNKYHQKHRVTITYKHLFTTGPPHDRRFTFQVIIDGEEFPEAEGKTKQEAKNAAAKLA | REAVR6 amino acid |
| | VDTLNANKANSHTDALEGSFTGNYIGLVNSYAQKEKLSVNYQQCAFNTQSPQRFCYKCIIGL | sequence |
| | KTYGIGSGATKQEAKQLAAKEAYQKLPEKSSMRVCRASSGLSKSSSSGHFNSWSTANNFTS | |
| | LSAPGSDFSETASNNHCVFFKSPLMNGLRENKRKPRVKLSSNDVQRNKYTTDSRFNEDFED | |
| | IEEIGSGGFGQVFKAKHRIDGKTYAIRRVKYNSEKEVREVKALAALSHVNIVQYHFCWLGQD | |
| | WEYDTEQSMNSATPRSKTECLFIQMEFCDKGTLEQWMTSVKQRKVDNDLFLELAEQITTG | |
| | VDYIHSKGLIHRDLKPSNIFLVDEKHIKIGDFGLVTALENDGNRTKNTGSLLYMSPEQLSLQE | |
| | YGKEVDIFALGLILAELLHICITIIEKSKFFTDLRDGIFHDDIFGSKEKRLLTKLLSKKPTDRP | |
| | SASEILMTLAEWKNISEVQKRHTHGSSEGGIYLGFYEKQEVAVKTFCEGSPRAQREVSCLQSSRE | |
| | NSHLVTFYGSESHRGHLFVCVTLCEQTLEACLDVHRGEDVENEEDEFARNVLSSIFKAVQELH | |
| | LSCGYTHQDLQPQNILIDSKKAAHLADFDKSIKWAGDPQEVKRDLEDLGRLVLYVVKKGSIS | |
| | FEDLKAQSNEEVVQLSPDEETKDLIHRLFHPGEHVRDCLSDLLGHPFFWTWESRYRTLRNV | |
| | GNESDIKTRKSESEILRLLQPGPSEHSKSFDKWTTKINECVMKKMNKFYEKRGNFYQNTVG | |
| | DLLKFIRNLGEHIDEEKHKKMKLKIGDPSLYFQKTFPDLVIYVYTKLQNTEYRKHFPQTHSPN | |
| | KPQCDGAGGASGLASPGC | |
| 51 | MASDTPGFYMDKLNKYRQMHGVAITYKELSTSGPPHDRRFTFQVLIDEKEFPEAKGKSKQE | REAVR7 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTKIADTSEGGIYLGFYEKQEVAVKTFCEGSPRAQREVSC | |
| | LQSSRENSHLVTFYGSESHRGHLFVCVTLCEQTLEACLDVHRGEDVENEEDEFARNVLSSIFK | |
| | AVQELHLSCGYTHQDLQPQNILIDSKKAAHLADFDKSIKWAGDPQEVKRDLEDLGRLVLYV | |
| | VKKGSISFEDLKAQSNEEVVQLSPDEETKDLIHRLFHPGEHVRDCLSDLLGHPFFWTWESRY | |
| | RTLRNVGNESDIKTRKSESEILRLLQPGPSEHSKSFDKWTTKINECVMKKMNKFYEKRGNFY | |
| | QNTVGDLLKFIRNLGEHIDEEKHKKMKLKIGDPSLYFQKTFPDLVIYVYTKLQNTEYRKHFPQ | |
| | THSPNKPQCDGAGGASGLASPGC | |
| 52 | MASDTPGFYMDKLNKYRQMHGVAITYKELSTSGPPHDRRFTFQVLIDEKEFPEAKGKSKQE | REAVR8 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTNGLGENKRKSGVKVSPDDVQRNKYTKIADTSEGGIYL | |
| | GFYEKQEVAVKTFCEGSPRAQREVSCLQSSRENSHLVTFYGSESHRGHLFVCVTLCEQTLEA | |
| | CLDVHRGEDVENEEDEFARNVLSSIFKAVQELHLSCGYTHQDLQPQNILIDSKKAAHLADFD | |
| | KSIKWAGDPQEVKRDLEDLGRLVLYVVKKGSISFEDLKAQSNEEVVQLSPDEETKDLIHRLFH | |
| | PGEHVRDCLSDLLGHPFFWTWESRYRTLRNVGNESDIKTRKSESEILRLLQPGPSEHSKSFD | |
| | KWTTKINECVMKKMNKFYEKRGNFYQNTVGDLLKFIRNLGEHIDEEKHKKMKLKIGDPSLY | |
| | FQKTFPDLVIYVYTKLQNTEYRKHFPQTHSPNKPQCDGAGGASGLASPGC | |
| 53 | MASDTPGFYMDKLNKYRQMHGVAITYKELSTSGPPHDRRFTFQVLIDEKEFPEAKGKSKQE | REAVR9 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTNGLGENKRKSGVKVSPDDVQRNKYTLDARFNSDFEDI | |
| | EEIGLGGFGQVFKAKHRIDGKRYAIKRVKYNTEKAEHEVQALAELNHVNIVQYHSCWEGVD | |
| | YDPEHSMSDTSRYKTRCLFIQMEFCDKGTLEQWMRNRNQSKVDKALILDLYEQIVTGVEYI | |
| | HSKGLIHRDLKPGNIFLVDERHIKIGDFGLATALENDGKSRTRRTGTLQYMSPEQLFLKHYGK | |
| | EVDIFALGLILAELLHTCFTESEKIKFFESLRKGDFSNDIFDNKEKSLLKKLLSEKPKDRPETS | |
| | EILKTLAEWRNISEKKKRNTCKIADTSEGGIYLGFYEKQEVAVKTFCEGSPRAQREVSCLQSSRE | |
| | NSHLVTFYGSESHRGHLFVCVTLCEQTLEACLDVHRGEDVENEEDEFARNVLSSIFKAVQELH | |
| | LSCGYTHQDLQPQNILIDSKKAAHLADFDKSIKWAGDPQEVKRDLEDLGRLVLYVVKKGSIS | |
| | FEDLKAQSNEEVVQLSPDEETKDLIHRLFHPGEHVRDCLSDLLGHPFFWTWESRYRTLRNV | |
| | GNESDIKTRKSESEILRLLQPGPSEHSKSFDKWTTKINECVMKKMNKFYEKRGNFYQNTVG | |
| | DLLKFIRNLGEHIDEEKHKKMKLKIGDPSLYFQKTFPDLVIYVYTKLQNTEYRKHFPQTHSPN | |
| | KPQCDGAGGASGLASPGC | |
| 54 | MASDTPGFYMDKLNKYRQMHGVAITYKELSTSGPPHDRRFTFQVLIDEKEFPEAKGKSKQE | REAVR10 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTKIQETSHGGIYLGFYKGKEVAVKRVPKGNNKELVCLEQ | |
| | LRKNNHLVRYCGYEEDSSCSYLCLFLYERNLEEHISNIKDVTEIQDMLQSIFLGLHELHEIGFC | |
| | HRDLHPSNILIDHEGKAYLADFSGSLKLEDSEDKEGLIKSDMKALGNLVLYVVTRGTACFQP | |
| | DRERDVTEACPDSVEDFIEARDLVNKLMSPDTEYSSVIDFLNHPFFWSHTSRYKFLQDVGN | |
| | ENDVKMRTEDSALFRTLNDSHVTMTKTFHPWGCKVDQEVLNDMNRFHSKKKIYYQDTVT | |
| | DLLKFIRNLGAHFDEKKTEIKEIIGDPCQYFLKKFPDLTVYVYETLRHLGKGYIKHFPNPVVLFY | |
| | NCMQLNNSGMT | |
| 55 | MASDTPGFYMDKLNKYRQMHGVAITYKELSTSGPPHDRRFTFQVLIDEKEFPEAKGKSKQE | REAVR11 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTRIQNTSQGGIYLGLHGGTEVAVRISRSTEGDKEKRFFE | |
| | QCGNCEHLLKLFQFEKARGYMYLCFPLWEKNLEEHLQEPEDQMDYKDALRMIFQAVRELH | |
| | SLGFAHQDLHPTNFFIDLGGKIYLADFDNKRKLIEGKKELVNSDLEALSRLVLYVLTGGRKPL | |
| | QQVSTKDLTADSPDYEEALDLVTSLVSHDERGLEGLSKHPYFWSKQTRFKFLKGIWNKIKDL | |
| | HNRKAVFKAANATERFPYPQWTEKIDKYVLKIMENPRKTKPLKYCNDVTDLLRLIRNLDEHP | |
| | DTRISNRIGDHAEYFLKRFPALTIYVYNSLRQNPKYSHFADIQDPSL | |
| 56 | MASDTPGFYMDKLNKYRQMHGVAITYKELSTSGPPHDRRFTFQVLIDEKEFPEAKGKSKQE | REAVR12 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTKITKDNITEVYLGFYNNVIEVAVKCHRNGSEEAKNEM | |
| | DCLRDPTINGSSLFVNLLHWESDEFCDYLCLNLYEYNLEECVTMWPKEIREKAPKIMKQLVD | |
| | AVQVLHGAGFAHRDLHPTNVLRDVKENFRLADFDRSVNCKHPAVTISGKATWEASEILQKL | |
| | ELWPTATFEADELFKADLQALGRLLHYVMTGGKDPCSSENDLCENNLSLNEDLCEPKNAEA | |
| | RDFIERLLAPAEKRATLSEIRQHPLLWKDLGKSQFVQGLANENGVAKRDKSSPIVLILNEVGN | |
| | NQERSFHKWTEKVHLKILNNMNKRQRTKYEDNTSDLLKFIRNLTQHFDEKEELHSLIKSPES | |
| | YFCELFPDLTIATYNAVKDTEWVKHFPKE | |
| 57 | MASDTPGFYMDKLNKYRQMHGVAITYKELSTSGPPHDRRFTFQVLIDEKEFPEAKGKSKQE | REAVR13 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTWIQRTTQGGVYLGFYAGEEVAVKIFWKGAENAKREK | |
| | VCLEKCRTSNHLVKFCGWEERKTCLYLCLSLCERNLQEYFKEEKKKTMESEEILRTVFQAVKE | |
| | LHTFGFGHQELHPSNILIDATGRIFLADFDKSREITDDIKYSIISEDLQGLQKLILYVVMRGQLD | |
| | FEDLPTECPENVKNHKEIEDLRGRLGLSDQSTPADDLLEKLINHPYFWSKEQKYGVLKKVGN | |
| | EIRAAKCCNKTSETNILEALKPMDNSFRDWKKKIDKTVLDSMFFLSTEKGQKGNQKGNKRS | |
| | YGPCITELLKLIRNMGEHYNEKDDKVKDIIRDPADYFLDRFPDLTIYIYQRLCNTEYSIHFPNA | |
| | NPSHS | |
| 58 | MASDTPGFYMDKLNKYRQMHGVAITYKELSTSGPPHDRRFTFQVLIDEKEFPEAKGKSKQE | REAVR14 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTKIAESSQGGVYLGFYGDGVEVAVKRMRKNSNEVKME | |
| | KLCLEHPKIMGNSCFVKLYGSETDATCHYLCLTLCECTLETYIQEEEGNLKTKVPHIFKQLIGA | |
| | VQILHEAGFAHTDLHPSNILFNVEGKLQLADFDKCLRFEDCPHKGQSERSDKTEEELKNEDI | |
| | QAIGNLLCYVTTEGKYVYKTEEEKLGDCYSAEVRDLIQKLSAPQGQPPPLKEALQHPFFWSQ | |
| | ESKYRFLQDIGNENEVKVRNDKHEIVKSLNEKAQDPSSSFREWTKKVNDIVFKNMDSRNRK | |
| | PELKYRNDTASLLKFIRNMGAHFEEKPLEIKNIVKNPCNYFLDLFPDLVISVYNVVKSTQWEE | |
| | HFPKAETS | |
| 59 | MASDTPGFYMDKLNKYRQMHGVAITYKELSTSGPPHDRRFTFQVLIDEKEFPEAKGKSKQE | REAVR15 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTKIADSSEGGVYLGFYGEQEVAVKRFYEGSTLGQKEVSC | |
| | LQSCREKSNLVTFYESERQSDCLYVCLTLCEQTLEEHFNARKRADVGNKEDKFARKVLSSIFK | |
| | AVKGLHQCGYAHQDLQPRNILIDSNDAICLADFDKSIKMTRKQEIKTDLKDLGLLVIYVVKK | |
| | GDIPFERLKTKSNEDVVRLSPDVETENLIHHLIFPKEEFTDLSGLLGHPFFWSWESRYRALSEV | |
| | GNENDIKIRKPNSKIFQLLEPKSSEHSKSFNRWKAKIDEYFMKQMDQFYKKDGRYQNTVGD | |
| | LLRFIRNTRQHLHEKKNEKVKLRIGEPSQYFQEKFPDFFLYVYERLHSTEYGKHFVQD | |
| 60 | MASDTPGFYMDKLNKYRQMHGVAITYKELSTSGPPHDRRFTFQVLIDEKEFPEAKGKSKQE | REAVR16 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTKIADTQGGGVYLGLLEDQEVAVKVFVQGSECAGRELS | |
| | ALKSGQDSSRLVTLLACESHEANLYVCMTLCEWTLEEFLEHRPQPAQGDEDKWARQVLHS | |
| | VFEAVRELHLQRAHAHQDLQPQNILIDAAHGIRLADFDQSVQLAEPTKVSQAVRTDLQAL | |
| | GRLVVYVLLKGDVPFEQLSAMHHRKVWRLCLERETRDLTRCLLTPQQAQEDPLGSLLGHPF | |
| | FWEWQSRYRTLRNVGNESDIKRCNLRGAIVQLLSLKPSEHSLSFAQWTSKVDSLVLGQMN | |
| | DFYKNKPHLCYTDSVQDLLRFIRNIGEHIDEDCNREMKARLGDPAIYFLRTFPDLLMHVYKQ | |
| | LQDSEFRRHFPQLPLPAGPGVLETEVPGGQALGADP | |
| 61 | MASDTPGFYMDKLNKYRQMHGVAITYKELSTSGPPHDRRFTFQVLIDEKEFPEAKGKSKQE | REAVR17 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTKVAETSEGGVYLGFYDGKEAAVKRFREDSKRAQQEM | |
| | DCILHCQKNSNLVMFYGRESKDNCLYLCFSLCERTLEEHVTTRALENEDFQNIFESLFKAVQE | |
| | LHRHDYLHQDLHPGNILIDSEGKVHLADFDKSEKLNENRHKIKDDLETLGRLVLYVVQLGKD | |
| | PFFKIQGQSIREVIDACINEEIKDLVQNLLMPGTQYQNLLSKMLSHPFFWNCNIRYRFLRDV | |
| | GNESDIKRRNYESEILKALNCTDVFAEWTKKIDEHIMKKMNSFYKKKPMYCYKNEVGDLLKF | |
| | IRNIGEHINEEQNKKMKLKIGEPAQYFQKMFPDLVIYVYKKLRNTVYEKHFPSVNST | |
| 62 | MASDTPGFYMDKLNKYRQMHGVAITYKELSTSGPPHDRRFTFQVLIDEKEFPEAKGKSKQE | REAVR18 amino acid |
| | ARNAAAKLAVDILDNENKVDCHTSASEQGLPYGNYIGLVNSFAQKKKLSVNYEQCEPNSEL | sequence |
| | PQRFICKCKIGQTMYGTGSGVTKQEAKQLAAKEAYQKLLKSPPKTAGTSSSVVTSTFSGFSS | |
| | SSSMTSNGVSQSAPGSFSSENVFTKIADTSEGGVYLGFFDGLETAVKIFRDDSERAEKEIDCIL | |
| | ECRENRHLVKLYGTEINNNCRYVCLSLCERNLEEHLKNHRDADVESTKGILKSLFKAVHELHQ | |
| | FGFVHQDLHPKNILIDSKDEVCLADFDKSCRFANDQTLVHGDLRALGRLVQYVVTKGEDPF | |
| | VEKGAAREPQELAGLAEIPDHMETLDLIQTLLSPGVTSQTQLSDILYHPFFWPCNMRYRFLR | |
| | DVGNESDIKTRNTENKTLEKLNRNPSKSYSKWTSKIDKYVMTEMNHFYKKKKNPYQDTVT | |
| | DLLKFIRNIGEHIDEKVNAQMKETIGDPAQYFQKTFPELVMHVYKNIQNTEFMKHIPNAQN | |
| | SLLDN | |
| 63 | MESLSGNYTSLLNEYQQKTQCTVEFEEGPTDGPSHNKRFTMRAIVNGQKFPDGTGKTKKE | REAVR21 amino acid |
| | AKQNAAKNALEGLKSTHSDEPTPSPVENISVSKIASHPNYTCWLNEHSQKSRLMFKACEST | sequence |
| | KMDPGNLTRLCTYVCKYVCDDKEFPEGYGKNKKEAKEAAALRVYEELNKTPNTEVLDENSN | |
| | RAQRSETTSYSLDRRSSVADDSRSSTPDNNYIAYLNNYCQKKKRVYDFKLVDRIGPPHNPIFV | |
| | YKVVMDGKEYPEAQGRNAKEAKQNAAQHAWSEIRDQSGWTTPSSEDGAFSQSQDTCKS | |
| | EDVQRSANASSTSNFISFRDSAKIADTSEGGIYLGFYEKQEVAVKTFCEGSPRAQREVSCLQS | |
| | SRENSHLVTFYGSESHRGHLFVCVTLCEQTLEACLDVHRGEDVENEEDEFARNVLSSIFKAV | |
| | QELHLSCGYTHQDLQPQNILIDSKKAAHLADFDKSIKWAGDPQEVKRDLEDLGRLVLYVVK | |
| | KGSISFEDLKAQSNEEVVQLSPDEETKDLIHRLFHPGEHVRDCLSDLLGHPFFWTWESRYRT | |
| | LRNVGNESDIKTRKSESEILRLLQPGPSEHSKSFDKWTTKINECVMKKMNKFYEKRGNFYQ | |
| | NTVGDLLKFIRNLGEHIDEEKHKKMKLKIGDPSLYFQKTFPDLVIYVYTKLQNTEYRKHFPQT | |
| | HSPNKPQCDGAGGASGLASPGC | |
| 64 | MAKMSAENEIEMRICDYLRRHGRSTVQDIFKELKLEKSTVNRHLYSLQASKQVFKTVEDNK | REAVR22 amino acid |
| | RPVWDLVESMNDEQKAKPEQIKPEMTRDAIEEKHVKDLLKSGGLKASHIASKLGQQTQST | sequence |
| | NKLLYGLMEKGEVRKCSKSHMWTLKNDQERNGSGSKIADTSEGGIYLGFYEKQEVAVKTFC | |
| | EGSPRAQREVSCLQSSRENSHLVTFYGSESHRGHLFVCVTLCEQTLEACLDVHRGEDVENEE | |
| | DEFARNVLSSIFKAVQELHLSCGYTHQDLQPQNILIDSKKAAHLADFDKSIKWAGDPQEVKR | |
| | DLEDLGRLVLYVVKKGSISFEDLKAQSNEEVVQLSPDEETKDLIHRLFHPGEHVRDCLSDLLG | |
| | HPFFWTWESRYRTLRNVGNESDIKTRKSESEILRLLQPGPSEHSKSFDKWTTKINECVMKK | |
| | MNKFYEKRGNFYQNTVGDLLKFIRNLGEHIDEEKHKKMKLKIGDPSLYFQKTFPDLVIYVYT | |
| | KLQNTEYRKHFPQTHSPNKPQCDGAGGASGLASPGC | |
| 65 | MAQAPADPGREGHLEQRILQVLTEAGSPVKLAQLVKECQAPKRELNQVLYRMKKELKVSL | REAVR23 amino acid |
| | TSPATWCLGGTDPEGEGPAELALSSPAERPQQHAATIPETPGPQFSQQREEDIYRFLKDNG | sequence |
| | PQRALVIAQALGMRTAKDVNRDLYRMKSRHLLDMDEQSKAWTIYRPEDSGRRAKSASKIA | |
| | DTSEGGIYLGFYEKQEVAVKTFCEGSPRAQREVSCLQSSRENSHLVTFYGSESHRGHLFVCV | |
| | TLCEQTLEACLDVHRGEDVENEEDEFARNVLSSIFKAVQELHLSCGYTHQDLQPQNILIDSKK | |
| | AAHLADFDKSIKWAGDPQEVKRDLEDLGRLVLYVVKKGSISFEDLKAQSNEEVVQLSPDEET | |
| | KDLIHRLFHPGEHVRDCLSDLLGHPFFWTWESRYRTLRNVGNESDIKTRKSESEILRLLQPGP | |
| | SEHSKSFDKWTTKINECVMKKMNKFYEKRGNFYQNTVGDLLKFIRNLGEHIDEEKHKKMKL | |
| | KIGDPSLYFQKTFPDLVIYVYTKLQNTEYRKHFPQTHSPNKPQCDGAGGASGLASPGC | |
| 66 | MAQAPADPGREGHLEQRILQVLTEAGSPVKLAQLVKECQAPKRELNQVLYRMKKELKVSL | REAVR24 amino acid |
| | TSPATWCLGGTDPEGEGPAELALSSPAERPQQHAATIPETPGPQFSQQREEDIYRFLKDNG | sequence |
| | PQRALVIAQALGMRTAKDVNRDLYRMKSRHLLDMDEQSKAWTIYRPEDSGRRAKSASIIY | |
| | QHNPINMICQNGPNSWISIANSKIADTSEGGIYLGFYEKQEVAVKTFCEGSPRAQREVSCLQ | |
| | SSRENSHLVTFYGSESHRGHLFVCVTLCEQTLEACLDVHRGEDVENEEDEFARNVLSSIFKA | |
| | VQELHLSCGYTHQDLQPQNILIDSKKAAHLADFDKSIKWAGDPQEVKRDLEDLGRLVLYVV | |
| | KKGSISFEDLKAQSNEEVVQLSPDEETKDLIHRLFHPGEHVRDCLSDLLGHPFFWTWESRYR | |
| | TLRNVGNESDIKTRKSESEILRLLQPGPSEHSKSFDKWTTKINECVMKKMNKFYEKRGNFYQ | |
| | NTVGDLLKFIRNLGEHIDEEKHKKMKLKIGDPSLYFQKTFPDLVIYVYTKLQNTEYRKHFPQT | |
| | HSPNKPQCDGAGGASGLASPGC | |
| 67 | MAEAPVDLSTGDNLEQKILQVLSDDGGPVKIGQLVKKCQVPKKTLNQVLYRLKKEDRVSSP | REAVR25 amino acid |
| | EPATWSIGGAASGDGAPAIPENSSAQPSLDERILRFLEANGPHRALHIAKALGMTTAKEVN | sequence |
| | PLLYSMRNKHLLSYDGQTWKIYHSRQEGQDIAHSGVTQESPAKIADTSEGGIYLGFYEKQEV | |
| | AVKTFCEGSPRAQREVSCLQSSRENSHLVTFYGSESHRGHLFVCVTLCEQTLEACLDVHRGE | |
| | DVENEEDEFARNVLSSIFKAVQELHLSCGYTHQDLQPQNILIDSKKAAHLADFDKSIKWAGD | |
| | PQEVKRDLEDLGRLVLYVVKKGSISFEDLKAQSNEEVVQLSPDEETKDLIHRLFHPGEHVRD | |
| | CLSDLLGHPFFWTWESRYRTLRNVGNESDIKTRKSESEILRLLQPGPSEHSKSFDKWTTKINE | |
| | CVMKKMNKFYEKRGNFYQNTVGDLLKFIRNLGEHIDEEKHKKMKLKIGDPSLYFQKTFPDL | |
| | VIYVYTKLQNTEYRKHFPQTHSPNKPQCDGAGGASGLASPGC | |
| 68 | MAEAPVDLSTGDNLEQKILQVLSDDGGPVKIGQLVKKCQVPKKTLNQVLYRLKKEDRVSSP | Exemplary REAVR1 |
| | EPATWSIGGAASGDGAPAIPENSSAQPSLDERILRFLEANGPHRALHIAKALGMTTAKEVN | DNA sequence |
| | PLLYSMRNKHLLSYDGQTWKIYHSRQEGQDIAHSGVTQESPAIICQHNPVNMICQQGANS | |
| | HISIANSKIADTSEGGIYLGFYEKQEVAVKTFCEGSPRAQREVSCLQSSRENSHLVTFYGSESH | |
| | RGHLFVCVTLCEQTLEACLDVHRGEDVENEEDEFARNVLSSIFKAVQELHLSCGYTHQDLQP | |
| | QNILIDSKKAAHLADFDKSIKWAGDPQEVKRDLEDLGRLVLYVVKKGSISFEDLKAQSNEEV | |
| | VQLSPDEETKDLIHRLFHPGEHVRDCLSDLLGHPFFWTWESRYRTLRNVGNESDIKTRKSES | |
| | EILRLLQPGPSEHSKSFDKWTTKINECVMKKMNKFYEKRGNFYQNTVGDLLKFIRNLGEHID | |
| | EEKHKKMKLKIGDPSLYFQKTFPDLVIYVYTKLQNTEYRKHFPQTHSPNKPQCDGAGGASG | |
| | LASPGC | |
| 69 | ATGGCCAGTGATACTCCCGGTTTCTACATGGACAAACTTAATAAATACCACCAGAAGCA | REAVR26 amino acid |
| | TAGAGTAACGATTACGTATAAACACCTTTTTACTACAGGACCTCCACATGACAGAAGGT | sequence |
| | TTACATTTCAAGTTATAATAGACGGAGAAGAATTTCCAGAAGCCGAAGGCAAAACAAA | |
| | GCAGGAAGCAAAAAACGCTGCTGCCAAATTGGCTGTTGATACACTTAACGCAAATAAG | |
| | GCAAATAGTCATACAGACGCTTTGGAAGGTTCATTCACTGGGAATTACATAGGCCTTGT | |
| | CAACAGTTATGCTCAGAAGGAGAAGTTGTCTGTAAATTACCAACAGTGTGCCTTCAATA | |
| | CCCAGTCACCCCAAAGATTTTGTTATAAATGCATAATTGGGCTGAAAACCTACGGTATT | |
| | GGTTCAGGCGCTACCAAGCAGGAGGCAAAGCAGCTGGCTGCTAAAGAGGCGTATCAG | |
| | AAGCTAGGATCCTCAGAAGGAGGCATCTACCTGGGGTTCTATGAGAAGCAAGAAGTA | |
| | GCTGTGAAGACGTTCTGTGAGGGCAGCCCACGTGCACAGCGGGAAGTCTCTTGTCTGC | |
| | AAAGCAGCCGAGAGAACAGTCACTTGGTGACATTCTATGGGAGTGAGAGCCACAGGG | |
| | GCCACTTGTTTGTGTGTGTCACCCTCTGTGAGCAGACTCTGGAAGCGTGTTTGGATGTG | |
| | CACAGAGGGGAAGATGTGGAAAATGAGGAAGATGAATTTGCCCGAAATGTCCTGTCA | |
| | TCTATATTTAAGGCTGTTCAAGAACTACACTTGTCCTGTGGATACACCCACCAGGATCTG | |
| | CAACCACAAAACATCTTAATAGATTCTAAGAAAGCTGCTCACCTGGCAGATTTTGATAA | |
| | GAGCATCAAGTGGGCTGGAGATCCACAGGAAGTCAAGAGAGATCTAGAGGACCTTGG | |
| | ACGGCTGGTCCTCTATGTGGTAAAGAAGGGAAGCATCTCATTTGAGGATCTGAAAGCT | |
| | CAAAGTAATGAAGAGGTGGTTCAACTTTCTCCAGATGAGGAAACTAAGGACCTCATTC | |
| | ATCGTCTCTTCCATCCTGGGGAACATGTGAGGGACTGTCTGAGTGACCTGCTGGGTCAT | |
| | CCCTTCTTTTGGACTTGGGAGAGCCGCTATAGGACGCTTCGGAATGTGGGAAATGAAT | |
| | CCGACATCAAAACACGAAAATCTGAAAGTGAGATCCTCAGACTACTGCAACCTGGGCC | |
| | TTCTGAACATTCCAAAAGTTTTGACAAGTGGACGACTAAGATTAATGAATGTGTTATGA | |
| | AAAAAATGAATAAGTTTTATGAAAAAAGAGGCAATTTCTACCAGAACACTGTGGGTGA | |
| | TCTGCTAAAGTTCATCCGGAATTTGGGAGAACACATTGATGAAGAAAAGCATAAAAAG | |
| | ATGAAATTAAAAATTGGAGACCCTTCCCTGTATTTTCAGAAGACATTTCCAGATCTGGT | |
| | GATCTATGTCTACACAAAACTACAGAACACAGAATATAGAAAGCATTTCCCCCAAACCC | |
| | ACAGTCCAAACAAGCCTCAGTGTGATGGAGCTGGTGGGGCCAGTGGGTTGGCCAGCC | |
| | CTGGGTGC | |
| 70 | ATGGACAAACTTAATAAATACCACCAGAAGCATAGAGTAACGATTACGTATAAACACCT | Exemplary REAVR2 |
| | TTTTACTACAGGACCTCCACATGACAGAAGGTTTACATTTCAAGTTATAATAGACGGAG | DNA sequence |
| | AAGAATTTCCAGAAGCCGAAGGCAAAACAAAGCAGGAAGCAAAAAACGCTGCTGCCA | |
| | AATTGGCTGTTGATACACTTAACGCAAATAAGGCAAATAGTCATACAGACGCTTTGGAA | |
| | GGTTCATTCACTGGGAATTACATAGGCCTTGTCAACAGTTATGCTCAGAAGGAGAAGTT | |
| | GTCTGTAAATTACCAACAGTGTGCCTTCAATACCCAGTCACCCCAAAGATTTTGTTATAA | |
| | ATGCATAATTGGGCTGAAAACCTACGGTATTGGTTCAGGCGCTACCAAGCAGGAGGCA | |
| | AAGCAGCTGGCTGCTAAAGAGGCGTATCAGAAGCTACCAGAGAAAAGCTCAATGAGA | |
| | GTTTGCAGAGCATCCTCTGGTCTTAGCAAATCTTCATCCAGTGGCCACTTCAACAGCGG | |
| | ATCCTCAGAAGGAGGCATCTACCTGGGGTTCTATGAGAAGCAAGAAGTAGCTGTGAAG | |
| | ACGTTCTGTGAGGGCAGCCCACGTGCACAGCGGGAAGTCTCTTGTCTGCAAAGCAGCC | |
| | GAGAGAACAGTCACTTGGTGACATTCTATGGGAGTGAGAGCCACAGGGGCCACTTGTT | |
| | TGTGTGTGTCACCCTCTGTGAGCAGACTCTGGAAGCGTGTTTGGATGTGCACAGAGGG | |
| | GAAGATGTGGAAAATGAGGAAGATGAATTTGCCCGAAATGTCCTGTCATCTATATTTAA | |
| | GGCTGTTCAAGAACTACACTTGTCCTGTGGATACACCCACCAGGATCTGCAACCACAAA | |
| | ACATCTTAATAGATTCTAAGAAAGCTGCTCACCTGGCAGATTTTGATAAGAGCATCAAG | |
| | TGGGCTGGAGATCCACAGGAAGTCAAGAGAGATCTAGAGGACCTTGGACGGCTGGTC | |
| | CTCTATGTGGTAAAGAAGGGAAGCATCTCATTTGAGGATCTGAAAGCTCAAAGTAATG | |
| | AAGAGGTGGTTCAACTTTCTCCAGATGAGGAAACTAAGGACCTCATTCATCGTCTCTTC | |
| | CATCCTGGGGAACATGTGAGGGACTGTCTGAGTGACCTGCTGGGTCATCCCTTCTTTTG | |
| | GACTTGGGAGAGCCGCTATAGGACGCTTCGGAATGTGGGAAATGAATCCGACATCAAA | |
| | ACACGAAAATCTGAAAGTGAGATCCTCAGACTACTGCAACCTGGGCCTTCTGAACATTC | |
| | CAAAAGTTTTGACAAGTGGACGACTAAGATTAATGAATGTGTTATGAAAAAAATGAAT | |
| | AAGTTTTATGAAAAAAGAGGCAATTTCTACCAGAACACTGTGGGTGATCTGCTAAAGTT | |
| | CATCCGGAATTTGGGAGAACACATTGATGAAGAAAAGCATAAAAAGATGAAATTAAAA | |
| | ATTGGAGACCCTTCCCTGTATTTTCAGAAGACATTTCCAGATCTGGTGATCTATGTCTAC | |
| | ACAAAACTACAGAACACAGAATATAGAAAGCATTTCCCCCAAACCCACAGTCCAAACAA | |
| | GCCTCAGTGTGATGGAGCTGGTGGGGCCAGTGGGTTGGCCAGCCCTGGGTGC | |
| 71 | ATGGACAAACTTAATAAATACCACCAGAAGCATAGAGTAACGATTACGTATAAACACCT | Exemplary REAVR3 |
| | TTTTACTACAGGACCTCCACATGACAGAAGGTTTACATTTCAAGTTATAATAGACGGAG | DNA sequence |
| | AAGAATTTCCAGAAGCCGAAGGCAAAACAAAGCAGGAAGCAAAAAACGCTGCTGCCA | |
| | AATTGGCTGTTGATACACTTAACGCAAATAAGGCAAATAGTCATACAGACGCTTTGGAA | |
| | GGTTCATTCACTGGGAATTACATAGGCCTTGTCAACAGTTATGCTCAGAAGGAGAAGTT | |
| | GTCTGTAAATTACCAACAGTGTGCCTTCAATACCCAGTCACCCCAAAGATTTTGTTATAA | |
| | ATGCATAATTGGGCTGAAAACCTACGGTATTGGTTCAGGCGCTACCAAGCAGGAGGCA | |
| | AAGCAGCTGGCTGCTAAAGAGGCGTATCAGAAGCTACCAGAGAAAAGCTCAATGAGA | |
| | GTTTGCAGAGCATCCTCTGGTCTTAGCAAATCTTCATCCAGTGGCCACTTCAACAGCTG | |
| | GTCCACAGCAAACAATTTTACTTCTCTGTCAGCGCCTGGAAGTGATTTCTCAGAGACTG | |
| | CATCAGGATCCTCAGAAGGAGGCATCTACCTGGGGTTCTATGAGAAGCAAGAAGTAGC | |
| | TGTGAAGACGTTCTGTGAGGGCAGCCCACGTGCACAGCGGGAAGTCTCTTGTCTGCAA | |
| | AGCAGCCGAGAGAACAGTCACTTGGTGACATTCTATGGGAGTGAGAGCCACAGGGGC | |
| | CACTTGTTTGTGTGTGTCACCCTCTGTGAGCAGACTCTGGAAGCGTGTTTGGATGTGCA | |
| | CAGAGGGGAAGATGTGGAAAATGAGGAAGATGAATTTGCCCGAAATGTCCTGTCATCT | |
| | ATATTTAAGGCTGTTCAAGAACTACACTTGTCCTGTGGATACACCCACCAGGATCTGCA | |
| | ACCACAAAACATCTTAATAGATTCTAAGAAAGCTGCTCACCTGGCAGATTTTGATAAGA | |
| | GCATCAAGTGGGCTGGAGATCCACAGGAAGTCAAGAGAGATCTAGAGGACCTTGGAC | |
| | GGCTGGTCCTCTATGTGGTAAAGAAGGGAAGCATCTCATTTGAGGATCTGAAAGCTCA | |
| | AAGTAATGAAGAGGTGGTTCAACTTTCTCCAGATGAGGAAACTAAGGACCTCATTCATC | |
| | GTCTCTTCCATCCTGGGGAACATGTGAGGGACTGTCTGAGTGACCTGCTGGGTCATCCC | |
| | TTCTTTTGGACTTGGGAGAGCCGCTATAGGACGCTTCGGAATGTGGGAAATGAATCCG | |
| | ACATCAAAACACGAAAATCTGAAAGTGAGATCCTCAGACTACTGCAACCTGGGCCTTCT | |
| | GAACATTCCAAAAGTTTTGACAAGTGGACGACTAAGATTAATGAATGTGTTATGAAAA | |
| | AAATGAATAAGTTTTATGAAAAAAGAGGCAATTTCTACCAGAACACTGTGGGTGATCT | |
| | GCTAAAGTTCATCCGGAATTTGGGAGAACACATTGATGAAGAAAAGCATAAAAAGATG | |
| | AAATTAAAAATTGGAGACCCTTCCCTGTATTTTCAGAAGACATTTCCAGATCTGGTGATC | |
| | TATGTCTACACAAAACTACAGAACACAGAATATAGAAAGCATTTCCCCCAAACCCACAG | |
| | TCCAAACAAGCCTCAGTGTGATGGAGCTGGTGGGGCCAGTGGGTTGGCCAGCCCTGG | |
| | GTGC | |
| 72 | ATGGACAAACTTAATAAATACCACCAGAAGCATAGAGTAACGATTACGTATAAACACCT | Exemplary REAVR4 |
| | TTTTACTACAGGACCTCCACATGACAGAAGGTTTACATTTCAAGTTATAATAGACGGAG | DNA sequence |
| | AAGAATTTCCAGAAGCCGAAGGCAAAACAAAGCAGGAAGCAAAAAACGCTGCTGCCA | |
| | AATTGGCTGTTGATACACTTAACGCAAATAAGGCAAATAGTCATACAGACGCTTTGGAA | |
| | GGTTCATTCACTGGGAATTACATAGGCCTTGTCAACAGTTATGCTCAGAAGGAGAAGTT | |
| | GTCTGTAAATTACCAACAGTGTGCCTTCAATACCCAGTCACCCCAAAGATTTTGTTATAA | |
| | ATGCATAATTGGGCTGAAAACCTACGGTATTGGTTCAGGCGCTACCAAGCAGGAGGCA | |
| | AAGCAGCTGGCTGCTAAAGAGGCGTATCAGAAGCTACCAGAGAAAAGCTCAATGAGA | |
| | GTTTGCAGAGCATCCTCTGGTCTTAGCAAATCTTCATCCAGTGGCCACTTCAACAGCTG | |
| | GTCCACAGCAAACAATTTTACTTCTCTGTCAGCGCCTGNAAGTGATTTCTCAGAGACTG | |
| | CATCAAACAACCACTGTGTTTTTTTCAAGTCTCCTCTCATGAATGGGCTCAGAGAAAATA | |
| | AAAGGAAGCCACGAGTAAAACTGTCATCTAATGATGTGCAAAGAAATAAATATACCGG | |
| | ATCCTCAGAAGGAGGCATCTACCTGGGGTTCTATGAGAAGCAAGAAGTAGCTGTGAAG | |
| | ACGTTCTGTGAGGGCAGCCCACGTGCACAGCGGGAAGTCTCTTGTCTGCAAAGCAGCC | |
| | GAGAGAACAGTCACTTGGTGACATTCTATGGGAGTGAGAGCCACAGGGGCCACTTGTT | |
| | TGTGTGTGTCACCCTCTGTGAGCAGACTCTGGAAGCGTGTTTGGATGTGCACAGAGGG | |
| | GAAGATGTGGAAAATGAGGAAGATGAATTTGCCCGAAATGTCCTGTCATCTATATTTAA | |
| | GGCTGTTCAAGAACTACACTTGTCCTGTGGATACACCCACCAGGATCTGCAACCACAAA | |
| | ACATCTTAATAGATTCTAAGAAAGCTGCTCACCTGGCAGATTTTGATAAGAGCATCAAG | |
| | TGGGCTGGAGATCCACAGGAAGTCAAGAGAGATCTAGAGGACCTTGGACGGCTGGTC | |
| | CTCTATGTGGTAAAGAAGGGAAGCATCTCATTTGAGGATCTGAAAGCTCAAAGTAATG | |
| | AAGAGGTGGTTCAACTTTCTCCAGATGAGGAAACTAAGGACCTCATTCATCGTCTCTTC | |
| | CATCCTGGGGAACATGTGAGGGACTGTCTGAGTGACCTGCTGGGTCATCCCTTCTTTTG | |
| | GACTTGGGAGAGCCGCTATAGGACGCTTCGGAATGTGGGAAATGAATCCGACATCAAA | |
| | ACACGAAAATCTGAAAGTGAGATCCTCAGACTACTGCAACCTGGGCCTTCTGAACATTC | |
| | CAAAAGTTTTGACAAGTGGACGACTAAGATTAATGAATGTGTTATGAAAAAAATGAAT | |
| | AAGTTTTATGAAAAAAGAGGCAATTTCTACCAGAACACTGTGGGTGATCTGCTAAAGTT | |
| | CATCCGGAATTTGGGAGAACACATTGATGAAGAAAAGCATAAAAAGATGAAATTAAAA | |
| | ATTGGAGACCCTTCCCTGTATTTTCAGAAGACATTTCCAGATCTGGTGATCTATGTCTAC | |
| | ACAAAACTACAGAACACAGAATATAGAAAGCATTTCCCCCAAACCCACAGTCCAAACAA | |
| | GCCTCAGTGTGATGGAGCTGGTGGGGCCAGTGGGTTGGCCAGCCCTGGGTGC | |
| 73 | ATGGACAAACTTAATAAATACCACCAGAAGCATAGAGTAACGATTACGTATAAACACCT | Exemplary REAVR5 |
| | TTTTACTACAGGACCTCCACATGACAGAAGGTTTACATTTCAAGTTATAATAGACGGAG | DNA sequence |
| | AAGAATTTCCAGAAGCCGAAGGCAAAACAAAGCAGGAAGCAAAAAACGCTGCTGCCA | |
| | AATTGGCTGTTGATACACTTAACGCAAATAAGGCAAATAGTCATACAGACGCTTTGGAA | |
| | GGTTCATTCACTGGGAATTACATAGGCCTTGTCAACAGTTATGCTCAGAAGGAGAAGTT | |
| | GTCTGTAAATTACCAACAGTGTGCCTTCAATACCCAGTCACCCCAAAGATTTTGTTATAA | |
| | ATGCATAATTGGGCTGAAAACCTACGGTATTGGTTCAGGCGCTACCAAGCAGGAGGCA | |
| | AAGCAGCTGGCTGCTAAAGAGGCGTATCAGAAGCTACCAGAGAAAAGCTCAATGAGA | |
| | GTTTGCAGAGCATCCTCTGGTCTTAGCAAATCTTCATCCAGTGGCCACTTCAACAGCTG | |
| | GTCCACAGCAAACAATTTTACTTCTCTGTCAGCGCCTGGAAGTGATTTCTCAGAGACTG | |
| | CATCAAACAACCACTGTGTTTTTTTCAAGTCTCCTCTCATGAATGGGCTCAGAGAAAATa | |
| | AAAGGAAGCCACGAGTAAAACTGTCATCTAATGATGTGCAAAGAAATAAATATACCAC | |
| | GGACTCCAGGTTTAACGAGGATTTTGAAGACATAGAAGAAATTGGCTCaGGTGGATTT | |
| | GGCCAAGTTTTCAAAGCAAAACACAGAATCGATGGGAAGACTTACGCTATTAAGCGAG | |
| | TTAAATATAACTCAGAGAAGGAAGTGCGTGAAGTAAAGGCGCTGGCGGCACTCAGCC | |
| | ATGTCAATATTGTCCAGTACCATTTTTGTTGGCTGGGACAGGACTGGGAGTACGATACA | |
| | GAGCAGAGCATGAACAGTGCCACCCCACGATCAAAGACAGAGTGCCTTTTCATTCAAA | |
| | TGGAATTCTGTGATAAAGGGACTCTGGAGCAATGGATGACAAGTGTCAAGCAGAGGA | |
| | AAGTAGACAATGATTTGTTTTTGGAATTAGCTGAACAAATAACAACAGGGGTGGATTAT | |
| | ATACACTCAAAAGGCTTAATTCACAGAGACCTTAAGCCAAGTAATATATTTTTAGTGGA | |
| | TGAAAAACACATAAAGATTGGAGACTTCGGCCTCGTAACAGCCCTGGAAAATGATGGA | |
| | AATCGAACAAAGAATACGGGATCTCTTCTATACATGAGCCCAGAACAGTTATCTTTACA | |
| | GGAATATGGAAAAGAAGTGGACATCTTTGCTCTAGGACTTATTCTAGCTGAACTCCTTC | |
| | ACATATGCATCACTATTATAGAAAAGTCAAAGTTTTTCACGGACCTAAGAGATGGCATC | |
| | TTCCATGACGACATATTTGGCAGCAAGGAAAAAAGGCTTCTAACGAAATTACTCTCGAA | |
| | GAAACCCACGGATCGACCTAGTGCTTCTGAAATTCTGATGACCTTGGCTGAATGGAAG | |
| | AATATCTCAGAAGTACAGAAACGACACACACATGGATCCTCAGAAGGAGGCATCTACC | |
| | TGGGGTTCTATGAGAAGCAAGAAGTAGCTGTGAAGACGTTCTGTGAGGGCAGCCCAC | |
| | GTGCACAGCGGGAAGTCTCTTGTCTGCAAAGCAGCCGAGAGAACAGTCACTTGGTGAC | |
| | ATTCTATGGGAGTGAGAGCCACAGGGGCCACTTGTTTGTGTGTGTCACCCTCTGTGAGC | |
| | AGACTCTGGAAGCGTGTTTGGATGTGCACAGAGGGGAAGATGTGGAAAATGAGGAAG | |
| | ATGAATTTGCCCGAAATGTCCTGTCATCTATATTTAAGGCTGTTCAAGAACTACACTTGT | |
| | CCTGTGGATACACCCACCAGGATCTGCAACCACAAAACATCTTAATAGATTCTAAGAAA | |
| | GCTGCTCACCTGGCAGATTTTGATAAGAGCATCAAGTGGGCTGGAGATCCACAGGAAG | |
| | TCAAGAGAGATCTAGAGGACCTTGGACGGCTGGTCCTCTATGTGGTAAAGAAGGGAA | |
| | GCATCTCATTTGAGGATCTGAAAGCTCAAAGTAATGAAGAGGTGGTTCAACTTTCTCCA | |
| | GATGAGGAAACTAAGGACCTCATTCATCGTCTCTTCCATCCTGGGGAACATGTGAGGG | |
| | ACTGTCTGAGTGACCTGCTGGGTCATCCCTTCTTTTGGACTTGGGAGAGCCGCTATAGG | |
| | ACGCTTCGGAATGTGGGAAATGAATCCGACATCAAAACACGAAAATCTGAAAGTGAGA | |
| | TCCTCAGACTACTGCAACCTGGGCCTTCTGAACATTCCAAAAGTTTTGACAAGTGGACG | |
| | ACTAAGATTAATGAATGTGTTATGAAAAAAATGAATAAGTTTTATGAAAAAAGAGGCA | |
| | ATTTCTACCAGAACACTGTGGGTGATCTGCTAAAGTTCATCCGGAATTTGGGAGAACAC | |
| | ATTGATGAAGAAAAGCATAAAAAGATGAAATTAAAAATTGGAGACCCTTCCCTGTATTT | |
| | TCAGAAGACATTTCCAGATCTGGTGATCTATGTCTACACAAAACTACAGAACACAGAAT | |
| | ATAGAAAGCATTTCCCCCAAACCCACAGTCCAAACAAGCCTCAGTGTGATGGAGCTGGT | |
| | GGGGCCAGTGGGTTGGCCAGCCCTGGGTGC | |
| 74 | ATGGACAAACTTAATAAATACCACCAGAAGCATAGAGTAACGATTACGTATAAACACCT | Exemplary REAVR6 |
| | TTTTACTACAGGACCTCCACATGACAGAAGGTTTACATTTCAAGTTATAATAGACGGAG | DNA sequence |
| | AAGAATTTCCAGAAGCCGAAGGCAAAACAAAGCAGGAAGCAAAAAACGCTGCTGCCA | |
| | AATTGGCTGTTGATACACTTAACGCAAATAAGGCAAATAGTCATACAGACGCTTTGGAA | |
| | GGTTCATTCACTGGGAATTACATAGGCCTTGTCAACAGTTATGCTCAGAAGGAGAAGTT | |
| | GTCTGTAAATTACCAACAGTGTGCCTTCAATACCCAGTCACCCCAAAGATTTTGTTATAA | |
| | ATGCATAATTGGGCTGAAAACCTACGGTATTGGTTCAGGCGCTACCAAGCAGGAGGCA | |
| | AAGCAGCTGGCTGCTAAAGAGGCGTATCAGAAGCTACCAGAGAAAAGCTCAATGAGA | |
| | GTTTGCAGAGCATCCTCTGGTCTTAGCAAATCTTCATCCAGTGGCCACTTCAACAGCTG | |
| | GTCCACAGCAAACAATTTTACTTCTCTGTCAGCGCCTGGAAGTGATTTCTCAGAGACTG | |
| | CATCAAACAACCACTGTGTTTTTTTCAAGTCTCCTCTCATGAATGGGCTCAGAGAAAATa | |
| | AAAGGAAGCCACGAGTAAAACTGTCATCTAATGATGTGCAAAGAAATAAATATACCAC | |
| | GGACTCCAGGTTTAACGAGGATTTTGAAGACATAGAAGAAATTGGCTCaGGTGGATTT | |
| | GGCCAAGTTTTCAAAGCAAAACACAGAATCGATGGGAAGACTTACGCTATTAgGCGAG | |
| | TTAAATATAACTCAGAGAAGGAAGTGCGTGAAGTAAAGGCGCTGGCGGCACTCAGCC | |
| | ATGTCAATATTGTCCAGTACCATTTTTGTTGGCTGGGACAGGACTGGGAGTACGATACA | |
| | GAGCAGAGCATGAACAGTGCCACCCCACGATCAAAGACAGAGTGCCTTTTCATTCAAA | |
| | TGGAATTCTGTGATAAAGGGACTCTGGAGCAATGGATGACAAGTGTCAAGCAGAGGA | |
| | AAGTAGACAATGATTTGTTTTTGGAATTAGCTGAACAAATAACAACAGGGGTGGATTAT | |
| | ATACACTCAAAAGGCTTAATTCACAGAGACCTTAAGCCAAGTAATATATTTTTAGTGGA | |
| | TGAAAAACACATAAAGATTGGAGACTTCGGCCTCGTAACAGCCCTGGAAAATGATGGA | |
| | AATCGAACAAAGAATACGGGATCTCTTCTATACATGAGCCCAGAACAGTTATCTTTACA | |
| | GGAATATGGAAAAGAAGTGGACATCTTTGCTCTAGGACTTATTCTAGCTGAACTCCTTC | |
| | ACATATGCATCACTATTATAGAAAAGTCAAAGTTTTTCACGGACCTAAGAGATGGCATC | |
| | TTCCATGACGACATATTTGGCAGCAAGGAAAAAAGGCTTCTAACGAAATTACTCTCGAA | |
| | GAAACCCACGGATCGACCTAGTGCTTCTGAAATTCTGATGACCTTGGCTGAATGGAAG | |
| | AATATCTCAGAAGTACAGAAACGACACACACATGGATCCTCAGAAGGAGGCATCTACC | |
| | TGGGGTTCTATGAGAAGCAAGAAGTAGCTGTGAAGACGTTCTGTGAGGGCAGCCCAC | |
| | GTGCACAGCGGGAAGTCTCTTGTCTGCAAAGCAGCCGAGAGAACAGTCACTTGGTGAC | |
| | ATTCTATGGGAGTGAGAGCCACAGGGGCCACTTGTTTGTGTGTGTCACCCTCTGTGAGC | |
| | AGACTCTGGAAGCGTGTTTGGATGTGCACAGAGGGGAAGATGTGGAAAATGAGGAAG | |
| | ATGAATTTGCCCGAAATGTCCTGTCATCTATATTTAAGGCTGTTCAAGAACTACACTTGT | |
| | CCTGTGGATACACCCACCAGGATCTGCAACCACAAAACATCTTAATAGATTCTAAGAAA | |
| | GCTGCTCACCTGGCAGATTTTGATAAGAGCATCAAGTGGGCTGGAGATCCACAGGAAG | |
| | TCAAGAGAGATCTAGAGGACCTTGGACGGCTGGTCCTCTATGTGGTAAAGAAGGGAA | |
| | GCATCTCATTTGAGGATCTGAAAGCTCAAAGTAATGAAGAGGTGGTTCAACTTTCTCCA | |
| | GATGAGGAAACTAAGGACCTCATTCATCGTCTCTTCCATCCTGGGGAACATGTGAGGG | |
| | ACTGTCTGAGTGACCTGCTGGGTCATCCCTTCTTTTGGACTTGGGAGAGCCGCTATAGG | |
| | ACGCTTCGGAATGTGGGAAATGAATCCGACATCAAAACACGAAAATCTGAAAGTGAGA | |
| | TCCTCAGACTACTGCAACCTGGGCCTTCTGAACATTCCAAAAGTTTTGACAAGTGGACG | |
| | ACTAAGATTAATGAATGTGTTATGAAAAAAATGAATAAGTTTTATGAAAAAAGAGGCA | |
| | ATTTCTACCAGAACACTGTGGGTGATCTGCTAAAGTTCATCCGGAATTTGGGAGAACAC | |
| | ATTGATGAAGAAAAGCATAAAAAGATGAAATTAAAAATTGGAGACCCTTCCCTGTATTT | |
| | TCAGAAGACATTTCCAGATCTGGTGATCTATGTCTACACAAAACTACAGAACACAGAAT | |
| | ATAGAAAGCATTTCCCCCAAACCCACAGTCCAAACAAGCCTCAGTGTGATGGAGCTGGT | |
| | GGGGCCAGTGGGTTGGCCAGCCCTGGGTGC | |
| 75 | ATGGCCAGTGATACCCCAGGTTTCTACATGGACAAACTTAATAAATACCGCCAGATGCA | Exemplary REAVR7 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGAAAATTGCTGATACTTCAGAA | |
| | GGAGGCATCTACCTGGGGTTCTATGAGAAGCAAGAAGTAGCTGTGAAGACGTTCTGTG | |
| | AGGGCAGCCCACGTGCACAGCGGGAAGTCTCTTGTCTGCAAAGCAGCCGAGAGAACA | |
| | GTCACTTGGTGACATTCTATGGGAGTGAGAGCCACAGGGGCCACTTGTTTGTGTGTGT | |
| | CACCCTCTGTGAGCAGACTCTGGAAGCGTGTTTGGATGTGCACAGAGGGGAAGATGTG | |
| | GAAAATGAGGAAGATGAATTTGCCCGAAATGTCCTGTCATCTATATTTAAGGCTGTTCA | |
| | AGAACTACACTTGTCCTGTGGATACACCCACCAGGATCTGCAACCACAAAACATCTTAA | |
| | TAGATTCTAAGAAAGCTGCTCACCTGGCAGATTTTGATAAGAGCATCAAGTGGGCTGG | |
| | AGATCCACAGGAAGTCAAGAGAGATCTAGAGGACCTTGGACGGCTGGTCCTCTATGTG | |
| | GTAAAGAAGGGAAGCATCTCATTTGAGGATCTGAAAGCTCAAAGTAATGAAGAGGTG | |
| | GTTCAACTTTCTCCAGATGAGGAAACTAAGGACCTCATTCATCGTCTCTTCCATCCTGGG | |
| | GAACATGTGAGGGACTGTCTGAGTGACCTGCTGGGTCATCCCTTCTTTTGGACTTGGGA | |
| | GAGCCGCTATAGGACGCTTCGGAATGTGGGAAATGAATCCGACATCAAAACACGAAAA | |
| | TCTGAAAGTGAGATCCTCAGACTACTGCAACCTGGGCCTTCTGAACATTCCAAAAGTTT | |
| | TGACAAGTGGACGACTAAGATTAATGAATGTGTTATGAAAAAAATGAATAAGTTTTAT | |
| | GAAAAAAGAGGCAATTTCTACCAGAACACTGTGGGTGATCTGCTAAAGTTCATCCGGA | |
| | ATTTGGGAGAACACATTGATGAAGAAAAGCATAAAAAGATGAAATTAAAAATTGGAGA | |
| | CCCTTCCCTGTATTTTCAGAAGACATTTCCAGATCTGGTGATCTATGTCTACACAAAACT | |
| | ACAGAACACAGAATATAGAAAGCATTTCCCCCAAACCCACAGTCCAAACAAGCCTCAGT | |
| | GTGATGGAGCTGGTGGGGCCAGTGGGTTGGCCAGCCCTGGGTGC | |
| 76 | ATGGCCAGTGATACCCCAGGTTTCTACATGGACAAACTTAATAAATACCGCCAGATGCA | Exemplary REAVR8 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGAACGGTCTCGGAGAAAATAA | |
| | AAGGAAATCAGGAGTAAAAGTATCCCCTGATGATGTGCAAAGAAATAAATATACCAAA | |
| | ATTGCTGATACTTCAGAAGGAGGCATCTACCTGGGGTTCTATGAGAAGCAAGAAGTAG | |
| | CTGTGAAGACGTTCTGTGAGGGCAGCCCACGTGCACAGCGGGAAGTCTCTTGTCTGCA | |
| | AAGCAGCCGAGAGAACAGTCACTTGGTGACATTCTATGGGAGTGAGAGCCACAGGGG | |
| | CCACTTGTTTGTGTGTGTCACCCTCTGTGAGCAGACTCTGGAAGCGTGTTTGGATGTGC | |
| | ACAGAGGGGAAGATGTGGAAAATGAGGAAGATGAATTTGCCCGAAATGTCCTGTCAT | |
| | CTATATTTAAGGCTGTTCAAGAACTACACTTGTCCTGTGGATACACCCACCAGGATCTG | |
| | CAACCACAAAACATCTTAATAGATTCTAAGAAAGCTGCTCACCTGGCAGATTTTGATAA | |
| | GAGCATCAAGTGGGCTGGAGATCCACAGGAAGTCAAGAGAGATCTAGAGGACCTTGG | |
| | ACGGCTGGTCCTCTATGTGGTAAAGAAGGGAAGCATCTCATTTGAGGATCTGAAAGCT | |
| | CAAAGTAATGAAGAGGTGGTTCAACTTTCTCCAGATGAGGAAACTAAGGACCTCATTC | |
| | ATCGTCTCTTCCATCCTGGGGAACATGTGAGGGACTGTCTGAGTGACCTGCTGGGTCAT | |
| | CCCTTCTTTTGGACTTGGGAGAGCCGCTATAGGACGCTTCGGAATGTGGGAAATGAAT | |
| | CCGACATCAAAACACGAAAATCTGAAAGTGAGATCCTCAGACTACTGCAACCTGGGCC | |
| | TTCTGAACATTCCAAAAGTTTTGACAAGTGGACGACTAAGATTAATGAATGTGTTATGA | |
| | AAAAAATGAATAAGTTTTATGAAAAAAGAGGCAATTTCTACCAGAACACTGTGGGTGA | |
| | TCTGCTAAAGTTCATCCGGAATTTGGGAGAACACATTGATGAAGAAAAGCATAAAAAG | |
| | ATGAAATTAAAAATTGGAGACCCTTCCCTGTATTTTCAGAAGACATTTCCAGATCTGGT | |
| | GATCTATGTCTACACAAAACTACAGAACACAGAATATAGAAAGCATTTCCCCCAAACCC | |
| | ACAGTCCAAACAAGCCTCAGTGTGATGGAGCTGGTGGGGCCAGTGGGTTGGCCAGCC | |
| | CTGGGTGC | |
| 77 | ATGGCCAGTGATACCCCAGGTTTCTACATGGACAAACTTAATAAATACCGCCAGATGCA | Exemplary REAVR9 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGAACGGTCTCGGAGAAAATAA | |
| | AAGGAAATCAGGAGTAAAAGTATCCCCTGATGATGTGCAAAGAAATAAATATACCTTG | |
| | GACGCCAGGTTTAACAGCGATTTTGAAGACATAGAAGAAATTGGCTTAGGTGGATTTG | |
| | GTCAAGTTTTCAAAGCGAAACACAGAATTGATGGAAAGAGATACGCTATTAAGCGCGT | |
| | TAAATATAACACGGAGAAGGCGGAGCACGAAGTACAAGCGCTGGCAGAACTCAATCA | |
| | CGTCAACATTGTCCAATACCATAGTTGTTGGGAGGGAGTTGACTATGATCCTGAGCACA | |
| | GCATGAGTGATACAAGTCGATACAAAACCCGGTGCCTCTTTATTCAAATGGAATTCTGT | |
| | GATAAAGGAACTTTGGAGCAATGGATGAGAAACAGAAATCAGAGTAAAGTGGACAAA | |
| | GCTTTGATTTTGGACTTATATGAACAAATCGTGACCGGAGTGGAGTATATACACTCGAA | |
| | AGGGTTAATTCACAGAGATCTTAAGCCAGGTAATATATTTTTAGTAGATGAAAGACACA | |
| | TTAAGATCGGAGACTTTGGCCTTGCAACAGCCCTGGAAAATGATGGAAAATCCCGAAC | |
| | AAGGAGAACAGGAACTCTTCAATATATGAGTCCAGAACAGTTATTTTTAAAGCACTATG | |
| | GAAAAGAAGTGGACATCTTTGCTTTGGGCCTTATTCTAGCTGAACTTCTTCACACGTGCT | |
| | TCACGGAGTCAGAGAAAATAAAGTTTTTCGAAAGTCTAAGAAAAGGCGACTTCTCTAAT | |
| | GATATATTCGACAACAAAGAAAAAAGCCTTCTAAAAAAACTACTCTCAGAGAAACCCAA | |
| | GGACCGACCTGAGACATCTGAAATCCTGAAGACCTTGGCTGAATGGAGGAACATCTCA | |
| | GAGAAAAAGAAAAGAAACACATGTAAAATTGCTGATACTTCAGAAGGAGGCATCTACC | |
| | TGGGGTTCTATGAGAAGCAAGAAGTAGCTGTGAAGACGTTCTGTGAGGGCAGCCCAC | |
| | GTGCACAGCGGGAAGTCTCTTGTCTGCAAAGCAGCCGAGAGAACAGTCACTTGGTGAC | |
| | ATTCTATGGGAGTGAGAGCCACAGGGGCCACTTGTTTGTGTGTGTCACCCTCTGTGAGC | |
| | AGACTCTGGAAGCGTGTTTGGATGTGCACAGAGGGGAAGATGTGGAAAATGAGGAAG | |
| | ATGAATTTGCCCGAAATGTCCTGTCATCTATATTTAAGGCTGTTCAAGAACTACACTTGT | |
| | CCTGTGGATACACCCACCAGGATCTGCAACCACAAAACATCTTAATAGATTCTAAGAAA | |
| | GCTGCTCACCTGGCAGATTTTGATAAGAGCATCAAGTGGGCTGGAGATCCACAGGAAG | |
| | TCAAGAGAGATCTAGAGGACCTTGGACGGCTGGTCCTCTATGTGGTAAAGAAGGGAA | |
| | GCATCTCATTTGAGGATCTGAAAGCTCAAAGTAATGAAGAGGTGGTTCAACTTTCTCCA | |
| | GATGAGGAAACTAAGGACCTCATTCATCGTCTCTTCCATCCTGGGGAACATGTGAGGG | |
| | ACTGTCTGAGTGACCTGCTGGGTCATCCCTTCTTTTGGACTTGGGAGAGCCGCTATAGG | |
| | ACGCTTCGGAATGTGGGAAATGAATCCGACATCAAAACACGAAAATCTGAAAGTGAGA | |
| | TCCTCAGACTACTGCAACCTGGGCCTTCTGAACATTCCAAAAGTTTTGACAAGTGGACG | |
| | ACTAAGATTAATGAATGTGTTATGAAAAAAATGAATAAGTTTTATGAAAAAAGAGGCA | |
| | ATTTCTACCAGAACACTGTGGGTGATCTGCTAAAGTTCATCCGGAATTTGGGAGAACAC | |
| | ATTGATGAAGAAAAGCATAAAAAGATGAAATTAAAAATTGGAGACCCTTCCCTGTATTT | |
| | TCAGAAGACATTTCCAGATCTGGTGATCTATGTCTACACAAAACTACAGAACACAGAAT | |
| | ATAGAAAGCATTTCCCCCAAACCCACAGTCCAAACAAGCCTCAGTGTGATGGAGCTGGT | |
| | GGGGCCAGTGGGTTGGCCAGCCCTGGGTGC | |
| 78 | ATGGCCAGTGATACCCCAGGTTTCTACATGGACAAACTTAATAAATACCGCCAGATGCA | Exemplary REAVR10 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGAAAATTCAAGAAACCTCCCAT | |
| | GGTGGCATCTACCTAGGTTTCTATAAAGGGAAAGAAGTAGCTGTGAAGAGGGTTCCAA | |
| | AAGGTAATAATAAAGAGCTGGTCTGTCTGGAGCAATTACGGAAGAATAATCATTTAGT | |
| | ACGTTACTGTGGATATGAAGAAGATAGCTCATGTTCATATCTATGTCTTTTTCTGTATGA | |
| | GAGAAACCTTGAAGAACACATTAGTAATATTAAAGATGTAACTGAGATCCAAGACATG | |
| | CTTCAGTCTATATTCCTGGGCTTGCATGAGCTACATGAGATAGGATTTTGCCATCGGGA | |
| | CCTTCATCCATCCAATATTTTAATTGATCATGAGGGTAAAGCTTACCTGGCAGATTTCAG | |
| | CGGGAGTCTGAAGTTGGAAGATTCTGAAGACAAGGAAGGGTTGATCAAGAGTGACAT | |
| | GAAGGCATTGGGAAACCTTGTCTTGTATGTAGTAACAAGAGGAACGGCCTGCTTCCAA | |
| | CCAGATAGAGAAAGGGATGTGACGGAAGCTTGTCCAGATTCAGTGGAAGATTTTATAG | |
| | AAGCAAGGGATCTAGTAAATAAATTAATGTCTCCAGATACAGAGTATTCATCTGTGATT | |
| | GATTTCCTTAACCATCCCTTCTTCTGGAGTCACACAAGCAGGTATAAGTTTCTTCAAGAT | |
| | GTTGGGAATGAGAATGATGTCAAAATGCGCACAGAGGACAGTGCCCTTTTCAGAACCC | |
| | TGAATGATTCTCATGTGACAATGACAAAAACCTTCCACCCCTGGGGTTGTAAGGTTGAC | |
| | CAGGAGGTCCTGAATGACATGAACAGGTTCCACAGTAAGAAGAAAATCTATTATCAGG | |
| | ACACAGTCACTGACCTGTTGAAGTTCATTAGAAACTTGGGTGCTCACTTCGATGAGAAA | |
| | AAAACAGAAATAAAGGAGATCATTGGAGATCCCTGCCAGTATTTTCTCAAGAAGTTTCC | |
| | AGATCTCACGGTTTATGTCTATGAAACTCTGCGACATTTGGGGAAGGGATACATTAAGC | |
| | ATTTTCCAAACCCCGTAGTTCTCTTCTATAATTGCATGCAGCTCAACAATTCGGGTATGA | |
| | CT | |
| 79 | ATGGCCAGTGATACCCCAGGTTTCTACATGGACAAACTTAATAAATACCGCCAGATGCA | Exemplary REAVR11 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGAGAATTCAGAACACTTCCCAG | |
| | GGTGGCATCTACTTGGGGCTCCACGGTGGGACGGAGGTAGCAGTAAGAATAAGCCGC | |
| | AGTACAGAGGGTGACAAAGAGAAAAGGTTCTTCGAACAATGTGGTAACTGTGAACATC | |
| | TACTGAAGCTCTTCCAGTTTGAGAAGGCAAGAGGCTACATGTACTTGTGCTTCCCCCTC | |
| | TGGGAGAAAAACCTTGAAGAACATCTGCAGGAACCAGAAGACCAAATGGATTACAAA | |
| | GATGCTCTGAGGATGATCTTCCAGGCAGTGAGAGAGCTGCACTCCCTTGGATTTGCTCA | |
| | CCAGGATCTGCATCCCACCAACTTTTTCATAGATTTAGGTGGCAAAATTTACTTGGCAG | |
| | ACTTTGATAATAAAAGAAAGTTGATTGAAGGCAAAAAAGAACTCGTAAACTCAGATTT | |
| | AGAGGCCCTCAGCAGGCTCGTGCTGTATGTCTTAACAGGGGGTAGGAAACCCCTTCAG | |
| | CAAGTCAGTACCAAGGATTTGACTGCTGATTCCCCAGATTATGAGGAGGCTCTGGACCT | |
| | TGTAACTAGCCTGGTCTCTCATGATGAACGAGGCTTGGAAGGTTTGAGCAAACATCCCT | |
| | ATTTCTGGAGCAAACAGACCAGGTTCAAGTTCCTGAAGGGTATATGGAATAAAATCAA | |
| | AGATCTCCACAACCGAAAAGCTGTCTTTAAAGCTGCTAATGCTACTGAACGTTTTCCTTA | |
| | TCCACAGTGGACCGAGAAGATTGACAAATATGTTCTGAAAATCATGGAAAATCCTAGG | |
| | AAAACAAAACCACTCAAATATTGCAATGATGTCACCGACCTACTGAGGCTCATCAGAAA | |
| | CCTGGATGAACACCCAGATACCAGGATCAGCAACAGAATAGGAGACCATGCTGAGTAT | |
| | TTCTTGAAGCGCTTTCCAGCACTTACCATTTATGTCTACAACAGTTTACGTCAGAATCCC | |
| | AAATACAGTCACTTTGCAGACATTCAGGACCCTTCTCTG | |
| 80 | ATGGCCAGTGATACCCCAGGTTTCTACATGGACAAACTTAATAAATACCGCCAGATGCA | Exemplary REAVR12 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGAAGATTACGAAAGACAACATA | |
| | ACGGAAGTCTACCTTGGTTTCTATAACAATGTGATTGAAGTGGCAGTGAAATGTCACAG | |
| | GAACGGCAGTGAAGAAGCCAAAAATGAAATGGATTGCCTCAGAGATCCTACGATCAAT | |
| | GGTAGCAGCCTGTTTGTTAACCTCCTGCATTGGGAAAGTGATGAGTTTTGCGATTACTT | |
| | ATGCCTGAATCTCTATGAGTATAATCTCGAGGAATGTGTGACCATGTGGCCTAAGGAAA | |
| | TTCGAGAGAAGGCTCCTAAAATAATGAAGCAGTTGGTTGACGCTGTTCAGGTTTTGCAT | |
| | GGAGCTGGATTTGCTCACCGGGATTTACATCCAACAAATGTCCTCAGAGATGTTAAAGA | |
| | AAATTTTCGCCTGGCAGACTTTGATAGGAGTGTGAATTGTAAGCATCCAGCTGTGACCA | |
| | TATCCGGCAAAGCGACCTGGGAGGCTTCCGAAATCCTTCAGAAACTGGAACTCTGGCC | |
| | GACAGCAACCTTCGAAGCGGATGAGCTTTTCAAAGCGGATTTACAGGCTCTTGGACGG | |
| | CTTTTGCATTATGTTATGACCGGAGGGAAGGATCCCTGTAGCAGTGAAAATGATCTGTG | |
| | CGAGAATAATCTTTCTCTCAATGAGGATCTTTGCGAACCCAAGAATGCCGAAGCCAGAG | |
| | ATTTCATCGAGCGCTTGTTGGCGCCAGCAGAGAAAAGAGCTACATTAAGTGAGATCAG | |
| | ACAACATCCGCTGCTGTGGAAAGATTTGGGAAAGAGCCAGTTTGTCCAAGGCCTTGCA | |
| | AATGAAAACGGTGTGGCAAAACGGGATAAATCCAGTCCGATAGTCCTGATTCTGAATG | |
| | AGGTTGGCAACAACCAGGAAAGATCATTTCACAAGTGGACAGAGAAGGTCCACCTTAA | |
| | AATCCTAAATAATATGAATAAGAGACAGCGCACAAAATATGAGGACAACACCAGTGAC | |
| | CTCCTGAAATTCATAAGAAACCTGACCCAGCATTTTGATGAAAAGGAAGAATTACACAG | |
| | TTTGATTAAAAGTCCCGAATCTTATTTCTGTGAATTATTTCCTGATCTCACCATCGCTACC | |
| | TACAATGCTGTGAAGGACACCGAGTGGGTAAAGCACTTTCCAAAAGAA | |
| 81 | ATGGCCAGTGATACCCCAGGTTTCTACATGGACAAACTTAATAAATACCGCCAGATGCA | Exemplary REAVR13 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGTGGATCCAGAGGACTACCCAA | |
| | GGAGGGGTCTACCTAGGATTCTATGCTGGAGAAGAGGTGGCTGTGAAGATATTCTGG | |
| | AAAGGTGCAGAAAATGCCAAGCGAGAGAAAGTGTGCCTTGAGAAATGTCGAACCAGC | |
| | AACCACCTGGTGAAGTTTTGCGGCTGGGAAGAGAGGAAGACCTGTCTCTACCTGTGCC | |
| | TCTCCTTGTGTGAGAGGAATCTTCAAGAGTACTTCAAGGAAGAAAAGAAAAAAACTAT | |
| | GGAAAGCGAAGAGATTCTTCGAACAGTATTTCAGGCCGTAAAGGAACTTCACACATTT | |
| | GGATTTGGCCATCAGGAACTGCACCCAAGTAACATTTTGATAGATGCCACTGGCAGGA | |
| | TTTTCCTAGCCGACTTTGATAAGAGCAGAGAAATAACTGATGATATAAAATATTCAATA | |
| | ATCTCAGAAGACCTACAGGGCCTCCAAAAACTTATCCTGTATGTTGTGATGAGAGGCCA | |
| | GTTGGACTTTGAAGATTTGCCTACAGAATGCCCAGAAAATGTAAAGAACCATAAGGAA | |
| | ATTGAAGACCTCCGAGGAAGGCTGGGACTCTCTGACCAAAGTACCCCAGCCGATGACC | |
| | TACTGGAAAAGCTGATCAATCATCCATATTTTTGGTCCAAGGAGCAAAAGTACGGAGT | |
| | GTTAAAAAAGGTCGGGAATGAGATACGTGCCGCTAAGTGCTGTAATAAAACCAGTGAA | |
| | ACAAATATTCTGGAAGCTTTGAAACCTATGGATAATTCTTTCAGGGACTGGAAAAAAAA | |
| | GATTGATAAAACAGTACTGGACTCTATGTTTTTTCTGTCCACTGAGAAGGGACAGAAGG | |
| | GGAATCAGAAGGGCAACAAACGAAGTTATGGCCCCTGCATAACAGAGTTACTAAAGTT | |
| | GATTAGGAATATGGGTGAGCACTACAATGAGAAGGATGACAAGGTGAAAGACATAAT | |
| | TCGGGACCCGGCAGACTATTTTCTGGATCGCTTTCCAGATTTAACCATTTATATCTATCA | |
| | GCGTTTGTGTAATACGGAATACAGTATACATTTTCCAAATGCTAAGAATCCTTCTCACTC | |
| | A | |
| 82 | ATGGCCAGTGATACCCCAGGTTTCTACATGGACAAACTTAATAAATACCGCCAGATGCA | Exemplary REAVR14 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGAAGATTGCTGAGAGCTCTCAA | |
| | GGGGGTGTCTACCTGGGCTTCTATGGGGATGGAGTTGAGGTTGCAGTGAAACGCATG | |
| | AGAAAGAACAGTAATGAAGTGAAAATGGAAAAGCTGTGTCTTGAACACCCAAAGATTA | |
| | TGGGCAACAGCTGTTTTGTTAAACTCTATGGCAGTGAAACGGACGCTACATGTCATTAT | |
| | TTGTGCTTGACCCTCTGTGAATGCACTCTAGAGACATACATACAAGAGGAAGAAGGAA | |
| | ATCTAAAGACTAAAGTGCCTCACATATTTAAGCAGCTAATCGGAGCAGTGCAGATATTA | |
| | CATGAAGCTGGATTTGCACACACAGACCTTCATCCTTCAAATATCCTATTTAATGTAGAA | |
| | GGGAAGCTTCAACTGGCTGATTTTGATAAGTGTTTAAGGTTTGAAGACTGTCCACATAA | |
| | AGGCCAATCAGAAAGAAGTGATAAAACTGAAGAGGAGCTGAAAAATGAGGACATTCA | |
| | GGCAATTGGAAATCTGCTTTGTTATGTCACCACTGAAGGAAAATATGTATACAAGACAG | |
| | AAGAGGAGAAGCTTGGAGACTGCTATAGTGCTGAGGTGAGGGATCTTATACAGAAAC | |
| | TTTCGGCCCCACAGGGACAACCACCTCCACTAAAGGAGGCCCTGCAACATCCATTCTTC | |
| | TGGAGTCAAGAAAGCAAATACAGATTTCTACAAGACATTGGTAATGAGAATGAAGTAA | |
| | AAGTCAGAAACGACAAACATGAAATAGTAAAGAGTCTTAATGAAAAGGCACAAGACCC | |
| | TTCTTCATCATTCCGTGAATGGACCAAAAAGGTAAATGACATTGTTTTTAAAAACATGG | |
| | ATAGTCGTAATAGGAAACCAGAACTCAAGTATAGGAATGACACTGCAAGCCTCTTGAA | |
| | GTTTATACGAAATATGGGAGCCCATTTTGAGGAGAAGCCACTAGAAATTAAGAATATT | |
| | GTCAAGAATCCGTGCAACTACTTCCTTGACTTGTTTCCAGATCTTGTTATTTCTGTCTACA | |
| | ATGTTGTGAAATCAACTCAATGGGAAGAACATTTTCCAAAAGCTGAAACTTCT | |
| 83 | ATGGCCAGTGATACCCCAGGTTTCTACATGGACAAACTTAATAAATACCGCCAGATGCA | Exemplary REAVR15 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGAAAATTGCAGACAGTTCTGAA | |
| | GGGGGTGTGTACTTGGGGTTCTATGGAGAACAGGAGGTAGCTGTGAAGCGATTCTAT | |
| | GAAGGCAGTACCCTGGGGCAAAAGGAAGTCTCCTGTTTGCAGAGTTGCCGGGAGAAG | |
| | AGTAACTTGGTGACATTCTATGAGAGTGAGAGGCAAAGTGACTGCCTGTATGTGTGCC | |
| | TCACCCTCTGCGAGCAGACACTGGAGGAGCACTTCAATGCCCGCAAAAGGGCAGACGT | |
| | GGGAAACAAGGAAGATAAGTTTGCCCGGAAAGTCCTCTCTTCTATATTTAAAGCAGTCA | |
| | AAGGGCTGCACCAGTGTGGATATGCTCACCAAGACCTGCAGCCACGAAACATCTTAAT | |
| | AGATTCCAATGATGCTATTTGCCTGGCAGATTTTGATAAAAGCATCAAGATGACTAGAA | |
| | AACAGGAAATCAAAACAGATCTAAAGGACCTCGGACTACTGGTCATATATGTTGTAAA | |
| | AAAGGGAGACATCCCTTTTGAAAGACTGAAGACTAAAAGTAATGAAGACGTGGTTCGA | |
| | CTTTCTCCAGATGTGGAAACTGAAAACCTCATTCACCATCTGATCTTTCCTAAGGAAGAA | |
| | TTTACAGACCTAAGTGGCCTGCTGGGGCATCCCTTCTTTTGGAGTTGGGAGAGTCGCTA | |
| | TAGGGCCCTGAGTGAAGTGGGAAATGAAAATGACATCAAAATTCGAAAGCCTAATAGC | |
| | AAGATCTTTCAACTGCTGGAGCCTAAATCTTCTGAACATTCCAAAAGTTTTAACCGCTGG | |
| | AAGGCTAAGATTGATGAGTATTTTATGAAACAAATGGATCAGTTTTACAAAAAAGACG | |
| | GTAGGTATCAGAACACTGTGGGTGATCTGCTAAGATTCATCCGGAATACAAGACAACA | |
| | TCTTCATGAAAAAAAGAATGAAAAGGTGAAGTTGAGAATTGGAGAACCTTCCCAATAT | |
| | TTTCAGGAAAAATTTCCGGATTTCTTTTTATATGTCTATGAAAGACTACACAGCACTGAA | |
| | TATGGAAAGCATTTTGTCCAAGAC | |
| 84 | ATGGCCAGTGATACCCCAGGTTTCTACATGGACAAACTTAATAAATACCGCCAGATGCA | Exemplary REAVR16 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGAAAATCGCCGACACGCAGGG | |
| | GGGCGGCGTCTACCTGGGGCTCCTCGAGGACCAGGAGGTGGCCGTGAAGGTCTTCGT | |
| | TCAGGGCAGTGAATGTGCCGGCAGGGAGCTGTCCGCCCTCAAGAGTGGCCAGGACAG | |
| | CAGCCGCCTGGTGACCCTGCTTGCCTGCGAGAGCCACGAAGCCAACCTGTATGTGTGT | |
| | ATGACCCTGTGTGAGTGGACGCTGGAGGAGTTCCTGGAGCACCGACCTCAGCCTGCCC | |
| | AAGGCGACGAGGACAAGTGGGCCCGCCAGGTCTTGCACTCCGTGTTTGAGGCAGTTCG | |
| | AGAGCTGCACCTACAGCGTGCTCATGCCCACCAGGACCTGCAGCCACAGAACATTCTCA | |
| | TAGACGCTGCGCACGGCATCCGCCTGGCTGACTTTGATCAGAGCGTCCAGCTGGCTGA | |
| | ACCCACCAAAGTCTCACAGGCCGTCAGGACAGACCTGCAGGCCCTGGGCCGGCTGGTC | |
| | GTGTACGTGCTGTTGAAGGGGGATGTTCCGTTTGAGCAGCTGAGTGCCATGCATCATC | |
| | GAAAGGTCTGGCGTCTCTGTCTTGAACGGGAGACCAGGGACCTCACGCGCTGCCTGCT | |
| | GACCCCCCAGCAGGCTCAGGAGGACCCTCTGGGCAGCCTGTTGGGCCACCCCTTCTTCT | |
| | GGGAGTGGCAGAGCCGCTACAGGACGCTCCGGAACGTGGGGAACGAATCTGACATCA | |
| | AAAGGTGCAATCTCAGGGGCGCCATCGTGCAGCTGCTGAGTCTGAAGCCCTCTGAGCA | |
| | CAGCCTCAGCTTCGCCCAGTGGACGAGCAAGGTGGACAGCCTCGTGCTGGGACAAATG | |
| | AACGACTTCTACAAGAACAAACCTCACCTGTGCTACACGGACAGCGTGCAGGACCTGCT | |
| | GAGGTTCATCCGCAACATTGGGGAGCACATTGACGAGGACTGCAACCGAGAGATGAA | |
| | GGCCCGGCTCGGGGACCCCGCTATCTACTTCCTGCGCACATTTCCAGACCTGCTGATGC | |
| | ACGTGTACAAGCAGCTGCAGGATTCGGAATTCCGGAGGCACTTCCCACAGCTCCCTCTG | |
| | CCTGCGGGCCCCGGCGTGTTGGAGACGGAGGTGCCAGGGGGCCAGGCGCTGGGGGC | |
| | TGATCCC | |
| 85 | ATGGCCAGTGATACCCCAGGTTTCTACATGGACAAACTTAATAAATACCGCCAGATGCA | Exemplary REAVR17 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGAAGGTTGCAGAAACTTCTGAA | |
| | GGTGGTGTGTACCTGGGGTTCTATGATGGGAAAGAGGCAGCCGTGAAGCGATTCCGA | |
| | GAAGACAGTAAACGGGCTCAGCAAGAGATGGACTGCATCCTCCATTGCCAAAAGAATA | |
| | GCAACTTGGTGATGTTTTATGGGAGAGAAAGCAAAGACAACTGTTTGTATTTGTGCTTT | |
| | TCTCTTTGTGAGAGGACTCTGGAGGAACATGTGACAACAAGAGCCTTGGAGAATGAGG | |
| | ACTTCCAAAACATTTTTGAGTCCTTATTTAAGGCAGTGCAAGAACTACACAGACACGAC | |
| | TATCTCCACCAGGACCTGCACCCAGGGAATATCTTGATAGATTCTGAAGGTAAAGTTCA | |
| | CTTGGCAGATTTTGATAAGAGTGAAAAATTGAATGAAAATCGGCATAAAATTAAGGAT | |
| | GATCTAGAGACTCTTGGAAGGCTGGTTCTATATGTGGTACAGTTGGGAAAAGACCCCT | |
| | TTTTCAAAATTCAAGGCCAAAGTATCCGTGAGGTCATAGATGCTTGTATAAATGAGGAA | |
| | ATTAAAGATCTTGTGCAAAACCTGCTTATGCCTGGAACACAATACCAAAACCTGCTAAG | |
| | TAAGATGCTCTCTCATCCTTTCTTCTGGAATTGTAATATTCGTTACAGATTCCTTCGAGAT | |
| | GTGGGGAATGAATCTGACATCAAAAGACGAAATTATGAGAGTGAAATCCTCAAGGCAC | |
| | TAAACTGTACTGATGTTTTTGCAGAGTGGACAAAAAAGATTGATGAACATATTATGAAA | |
| | AAAATGAATTCATTTTATAAAAAGAAGCCTATGTACTGCTACAAGAATGAAGTTGGAGA | |
| | CTTGCTAAAATTCATCCGTAATATAGGTGAACACATTAATGAAGAACAAAATAAAAAAA | |
| | TGAAGTTAAAAATTGGGGAACCTGCTCAGTATTTTCAGAAGATGTTTCCTGATCTGGTG | |
| | ATTTATGTTTACAAGAAACTGCGGAATACGGTCTACGAGAAGCATTTTCCCAGTGTTAA | |
| | CAGTACT | |
| 86 | ATGGCCAGTGATACCCCAGGTTTCTACATGGACAAACTTAATAAATACCGCCAGATGCA | Exemplary REAVR18 |
| | CGGAGTAGCCATTACGTATAAAGAACTTAGTACTTCGGGACCTCCACATGACAGAAGG | DNA sequence |
| | TTTACATTTCAAGTTTTAATAGATGAGAAGGAATTTCCAGAAGCCAAAGGTAAATCAAA | |
| | GCAGGAGGCAAGAAACGCTGCAGCCAAATTAGCTGTTGATATACTTGATAACGAAAAC | |
| | AAGGTGGATTGTCACACGAGTGCATCTGAGCAAGGCTTGCCCTATGGTAACTACATAG | |
| | GCCTTGTCAATAGCTTTGCCCAGAAGAAAAAGCTGTCTGTAAATTATGAACAGTGTGAG | |
| | CCCAACTCTGAGTTGCCTCAAAGATTTATTTGTAAATGCAAAATTGGGCAGACGATGTA | |
| | TGGTACTGGTTCAGGTGTCACCAAACAGGAGGCAAAGCAGTTGGCTGCGAAAGAAGC | |
| | CTATCAGAAGCTGTTAAAGAGCCCGCCGAAAACTGCCGGAACATCCTCTAGCGTTGTCA | |
| | CATCTACATTCAGTGGCTTTTCCAGCAGCTCGTCTATGACAAGTAATGGTGTTTCCCAGT | |
| | CAGCACCTGGAAGTTTTTCCTCAGAGAACGTGTTTACGAAGATTGCAGACACCTCTGAA | |
| | GGTGGTGTCTACTTGGGGTTCTTTGACGGACTAGAGACGGCTGTGAAGATATTCCGTG | |
| | ATGACAGTGAAAGAGCCGAGAAGGAAATCGACTGTATCCTAGAGTGCCGAGAGAACC | |
| | GCCATCTTGTAAAGCTCTATGGAACCGAGATAAATAACAACTGTAGGTACGTGTGCCTC | |
| | TCCCTCTGTGAGAGGAACTTGGAGGAGCACCTGAAAAATCACAGAGATGCAGATGTCG | |
| | AATCCACCAAAGGCATTCTCAAGTCCCTGTTTAAGGCAGTGCACGAATTACATCAGTTT | |
| | GGATTCGTCCATCAGGACCTGCATCCAAAGAACATCTTGATAGATTCTAAAGATGAAGT | |
| | GTGCCTGGCAGATTTTGATAAAAGCTGCAGGTTTGCTAATGATCAGACATTAGTCCATG | |
| | GAGACCTCCGGGCACTTGGACGACTGGTCCAGTATGTTGTAACCAAGGGAGAGGACCC | |
| | CTTTGTGGAGAAAGGGGCTGCAAGAGAGCCTCAGGAGCTAGCGGGTCTAGCTGAAAT | |
| | CCCGGATCACATGGAGACGCTGGACCTCATCCAGACACTGCTCTCCCCTGGGGTCACCA | |
| | GCCAGACTCAGCTGAGCGACATTCTCTACCATCCCTTCTTCTGGCCCTGTAACATGCGCT | |
| | ACAGGTTCCTAAGGGATGTTGGGAACGAATCAGACATCAAAACTCGGAATACTGAAAA | |
| | CAAGACCCTGGAAAAATTGAATCGTAATCCTTCTAAGAGTTACTCAAAATGGACTTCAA | |
| | AGATTGATAAATATGTTATGACTGAGATGAATCATTTCTACAAAAAGAAAAAAAACCCC | |
| | TATCAAGACACAGTGACTGACCTGCTGAAATTCATCCGCAACATAGGCGAGCACATTGA | |
| | TGAGAAAGTCAATGCACAAATGAAGGAAACTATCGGGGATCCTGCTCAGTATTTTCAG | |
| | AAGACCTTTCCTGAGCTGGTGATGCACGTCTACAAGAACATACAGAACACAGAATTCAT | |
| | GAAACATATTCCGAATGCCCAGAATTCTCTCCTGGATAAC | |
| 87 | ATGGAGTCTCTGTCAGGGAATTATACTTCTCTGCTGAATGAGTATCAGCAGAAAACCCA | Exemplary REAVR21 |
| | ATGCACTGTTGAGTTTGAGGAGGGACCCACTGATGGACCAAGCCACAATAAAAGGTTT | DNA sequence |
| | ACTATGAGAGCTATTGTGAATGGACAGAAGTTTCCTGATGGTACTGGTAAAACCAAGA | |
| | AGGAGGCAAAGCAAAATGCTGCTAAAAACGCACTGGAAGGCTTGAAGAGCACTCACA | |
| | GTGATGAACCGACACCTTCACCCGTTGAAAACATTTCAGTTAGTAAAATAGCATCTCAT | |
| | CCGAATTACACCTGCTGGCTCAATGAGCATTCGCAGAAGAGCAGGTTAATGTTCAAGG | |
| | CCTGCGAGTCAACTAAGATGGATCCAGGAAACCTCACTCGGTTATGCACGTATGTGTGT | |
| | AAGTATGTTTGCGATGATAAAGAGTTTCCTGAAGGTTATGGGAAAAATAAGAAGGAAG | |
| | CCAAAGAGGCAGCGGCTCTTCGAGTTTATGAAGAGCTCAACAAAACACCAAATACAGA | |
| | GGTCTTGGATGAGAACAGCAATAGAGCTCAGAGGTCAGAAACTACATCTTATAGCCTG | |
| | GACCGCCGTTCGAGTGTTGCAGATGATTCTCGAAGCAGCACGCCTGACAACAACTACAT | |
| | CGCATATCTCAATAACTACTGCCAGAAGAAAAAACGTGTATATGACTTCAAATTAGTGG | |
| | ACAGAATAGGTCCTCCTCACAACCCAATATTTGTTTATAAAGTTGTGATGGATGGGAAG | |
| | GAATACCCTGAAGCACAGGGAAGAAACGCTAAAGAAGCGAAACAAAATGCGGCTCAA | |
| | CACGCTTGGAGTGAAATTCGGGATCAGTCTGGCTGGACCACACCGAGTTCTGAAGATG | |
| | GCGCATTCTCACAATCCCAAGATACATGTAAATCTGAAGACGTCCAGCGATCAGCGAAC | |
| | GCCTCCAGCACAAGTAATTTCATCTCCTTCAGAGACTCTGCTAAAATTGCTGATACTTCA | |
| | GAAGGAGGCATCTACCTGGGGTTCTATGAGAAGCAAGAAGTAGCTGTGAAGACGTTCT | |
| | GTGAGGGCAGCCCACGTGCACAGCGGGAAGTCTCTTGTCTGCAAAGCAGCCGAGAGA | |
| | ACAGTCACTTGGTGACATTCTATGGGAGTGAGAGCCACAGGGGCCACTTGTTTGTGTG | |
| | TGTCACCCTCTGTGAGCAGACTCTGGAAGCGTGTTTGGATGTGCACAGAGGGGAAGAT | |
| | GTGGAAAATGAGGAAGATGAATTTGCCCGAAATGTCCTGTCATCTATATTTAAGGCTGT | |
| | TCAAGAACTACACTTGTCCTGTGGATACACCCACCAGGATCTGCAACCACAAAACATCT | |
| | TAATAGATTCTAAGAAAGCTGCTCACCTGGCAGATTTTGATAAGAGCATCAAGTGGGCT | |
| | GGAGATCCACAGGAAGTCAAGAGAGATCTAGAGGACCTTGGACGGCTGGTCCTCTATG | |
| | TGGTAAAGAAGGGAAGCATCTCATTTGAGGATCTGAAAGCTCAAAGTAATGAAGAGGT | |
| | GGTTCAACTTTCTCCAGATGAGGAAACTAAGGACCTCATTCATCGTCTCTTCCATCCTGG | |
| | GGAACATGTGAGGGACTGTCTGAGTGACCTGCTGGGTCATCCCTTCTTTTGGACTTGGG | |
| | AGAGCCGCTATAGGACGCTTCGGAATGTGGGAAATGAATCCGACATCAAAACACGAAA | |
| | ATCTGAAAGTGAGATCCTCAGACTACTGCAACCTGGGCCTTCTGAACATTCCAAAAGTT | |
| | TTGACAAGTGGACGACTAAGATTAATGAATGTGTTATGAAAAAAATGAATAAGTTTTAT | |
| | GAAAAAAGAGGCAATTTCTACCAGAACACTGTGGGTGATCTGCTAAAGTTCATCCGGA | |
| | ATTTGGGAGAACACATTGATGAAGAAAAGCATAAAAAGATGAAATTAAAAATTGGAGA | |
| | CCCTTCCCTGTATTTTCAGAAGACATTTCCAGATCTGGTGATCTATGTCTACACAAAACT | |
| | ACAGAACACAGAATATAGAAAGCATTTCCCCCAAACCCACAGTCCAAACAAGCCTCAGT | |
| | GTGATGGAGCTGGTGGGGCCAGTGGGTTGGCCAGCCCTGGGTGC | |
| 88 | ATGGCAAAAATGTCCGCAGAAAATGAAATAGAGATGAGAATCTGTGATTACCTGAGGA | Exemplary REAVR22 |
| | GACACGGCAGGAGCACCGTACAGGACATTTTCAAGGAATTAAAATTAGAGAAATCCAC | DNA sequence |
| | CGTGAACAGGCATCTATACAGCCTACAGGCGTCAAAGCAGGTTTTTAAAACTGTGGAA | |
| | GATAATAAGCGGCCGGTCTGGGATCTGGTGGAGAGCATGAATGACGAACAAAAAGCC | |
| | AAACCAGAGCAAATAAAGCCCGAGATGACAAGAGACGCAATTGAAGAAAAACATGTG | |
| | AAGGATCTGCTGAAATCAGGAGGTTTAAAAGCCTCTCATATCGCCAGCAAACTGGGAC | |
| | AACAAACACAATCAACTAACAAACTACTGTACGGTTTGATGGAGAAAGGTGAAGTGCG | |
| | CAAATGCAGTAAAAGCCACATGTGGACTCTGAAAAATGACCAGGAGAGGAATGGAAG | |
| | TGGCAGTAAAATTGCTGATACTTCAGAAGGAGGCATCTACCTGGGGTTCTATGAGAAG | |
| | CAAGAAGTAGCTGTGAAGACGTTCTGTGAGGGCAGCCCACGTGCACAGCGGGAAGTC | |
| | TCTTGTCTGCAAAGCAGCCGAGAGAACAGTCACTTGGTGACATTCTATGGGAGTGAGA | |
| | GCCACAGGGGCCACTTGTTTGTGTGTGTCACCCTCTGTGAGCAGACTCTGGAAGCGTGT | |
| | TTGGATGTGCACAGAGGGGAAGATGTGGAAAATGAGGAAGATGAATTTGCCCGAAAT | |
| | GTCCTGTCATCTATATTTAAGGCTGTTCAAGAACTACACTTGTCCTGTGGATACACCCAC | |
| | CAGGATCTGCAACCACAAAACATCTTAATAGATTCTAAGAAAGCTGCTCACCTGGCAGA | |
| | TTTTGATAAGAGCATCAAGTGGGCTGGAGATCCACAGGAAGTCAAGAGAGATCTAGA | |
| | GGACCTTGGACGGCTGGTCCTCTATGTGGTAAAGAAGGGAAGCATCTCATTTGAGGAT | |
| | CTGAAAGCTCAAAGTAATGAAGAGGTGGTTCAACTTTCTCCAGATGAGGAAACTAAGG | |
| | ACCTCATTCATCGTCTCTTCCATCCTGGGGAACATGTGAGGGACTGTCTGAGTGACCTG | |
| | CTGGGTCATCCCTTCTTTTGGACTTGGGAGAGCCGCTATAGGACGCTTCGGAATGTGG | |
| | GAAATGAATCCGACATCAAAACACGAAAATCTGAAAGTGAGATCCTCAGACTACTGCA | |
| | ACCTGGGCCTTCTGAACATTCCAAAAGTTTTGACAAGTGGACGACTAAGATTAATGAAT | |
| | GTGTTATGAAAAAAATGAATAAGTTTTATGAAAAAAGAGGCAATTTCTACCAGAACACT | |
| | GTGGGTGATCTGCTAAAGTTCATCCGGAATTTGGGAGAACACATTGATGAAGAAAAGC | |
| | ATAAAAAGATGAAATTAAAAATTGGAGACCCTTCCCTGTATTTTCAGAAGACATTTCCA | |
| | GATCTGGTGATCTATGTCTACACAAAACTACAGAACACAGAATATAGAAAGCATTTCCC | |
| | CCAAACCCACAGTCCAAACAAGCCTCAGTGTGATGGAGCTGGTGGGGCCAGTGGGTTG | |
| | GCCAGCCCTGGGTGC | |
| 89 | ATGGCCCAGGCTCCTGCTGACCCGGGCAGAGAAGGCCACCTTGAACAAAGAATCCTGC | Exemplary REAVR23 |
| | AGGTGCTGACAGAGGCTGGCTCCCCGGTGAAACTTGCCCAGCTGGTGAAGGAATGCC | DNA sequence |
| | AAGCACCCAAGAGGGAGCTCAACCAAGTCCTCTACCGAATGAAAAAGGAGTTGAAAGT | |
| | CTCCCTCACATCCCCTGCCACCTGGTGCTTGGGCGGGACTGATCCTGAAGGCGAGGGT | |
| | CCTGCAGAGCTGGCCTTGTCCAGCCCTGCCGAGAGGCCCCAGCAACATGCAGCTACAA | |
| | TTCCAGAGACCCCTGGCCCTCAGTTCAGCCAACAACGGGAGGAAGACATCTACAGGTT | |
| | TCTCAAAGACAATGGTCCCCAGAGGGCCCTGGTCATCGCCCAAGCACTGGGAATGAGG | |
| | ACAGCAAAAGATGTGAACCGAGACTTGTACAGGATGAAGAGCAGGCACCTTCTGGAC | |
| | ATGGATGAGCAGTCCAAAGCATGGACGATTTACCGCCCAGAAGATTCTGGAAGAAGA | |
| | GCAAAGTCAGCCTCAAAAATTGCTGATACTTCAGAAGGAGGCATCTACCTGGGGTTCTA | |
| | TGAGAAGCAAGAAGTAGCTGTGAAGACGTTCTGTGAGGGCAGCCCACGTGCACAGCG | |
| | GGAAGTCTCTTGTCTGCAAAGCAGCCGAGAGAACAGTCACTTGGTGACATTCTATGGG | |
| | AGTGAGAGCCACAGGGGCCACTTGTTTGTGTGTGTCACCCTCTGTGAGCAGACTCTGG | |
| | AAGCGTGTTTGGATGTGCACAGAGGGGAAGATGTGGAAAATGAGGAAGATGAATTTG | |
| | CCCGAAATGTCCTGTCATCTATATTTAAGGCTGTTCAAGAACTACACTTGTCCTGTGGAT | |
| | ACACCCACCAGGATCTGCAACCACAAAACATCTTAATAGATTCTAAGAAAGCTGCTCAC | |
| | CTGGCAGATTTTGATAAGAGCATCAAGTGGGCTGGAGATCCACAGGAAGTCAAGAGA | |
| | GATCTAGAGGACCTTGGACGGCTGGTCCTCTATGTGGTAAAGAAGGGAAGCATCTCAT | |
| | TTGAGGATCTGAAAGCTCAAAGTAATGAAGAGGTGGTTCAACTTTCTCCAGATGAGGA | |
| | AACTAAGGACCTCATTCATCGTCTCTTCCATCCTGGGGAACATGTGAGGGACTGTCTGA | |
| | GTGACCTGCTGGGTCATCCCTTCTTTTGGACTTGGGAGAGCCGCTATAGGACGCTTCGG | |
| | AATGTGGGAAATGAATCCGACATCAAAACACGAAAATCTGAAAGTGAGATCCTCAGAC | |
| | TACTGCAACCTGGGCCTTCTGAACATTCCAAAAGTTTTGACAAGTGGACGACTAAGATT | |
| | AATGAATGTGTTATGAAAAAAATGAATAAGTTTTATGAAAAAAGAGGCAATTTCTACCA | |
| | GAACACTGTGGGTGATCTGCTAAAGTTCATCCGGAATTTGGGAGAACACATTGATGAA | |
| | GAAAAGCATAAAAAGATGAAATTAAAAATTGGAGACCCTTCCCTGTATTTTCAGAAGAC | |
| | ATTTCCAGATCTGGTGATCTATGTCTACACAAAACTACAGAACACAGAATATAGAAAGC | |
| | ATTTCCCCCAAACCCACAGTCCAAACAAGCCTCAGTGTGATGGAGCTGGTGGGGCCAG | |
| | TGGGTTGGCCAGCCCTGGGTGC | |
| 90 | ATGGCCCAGGCTCCTGCTGACCCGGGCAGAGAAGGCCACCTTGAACAAAGAATCCTGC | Exemplary REAVR24 |
| | AGGTGCTGACAGAGGCTGGCTCCCCGGTGAAACTTGCCCAGCTGGTGAAGGAATGCC | DNA sequence |
| | AAGCACCCAAGAGGGAGCTCAACCAAGTCCTCTACCGAATGAAAAAGGAGTTGAAAGT | |
| | CTCCCTCACATCCCCTGCCACCTGGTGCTTGGGCGGGACTGATCCTGAAGGCGAGGGT | |
| | CCTGCAGAGCTGGCCTTGTCCAGCCCTGCCGAGAGGCCCCAGCAACATGCAGCTACAA | |
| | TTCCAGAGACCCCTGGCCCTCAGTTCAGCCAACAACGGGAGGAAGACATCTACAGGTT | |
| | TCTCAAAGACAATGGTCCCCAGAGGGCCCTGGTCATCGCCCAAGCACTGGGAATGAGG | |
| | ACAGCAAAAGATGTGAACCGAGACTTGTACAGGATGAAGAGCAGGCACCTTCTGGAC | |
| | ATGGATGAGCAGTCCAAAGCATGGACGATTTACCGCCCAGAAGATTCTGGAAGAAGA | |
| | GCAAAGTCAGCCTCAATTATTTACCAGCACAATCCAATCAACATGATCTGCCAGAATGG | |
| | ACCCAACAGCTGGATTTCCATTGCAAACTCCAAAATTGCTGATACTTCAGAAGGAGGCA | |
| | TCTACCTGGGGTTCTATGAGAAGCAAGAAGTAGCTGTGAAGACGTTCTGTGAGGGCAG | |
| | CCCACGTGCACAGCGGGAAGTCTCTTGTCTGCAAAGCAGCCGAGAGAACAGTCACTTG | |
| | GTGACATTCTATGGGAGTGAGAGCCACAGGGGCCACTTGTTTGTGTGTGTCACCCTCTG | |
| | TGAGCAGACTCTGGAAGCGTGTTTGGATGTGCACAGAGGGGAAGATGTGGAAAATGA | |
| | GGAAGATGAATTTGCCCGAAATGTCCTGTCATCTATATTTAAGGCTGTTCAAGAACTAC | |
| | ACTTGTCCTGTGGATACACCCACCAGGATCTGCAACCACAAAACATCTTAATAGATTCTA | |
| | AGAAAGCTGCTCACCTGGCAGATTTTGATAAGAGCATCAAGTGGGCTGGAGATCCACA | |
| | GGAAGTCAAGAGAGATCTAGAGGACCTTGGACGGCTGGTCCTCTATGTGGTAAAGAA | |
| | GGGAAGCATCTCATTTGAGGATCTGAAAGCTCAAAGTAATGAAGAGGTGGTTCAACTT | |
| | TCTCCAGATGAGGAAACTAAGGACCTCATTCATCGTCTCTTCCATCCTGGGGAACATGT | |
| | GAGGGACTGTCTGAGTGACCTGCTGGGTCATCCCTTCTTTTGGACTTGGGAGAGCCGCT | |
| | ATAGGACGCTTCGGAATGTGGGAAATGAATCCGACATCAAAACACGAAAATCTGAAAG | |
| | TGAGATCCTCAGACTACTGCAACCTGGGCCTTCTGAACATTCCAAAAGTTTTGACAAGT | |
| | GGACGACTAAGATTAATGAATGTGTTATGAAAAAAATGAATAAGTTTTATGAAAAAAG | |
| | AGGCAATTTCTACCAGAACACTGTGGGTGATCTGCTAAAGTTCATCCGGAATTTGGGA | |
| | GAACACATTGATGAAGAAAAGCATAAAAAGATGAAATTAAAAATTGGAGACCCTTCCC | |
| | TGTATTTTCAGAAGACATTTCCAGATCTGGTGATCTATGTCTACACAAAACTACAGAACA | |
| | CAGAATATAGAAAGCATTTCCCCCAAACCCACAGTCCAAACAAGCCTCAGTGTGATGGA | |
| | GCTGGTGGGGCCAGTGGGTTGGCCAGCCCTGGGTGC | |
| 91 | ATGGCAGAAGCTCCTGTTGACTTGAGCACAGGAGACAATCTGGAGCAAAAGATCCTGC | Exemplary REAVR25 |
| | AGGTGTTGAGCGATGACGGCGGCCCTGTGAAGATTGGCCAGCTGGTGAAGAAATGCC | DNA sequence |
| | AAGTGCCCAAGAAAACCCTCAATCAAGTCCTTTACCGCCTGAAGAAGGAGGACAGAGT | |
| | GTCCTCCCCAGAGCCTGCAACATGGAGCATAGGCGGGGCTGCTTCTGGAGATGGGGCT | |
| | CCTGCAATCCCTGAGAACTCCAGTGCCCAGCCTAGCCTTGATGAAAGAATATTAAGATT | |
| | CCTGGAAGCCAATGGGCCTCACAGGGCCCTGCACATCGCCAAGGCTCTGGGAATGACG | |
| | ACAGCCAAAGAAGTGAACCCACTCCTGTATTCCATGAGAAATAAGCACCTTCTGAGCTA | |
| | TGACGGACAGACGTGGAAGATCTACCACTCACGTCAGGAAGGCCAAGACATAGCTCAT | |
| | TCTGGAGTCACACAAGAGTCCCCTGCGAAAATTGCTGATACTTCAGAAGGAGGCATCT | |
| | ACCTGGGGTTCTATGAGAAGCAAGAAGTAGCTGTGAAGACGTTCTGTGAGGGCAGCCC | |
| | ACGTGCACAGCGGGAAGTCTCTTGTCTGCAAAGCAGCCGAGAGAACAGTCACTTGGTG | |
| | ACATTCTATGGGAGTGAGAGCCACAGGGGCCACTTGTTTGTGTGTGTCACCCTCTGTGA | |
| | GCAGACTCTGGAAGCGTGTTTGGATGTGCACAGAGGGGAAGATGTGGAAAATGAGGA | |
| | AGATGAATTTGCCCGAAATGTCCTGTCATCTATATTTAAGGCTGTTCAAGAACTACACTT | |
| | GTCCTGTGGATACACCCACCAGGATCTGCAACCACAAAACATCTTAATAGATTCTAAGA | |
| | AAGCTGCTCACCTGGCAGATTTTGATAAGAGCATCAAGTGGGCTGGAGATCCACAGGA | |
| | AGTCAAGAGAGATCTAGAGGACCTTGGACGGCTGGTCCTCTATGTGGTAAAGAAGGG | |
| | AAGCATCTCATTTGAGGATCTGAAAGCTCAAAGTAATGAAGAGGTGGTTCAACTTTCTC | |
| | CAGATGAGGAAACTAAGGACCTCATTCATCGTCTCTTCCATCCTGGGGAACATGTGAGG | |
| | GACTGTCTGAGTGACCTGCTGGGTCATCCCTTCTTTTGGACTTGGGAGAGCCGCTATAG | |
| | GACGCTTCGGAATGTGGGAAATGAATCCGACATCAAAACACGAAAATCTGAAAGTGAG | |
| | ATCCTCAGACTACTGCAACCTGGGCCTTCTGAACATTCCAAAAGTTTTGACAAGTGGAC | |
| | GACTAAGATTAATGAATGTGTTATGAAAAAAATGAATAAGTTTTATGAAAAAAGAGGC | |
| | AATTTCTACCAGAACACTGTGGGTGATCTGCTAAAGTTCATCCGGAATTTGGGAGAACA | |
| | CATTGATGAAGAAAAGCATAAAAAGATGAAATTAAAAATTGGAGACCCTTCCCTGTATT | |
| | TTCAGAAGACATTTCCAGATCTGGTGATCTATGTCTACACAAAACTACAGAACACAGAA | |
| | TATAGAAAGCATTTCCCCCAAACCCACAGTCCAAACAAGCCTCAGTGTGATGGAGCTGG | |
| | TGGGGCCAGTGGGTTGGCCAGCCCTGGGTGC | |
| 92 | ATGGCAGAAGCTCCTGTTGACTTGAGCACAGGAGACAATCTGGAGCAAAAGATCCTGC | Exemplary REAVR26 |
| | AGGTGTTGAGCGATGACGGCGGCCCTGTGAAGATTGGCCAGCTGGTGAAGAAATGCC | DNA sequence |
| | AAGTGCCCAAGAAAACCCTCAATCAAGTCCTTTACCGCCTGAAGAAGGAGGACAGAGT | |
| | GTCCTCCCCAGAGCCTGCAACATGGAGCATAGGCGGGGCTGCTTCTGGAGATGGGGCT | |
| | CCTGCAATCCCTGAGAACTCCAGTGCCCAGCCTAGCCTTGATGAAAGAATATTAAGATT | |
| | CCTGGAAGCCAATGGGCCTCACAGGGCCCTGCACATCGCCAAGGCTCTGGGAATGACG | |
| | ACAGCCAAAGAAGTGAACCCACTCCTGTATTCCATGAGAAATAAGCACCTTCTGAGCTA | |
| | TGACGGACAGACGTGGAAGATCTACCACTCACGTCAGGAAGGCCAAGACATAGCTCAT | |
| | TCTGGAGTCACACAAGAGTCCCCTGCGATTATTTGTCAGCACAATCCGGTCAACATGAT | |
| | CTGCCAACAAGGAGCCAACAGCCACATCTCCATTGCCAATTCAAAAATTGCTGATACTT | |
| | CAGAAGGAGGCATCTACCTGGGGTTCTATGAGAAGCAAGAAGTAGCTGTGAAGACGT | |
| | TCTGTGAGGGCAGCCCACGTGCACAGCGGGAAGTCTCTTGTCTGCAAAGCAGCCGAGA | |
| | GAACAGTCACTTGGTGACATTCTATGGGAGTGAGAGCCACAGGGGCCACTTGTTTGTG | |
| | TGTGTCACCCTCTGTGAGCAGACTCTGGAAGCGTGTTTGGATGTGCACAGAGGGGAAG | |
| | ATGTGGAAAATGAGGAAGATGAATTTGCCCGAAATGTCCTGTCATCTATATTTAAGGCT | |
| | GTTCAAGAACTACACTTGTCCTGTGGATACACCCACCAGGATCTGCAACCACAAAACAT | |
| | CTTAATAGATTCTAAGAAAGCTGCTCACCTGGCAGATTTTGATAAGAGCATCAAGTGGG | |
| | CTGGAGATCCACAGGAAGTCAAGAGAGATCTAGAGGACCTTGGACGGCTGGTCCTCTA | |
| | TGTGGTAAAGAAGGGAAGCATCTCATTTGAGGATCTGAAAGCTCAAAGTAATGAAGAG | |
| | GTGGTTCAACTTTCTCCAGATGAGGAAACTAAGGACCTCATTCATCGTCTCTTCCATCCT | |
| | GGGGAACATGTGAGGGACTGTCTGAGTGACCTGCTGGGTCATCCCTTCTTTTGGACTTG | |
| | GGAGAGCCGCTATAGGACGCTTCGGAATGTGGGAAATGAATCCGACATCAAAACACG | |
| | AAAATCTGAAAGTGAGATCCTCAGACTACTGCAACCTGGGCCTTCTGAACATTCCAAAA | |
| | GTTTTGACAAGTGGACGACTAAGATTAATGAATGTGTTATGAAAAAAATGAATAAGTTT | |
| | TATGAAAAAAGAGGCAATTTCTACCAGAACACTGTGGGTGATCTGCTAAAGTTCATCCG | |
| | GAATTTGGGAGAACACATTGATGAAGAAAAGCATAAAAAGATGAAATTAAAAATTGGA | |
| | GACCCTTCCCTGTATTTTCAGAAGACATTTCCAGATCTGGTGATCTATGTCTACACAAAA | |
| | CTACAGAACACAGAATATAGAAAGCATTTCCCCCAAACCCACAGTCCAAACAAGCCTCA | |
| | GTGTGATGGAGCTGGTGGGGCCAGTGGGTTGGCCAGCCCTGGGTGC | |
| 93 | AUGGCCAGUGAUACUCCCGGUUUCUACAUGGACAAACUUAAUAAAUACCACCAGA | Exemplary REAVR1 |
| | AGCAUAGAGUAACGAUUACGUAUAAACACCUUUUUACUACAGGACCUCCACAUGA | mRNA sequence |
| | CAGAAGGUUUACAUUUCAAGUUAUAAUAGACGGAGAAGAAUUUCCAGAAGCCGAA | |
| | GGCAAAACAAAGCAGGAAGCAAAAAACGCUGCUGCCAAAUUGGCUGUUGAUACAC | |
| | UUAACGCAAAUAAGGCAAAUAGUCAUACAGACGCUUUGGAAGGUUCAUUCACUGG | |
| | GAAUUACAUAGGCCUUGUCAACAGUUAUGCUCAGAAGGAGAAGUUGUCUGUAAA | |
| | UUACCAACAGUGUGCCUUCAAUACCCAGUCACCCCAAAGAUUUUGUUAUAAAUGC | |
| | AUAAUUGGGCUGAAAACCUACGGUAUUGGUUCAGGCGCUACCAAGCAGGAGGCAA | |
| | AGCAGCUGGCUGCUAAAGAGGCGUAUCAGAAGCUAGGAUCCUCAGAAGGAGGCAU | |
| | CUACCUGGGGUUCUAUGAGAAGCAAGAAGUAGCUGUGAAGACGUUCUGUGAGGG | |
| | CAGCCCACGUGCACAGCGGGAAGUCUCUUGUCUGCAAAGCAGCCGAGAGAACAGUC | |
| | ACUUGGUGACAUUCUAUGGGAGUGAGAGCCACAGGGGCCACUUGUUUGUGUGUG | |
| | UCACCCUCUGUGAGCAGACUCUGGAAGCGUGUUUGGAUGUGCACAGAGGGGAAG | |
| | AUGUGGAAAAUGAGGAAGAUGAAUUUGCCCGAAAUGUCCUGUCAUCUAUAUUUA | |
| | AGGCUGUUCAAGAACUACACUUGUCCUGUGGAUACACCCACCAGGAUCUGCAACCA | |
| | CAAAACAUCUUAAUAGAUUCUAAGAAAGCUGCUCACCUGGCAGAUUUUGAUAAGA | |
| | GCAUCAAGUGGGCUGGAGAUCCACAGGAAGUCAAGAGAGAUCUAGAGGACCUUGG | |
| | ACGGCUGGUCCUCUAUGUGGUAAAGAAGGGAAGCAUCUCAUUUGAGGAUCUGAA | |
| | AGCUCAAAGUAAUGAAGAGGUGGUUCAACUUUCUCCAGAUGAGGAAACUAAGGAC | |
| | CUCAUUCAUCGUCUCUUCCAUCCUGGGGAACAUGUGAGGGACUGUCUGAGUGACC | |
| | UGCUGGGUCAUCCCUUCUUUUGGACUUGGGAGAGCCGCUAUAGGACGCUUCGGA | |
| | AUGUGGGAAAUGAAUCCGACAUCAAAACACGAAAAUCUGAAAGUGAGAUCCUCAG | |
| | ACUACUGCAACCUGGGCCUUCUGAACAUUCCAAAAGUUUUGACAAGUGGACGACU | |
| | AAGAUUAAUGAAUGUGUUAUGAAAAAAAUGAAUAAGUUUUAUGAAAAAAGAGGC | |
| | AAUUUCUACCAGAACACUGUGGGUGAUCUGCUAAAGUUCAUCCGGAAUUUGGGA | |
| | GAACACAUUGAUGAAGAAAAGCAUAAAAAGAUGAAAUUAAAAAUUGGAGACCCUU | |
| | CCCUGUAUUUUCAGAAGACAUUUCCAGAUCUGGUGAUCUAUGUCUACACAAAACU | |
| | ACAGAACACAGAAUAUAGAAAGCAUUUCCCCCAAACCCACAGUCCAAACAAGCCUCA | |
| | GUGUGAUGGAGCUGGUGGGGCCAGUGGGUUGGCCAGCCCUGGGUGC | |
| 94 | AUGGACAAACUUAAUAAAUACCACCAGAAGCAUAGAGUAACGAUUACGUAUAAAC | Exemplary REAVR2 |
| | ACCUUUUUACUACAGGACCUCCACAUGACAGAAGGUUUACAUUUCAAGUUAUAAU | mRNA sequence |
| | AGACGGAGAAGAAUUUCCAGAAGCCGAAGGCAAAACAAAGCAGGAAGCAAAAAACG | |
| | CUGCUGCCAAAUUGGCUGUUGAUACACUUAACGCAAAUAAGGCAAAUAGUCAUAC | |
| | AGACGCUUUGGAAGGUUCAUUCACUGGGAAUUACAUAGGCCUUGUCAACAGUUA | |
| | UGCUCAGAAGGAGAAGUUGUCUGUAAAUUACCAACAGUGUGCCUUCAAUACCCAG | |
| | UCACCCCAAAGAUUUUGUUAUAAAUGCAUAAUUGGGCUGAAAACCUACGGUAUUG | |
| | GUUCAGGCGCUACCAAGCAGGAGGCAAAGCAGCUGGCUGCUAAAGAGGCGUAUCA | |
| | GAAGCUACCAGAGAAAAGCUCAAUGAGAGUUUGCAGAGCAUCCUCUGGUCUUAGC | |
| | AAAUCUUCAUCCAGUGGCCACUUCAACAGCGGAUCCUCAGAAGGAGGCAUCUACCU | |
| | GGGGUUCUAUGAGAAGCAAGAAGUAGCUGUGAAGACGUUCUGUGAGGGCAGCCC | |
| | ACGUGCACAGCGGGAAGUCUCUUGUCUGCAAAGCAGCCGAGAGAACAGUCACUUG | |
| | GUGACAUUCUAUGGGAGUGAGAGCCACAGGGGCCACUUGUUUGUGUGUGUCACC | |
| | CUCUGUGAGCAGACUCUGGAAGCGUGUUUGGAUGUGCACAGAGGGGAAGAUGUG | |
| | GAAAAUGAGGAAGAUGAAUUUGCCCGAAAUGUCCUGUCAUCUAUAUUUAAGGCU | |
| | GUUCAAGAACUACACUUGUCCUGUGGAUACACCCACCAGGAUCUGCAACCACAAAA | |
| | CAUCUUAAUAGAUUCUAAGAAAGCUGCUCACCUGGCAGAUUUUGAUAAGAGCAUC | |
| | AAGUGGGCUGGAGAUCCACAGGAAGUCAAGAGAGAUCUAGAGGACCUUGGACGGC | |
| | UGGUCCUCUAUGUGGUAAAGAAGGGAAGCAUCUCAUUUGAGGAUCUGAAAGCUC | |
| | AAAGUAAUGAAGAGGUGGUUCAACUUUCUCCAGAUGAGGAAACUAAGGACCUCAU | |
| | UCAUCGUCUCUUCCAUCCUGGGGAACAUGUGAGGGACUGUCUGAGUGACCUGCU | |
| | GGGUCAUCCCUUCUUUUGGACUUGGGAGAGCCGCUAUAGGACGCUUCGGAAUGU | |
| | GGGAAAUGAAUCCGACAUCAAAACACGAAAAUCUGAAAGUGAGAUCCUCAGACUAC | |
| | UGCAACCUGGGCCUUCUGAACAUUCCAAAAGUUUUGACAAGUGGACGACUAAGAU | |
| | UAAUGAAUGUGUUAUGAAAAAAAUGAAUAAGUUUUAUGAAAAAAGAGGCAAUUU | |
| | CUACCAGAACACUGUGGGUGAUCUGCUAAAGUUCAUCCGGAAUUUGGGAGAACAC | |
| | AUUGAUGAAGAAAAGCAUAAAAAGAUGAAAUUAAAAAUUGGAGACCCUUCCCUGU | |
| | AUUUUCAGAAGACAUUUCCAGAUCUGGUGAUCUAUGUCUACACAAAACUACAGAA | |
| | CACAGAAUAUAGAAAGCAUUUCCCCCAAACCCACAGUCCAAACAAGCCUCAGUGUG | |
| | AUGGAGCUGGUGGGGCCAGUGGGUUGGCCAGCCCUGGGUGC | |
| 95 | AUGGACAAACUUAAUAAAUACCACCAGAAGCAUAGAGUAACGAUUACGUAUAAAC | Exemplary REAVR3 |
| | ACCUUUUUACUACAGGACCUCCACAUGACAGAAGGUUUACAUUUCAAGUUAUAAU | mRNA sequence |
| | AGACGGAGAAGAAUUUCCAGAAGCCGAAGGCAAAACAAAGCAGGAAGCAAAAAACG | |
| | CUGCUGCCAAAUUGGCUGUUGAUACACUUAACGCAAAUAAGGCAAAUAGUCAUAC | |
| | AGACGCUUUGGAAGGUUCAUUCACUGGGAAUUACAUAGGCCUUGUCAACAGUUA | |
| | UGCUCAGAAGGAGAAGUUGUCUGUAAAUUACCAACAGUGUGCCUUCAAUACCCAG | |
| | UCACCCCAAAGAUUUUGUUAUAAAUGCAUAAUUGGGCUGAAAACCUACGGUAUUG | |
| | GUUCAGGCGCUACCAAGCAGGAGGCAAAGCAGCUGGCUGCUAAAGAGGCGUAUCA | |
| | GAAGCUACCAGAGAAAAGCUCAAUGAGAGUUUGCAGAGCAUCCUCUGGUCUUAGC | |
| | AAAUCUUCAUCCAGUGGCCACUUCAACAGCUGGUCCACAGCAAACAAUUUUACUUC | |
| | UCUGUCAGCGCCUGGAAGUGAUUUCUCAGAGACUGCAUCAGGAUCCUCAGAAGGA | |
| | GGCAUCUACCUGGGGUUCUAUGAGAAGCAAGAAGUAGCUGUGAAGACGUUCUGU | |
| | GAGGGCAGCCCACGUGCACAGCGGGAAGUCUCUUGUCUGCAAAGCAGCCGAGAGA | |
| | ACAGUCACUUGGUGACAUUCUAUGGGAGUGAGAGCCACAGGGGCCACUUGUUUG | |
| | UGUGUGUCACCCUCUGUGAGCAGACUCUGGAAGCGUGUUUGGAUGUGCACAGAG | |
| | GGGAAGAUGUGGAAAAUGAGGAAGAUGAAUUUGCCCGAAAUGUCCUGUCAUCUA | |
| | UAUUUAAGGCUGUUCAAGAACUACACUUGUCCUGUGGAUACACCCACCAGGAUCU | |
| | GCAACCACAAAACAUCUUAAUAGAUUCUAAGAAAGCUGCUCACCUGGCAGAUUUU | |
| | GAUAAGAGCAUCAAGUGGGCUGGAGAUCCACAGGAAGUCAAGAGAGAUCUAGAGG | |
| | ACCUUGGACGGCUGGUCCUCUAUGUGGUAAAGAAGGGAAGCAUCUCAUUUGAGG | |
| | AUCUGAAAGCUCAAAGUAAUGAAGAGGUGGUUCAACUUUCUCCAGAUGAGGAAAC | |
| | UAAGGACCUCAUUCAUCGUCUCUUCCAUCCUGGGGAACAUGUGAGGGACUGUCUG | |
| | AGUGACCUGCUGGGUCAUCCCUUCUUUUGGACUUGGGAGAGCCGCUAUAGGACG | |
| | CUUCGGAAUGUGGGAAAUGAAUCCGACAUCAAAACACGAAAAUCUGAAAGUGAGA | |
| | UCCUCAGACUACUGCAACCUGGGCCUUCUGAACAUUCCAAAAGUUUUGACAAGUG | |
| | GACGACUAAGAUUAAUGAAUGUGUUAUGAAAAAAAUGAAUAAGUUUUAUGAAAA | |
| | AAGAGGCAAUUUCUACCAGAACACUGUGGGUGAUCUGCUAAAGUUCAUCCGGAAU | |
| | UUGGGAGAACACAUUGAUGAAGAAAAGCAUAAAAAGAUGAAAUUAAAAAUUGGA | |
| | GACCCUUCCCUGUAUUUUCAGAAGACAUUUCCAGAUCUGGUGAUCUAUGUCUACA | |
| | CAAAACUACAGAACACAGAAUAUAGAAAGCAUUUCCCCCAAACCCACAGUCCAAACA | |
| | AGCCUCAGUGUGAUGGAGCUGGUGGGGCCAGUGGGUUGGCCAGCCCUGGGUGC | |
| 96 | AUGGACAAACUUAAUAAAUACCACCAGAAGCAUAGAGUAACGAUUACGUAUAAAC | Exemplary REAVR4 |
| | ACCUUUUUACUACAGGACCUCCACAUGACAGAAGGUUUACAUUUCAAGUUAUAAU | mRNA sequence |
| | AGACGGAGAAGAAUUUCCAGAAGCCGAAGGCAAAACAAAGCAGGAAGCAAAAAACG | |
| | CUGCUGCCAAAUUGGCUGUUGAUACACUUAACGCAAAUAAGGCAAAUAGUCAUAC | |
| | AGACGCUUUGGAAGGUUCAUUCACUGGGAAUUACAUAGGCCUUGUCAACAGUUA | |
| | UGCUCAGAAGGAGAAGUUGUCUGUAAAUUACCAACAGUGUGCCUUCAAUACCCAG | |
| | UCACCCCAAAGAUUUUGUUAUAAAUGCAUAAUUGGGCUGAAAACCUACGGUAUUG | |
| | GUUCAGGCGCUACCAAGCAGGAGGCAAAGCAGCUGGCUGCUAAAGAGGCGUAUCA | |
| | GAAGCUACCAGAGAAAAGCUCAAUGAGAGUUUGCAGAGCAUCCUCUGGUCUUAGC | |
| | AAAUCUUCAUCCAGUGGCCACUUCAACAGCUGGUCCACAGCAAACAAUUUUACUUC | |
| | UCUGUCAGCGCCUGNAAGUGAUUUCUCAGAGACUGCAUCAAACAACCACUGUGUU | |
| | UUUUUCAAGUCUCCUCUCAUGAAUGGGCUCAGAGAAAAUAAAAGGAAGCCACGAG | |
| | UAAAACUGUCAUCUAAUGAUGUGCAAAGAAAUAAAUAUACCGGAUCCUCAGAAGG | |
| | AGGCAUCUACCUGGGGUUCUAUGAGAAGCAAGAAGUAGCUGUGAAGACGUUCUG | |
| | UGAGGGCAGCCCACGUGCACAGCGGGAAGUCUCUUGUCUGCAAAGCAGCCGAGAG | |
| | AACAGUCACUUGGUGACAUUCUAUGGGAGUGAGAGCCACAGGGGCCACUUGUUU | |
| | GUGUGUGUCACCCUCUGUGAGCAGACUCUGGAAGCGUGUUUGGAUGUGCACAGA | |
| | GGGGAAGAUGUGGAAAAUGAGGAAGAUGAAUUUGCCCGAAAUGUCCUGUCAUCU | |
| | AUAUUUAAGGCUGUUCAAGAACUACACUUGUCCUGUGGAUACACCCACCAGGAUC | |
| | UGCAACCACAAAACAUCUUAAUAGAUUCUAAGAAAGCUGCUCACCUGGCAGAUUU | |
| | UGAUAAGAGCAUCAAGUGGGCUGGAGAUCCACAGGAAGUCAAGAGAGAUCUAGAG | |
| | GACCUUGGACGGCUGGUCCUCUAUGUGGUAAAGAAGGGAAGCAUCUCAUUUGAG | |
| | GAUCUGAAAGCUCAAAGUAAUGAAGAGGUGGUUCAACUUUCUCCAGAUGAGGAA | |
| | ACUAAGGACCUCAUUCAUCGUCUCUUCCAUCCUGGGGAACAUGUGAGGGACUGUC | |
| | UGAGUGACCUGCUGGGUCAUCCCUUCUUUUGGACUUGGGAGAGCCGCUAUAGGA | |
| | CGCUUCGGAAUGUGGGAAAUGAAUCCGACAUCAAAACACGAAAAUCUGAAAGUGA | |
| | GAUCCUCAGACUACUGCAACCUGGGCCUUCUGAACAUUCCAAAAGUUUUGACAAG | |
| | UGGACGACUAAGAUUAAUGAAUGUGUUAUGAAAAAAAUGAAUAAGUUUUAUGAA | |
| | AAAAGAGGCAAUUUCUACCAGAACACUGUGGGUGAUCUGCUAAAGUUCAUCCGGA | |
| | AUUUGGGAGAACACAUUGAUGAAGAAAAGCAUAAAAAGAUGAAAUUAAAAAUUG | |
| | GAGACCCUUCCCUGUAUUUUCAGAAGACAUUUCCAGAUCUGGUGAUCUAUGUCUA | |
| | CACAAAACUACAGAACACAGAAUAUAGAAAGCAUUUCCCCCAAACCCACAGUCCAAA | |
| | CAAGCCUCAGUGUGAUGGAGCUGGUGGGGCCAGUGGGUUGGCCAGCCCUGGGUG | |
| | C | |
| 97 | AUGGACAAACUUAAUAAAUACCACCAGAAGCAUAGAGUAACGAUUACGUAUAAAC | Exemplary REAVR5 |
| | ACCUUUUUACUACAGGACCUCCACAUGACAGAAGGUUUACAUUUCAAGUUAUAAU | mRNA sequence |
| | AGACGGAGAAGAAUUUCCAGAAGCCGAAGGCAAAACAAAGCAGGAAGCAAAAAACG | |
| | CUGCUGCCAAAUUGGCUGUUGAUACACUUAACGCAAAUAAGGCAAAUAGUCAUAC | |
| | AGACGCUUUGGAAGGUUCAUUCACUGGGAAUUACAUAGGCCUUGUCAACAGUUA | |
| | UGCUCAGAAGGAGAAGUUGUCUGUAAAUUACCAACAGUGUGCCUUCAAUACCCAG | |
| | UCACCCCAAAGAUUUUGUUAUAAAUGCAUAAUUGGGCUGAAAACCUACGGUAUUG | |
| | GUUCAGGCGCUACCAAGCAGGAGGCAAAGCAGCUGGCUGCUAAAGAGGCGUAUCA | |
| | GAAGCUACCAGAGAAAAGCUCAAUGAGAGUUUGCAGAGCAUCCUCUGGUCUUAGC | |
| | AAAUCUUCAUCCAGUGGCCACUUCAACAGCUGGUCCACAGCAAACAAUUUUACUUC | |
| | UCUGUCAGCGCCUGGAAGUGAUUUCUCAGAGACUGCAUCAAACAACCACUGUGUU | |
| | UUUUUCAAGUCUCCUCUCAUGAAUGGGCUCAGAGAAAAUAAAAGGAAGCCACGAG | |
| | UAAAACUGUCAUCUAAUGAUGUGCAAAGAAAUAAAUAUACCACGGACUCCAGGUU | |
| | UAACGAGGAUUUUGAAGACAUAGAAGAAAUUGGCUCAGGUGGAUUUGGCCAAGU | |
| | UUUCAAAGCAAAACACAGAAUCGAUGGGAAGACUUACGCUAUUAAGCGAGUUAAA | |
| | UAUAACUCAGAGAAGGAAGUGCGUGAAGUAAAGGCGCUGGCGGCACUCAGCCAUG | |
| | UCAAUAUUGUCCAGUACCAUUUUUGUUGGCUGGGACAGGACUGGGAGUACGAUA | |
| | CAGAGCAGAGCAUGAACAGUGCCACCCCACGAUCAAAGACAGAGUGCCUUUUCAUU | |
| | CAAAUGGAAUUCUGUGAUAAAGGGACUCUGGAGCAAUGGAUGACAAGUGUCAAG | |
| | CAGAGGAAAGUAGACAAUGAUUUGUUUUUGGAAUUAGCUGAACAAAUAACAACA | |
| | GGGGUGGAUUAUAUACACUCAAAAGGCUUAAUUCACAGAGACCUUAAGCCAAGUA | |
| | AUAUAUUUUUAGUGGAUGAAAAACACAUAAAGAUUGGAGACUUCGGCCUCGUAA | |
| | CAGCCCUGGAAAAUGAUGGAAAUCGAACAAAGAAUACGGGAUCUCUUCUAUACAU | |
| | GAGCCCAGAACAGUUAUCUUUACAGGAAUAUGGAAAAGAAGUGGACAUCUUUGCU | |
| | CUAGGACUUAUUCUAGCUGAACUCCUUCACAUAUGCAUCACUAUUAUAGAAAAGU | |
| | CAAAGUUUUUCACGGACCUAAGAGAUGGCAUCUUCCAUGACGACAUAUUUGGCAG | |
| | CAAGGAAAAAAGGCUUCUAACGAAAUUACUCUCGAAGAAACCCACGGAUCGACCUA | |
| | GUGCUUCUGAAAUUCUGAUGACCUUGGCUGAAUGGAAGAAUAUCUCAGAAGUAC | |
| | AGAAACGACACACACAUGGAUCCUCAGAAGGAGGCAUCUACCUGGGGUUCUAUGA | |
| | GAAGCAAGAAGUAGCUGUGAAGACGUUCUGUGAGGGCAGCCCACGUGCACAGCGG | |
| | GAAGUCUCUUGUCUGCAAAGCAGCCGAGAGAACAGUCACUUGGUGACAUUCUAUG | |
| | GGAGUGAGAGCCACAGGGGCCACUUGUUUGUGUGUGUCACCCUCUGUGAGCAGA | |
| | CUCUGGAAGCGUGUUUGGAUGUGCACAGAGGGGAAGAUGUGGAAAAUGAGGAAG | |
| | AUGAAUUUGCCCGAAAUGUCCUGUCAUCUAUAUUUAAGGCUGUUCAAGAACUACA | |
| | CUUGUCCUGUGGAUACACCCACCAGGAUCUGCAACCACAAAACAUCUUAAUAGAUU | |
| | CUAAGAAAGCUGCUCACCUGGCAGAUUUUGAUAAGAGCAUCAAGUGGGCUGGAGA | |
| | UCCACAGGAAGUCAAGAGAGAUCUAGAGGACCUUGGACGGCUGGUCCUCUAUGUG | |
| | GUAAAGAAGGGAAGCAUCUCAUUUGAGGAUCUGAAAGCUCAAAGUAAUGAAGAG | |
| | GUGGUUCAACUUUCUCCAGAUGAGGAAACUAAGGACCUCAUUCAUCGUCUCUUCC | |
| | AUCCUGGGGAACAUGUGAGGGACUGUCUGAGUGACCUGCUGGGUCAUCCCUUCU | |
| | UUUGGACUUGGGAGAGCCGCUAUAGGACGCUUCGGAAUGUGGGAAAUGAAUCCG | |
| | ACAUCAAAACACGAAAAUCUGAAAGUGAGAUCCUCAGACUACUGCAACCUGGGCCU | |
| | UCUGAACAUUCCAAAAGUUUUGACAAGUGGACGACUAAGAUUAAUGAAUGUGUU | |
| | AUGAAAAAAAUGAAUAAGUUUUAUGAAAAAAGAGGCAAUUUCUACCAGAACACUG | |
| | UGGGUGAUCUGCUAAAGUUCAUCCGGAAUUUGGGAGAACACAUUGAUGAAGAAA | |
| | AGCAUAAAAAGAUGAAAUUAAAAAUUGGAGACCCUUCCCUGUAUUUUCAGAAGAC | |
| | AUUUCCAGAUCUGGUGAUCUAUGUCUACACAAAACUACAGAACACAGAAUAUAGA | |
| | AAGCAUUUCCCCCAAACCCACAGUCCAAACAAGCCUCAGUGUGAUGGAGCUGGUGG | |
| | GGCCAGUGGGUUGGCCAGCCCUGGGUGC | |
| 98 | AUGGACAAACUUAAUAAAUACCACCAGAAGCAUAGAGUAACGAUUACGUAUAAAC | Exemplary REAVR6 |
| | ACCUUUUUACUACAGGACCUCCACAUGACAGAAGGUUUACAUUUCAAGUUAUAAU | mRNA sequence |
| | AGACGGAGAAGAAUUUCCAGAAGCCGAAGGCAAAACAAAGCAGGAAGCAAAAAACG | |
| | CUGCUGCCAAAUUGGCUGUUGAUACACUUAACGCAAAUAAGGCAAAUAGUCAUAC | |
| | AGACGCUUUGGAAGGUUCAUUCACUGGGAAUUACAUAGGCCUUGUCAACAGUUA | |
| | UGCUCAGAAGGAGAAGUUGUCUGUAAAUUACCAACAGUGUGCCUUCAAUACCCAG | |
| | UCACCCCAAAGAUUUUGUUAUAAAUGCAUAAUUGGGCUGAAAACCUACGGUAUUG | |
| | GUUCAGGCGCUACCAAGCAGGAGGCAAAGCAGCUGGCUGCUAAAGAGGCGUAUCA | |
| | GAAGCUACCAGAGAAAAGCUCAAUGAGAGUUUGCAGAGCAUCCUCUGGUCUUAGC | |
| | AAAUCUUCAUCCAGUGGCCACUUCAACAGCUGGUCCACAGCAAACAAUUUUACUUC | |
| | UCUGUCAGCGCCUGGAAGUGAUUUCUCAGAGACUGCAUCAAACAACCACUGUGUU | |
| | UUUUUCAAGUCUCCUCUCAUGAAUGGGCUCAGAGAAAAUAAAAGGAAGCCACGAG | |
| | UAAAACUGUCAUCUAAUGAUGUGCAAAGAAAUAAAUAUACCACGGACUCCAGGUU | |
| | UAACGAGGAUUUUGAAGACAUAGAAGAAAUUGGCUCAGGUGGAUUUGGCCAAGU | |
| | UUUCAAAGCAAAACACAGAAUCGAUGGGAAGACUUACGCUAUUAGGCGAGUUAAA | |
| | UAUAACUCAGAGAAGGAAGUGCGUGAAGUAAAGGCGCUGGCGGCACUCAGCCAUG | |
| | UCAAUAUUGUCCAGUACCAUUUUUGUUGGCUGGGACAGGACUGGGAGUACGAUA | |
| | CAGAGCAGAGCAUGAACAGUGCCACCCCACGAUCAAAGACAGAGUGCCUUUUCAUU | |
| | CAAAUGGAAUUCUGUGAUAAAGGGACUCUGGAGCAAUGGAUGACAAGUGUCAAG | |
| | CAGAGGAAAGUAGACAAUGAUUUGUUUUUGGAAUUAGCUGAACAAAUAACAACA | |
| | GGGGUGGAUUAUAUACACUCAAAAGGCUUAAUUCACAGAGACCUUAAGCCAAGUA | |
| | AUAUAUUUUUAGUGGAUGAAAAACACAUAAAGAUUGGAGACUUCGGCCUCGUAA | |
| | CAGCCCUGGAAAAUGAUGGAAAUCGAACAAAGAAUACGGGAUCUCUUCUAUACAU | |
| | GAGCCCAGAACAGUUAUCUUUACAGGAAUAUGGAAAAGAAGUGGACAUCUUUGCU | |
| | CUAGGACUUAUUCUAGCUGAACUCCUUCACAUAUGCAUCACUAUUAUAGAAAAGU | |
| | CAAAGUUUUUCACGGACCUAAGAGAUGGCAUCUUCCAUGACGACAUAUUUGGCAG | |
| | CAAGGAAAAAAGGCUUCUAACGAAAUUACUCUCGAAGAAACCCACGGAUCGACCUA | |
| | GUGCUUCUGAAAUUCUGAUGACCUUGGCUGAAUGGAAGAAUAUCUCAGAAGUAC | |
| | AGAAACGACACACACAUGGAUCCUCAGAAGGAGGCAUCUACCUGGGGUUCUAUGA | |
| | GAAGCAAGAAGUAGCUGUGAAGACGUUCUGUGAGGGCAGCCCACGUGCACAGCGG | |
| | GAAGUCUCUUGUCUGCAAAGCAGCCGAGAGAACAGUCACUUGGUGACAUUCUAUG | |
| | GGAGUGAGAGCCACAGGGGCCACUUGUUUGUGUGUGUCACCCUCUGUGAGCAGA | |
| | CUCUGGAAGCGUGUUUGGAUGUGCACAGAGGGGAAGAUGUGGAAAAUGAGGAAG | |
| | AUGAAUUUGCCCGAAAUGUCCUGUCAUCUAUAUUUAAGGCUGUUCAAGAACUACA | |
| | CUUGUCCUGUGGAUACACCCACCAGGAUCUGCAACCACAAAACAUCUUAAUAGAUU | |
| | CUAAGAAAGCUGCUCACCUGGCAGAUUUUGAUAAGAGCAUCAAGUGGGCUGGAGA | |
| | UCCACAGGAAGUCAAGAGAGAUCUAGAGGACCUUGGACGGCUGGUCCUCUAUGUG | |
| | GUAAAGAAGGGAAGCAUCUCAUUUGAGGAUCUGAAAGCUCAAAGUAAUGAAGAG | |
| | GUGGUUCAACUUUCUCCAGAUGAGGAAACUAAGGACCUCAUUCAUCGUCUCUUCC | |
| | AUCCUGGGGAACAUGUGAGGGACUGUCUGAGUGACCUGCUGGGUCAUCCCUUCU | |
| | UUUGGACUUGGGAGAGCCGCUAUAGGACGCUUCGGAAUGUGGGAAAUGAAUCCG | |
| | ACAUCAAAACACGAAAAUCUGAAAGUGAGAUCCUCAGACUACUGCAACCUGGGCCU | |
| | UCUGAACAUUCCAAAAGUUUUGACAAGUGGACGACUAAGAUUAAUGAAUGUGUU | |
| | AUGAAAAAAAUGAAUAAGUUUUAUGAAAAAAGAGGCAAUUUCUACCAGAACACUG | |
| | UGGGUGAUCUGCUAAAGUUCAUCCGGAAUUUGGGAGAACACAUUGAUGAAGAAA | |
| | AGCAUAAAAAGAUGAAAUUAAAAAUUGGAGACCCUUCCCUGUAUUUUCAGAAGAC | |
| | AUUUCCAGAUCUGGUGAUCUAUGUCUACACAAAACUACAGAACACAGAAUAUAGA | |
| | AAGCAUUUCCCCCAAACCCACAGUCCAAACAAGCCUCAGUGUGAUGGAGCUGGUGG | |
| | GGCCAGUGGGUUGGCCAGCCCUGGGUGC | |
| 99 | AUGGCCAGUGAUACCCCAGGUUUCUACAUGGACAAACUUAAUAAAUACCGCCAGA | Exemplary REAVR7 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGAAAAUUGCUGAUACUUCAGAAGGAGGCAUCUAC | |
| | CUGGGGUUCUAUGAGAAGCAAGAAGUAGCUGUGAAGACGUUCUGUGAGGGCAGC | |
| | CCACGUGCACAGCGGGAAGUCUCUUGUCUGCAAAGCAGCCGAGAGAACAGUCACU | |
| | UGGUGACAUUCUAUGGGAGUGAGAGCCACAGGGGCCACUUGUUUGUGUGUGUCA | |
| | CCCUCUGUGAGCAGACUCUGGAAGCGUGUUUGGAUGUGCACAGAGGGGAAGAUG | |
| | UGGAAAAUGAGGAAGAUGAAUUUGCCCGAAAUGUCCUGUCAUCUAUAUUUAAGG | |
| | CUGUUCAAGAACUACACUUGUCCUGUGGAUACACCCACCAGGAUCUGCAACCACAA | |
| | AACAUCUUAAUAGAUUCUAAGAAAGCUGCUCACCUGGCAGAUUUUGAUAAGAGCA | |
| | UCAAGUGGGCUGGAGAUCCACAGGAAGUCAAGAGAGAUCUAGAGGACCUUGGACG | |
| | GCUGGUCCUCUAUGUGGUAAAGAAGGGAAGCAUCUCAUUUGAGGAUCUGAAAGC | |
| | UCAAAGUAAUGAAGAGGUGGUUCAACUUUCUCCAGAUGAGGAAACUAAGGACCUC | |
| | AUUCAUCGUCUCUUCCAUCCUGGGGAACAUGUGAGGGACUGUCUGAGUGACCUGC | |
| | UGGGUCAUCCCUUCUUUUGGACUUGGGAGAGCCGCUAUAGGACGCUUCGGAAUG | |
| | UGGGAAAUGAAUCCGACAUCAAAACACGAAAAUCUGAAAGUGAGAUCCUCAGACU | |
| | ACUGCAACCUGGGCCUUCUGAACAUUCCAAAAGUUUUGACAAGUGGACGACUAAG | |
| | AUUAAUGAAUGUGUUAUGAAAAAAAUGAAUAAGUUUUAUGAAAAAAGAGGCAAU | |
| | UUCUACCAGAACACUGUGGGUGAUCUGCUAAAGUUCAUCCGGAAUUUGGGAGAAC | |
| | ACAUUGAUGAAGAAAAGCAUAAAAAGAUGAAAUUAAAAAUUGGAGACCCUUCCCU | |
| | GUAUUUUCAGAAGACAUUUCCAGAUCUGGUGAUCUAUGUCUACACAAAACUACAG | |
| | AACACAGAAUAUAGAAAGCAUUUCCCCCAAACCCACAGUCCAAACAAGCCUCAGUG | |
| | UGAUGGAGCUGGUGGGGCCAGUGGGUUGGCCAGCCCUGGGUGC | |
| 100 | AUGGCCAGUGAUACCCCAGGUUUCUACAUGGACAAACUUAAUAAAUACCGCCAGA | Exemplary REAVR8 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGAACGGUCUCGGAGAAAAUAAAAGGAAAUCAGGA | |
| | GUAAAAGUAUCCCCUGAUGAUGUGCAAAGAAAUAAAUAUACCAAAAUUGCUGAUA | |
| | CUUCAGAAGGAGGCAUCUACCUGGGGUUCUAUGAGAAGCAAGAAGUAGCUGUGA | |
| | AGACGUUCUGUGAGGGCAGCCCACGUGCACAGCGGGAAGUCUCUUGUCUGCAAAG | |
| | CAGCCGAGAGAACAGUCACUUGGUGACAUUCUAUGGGAGUGAGAGCCACAGGGGC | |
| | CACUUGUUUGUGUGUGUCACCCUCUGUGAGCAGACUCUGGAAGCGUGUUUGGAU | |
| | GUGCACAGAGGGGAAGAUGUGGAAAAUGAGGAAGAUGAAUUUGCCCGAAAUGUC | |
| | CUGUCAUCUAUAUUUAAGGCUGUUCAAGAACUACACUUGUCCUGUGGAUACACCC | |
| | ACCAGGAUCUGCAACCACAAAACAUCUUAAUAGAUUCUAAGAAAGCUGCUCACCUG | |
| | GCAGAUUUUGAUAAGAGCAUCAAGUGGGCUGGAGAUCCACAGGAAGUCAAGAGA | |
| | GAUCUAGAGGACCUUGGACGGCUGGUCCUCUAUGUGGUAAAGAAGGGAAGCAUC | |
| | UCAUUUGAGGAUCUGAAAGCUCAAAGUAAUGAAGAGGUGGUUCAACUUUCUCCA | |
| | GAUGAGGAAACUAAGGACCUCAUUCAUCGUCUCUUCCAUCCUGGGGAACAUGUGA | |
| | GGGACUGUCUGAGUGACCUGCUGGGUCAUCCCUUCUUUUGGACUUGGGAGAGCC | |
| | GCUAUAGGACGCUUCGGAAUGUGGGAAAUGAAUCCGACAUCAAAACACGAAAAUC | |
| | UGAAAGUGAGAUCCUCAGACUACUGCAACCUGGGCCUUCUGAACAUUCCAAAAGU | |
| | UUUGACAAGUGGACGACUAAGAUUAAUGAAUGUGUUAUGAAAAAAAUGAAUAAG | |
| | UUUUAUGAAAAAAGAGGCAAUUUCUACCAGAACACUGUGGGUGAUCUGCUAAAG | |
| | UUCAUCCGGAAUUUGGGAGAACACAUUGAUGAAGAAAAGCAUAAAAAGAUGAAAU | |
| | UAAAAAUUGGAGACCCUUCCCUGUAUUUUCAGAAGACAUUUCCAGAUCUGGUGAU | |
| | CUAUGUCUACACAAAACUACAGAACACAGAAUAUAGAAAGCAUUUCCCCCAAACCC | |
| | ACAGUCCAAACAAGCCUCAGUGUGAUGGAGCUGGUGGGGCCAGUGGGUUGGCCAG | |
| | CCCUGGGUGC | |
| 101 | AUGGCCAGUGAUACCCCAGGUUUCUACAUGGACAAACUUAAUAAAUACCGCCAGA | Exemplary REAVR9 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGAACGGUCUCGGAGAAAAUAAAAGGAAAUCAGGA | |
| | GUAAAAGUAUCCCCUGAUGAUGUGCAAAGAAAUAAAUAUACCUUGGACGCCAGGU | |
| | UUAACAGCGAUUUUGAAGACAUAGAAGAAAUUGGCUUAGGUGGAUUUGGUCAAG | |
| | UUUUCAAAGCGAAACACAGAAUUGAUGGAAAGAGAUACGCUAUUAAGCGCGUUAA | |
| | AUAUAACACGGAGAAGGCGGAGCACGAAGUACAAGCGCUGGCAGAACUCAAUCAC | |
| | GUCAACAUUGUCCAAUACCAUAGUUGUUGGGAGGGAGUUGACUAUGAUCCUGAG | |
| | CACAGCAUGAGUGAUACAAGUCGAUACAAAACCCGGUGCCUCUUUAUUCAAAUGG | |
| | AAUUCUGUGAUAAAGGAACUUUGGAGCAAUGGAUGAGAAACAGAAAUCAGAGUA | |
| | AAGUGGACAAAGCUUUGAUUUUGGACUUAUAUGAACAAAUCGUGACCGGAGUGG | |
| | AGUAUAUACACUCGAAAGGGUUAAUUCACAGAGAUCUUAAGCCAGGUAAUAUAUU | |
| | UUUAGUAGAUGAAAGACACAUUAAGAUCGGAGACUUUGGCCUUGCAACAGCCCUG | |
| | GAAAAUGAUGGAAAAUCCCGAACAAGGAGAACAGGAACUCUUCAAUAUAUGAGUC | |
| | CAGAACAGUUAUUUUUAAAGCACUAUGGAAAAGAAGUGGACAUCUUUGCUUUGG | |
| | GCCUUAUUCUAGCUGAACUUCUUCACACGUGCUUCACGGAGUCAGAGAAAAUAAA | |
| | GUUUUUCGAAAGUCUAAGAAAAGGCGACUUCUCUAAUGAUAUAUUCGACAACAAA | |
| | GAAAAAAGCCUUCUAAAAAAACUACUCUCAGAGAAACCCAAGGACCGACCUGAGAC | |
| | AUCUGAAAUCCUGAAGACCUUGGCUGAAUGGAGGAACAUCUCAGAGAAAAAGAAA | |
| | AGAAACACAUGUAAAAUUGCUGAUACUUCAGAAGGAGGCAUCUACCUGGGGUUCU | |
| | AUGAGAAGCAAGAAGUAGCUGUGAAGACGUUCUGUGAGGGCAGCCCACGUGCACA | |
| | GCGGGAAGUCUCUUGUCUGCAAAGCAGCCGAGAGAACAGUCACUUGGUGACAUUC | |
| | UAUGGGAGUGAGAGCCACAGGGGCCACUUGUUUGUGUGUGUCACCCUCUGUGAG | |
| | CAGACUCUGGAAGCGUGUUUGGAUGUGCACAGAGGGGAAGAUGUGGAAAAUGAG | |
| | GAAGAUGAAUUUGCCCGAAAUGUCCUGUCAUCUAUAUUUAAGGCUGUUCAAGAA | |
| | CUACACUUGUCCUGUGGAUACACCCACCAGGAUCUGCAACCACAAAACAUCUUAAU | |
| | AGAUUCUAAGAAAGCUGCUCACCUGGCAGAUUUUGAUAAGAGCAUCAAGUGGGCU | |
| | GGAGAUCCACAGGAAGUCAAGAGAGAUCUAGAGGACCUUGGACGGCUGGUCCUCU | |
| | AUGUGGUAAAGAAGGGAAGCAUCUCAUUUGAGGAUCUGAAAGCUCAAAGUAAUG | |
| | AAGAGGUGGUUCAACUUUCUCCAGAUGAGGAAACUAAGGACCUCAUUCAUCGUCU | |
| | CUUCCAUCCUGGGGAACAUGUGAGGGACUGUCUGAGUGACCUGCUGGGUCAUCCC | |
| | UUCUUUUGGACUUGGGAGAGCCGCUAUAGGACGCUUCGGAAUGUGGGAAAUGAA | |
| | UCCGACAUCAAAACACGAAAAUCUGAAAGUGAGAUCCUCAGACUACUGCAACCUGG | |
| | GCCUUCUGAACAUUCCAAAAGUUUUGACAAGUGGACGACUAAGAUUAAUGAAUGU | |
| | GUUAUGAAAAAAAUGAAUAAGUUUUAUGAAAAAAGAGGCAAUUUCUACCAGAACA | |
| | CUGUGGGUGAUCUGCUAAAGUUCAUCCGGAAUUUGGGAGAACACAUUGAUGAAG | |
| | AAAAGCAUAAAAAGAUGAAAUUAAAAAUUGGAGACCCUUCCCUGUAUUUUCAGAA | |
| | GACAUUUCCAGAUCUGGUGAUCUAUGUCUACACAAAACUACAGAACACAGAAUAU | |
| | AGAAAGCAUUUCCCCCAAACCCACAGUCCAAACAAGCCUCAGUGUGAUGGAGCUGG | |
| | UGGGGCCAGUGGGUUGGCCAGCCCUGGGUGC | |
| 102 | AUGGCCAGUGAUACCCCAGGUUUCUACAUGGACAAACUUAAUAAAUACCGCCAGA | Exemplary REAVR10 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGAAAAUUCAAGAAACCUCCCAUGGUGGCAUCUAC | |
| | CUAGGUUUCUAUAAAGGGAAAGAAGUAGCUGUGAAGAGGGUUCCAAAAGGUAAU | |
| | AAUAAAGAGCUGGUCUGUCUGGAGCAAUUACGGAAGAAUAAUCAUUUAGUACGU | |
| | UACUGUGGAUAUGAAGAAGAUAGCUCAUGUUCAUAUCUAUGUCUUUUUCUGUAU | |
| | GAGAGAAACCUUGAAGAACACAUUAGUAAUAUUAAAGAUGUAACUGAGAUCCAAG | |
| | ACAUGCUUCAGUCUAUAUUCCUGGGCUUGCAUGAGCUACAUGAGAUAGGAUUUU | |
| | GCCAUCGGGACCUUCAUCCAUCCAAUAUUUUAAUUGAUCAUGAGGGUAAAGCUUA | |
| | CCUGGCAGAUUUCAGCGGGAGUCUGAAGUUGGAAGAUUCUGAAGACAAGGAAGG | |
| | GUUGAUCAAGAGUGACAUGAAGGCAUUGGGAAACCUUGUCUUGUAUGUAGUAAC | |
| | AAGAGGAACGGCCUGCUUCCAACCAGAUAGAGAAAGGGAUGUGACGGAAGCUUGU | |
| | CCAGAUUCAGUGGAAGAUUUUAUAGAAGCAAGGGAUCUAGUAAAUAAAUUAAUG | |
| | UCUCCAGAUACAGAGUAUUCAUCUGUGAUUGAUUUCCUUAACCAUCCCUUCUUCU | |
| | GGAGUCACACAAGCAGGUAUAAGUUUCUUCAAGAUGUUGGGAAUGAGAAUGAUG | |
| | UCAAAAUGCGCACAGAGGACAGUGCCCUUUUCAGAACCCUGAAUGAUUCUCAUGU | |
| | GACAAUGACAAAAACCUUCCACCCCUGGGGUUGUAAGGUUGACCAGGAGGUCCUG | |
| | AAUGACAUGAACAGGUUCCACAGUAAGAAGAAAAUCUAUUAUCAGGACACAGUCA | |
| | CUGACCUGUUGAAGUUCAUUAGAAACUUGGGUGCUCACUUCGAUGAGAAAAAAAC | |
| | AGAAAUAAAGGAGAUCAUUGGAGAUCCCUGCCAGUAUUUUCUCAAGAAGUUUCCA | |
| | GAUCUCACGGUUUAUGUCUAUGAAACUCUGCGACAUUUGGGGAAGGGAUACAUU | |
| | AAGCAUUUUCCAAACCCCGUAGUUCUCUUCUAUAAUUGCAUGCAGCUCAACAAUU | |
| | CGGGUAUGACU | |
| 103 | AUGGCCAGUGAUACCCCAGGUUUCUACAUGGACAAACUUAAUAAAUACCGCCAGA | Exemplary REAVR11 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGAGAAUUCAGAACACUUCCCAGGGUGGCAUCUAC | |
| | UUGGGGCUCCACGGUGGGACGGAGGUAGCAGUAAGAAUAAGCCGCAGUACAGAG | |
| | GGUGACAAAGAGAAAAGGUUCUUCGAACAAUGUGGUAACUGUGAACAUCUACUG | |
| | AAGCUCUUCCAGUUUGAGAAGGCAAGAGGCUACAUGUACUUGUGCUUCCCCCUCU | |
| | GGGAGAAAAACCUUGAAGAACAUCUGCAGGAACCAGAAGACCAAAUGGAUUACAA | |
| | AGAUGCUCUGAGGAUGAUCUUCCAGGCAGUGAGAGAGCUGCACUCCCUUGGAUU | |
| | UGCUCACCAGGAUCUGCAUCCCACCAACUUUUUCAUAGAUUUAGGUGGCAAAAUU | |
| | UACUUGGCAGACUUUGAUAAUAAAAGAAAGUUGAUUGAAGGCAAAAAAGAACUCG | |
| | UAAACUCAGAUUUAGAGGCCCUCAGCAGGCUCGUGCUGUAUGUCUUAACAGGGGG | |
| | UAGGAAACCCCUUCAGCAAGUCAGUACCAAGGAUUUGACUGCUGAUUCCCCAGAU | |
| | UAUGAGGAGGCUCUGGACCUUGUAACUAGCCUGGUCUCUCAUGAUGAACGAGGC | |
| | UUGGAAGGUUUGAGCAAACAUCCCUAUUUCUGGAGCAAACAGACCAGGUUCAAGU | |
| | UCCUGAAGGGUAUAUGGAAUAAAAUCAAAGAUCUCCACAACCGAAAAGCUGUCUU | |
| | UAAAGCUGCUAAUGCUACUGAACGUUUUCCUUAUCCACAGUGGACCGAGAAGAUU | |
| | GACAAAUAUGUUCUGAAAAUCAUGGAAAAUCCUAGGAAAACAAAACCACUCAAAUA | |
| | UUGCAAUGAUGUCACCGACCUACUGAGGCUCAUCAGAAACCUGGAUGAACACCCAG | |
| | AUACCAGGAUCAGCAACAGAAUAGGAGACCAUGCUGAGUAUUUCUUGAAGCGCUU | |
| | UCCAGCACUUACCAUUUAUGUCUACAACAGUUUACGUCAGAAUCCCAAAUACAGUC | |
| | ACUUUGCAGACAUUCAGGACCCUUCUCUG | |
| 104 | AUGGCCAGUGAUACCCCAGGUUUCUACAUGGACAAACUUAAUAAAUACCGCCAGA | Exemplary REAVR12 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGAAGAUUACGAAAGACAACAUAACGGAAGUCUAC | |
| | CUUGGUUUCUAUAACAAUGUGAUUGAAGUGGCAGUGAAAUGUCACAGGAACGGC | |
| | AGUGAAGAAGCCAAAAAUGAAAUGGAUUGCCUCAGAGAUCCUACGAUCAAUGGUA | |
| | GCAGCCUGUUUGUUAACCUCCUGCAUUGGGAAAGUGAUGAGUUUUGCGAUUACU | |
| | UAUGCCUGAAUCUCUAUGAGUAUAAUCUCGAGGAAUGUGUGACCAUGUGGCCUA | |
| | AGGAAAUUCGAGAGAAGGCUCCUAAAAUAAUGAAGCAGUUGGUUGACGCUGUUC | |
| | AGGUUUUGCAUGGAGCUGGAUUUGCUCACCGGGAUUUACAUCCAACAAAUGUCC | |
| | UCAGAGAUGUUAAAGAAAAUUUUCGCCUGGCAGACUUUGAUAGGAGUGUGAAUU | |
| | GUAAGCAUCCAGCUGUGACCAUAUCCGGCAAAGCGACCUGGGAGGCUUCCGAAAU | |
| | CCUUCAGAAACUGGAACUCUGGCCGACAGCAACCUUCGAAGCGGAUGAGCUUUUC | |
| | AAAGCGGAUUUACAGGCUCUUGGACGGCUUUUGCAUUAUGUUAUGACCGGAGGG | |
| | AAGGAUCCCUGUAGCAGUGAAAAUGAUCUGUGCGAGAAUAAUCUUUCUCUCAAUG | |
| | AGGAUCUUUGCGAACCCAAGAAUGCCGAAGCCAGAGAUUUCAUCGAGCGCUUGUU | |
| | GGCGCCAGCAGAGAAAAGAGCUACAUUAAGUGAGAUCAGACAACAUCCGCUGCUG | |
| | UGGAAAGAUUUGGGAAAGAGCCAGUUUGUCCAAGGCCUUGCAAAUGAAAACGGU | |
| | GUGGCAAAACGGGAUAAAUCCAGUCCGAUAGUCCUGAUUCUGAAUGAGGUUGGC | |
| | AACAACCAGGAAAGAUCAUUUCACAAGUGGACAGAGAAGGUCCACCUUAAAAUCCU | |
| | AAAUAAUAUGAAUAAGAGACAGCGCACAAAAUAUGAGGACAACACCAGUGACCUCC | |
| | UGAAAUUCAUAAGAAACCUGACCCAGCAUUUUGAUGAAAAGGAAGAAUUACACAG | |
| | UUUGAUUAAAAGUCCCGAAUCUUAUUUCUGUGAAUUAUUUCCUGAUCUCACCAU | |
| | CGCUACCUACAAUGCUGUGAAGGACACCGAGUGGGUAAAGCACUUUCCAAAAGAA | |
| 105 | AUGGCCAGUGAUACCCCAGGUUUCUACAUGGACAAACUUAAUAAAUACCGCCAGA | Exemplary REAVR13 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGUGGAUCCAGAGGACUACCCAAGGAGGGGUCUAC | |
| | CUAGGAUUCUAUGCUGGAGAAGAGGUGGCUGUGAAGAUAUUCUGGAAAGGUGCA | |
| | GAAAAUGCCAAGCGAGAGAAAGUGUGCCUUGAGAAAUGUCGAACCAGCAACCACC | |
| | UGGUGAAGUUUUGCGGCUGGGAAGAGAGGAAGACCUGUCUCUACCUGUGCCUCU | |
| | CCUUGUGUGAGAGGAAUCUUCAAGAGUACUUCAAGGAAGAAAAGAAAAAAACUAU | |
| | GGAAAGCGAAGAGAUUCUUCGAACAGUAUUUCAGGCCGUAAAGGAACUUCACACA | |
| | UUUGGAUUUGGCCAUCAGGAACUGCACCCAAGUAACAUUUUGAUAGAUGCCACUG | |
| | GCAGGAUUUUCCUAGCCGACUUUGAUAAGAGCAGAGAAAUAACUGAUGAUAUAAA | |
| | AUAUUCAAUAAUCUCAGAAGACCUACAGGGCCUCCAAAAACUUAUCCUGUAUGUU | |
| | GUGAUGAGAGGCCAGUUGGACUUUGAAGAUUUGCCUACAGAAUGCCCAGAAAAU | |
| | GUAAAGAACCAUAAGGAAAUUGAAGACCUCCGAGGAAGGCUGGGACUCUCUGACC | |
| | AAAGUACCCCAGCCGAUGACCUACUGGAAAAGCUGAUCAAUCAUCCAUAUUUUUG | |
| | GUCCAAGGAGCAAAAGUACGGAGUGUUAAAAAAGGUCGGGAAUGAGAUACGUGCC | |
| | GCUAAGUGCUGUAAUAAAACCAGUGAAACAAAUAUUCUGGAAGCUUUGAAACCUA | |
| | UGGAUAAUUCUUUCAGGGACUGGAAAAAAAAGAUUGAUAAAACAGUACUGGACU | |
| | CUAUGUUUUUUCUGUCCACUGAGAAGGGACAGAAGGGGAAUCAGAAGGGCAACAA | |
| | ACGAAGUUAUGGCCCCUGCAUAACAGAGUUACUAAAGUUGAUUAGGAAUAUGGG | |
| | UGAGCACUACAAUGAGAAGGAUGACAAGGUGAAAGACAUAAUUCGGGACCCGGCA | |
| | GACUAUUUUCUGGAUCGCUUUCCAGAUUUAACCAUUUAUAUCUAUCAGCGUUUG | |
| | UGUAAUACGGAAUACAGUAUACAUUUUCCAAAUGCUAAGAAUCCUUCUCACUCA | |
| 106 | AUGGCCAGUGAUACCCCAGGUUUCUACAUGGACAAACUUAAUAAAUACCGCCAGA | Exemplary REAVR14 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGAAGAUUGCUGAGAGCUCUCAAGGGGGUGUCUA | |
| | CCUGGGCUUCUAUGGGGAUGGAGUUGAGGUUGCAGUGAAACGCAUGAGAAAGAA | |
| | CAGUAAUGAAGUGAAAAUGGAAAAGCUGUGUCUUGAACACCCAAAGAUUAUGGGC | |
| | AACAGCUGUUUUGUUAAACUCUAUGGCAGUGAAACGGACGCUACAUGUCAUUAU | |
| | UUGUGCUUGACCCUCUGUGAAUGCACUCUAGAGACAUACAUACAAGAGGAAGAAG | |
| | GAAAUCUAAAGACUAAAGUGCCUCACAUAUUUAAGCAGCUAAUCGGAGCAGUGCA | |
| | GAUAUUACAUGAAGCUGGAUUUGCACACACAGACCUUCAUCCUUCAAAUAUCCUA | |
| | UUUAAUGUAGAAGGGAAGCUUCAACUGGCUGAUUUUGAUAAGUGUUUAAGGUU | |
| | UGAAGACUGUCCACAUAAAGGCCAAUCAGAAAGAAGUGAUAAAACUGAAGAGGAG | |
| | CUGAAAAAUGAGGACAUUCAGGCAAUUGGAAAUCUGCUUUGUUAUGUCACCACUG | |
| | AAGGAAAAUAUGUAUACAAGACAGAAGAGGAGAAGCUUGGAGACUGCUAUAGUG | |
| | CUGAGGUGAGGGAUCUUAUACAGAAACUUUCGGCCCCACAGGGACAACCACCUCCA | |
| | CUAAAGGAGGCCCUGCAACAUCCAUUCUUCUGGAGUCAAGAAAGCAAAUACAGAU | |
| | UUCUACAAGACAUUGGUAAUGAGAAUGAAGUAAAAGUCAGAAACGACAAACAUGA | |
| | AAUAGUAAAGAGUCUUAAUGAAAAGGCACAAGACCCUUCUUCAUCAUUCCGUGAA | |
| | UGGACCAAAAAGGUAAAUGACAUUGUUUUUAAAAACAUGGAUAGUCGUAAUAGG | |
| | AAACCAGAACUCAAGUAUAGGAAUGACACUGCAAGCCUCUUGAAGUUUAUACGAA | |
| | AUAUGGGAGCCCAUUUUGAGGAGAAGCCACUAGAAAUUAAGAAUAUUGUCAAGAA | |
| | UCCGUGCAACUACUUCCUUGACUUGUUUCCAGAUCUUGUUAUUUCUGUCUACAA | |
| | UGUUGUGAAAUCAACUCAAUGGGAAGAACAUUUUCCAAAAGCUGAAACUUCU | |
| 107 | AUGGCCAGUGAUACCCCAGGUUUCUACAUGGACAAACUUAAUAAAUACCGCCAGA | Exemplary REAVR15 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGAAAAUUGCAGACAGUUCUGAAGGGGGUGUGUA | |
| | CUUGGGGUUCUAUGGAGAACAGGAGGUAGCUGUGAAGCGAUUCUAUGAAGGCAG | |
| | UACCCUGGGGCAAAAGGAAGUCUCCUGUUUGCAGAGUUGCCGGGAGAAGAGUAAC | |
| | UUGGUGACAUUCUAUGAGAGUGAGAGGCAAAGUGACUGCCUGUAUGUGUGCCUC | |
| | ACCCUCUGCGAGCAGACACUGGAGGAGCACUUCAAUGCCCGCAAAAGGGCAGACGU | |
| | GGGAAACAAGGAAGAUAAGUUUGCCCGGAAAGUCCUCUCUUCUAUAUUUAAAGCA | |
| | GUCAAAGGGCUGCACCAGUGUGGAUAUGCUCACCAAGACCUGCAGCCACGAAACAU | |
| | CUUAAUAGAUUCCAAUGAUGCUAUUUGCCUGGCAGAUUUUGAUAAAAGCAUCAA | |
| | GAUGACUAGAAAACAGGAAAUCAAAACAGAUCUAAAGGACCUCGGACUACUGGUC | |
| | AUAUAUGUUGUAAAAAAGGGAGACAUCCCUUUUGAAAGACUGAAGACUAAAAGUA | |
| | AUGAAGACGUGGUUCGACUUUCUCCAGAUGUGGAAACUGAAAACCUCAUUCACCA | |
| | UCUGAUCUUUCCUAAGGAAGAAUUUACAGACCUAAGUGGCCUGCUGGGGCAUCCC | |
| | UUCUUUUGGAGUUGGGAGAGUCGCUAUAGGGCCCUGAGUGAAGUGGGAAAUGAA | |
| | AAUGACAUCAAAAUUCGAAAGCCUAAUAGCAAGAUCUUUCAACUGCUGGAGCCUA | |
| | AAUCUUCUGAACAUUCCAAAAGUUUUAACCGCUGGAAGGCUAAGAUUGAUGAGUA | |
| | UUUUAUGAAACAAAUGGAUCAGUUUUACAAAAAAGACGGUAGGUAUCAGAACACU | |
| | GUGGGUGAUCUGCUAAGAUUCAUCCGGAAUACAAGACAACAUCUUCAUGAAAAAA | |
| | AGAAUGAAAAGGUGAAGUUGAGAAUUGGAGAACCUUCCCAAUAUUUUCAGGAAA | |
| | AAUUUCCGGAUUUCUUUUUAUAUGUCUAUGAAAGACUACACAGCACUGAAUAUG | |
| | GAAAGCAUUUUGUCCAAGAC | |
| 108 | AUGGCCAGUGAUACCCCAGGUUUCUACAUGGACAAACUUAAUAAAUACCGCCAGA | Exemplary REAVR16 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGAAAAUCGCCGACACGCAGGGGGGCGGCGUCUAC | |
| | CUGGGGCUCCUCGAGGACCAGGAGGUGGCCGUGAAGGUCUUCGUUCAGGGCAGU | |
| | GAAUGUGCCGGCAGGGAGCUGUCCGCCCUCAAGAGUGGCCAGGACAGCAGCCGCC | |
| | UGGUGACCCUGCUUGCCUGCGAGAGCCACGAAGCCAACCUGUAUGUGUGUAUGAC | |
| | CCUGUGUGAGUGGACGCUGGAGGAGUUCCUGGAGCACCGACCUCAGCCUGCCCAA | |
| | GGCGACGAGGACAAGUGGGCCCGCCAGGUCUUGCACUCCGUGUUUGAGGCAGUUC | |
| | GAGAGCUGCACCUACAGCGUGCUCAUGCCCACCAGGACCUGCAGCCACAGAACAUU | |
| | CUCAUAGACGCUGCGCACGGCAUCCGCCUGGCUGACUUUGAUCAGAGCGUCCAGC | |
| | UGGCUGAACCCACCAAAGUCUCACAGGCCGUCAGGACAGACCUGCAGGCCCUGGGC | |
| | CGGCUGGUCGUGUACGUGCUGUUGAAGGGGGAUGUUCCGUUUGAGCAGCUGAGU | |
| | GCCAUGCAUCAUCGAAAGGUCUGGCGUCUCUGUCUUGAACGGGAGACCAGGGACC | |
| | UCACGCGCUGCCUGCUGACCCCCCAGCAGGCUCAGGAGGACCCUCUGGGCAGCCUG | |
| | UUGGGCCACCCCUUCUUCUGGGAGUGGCAGAGCCGCUACAGGACGCUCCGGAACG | |
| | UGGGGAACGAAUCUGACAUCAAAAGGUGCAAUCUCAGGGGCGCCAUCGUGCAGCU | |
| | GCUGAGUCUGAAGCCCUCUGAGCACAGCCUCAGCUUCGCCCAGUGGACGAGCAAG | |
| | GUGGACAGCCUCGUGCUGGGACAAAUGAACGACUUCUACAAGAACAAACCUCACCU | |
| | GUGCUACACGGACAGCGUGCAGGACCUGCUGAGGUUCAUCCGCAACAUUGGGGAG | |
| | CACAUUGACGAGGACUGCAACCGAGAGAUGAAGGCCCGGCUCGGGGACCCCGCUA | |
| | UCUACUUCCUGCGCACAUUUCCAGACCUGCUGAUGCACGUGUACAAGCAGCUGCA | |
| | GGAUUCGGAAUUCCGGAGGCACUUCCCACAGCUCCCUCUGCCUGCGGGCCCCGGCG | |
| | UGUUGGAGACGGAGGUGCCAGGGGGCCAGGCGCUGGGGGCUGAUCCC | |
| 109 | AUGGCCAGUGAUACCCCAGGUUUCUACAUGGACAAACUUAAUAAAUACCGCCAGA | Exemplary REAVR17 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGAAGGUUGCAGAAACUUCUGAAGGUGGUGUGUA | |
| | CCUGGGGUUCUAUGAUGGGAAAGAGGCAGCCGUGAAGCGAUUCCGAGAAGACAG | |
| | UAAACGGGCUCAGCAAGAGAUGGACUGCAUCCUCCAUUGCCAAAAGAAUAGCAAC | |
| | UUGGUGAUGUUUUAUGGGAGAGAAAGCAAAGACAACUGUUUGUAUUUGUGCUU | |
| | UUCUCUUUGUGAGAGGACUCUGGAGGAACAUGUGACAACAAGAGCCUUGGAGAA | |
| | UGAGGACUUCCAAAACAUUUUUGAGUCCUUAUUUAAGGCAGUGCAAGAACUACAC | |
| | AGACACGACUAUCUCCACCAGGACCUGCACCCAGGGAAUAUCUUGAUAGAUUCUGA | |
| | AGGUAAAGUUCACUUGGCAGAUUUUGAUAAGAGUGAAAAAUUGAAUGAAAAUCG | |
| | GCAUAAAAUUAAGGAUGAUCUAGAGACUCUUGGAAGGCUGGUUCUAUAUGUGGU | |
| | ACAGUUGGGAAAAGACCCCUUUUUCAAAAUUCAAGGCCAAAGUAUCCGUGAGGUC | |
| | AUAGAUGCUUGUAUAAAUGAGGAAAUUAAAGAUCUUGUGCAAAACCUGCUUAUG | |
| | CCUGGAACACAAUACCAAAACCUGCUAAGUAAGAUGCUCUCUCAUCCUUUCUUCUG | |
| | GAAUUGUAAUAUUCGUUACAGAUUCCUUCGAGAUGUGGGGAAUGAAUCUGACAU | |
| | CAAAAGACGAAAUUAUGAGAGUGAAAUCCUCAAGGCACUAAACUGUACUGAUGUU | |
| | UUUGCAGAGUGGACAAAAAAGAUUGAUGAACAUAUUAUGAAAAAAAUGAAUUCA | |
| | UUUUAUAAAAAGAAGCCUAUGUACUGCUACAAGAAUGAAGUUGGAGACUUGCUA | |
| | AAAUUCAUCCGUAAUAUAGGUGAACACAUUAAUGAAGAACAAAAUAAAAAAAUGA | |
| | AGUUAAAAAUUGGGGAACCUGCUCAGUAUUUUCAGAAGAUGUUUCCUGAUCUGG | |
| | UGAUUUAUGUUUACAAGAAACUGCGGAAUACGGUCUACGAGAAGCAUUUUCCCAG | |
| | UGUUAACAGUACU | |
| 110 | AUGGCCAGUGAUACCCCAGGUUUCUACAUGGACAAACUUAAUAAAUACCGCCAGA | Exemplary REAVR18 |
| | UGCACGGAGUAGCCAUUACGUAUAAAGAACUUAGUACUUCGGGACCUCCACAUGA | mRNA sequence |
| | CAGAAGGUUUACAUUUCAAGUUUUAAUAGAUGAGAAGGAAUUUCCAGAAGCCAA | |
| | AGGUAAAUCAAAGCAGGAGGCAAGAAACGCUGCAGCCAAAUUAGCUGUUGAUAUA | |
| | CUUGAUAACGAAAACAAGGUGGAUUGUCACACGAGUGCAUCUGAGCAAGGCUUGC | |
| | CCUAUGGUAACUACAUAGGCCUUGUCAAUAGCUUUGCCCAGAAGAAAAAGCUGUC | |
| | UGUAAAUUAUGAACAGUGUGAGCCCAACUCUGAGUUGCCUCAAAGAUUUAUUUG | |
| | UAAAUGCAAAAUUGGGCAGACGAUGUAUGGUACUGGUUCAGGUGUCACCAAACA | |
| | GGAGGCAAAGCAGUUGGCUGCGAAAGAAGCCUAUCAGAAGCUGUUAAAGAGCCCG | |
| | CCGAAAACUGCCGGAACAUCCUCUAGCGUUGUCACAUCUACAUUCAGUGGCUUUU | |
| | CCAGCAGCUCGUCUAUGACAAGUAAUGGUGUUUCCCAGUCAGCACCUGGAAGUUU | |
| | UUCCUCAGAGAACGUGUUUACGAAGAUUGCAGACACCUCUGAAGGUGGUGUCUAC | |
| | UUGGGGUUCUUUGACGGACUAGAGACGGCUGUGAAGAUAUUCCGUGAUGACAGU | |
| | GAAAGAGCCGAGAAGGAAAUCGACUGUAUCCUAGAGUGCCGAGAGAACCGCCAUC | |
| | UUGUAAAGCUCUAUGGAACCGAGAUAAAUAACAACUGUAGGUACGUGUGCCUCUC | |
| | CCUCUGUGAGAGGAACUUGGAGGAGCACCUGAAAAAUCACAGAGAUGCAGAUGUC | |
| | GAAUCCACCAAAGGCAUUCUCAAGUCCCUGUUUAAGGCAGUGCACGAAUUACAUC | |
| | AGUUUGGAUUCGUCCAUCAGGACCUGCAUCCAAAGAACAUCUUGAUAGAUUCUAA | |
| | AGAUGAAGUGUGCCUGGCAGAUUUUGAUAAAAGCUGCAGGUUUGCUAAUGAUCA | |
| | GACAUUAGUCCAUGGAGACCUCCGGGCACUUGGACGACUGGUCCAGUAUGUUGUA | |
| | ACCAAGGGAGAGGACCCCUUUGUGGAGAAAGGGGCUGCAAGAGAGCCUCAGGAGC | |
| | UAGCGGGUCUAGCUGAAAUCCCGGAUCACAUGGAGACGCUGGACCUCAUCCAGAC | |
| | ACUGCUCUCCCCUGGGGUCACCAGCCAGACUCAGCUGAGCGACAUUCUCUACCAUC | |
| | CCUUCUUCUGGCCCUGUAACAUGCGCUACAGGUUCCUAAGGGAUGUUGGGAACGA | |
| | AUCAGACAUCAAAACUCGGAAUACUGAAAACAAGACCCUGGAAAAAUUGAAUCGUA | |
| | AUCCUUCUAAGAGUUACUCAAAAUGGACUUCAAAGAUUGAUAAAUAUGUUAUGA | |
| | CUGAGAUGAAUCAUUUCUACAAAAAGAAAAAAAACCCCUAUCAAGACACAGUGACU | |
| | GACCUGCUGAAAUUCAUCCGCAACAUAGGCGAGCACAUUGAUGAGAAAGUCAAUG | |
| | CACAAAUGAAGGAAACUAUCGGGGAUCCUGCUCAGUAUUUUCAGAAGACCUUUCC | |
| | UGAGCUGGUGAUGCACGUCUACAAGAACAUACAGAACACAGAAUUCAUGAAACAU | |
| | AUUCCGAAUGCCCAGAAUUCUCUCCUGGAUAAC | |
| 111 | AUGGAGUCUCUGUCAGGGAAUUAUACUUCUCUGCUGAAUGAGUAUCAGCAGAAA | Exemplary REAVR21 |
| | ACCCAAUGCACUGUUGAGUUUGAGGAGGGACCCACUGAUGGACCAAGCCACAAUA | mRNA sequence |
| | AAAGGUUUACUAUGAGAGCUAUUGUGAAUGGACAGAAGUUUCCUGAUGGUACUG | |
| | GUAAAACCAAGAAGGAGGCAAAGCAAAAUGCUGCUAAAAACGCACUGGAAGGCUU | |
| | GAAGAGCACUCACAGUGAUGAACCGACACCUUCACCCGUUGAAAACAUUUCAGUUA | |
| | GUAAAAUAGCAUCUCAUCCGAAUUACACCUGCUGGCUCAAUGAGCAUUCGCAGAA | |
| | GAGCAGGUUAAUGUUCAAGGCCUGCGAGUCAACUAAGAUGGAUCCAGGAAACCUC | |
| | ACUCGGUUAUGCACGUAUGUGUGUAAGUAUGUUUGCGAUGAUAAAGAGUUUCCU | |
| | GAAGGUUAUGGGAAAAAUAAGAAGGAAGCCAAAGAGGCAGCGGCUCUUCGAGUU | |
| | UAUGAAGAGCUCAACAAAACACCAAAUACAGAGGUCUUGGAUGAGAACAGCAAUA | |
| | GAGCUCAGAGGUCAGAAACUACAUCUUAUAGCCUGGACCGCCGUUCGAGUGUUGC | |
| | AGAUGAUUCUCGAAGCAGCACGCCUGACAACAACUACAUCGCAUAUCUCAAUAACU | |
| | ACUGCCAGAAGAAAAAACGUGUAUAUGACUUCAAAUUAGUGGACAGAAUAGGUCC | |
| | UCCUCACAACCCAAUAUUUGUUUAUAAAGUUGUGAUGGAUGGGAAGGAAUACCC | |
| | UGAAGCACAGGGAAGAAACGCUAAAGAAGCGAAACAAAAUGCGGCUCAACACGCUU | |
| | GGAGUGAAAUUCGGGAUCAGUCUGGCUGGACCACACCGAGUUCUGAAGAUGGCGC | |
| | AUUCUCACAAUCCCAAGAUACAUGUAAAUCUGAAGACGUCCAGCGAUCAGCGAACG | |
| | CCUCCAGCACAAGUAAUUUCAUCUCCUUCAGAGACUCUGCUAAAAUUGCUGAUAC | |
| | UUCAGAAGGAGGCAUCUACCUGGGGUUCUAUGAGAAGCAAGAAGUAGCUGUGAA | |
| | GACGUUCUGUGAGGGCAGCCCACGUGCACAGCGGGAAGUCUCUUGUCUGCAAAGC | |
| | AGCCGAGAGAACAGUCACUUGGUGACAUUCUAUGGGAGUGAGAGCCACAGGGGCC | |
| | ACUUGUUUGUGUGUGUCACCCUCUGUGAGCAGACUCUGGAAGCGUGUUUGGAUG | |
| | UGCACAGAGGGGAAGAUGUGGAAAAUGAGGAAGAUGAAUUUGCCCGAAAUGUCC | |
| | UGUCAUCUAUAUUUAAGGCUGUUCAAGAACUACACUUGUCCUGUGGAUACACCCA | |
| | CCAGGAUCUGCAACCACAAAACAUCUUAAUAGAUUCUAAGAAAGCUGCUCACCUGG | |
| | CAGAUUUUGAUAAGAGCAUCAAGUGGGCUGGAGAUCCACAGGAAGUCAAGAGAGA | |
| | UCUAGAGGACCUUGGACGGCUGGUCCUCUAUGUGGUAAAGAAGGGAAGCAUCUC | |
| | AUUUGAGGAUCUGAAAGCUCAAAGUAAUGAAGAGGUGGUUCAACUUUCUCCAGA | |
| | UGAGGAAACUAAGGACCUCAUUCAUCGUCUCUUCCAUCCUGGGGAACAUGUGAGG | |
| | GACUGUCUGAGUGACCUGCUGGGUCAUCCCUUCUUUUGGACUUGGGAGAGCCGC | |
| | UAUAGGACGCUUCGGAAUGUGGGAAAUGAAUCCGACAUCAAAACACGAAAAUCUG | |
| | AAAGUGAGAUCCUCAGACUACUGCAACCUGGGCCUUCUGAACAUUCCAAAAGUUU | |
| | UGACAAGUGGACGACUAAGAUUAAUGAAUGUGUUAUGAAAAAAAUGAAUAAGUU | |
| | UUAUGAAAAAAGAGGCAAUUUCUACCAGAACACUGUGGGUGAUCUGCUAAAGUUC | |
| | AUCCGGAAUUUGGGAGAACACAUUGAUGAAGAAAAGCAUAAAAAGAUGAAAUUAA | |
| | AAAUUGGAGACCCUUCCCUGUAUUUUCAGAAGACAUUUCCAGAUCUGGUGAUCUA | |
| | UGUCUACACAAAACUACAGAACACAGAAUAUAGAAAGCAUUUCCCCCAAACCCACA | |
| | GUCCAAACAAGCCUCAGUGUGAUGGAGCUGGUGGGGCCAGUGGGUUGGCCAGCCC | |
| | UGGGUGC | |
| 112 | AUGGCAAAAAUGUCCGCAGAAAAUGAAAUAGAGAUGAGAAUCUGUGAUUACCUGA | Exemplary REAVR22 |
| | GGAGACACGGCAGGAGCACCGUACAGGACAUUUUCAAGGAAUUAAAAUUAGAGAA | mRNA sequence |
| | AUCCACCGUGAACAGGCAUCUAUACAGCCUACAGGCGUCAAAGCAGGUUUUUAAA | |
| | ACUGUGGAAGAUAAUAAGCGGCCGGUCUGGGAUCUGGUGGAGAGCAUGAAUGAC | |
| | GAACAAAAAGCCAAACCAGAGCAAAUAAAGCCCGAGAUGACAAGAGACGCAAUUGA | |
| | AGAAAAACAUGUGAAGGAUCUGCUGAAAUCAGGAGGUUUAAAAGCCUCUCAUAUC | |
| | GCCAGCAAACUGGGACAACAAACACAAUCAACUAACAAACUACUGUACGGUUUGAU | |
| | GGAGAAAGGUGAAGUGCGCAAAUGCAGUAAAAGCCACAUGUGGACUCUGAAAAAU | |
| | GACCAGGAGAGGAAUGGAAGUGGCAGUAAAAUUGCUGAUACUUCAGAAGGAGGC | |
| | AUCUACCUGGGGUUCUAUGAGAAGCAAGAAGUAGCUGUGAAGACGUUCUGUGAG | |
| | GGCAGCCCACGUGCACAGCGGGAAGUCUCUUGUCUGCAAAGCAGCCGAGAGAACA | |
| | GUCACUUGGUGACAUUCUAUGGGAGUGAGAGCCACAGGGGCCACUUGUUUGUGU | |
| | GUGUCACCCUCUGUGAGCAGACUCUGGAAGCGUGUUUGGAUGUGCACAGAGGGG | |
| | AAGAUGUGGAAAAUGAGGAAGAUGAAUUUGCCCGAAAUGUCCUGUCAUCUAUAU | |
| | UUAAGGCUGUUCAAGAACUACACUUGUCCUGUGGAUACACCCACCAGGAUCUGCA | |
| | ACCACAAAACAUCUUAAUAGAUUCUAAGAAAGCUGCUCACCUGGCAGAUUUUGAU | |
| | AAGAGCAUCAAGUGGGCUGGAGAUCCACAGGAAGUCAAGAGAGAUCUAGAGGACC | |
| | UUGGACGGCUGGUCCUCUAUGUGGUAAAGAAGGGAAGCAUCUCAUUUGAGGAUC | |
| | UGAAAGCUCAAAGUAAUGAAGAGGUGGUUCAACUUUCUCCAGAUGAGGAAACUAA | |
| | GGACCUCAUUCAUCGUCUCUUCCAUCCUGGGGAACAUGUGAGGGACUGUCUGAGU | |
| | GACCUGCUGGGUCAUCCCUUCUUUUGGACUUGGGAGAGCCGCUAUAGGACGCUUC | |
| | GGAAUGUGGGAAAUGAAUCCGACAUCAAAACACGAAAAUCUGAAAGUGAGAUCCU | |
| | CAGACUACUGCAACCUGGGCCUUCUGAACAUUCCAAAAGUUUUGACAAGUGGACG | |
| | ACUAAGAUUAAUGAAUGUGUUAUGAAAAAAAUGAAUAAGUUUUAUGAAAAAAGA | |
| | GGCAAUUUCUACCAGAACACUGUGGGUGAUCUGCUAAAGUUCAUCCGGAAUUUG | |
| | GGAGAACACAUUGAUGAAGAAAAGCAUAAAAAGAUGAAAUUAAAAAUUGGAGACC | |
| | CUUCCCUGUAUUUUCAGAAGACAUUUCCAGAUCUGGUGAUCUAUGUCUACACAAA | |
| | ACUACAGAACACAGAAUAUAGAAAGCAUUUCCCCCAAACCCACAGUCCAAACAAGCC | |
| | UCAGUGUGAUGGAGCUGGUGGGGCCAGUGGGUUGGCCAGCCCUGGGUGC | |
| 113 | AUGGCCCAGGCUCCUGCUGACCCGGGCAGAGAAGGCCACCUUGAACAAAGAAUCCU | Exemplary REAVR23 |
| | GCAGGUGCUGACAGAGGCUGGCUCCCCGGUGAAACUUGCCCAGCUGGUGAAGGAA | mRNA sequence |
| | UGCCAAGCACCCAAGAGGGAGCUCAACCAAGUCCUCUACCGAAUGAAAAAGGAGUU | |
| | GAAAGUCUCCCUCACAUCCCCUGCCACCUGGUGCUUGGGCGGGACUGAUCCUGAA | |
| | GGCGAGGGUCCUGCAGAGCUGGCCUUGUCCAGCCCUGCCGAGAGGCCCCAGCAACA | |
| | UGCAGCUACAAUUCCAGAGACCCCUGGCCCUCAGUUCAGCCAACAACGGGAGGAAG | |
| | ACAUCUACAGGUUUCUCAAAGACAAUGGUCCCCAGAGGGCCCUGGUCAUCGCCCAA | |
| | GCACUGGGAAUGAGGACAGCAAAAGAUGUGAACCGAGACUUGUACAGGAUGAAGA | |
| | GCAGGCACCUUCUGGACAUGGAUGAGCAGUCCAAAGCAUGGACGAUUUACCGCCC | |
| | AGAAGAUUCUGGAAGAAGAGCAAAGUCAGCCUCAAAAAUUGCUGAUACUUCAGAA | |
| | GGAGGCAUCUACCUGGGGUUCUAUGAGAAGCAAGAAGUAGCUGUGAAGACGUUC | |
| | UGUGAGGGCAGCCCACGUGCACAGCGGGAAGUCUCUUGUCUGCAAAGCAGCCGAG | |
| | AGAACAGUCACUUGGUGACAUUCUAUGGGAGUGAGAGCCACAGGGGCCACUUGU | |
| | UUGUGUGUGUCACCCUCUGUGAGCAGACUCUGGAAGCGUGUUUGGAUGUGCACA | |
| | GAGGGGAAGAUGUGGAAAAUGAGGAAGAUGAAUUUGCCCGAAAUGUCCUGUCAU | |
| | CUAUAUUUAAGGCUGUUCAAGAACUACACUUGUCCUGUGGAUACACCCACCAGGA | |
| | UCUGCAACCACAAAACAUCUUAAUAGAUUCUAAGAAAGCUGCUCACCUGGCAGAU | |
| | UUUGAUAAGAGCAUCAAGUGGGCUGGAGAUCCACAGGAAGUCAAGAGAGAUCUA | |
| | GAGGACCUUGGACGGCUGGUCCUCUAUGUGGUAAAGAAGGGAAGCAUCUCAUUU | |
| | GAGGAUCUGAAAGCUCAAAGUAAUGAAGAGGUGGUUCAACUUUCUCCAGAUGAG | |
| | GAAACUAAGGACCUCAUUCAUCGUCUCUUCCAUCCUGGGGAACAUGUGAGGGACU | |
| | GUCUGAGUGACCUGCUGGGUCAUCCCUUCUUUUGGACUUGGGAGAGCCGCUAUA | |
| | GGACGCUUCGGAAUGUGGGAAAUGAAUCCGACAUCAAAACACGAAAAUCUGAAAG | |
| | UGAGAUCCUCAGACUACUGCAACCUGGGCCUUCUGAACAUUCCAAAAGUUUUGAC | |
| | AAGUGGACGACUAAGAUUAAUGAAUGUGUUAUGAAAAAAAUGAAUAAGUUUUAU | |
| | GAAAAAAGAGGCAAUUUCUACCAGAACACUGUGGGUGAUCUGCUAAAGUUCAUCC | |
| | GGAAUUUGGGAGAACACAUUGAUGAAGAAAAGCAUAAAAAGAUGAAAUUAAAAAU | |
| | UGGAGACCCUUCCCUGUAUUUUCAGAAGACAUUUCCAGAUCUGGUGAUCUAUGUC | |
| | UACACAAAACUACAGAACACAGAAUAUAGAAAGCAUUUCCCCCAAACCCACAGUCCA | |
| | AACAAGCCUCAGUGUGAUGGAGCUGGUGGGGCCAGUGGGUUGGCCAGCCCUGGG | |
| | UGC | |
| 114 | AUGGCCCAGGCUCCUGCUGACCCGGGCAGAGAAGGCCACCUUGAACAAAGAAUCCU | Exemplary REAVR24 |
| | GCAGGUGCUGACAGAGGCUGGCUCCCCGGUGAAACUUGCCCAGCUGGUGAAGGAA | mRNA sequence |
| | UGCCAAGCACCCAAGAGGGAGCUCAACCAAGUCCUCUACCGAAUGAAAAAGGAGUU | |
| | GAAAGUCUCCCUCACAUCCCCUGCCACCUGGUGCUUGGGCGGGACUGAUCCUGAA | |
| | GGCGAGGGUCCUGCAGAGCUGGCCUUGUCCAGCCCUGCCGAGAGGCCCCAGCAACA | |
| | UGCAGCUACAAUUCCAGAGACCCCUGGCCCUCAGUUCAGCCAACAACGGGAGGAAG | |
| | ACAUCUACAGGUUUCUCAAAGACAAUGGUCCCCAGAGGGCCCUGGUCAUCGCCCAA | |
| | GCACUGGGAAUGAGGACAGCAAAAGAUGUGAACCGAGACUUGUACAGGAUGAAGA | |
| | GCAGGCACCUUCUGGACAUGGAUGAGCAGUCCAAAGCAUGGACGAUUUACCGCCC | |
| | AGAAGAUUCUGGAAGAAGAGCAAAGUCAGCCUCAAUUAUUUACCAGCACAAUCCA | |
| | AUCAACAUGAUCUGCCAGAAUGGACCCAACAGCUGGAUUUCCAUUGCAAACUCCAA | |
| | AAUUGCUGAUACUUCAGAAGGAGGCAUCUACCUGGGGUUCUAUGAGAAGCAAGA | |
| | AGUAGCUGUGAAGACGUUCUGUGAGGGCAGCCCACGUGCACAGCGGGAAGUCUCU | |
| | UGUCUGCAAAGCAGCCGAGAGAACAGUCACUUGGUGACAUUCUAUGGGAGUGAG | |
| | AGCCACAGGGGCCACUUGUUUGUGUGUGUCACCCUCUGUGAGCAGACUCUGGAAG | |
| | CGUGUUUGGAUGUGCACAGAGGGGAAGAUGUGGAAAAUGAGGAAGAUGAAUUU | |
| | GCCCGAAAUGUCCUGUCAUCUAUAUUUAAGGCUGUUCAAGAACUACACUUGUCCU | |
| | GUGGAUACACCCACCAGGAUCUGCAACCACAAAACAUCUUAAUAGAUUCUAAGAAA | |
| | GCUGCUCACCUGGCAGAUUUUGAUAAGAGCAUCAAGUGGGCUGGAGAUCCACAGG | |
| | AAGUCAAGAGAGAUCUAGAGGACCUUGGACGGCUGGUCCUCUAUGUGGUAAAGA | |
| | AGGGAAGCAUCUCAUUUGAGGAUCUGAAAGCUCAAAGUAAUGAAGAGGUGGUUC | |
| | AACUUUCUCCAGAUGAGGAAACUAAGGACCUCAUUCAUCGUCUCUUCCAUCCUGG | |
| | GGAACAUGUGAGGGACUGUCUGAGUGACCUGCUGGGUCAUCCCUUCUUUUGGAC | |
| | UUGGGAGAGCCGCUAUAGGACGCUUCGGAAUGUGGGAAAUGAAUCCGACAUCAAA | |
| | ACACGAAAAUCUGAAAGUGAGAUCCUCAGACUACUGCAACCUGGGCCUUCUGAACA | |
| | UUCCAAAAGUUUUGACAAGUGGACGACUAAGAUUAAUGAAUGUGUUAUGAAAAA | |
| | AAUGAAUAAGUUUUAUGAAAAAAGAGGCAAUUUCUACCAGAACACUGUGGGUGA | |
| | UCUGCUAAAGUUCAUCCGGAAUUUGGGAGAACACAUUGAUGAAGAAAAGCAUAAA | |
| | AAGAUGAAAUUAAAAAUUGGAGACCCUUCCCUGUAUUUUCAGAAGACAUUUCCAG | |
| | AUCUGGUGAUCUAUGUCUACACAAAACUACAGAACACAGAAUAUAGAAAGCAUUU | |
| | CCCCCAAACCCACAGUCCAAACAAGCCUCAGUGUGAUGGAGCUGGUGGGGCCAGUG | |
| | GGUUGGCCAGCCCUGGGUGC | |
| 115 | AUGGCAGAAGCUCCUGUUGACUUGAGCACAGGAGACAAUCUGGAGCAAAAGAUCC | Exemplary REAVR25 |
| | UGCAGGUGUUGAGCGAUGACGGCGGCCCUGUGAAGAUUGGCCAGCUGGUGAAGA | mRNA sequence |
| | AAUGCCAAGUGCCCAAGAAAACCCUCAAUCAAGUCCUUUACCGCCUGAAGAAGGAG | |
| | GACAGAGUGUCCUCCCCAGAGCCUGCAACAUGGAGCAUAGGCGGGGCUGCUUCUG | |
| | GAGAUGGGGCUCCUGCAAUCCCUGAGAACUCCAGUGCCCAGCCUAGCCUUGAUGA | |
| | AAGAAUAUUAAGAUUCCUGGAAGCCAAUGGGCCUCACAGGGCCCUGCACAUCGCCA | |
| | AGGCUCUGGGAAUGACGACAGCCAAAGAAGUGAACCCACUCCUGUAUUCCAUGAG | |
| | AAAUAAGCACCUUCUGAGCUAUGACGGACAGACGUGGAAGAUCUACCACUCACGU | |
| | CAGGAAGGCCAAGACAUAGCUCAUUCUGGAGUCACACAAGAGUCCCCUGCGAAAAU | |
| | UGCUGAUACUUCAGAAGGAGGCAUCUACCUGGGGUUCUAUGAGAAGCAAGAAGU | |
| | AGCUGUGAAGACGUUCUGUGAGGGCAGCCCACGUGCACAGCGGGAAGUCUCUUGU | |
| | CUGCAAAGCAGCCGAGAGAACAGUCACUUGGUGACAUUCUAUGGGAGUGAGAGCC | |
| | ACAGGGGCCACUUGUUUGUGUGUGUCACCCUCUGUGAGCAGACUCUGGAAGCGU | |
| | GUUUGGAUGUGCACAGAGGGGAAGAUGUGGAAAAUGAGGAAGAUGAAUUUGCCC | |
| | GAAAUGUCCUGUCAUCUAUAUUUAAGGCUGUUCAAGAACUACACUUGUCCUGUG | |
| | GAUACACCCACCAGGAUCUGCAACCACAAAACAUCUUAAUAGAUUCUAAGAAAGCU | |
| | GCUCACCUGGCAGAUUUUGAUAAGAGCAUCAAGUGGGCUGGAGAUCCACAGGAAG | |
| | UCAAGAGAGAUCUAGAGGACCUUGGACGGCUGGUCCUCUAUGUGGUAAAGAAGG | |
| | GAAGCAUCUCAUUUGAGGAUCUGAAAGCUCAAAGUAAUGAAGAGGUGGUUCAAC | |
| | UUUCUCCAGAUGAGGAAACUAAGGACCUCAUUCAUCGUCUCUUCCAUCCUGGGGA | |
| | ACAUGUGAGGGACUGUCUGAGUGACCUGCUGGGUCAUCCCUUCUUUUGGACUUG | |
| | GGAGAGCCGCUAUAGGACGCUUCGGAAUGUGGGAAAUGAAUCCGACAUCAAAACA | |
| | CGAAAAUCUGAAAGUGAGAUCCUCAGACUACUGCAACCUGGGCCUUCUGAACAUU | |
| | CCAAAAGUUUUGACAAGUGGACGACUAAGAUUAAUGAAUGUGUUAUGAAAAAAA | |
| | UGAAUAAGUUUUAUGAAAAAAGAGGCAAUUUCUACCAGAACACUGUGGGUGAUC | |
| | UGCUAAAGUUCAUCCGGAAUUUGGGAGAACACAUUGAUGAAGAAAAGCAUAAAAA | |
| | GAUGAAAUUAAAAAUUGGAGACCCUUCCCUGUAUUUUCAGAAGACAUUUCCAGAU | |
| | CUGGUGAUCUAUGUCUACACAAAACUACAGAACACAGAAUAUAGAAAGCAUUUCC | |
| | CCCAAACCCACAGUCCAAACAAGCCUCAGUGUGAUGGAGCUGGUGGGGCCAGUGG | |
| | GUUGGCCAGCCCUGGGUGC | |
| 116 | AUGGCAGAAGCUCCUGUUGACUUGAGCACAGGAGACAAUCUGGAGCAAAAGAUCC | Exemplary REAVR26 |
| | UGCAGGUGUUGAGCGAUGACGGCGGCCCUGUGAAGAUUGGCCAGCUGGUGAAGA | mRNA sequence |
| | AAUGCCAAGUGCCCAAGAAAACCCUCAAUCAAGUCCUUUACCGCCUGAAGAAGGAG | |
| | GACAGAGUGUCCUCCCCAGAGCCUGCAACAUGGAGCAUAGGCGGGGCUGCUUCUG | |
| | GAGAUGGGGCUCCUGCAAUCCCUGAGAACUCCAGUGCCCAGCCUAGCCUUGAUGA | |
| | AAGAAUAUUAAGAUUCCUGGAAGCCAAUGGGCCUCACAGGGCCCUGCACAUCGCCA | |
| | AGGCUCUGGGAAUGACGACAGCCAAAGAAGUGAACCCACUCCUGUAUUCCAUGAG | |
| | AAAUAAGCACCUUCUGAGCUAUGACGGACAGACGUGGAAGAUCUACCACUCACGU | |
| | CAGGAAGGCCAAGACAUAGCUCAUUCUGGAGUCACACAAGAGUCCCCUGCGAUUA | |
| | UUUGUCAGCACAAUCCGGUCAACAUGAUCUGCCAACAAGGAGCCAACAGCCACAUC | |
| | UCCAUUGCCAAUUCAAAAAUUGCUGAUACUUCAGAAGGAGGCAUCUACCUGGGGU | |
| | UCUAUGAGAAGCAAGAAGUAGCUGUGAAGACGUUCUGUGAGGGCAGCCCACGUGC | |
| | ACAGCGGGAAGUCUCUUGUCUGCAAAGCAGCCGAGAGAACAGUCACUUGGUGACA | |
| | UUCUAUGGGAGUGAGAGCCACAGGGGCCACUUGUUUGUGUGUGUCACCCUCUGU | |
| | GAGCAGACUCUGGAAGCGUGUUUGGAUGUGCACAGAGGGGAAGAUGUGGAAAAU | |
| | GAGGAAGAUGAAUUUGCCCGAAAUGUCCUGUCAUCUAUAUUUAAGGCUGUUCAA | |
| | GAACUACACUUGUCCUGUGGAUACACCCACCAGGAUCUGCAACCACAAAACAUCUU | |
| | AAUAGAUUCUAAGAAAGCUGCUCACCUGGCAGAUUUUGAUAAGAGCAUCAAGUGG | |
| | GCUGGAGAUCCACAGGAAGUCAAGAGAGAUCUAGAGGACCUUGGACGGCUGGUCC | |
| | UCUAUGUGGUAAAGAAGGGAAGCAUCUCAUUUGAGGAUCUGAAAGCUCAAAGUA | |
| | AUGAAGAGGUGGUUCAACUUUCUCCAGAUGAGGAAACUAAGGACCUCAUUCAUCG | |
| | UCUCUUCCAUCCUGGGGAACAUGUGAGGGACUGUCUGAGUGACCUGCUGGGUCA | |
| | UCCCUUCUUUUGGACUUGGGAGAGCCGCUAUAGGACGCUUCGGAAUGUGGGAAA | |
| | UGAAUCCGACAUCAAAACACGAAAAUCUGAAAGUGAGAUCCUCAGACUACUGCAAC | |
| | CUGGGCCUUCUGAACAUUCCAAAAGUUUUGACAAGUGGACGACUAAGAUUAAUGA | |
| | AUGUGUUAUGAAAAAAAUGAAUAAGUUUUAUGAAAAAAGAGGCAAUUUCUACCA | |
| | GAACACUGUGGGUGAUCUGCUAAAGUUCAUCCGGAAUUUGGGAGAACACAUUGA | |
| | UGAAGAAAAGCAUAAAAAGAUGAAAUUAAAAAUUGGAGACCCUUCCCUGUAUUUU | |
| | CAGAAGACAUUUCCAGAUCUGGUGAUCUAUGUCUACACAAAACUACAGAACACAG | |
| | AAUAUAGAAAGCAUUUCCCCCAAACCCACAGUCCAAACAAGCCUCAGUGUGAUGGA | |
| | GCUGGUGGGGCCAGUGGGUUGGCCAGCCCUGGGUGC | |

1. A fusion protein comprising:
a sensor domain that binds double-stranded RNA or Z-DNA; and
an effector domain that cleaves RNA, wherein the effector domain has a length of less than 385 amino acids, and wherein the fusion protein is not naturally occurring.

2. The fusion protein of claim 1, wherein the effector domain has a length greater than 225 amino acids.

3. The fusion protein of claim 1, wherein the effector domain cleaves single-stranded RNA.

4. The fusion protein of claim 3, wherein the effector domain is an RNase L domain.

5-6. (canceled)

7. The fusion protein of claim 1, wherein the effector domain has at least 80% sequence identity with an amino acid sequence selected from the group consisting of SEQ ID NOS: 1-17.

8. (canceled)

9. The fusion protein of claim 1, wherein the sensor domain is a protein kinase R domain, a Z-DNA binding protein domain, or a protein kinase Z domain.

10-11. (canceled)

12. The fusion protein of claim 1, wherein the sensor domain has at least 80% sequence identity with an amino acid sequence selected from the group consisting of SEQ ID NOS: 18-32.

13. The fusion protein of claim 1, wherein the fusion protein further comprises a linker region connecting the sensor domain and the effector domain, the linker region having a length between 2 amino acids and 112 amino acids.

14. (canceled)

15. The fusion protein of claim 13, wherein the linker region has at least 80% sequence identity with an amino acid sequence selected from the group consisting of SEQ ID NOS: 33-44.

16. The fusion protein of claim 1, wherein the fusion protein has at least 80% sequence identity with an amino acid sequence selected from the group consisting of SEQ ID NOS: 45-68.

17. A nucleic acid encoding the fusion protein of claim 1.

18. The nucleic acid of claim 17, wherein the nucleic acid comprises DNA having at least 80% sequence identity with a nucleotide sequence selected from the group consisting of SEQ ID NOS: 69-92.

19. (canceled)

20. The nucleic acid of claim 17, wherein the nucleic acid comprises mRNA having at least 80% sequence identity with a nucleotide sequence selected from the group consisting of SEQ ID NOS: 93-116.

21. (canceled)

22. A vector comprising the nucleic acid of claim 17.

23. The vector of claim 22, wherein the vector further comprises a promoter regulating expression of the nucleic acid.

24-27. (canceled)

28. The vector of claim 22, wherein:
the nucleic acid is a first nucleic acid, the fusion protein is a first fusion protein, the sensor domain is a first sensor domain, and the effector domain is a first effector domain;
the vector further comprises a second nucleic acid encoding a second fusion protein;
the second fusion protein comprises a second sensor domain that binds double-stranded nucleic acids, and a second effector domain that cleaves RNA;
the second fusion protein has a different amino acid sequence than the first fusion protein; and
the second fusion protein is not naturally occurring.

29. A cell comprising the fusion protein of claim 1.

30. A method of treating a subject infected with a virus, the method comprising administering to the subject a therapeutically effective amount of the fusion protein of claim 1.

31. The method of claim 30, wherein the virus is an arbovirus.

32-33. (canceled)

34. A multicellular organism comprising the fusion protein of claim 1.

35-41. (canceled)
